(19) United States
(12) Patent Application Publication
(10) Pub. No.: US 2026/0242356 A1
(43) Pub. Date: Aug. 20, 2026

(54) BICYCLIC HETEROCYCLIC AMIDE INHIBITORS OF NAv1.8 FOR THE TREATMENT OF PAIN

(71) Applicant: SiteOne Therapeutics, Inc., South San Francisco, CA (US)

(72) Inventors: Jie ZHU, South San Francisco, CA (US); John MULCAHY, Redwood City, CA (US); Hari PRAKASH, Uttar Pradesh (IN); Puspesh Kumar UPADHYAY, Uttar Pradesh (IN)

(21) Appl. No.: 18/859,770

(22) PCT Filed: Apr. 25, 2023
(86) PCT No.: PCT/US2023/019879

§ 371 Date: Oct. 24, 2024

(30) Foreign Application Priority Data
Apr. 25, 2022 (IN) 202211024240
Feb. 27, 2023 (IN) 202311013223

(51) Int. Cl. C07D 403/04; A61K 31/4545; A61K 31/4709; A61K 31/498; A61K 31/55; A61K 31/69; C07D 401/04; C07D 401/14; C07D 405/14; C07D 409/14; C07D 413/14; C07D 417/14; C07D 471/04; C07D 491/052; C07D 491/056; C07F 5/02

(52) U.S. Cl. CPC C07D 403/04; A61K 31/4545; A61K 31/4709; A61K 31/498; A61K 31/55; A61K 31/69; C07D 401/04; C07D 401/14; C07D 405/14; C07D 409/14; C07D 413/14; C07D 417/14; C07D 471/04; C07D 491/052; C07D 491/056; C07F 5/025

(57) ABSTRACT

Provided herein are compounds, pharmaceutical compositions comprising the compounds, methods of preparing the compounds, and methods of using the compounds and compositions in treating conditions associated with voltage-gated sodium channel function where the compounds are 1-98.

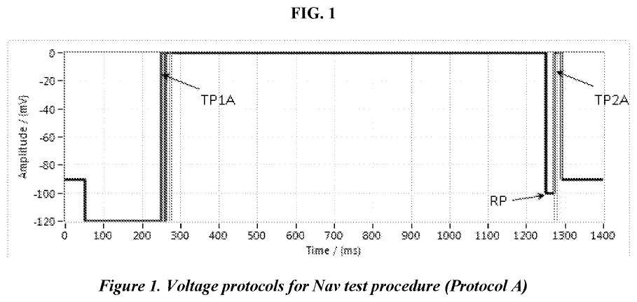

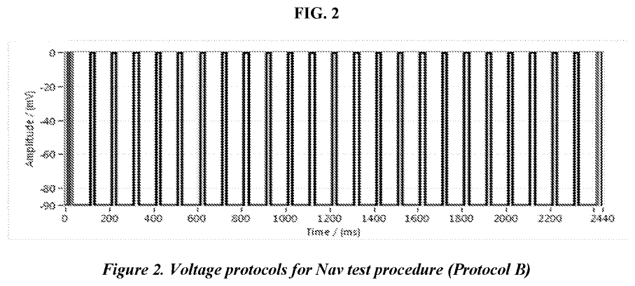

### CROSS-REFERENCE TO RELATED APPLICATIONS

[RESERVED]

### FIELD

Provided herein are compounds, methods of making the compounds, pharmaceutical compositions comprising the compounds, and methods of using the compounds and compositions in treating conditions associated with voltage-gated sodium channel function, in particular Na_{V}1.8, for example pain and conditions associated with pain. Also provided herein are methods of treating pain in a subject comprising administering a therapeutically or prophylactically effective amount of a or composition to a subject.

### BACKGROUND

Voltage-gated sodium channels are large integral membrane protein complexes present in neurons and excitable tissues where they contribute to processes such as membrane excitability and muscle contraction (Ogata et al., Jpn. J. Pharmacol. (2002) 88(4) 365-77). They have been identified as a primary target for the treatment of pain. Genes encoding for nine distinct mammalian isoforms of Na_{V} channels (Na_{V} isoforms 1.1-1.9) have been sequenced. Variation in the gating properties of different Na_{V} isoforms, cellular distributions, and expression levels influence the physiology of nerve cell conduction. A mounting body of evidence suggests that individual Na_{V} isoforms Na_{V} 1.3, 1.7, 1.8 and 1.9 are disproportionately involved in pain signaling and nociception, and that an isoform-specific inhibitor of Na_{V} could provide pain relief without the accompanying undesirable effects of a non-specific Na_{V} antagonist or an opioid drug (Momin et al., Curr Opin Neurobiol. 18(4): 383-8, 2008; Rush et al., J. Physiol. 579(Pt 1): 1-14, 2007).

Naᵥ1.8 is selectively expressed in dorsal root ganglion (DRG) neurons, a type of pseudo-unipolar neuron that project both centrally and peripherally, which are implicated in nociception. As a result of its sensory neuron specificity, Na_{V}1.8 is particularly important in the pathophysiology of pain. The design of a drug which selectively inhibits Na_{V} 1.8 over the other Na_{V} channels is therefore desirable. Such a drug design is challenging given the high structural homology (75-96%) of the mammalian Na_{V} isoforms.

Isoform-selective selective inhibitors have been sought by a number of research groups and certain compounds have advanced to clinical development. Isoform-selective small molecule inhibitors of Na_{V} 1.8 are disclosed in the following patent applications and publications: WO 2021/257490; WO 2021/257418; WO 2021/257420; WO 2021/113627; WO 2021/032074; WO 2020/261114; WO 2020/219867; WO 2020/146682; WO 2020/092187; WO 2020/092667; WO 2020/014246; WO 2020/014243; WO 2019/157505; WO 2019/014352; WO 2018/213426; WO 2015/089361; WO 2015/010065; WO 2014/120820; WO 2014/120815; WO 2014/120808; WO 2013/114250; WO 2013/061205; WO 2008/135830; Brown et al., Bioorg Med Chem. 27(1):230-239, 2019; Bagal et al., Med Chem Comm. 7(10), 2016; Bagal et al., ACS Med Chem Lett. 6(6):650-654, 2015; Jarvis et al., PNAS 104(20):8520-8525, 2007.

Previous efforts to develop isoform-selective inhibitors of Na_{V} 1.8 as human therapeutics have encountered a number of challenges. These include insufficient selectivity over off-target human Na_{V} 1.x isforms, limited target engagement in vivo (due to insufficient potency for human Na_{V}1.8, high protein or tissue binding, state-dependent inhibition of Na_{V} 1.8, safety concerns that preclude dose escalation, or a combination thereof), physico-chemical properties such as poor solubility that impeded oral dosing, and compound-specific issues such as toxicological findings or formation of metabolites that could pose safety risks to humans. Certain compounds also exhibit species variation in Nay 1.8 potency (see e.g. Bagal et al., Med Chem Comm. 7(10), 2016) such that it is challenging to evaluate their on-target pharmacodynamic effects in standard preclinical species i.e. mouse and rat.

There exists a need for compounds which treat pain and conditions associated with voltage-gated sodium channel function, particularly which selectively inhibit Na_{V} 1.8 over other Nay isoforms.

### SUMMARY

Provided herein are compounds, methods of making the compounds, pharmaceutical compositions comprising the compounds, and methods of using the compounds and compositions for the treatment of conditions modulated by voltage-gated sodium channels, in particular, Na_{V} 1.8, and in some or any embodiments, in the treatment of pain. Also provided herein are methods of treating pain and/or conditions modulated by voltage-gated sodium channels in a subject comprising administering a therapeutically or prophylactically effective amount of a compound or composition to a subject. In some or any embodiments, the subject is a human.

In one aspect, provided herein is a Compound of Formula (I):

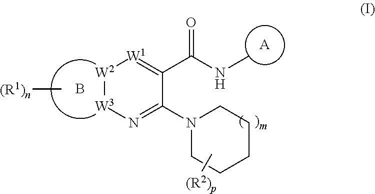

wherein
R¹, independently in each instance, is selected from hydrogen, C₁-C₆ alkyl, C₁-C₆ alkoxy, halogen, C₁-C₆ haloalkyl, halo-C₁-C₆ alkoxy, C₃-C₆-cycloalkyl, and C₃-C₆-cycloalkylC₁-C₃-alkyl; or
wherein two R¹ are attached on adjacent ring carbons in

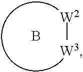

and together with the adjacent carbons to which they are attached form

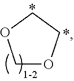

where * indicate the shared carbons in

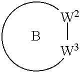

and any remaining R¹ are independently selected from hydrogen, C₁-C₆ alkyl, C₁-C₆ alkoxy, halogen, C₁-C₆ haloalkyl, halo-C₁-C₆ alkoxy, C₃-C₆-cycloalkyl, or C₃-C₆-cycloalkylC₁-C₃-alkyl;
A is C₆-C₁₀ aryl substituted with R³ and optionally substituted with (R^{3a})_{q}; 5- to 10-membered heteroaryl substituted with R³ and optionally substituted with (R^{3a})_{q}; or

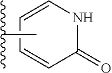

optionally substituted with (R^{3a})_{q1};
W¹ is —N═, —C(H)═, —C(halogen)=, —C(C₁-C₆ alkyl)=, or —C(cyclopropyl)=;
W² and W³ are each —C—, the dashed bond between W² and W³ is a double bond, and

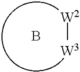

is a partially unsaturated 5 to 7-membered carbocyclic ring, a benzo ring, a partially unsaturated 5 to 7-membered heterocyclic ring, or a 5 or 6-membered heteroaromatic ring; or
one of W² and W³ is —C— and the other is —N—, the dashed bond between W² and W³ is a single bond, and

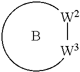

is a 5 or 6-membered heterocyclic ring optionally comprising an additional N and where the remaining ring atoms are C;
R², independently in each instance, is hydrogen, halogen, or C₁-C₆ alkyl; or
two R² are attached on adjacent carbons and together with the carbons to which they are attached form a 4- to 7-membered carbocyclic ring and the remaining R², independently in each instance, is hydrogen, halogen, or C₁-C₆alkyl; or
two R² are attached on the same carbon and together with the carbon to which they are attached form a 4- to 7-membered carbocyclic ring and the remaining R², independently in each instance, is hydrogen, halogen, or C₁-C₆alkyl;
R³ is hydrogen, —C(O)NH₂, —NH₂, —S(O)₂NHR, —S(O)₂C₁-C₆-alkyl, or

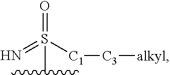

wherein R is hydrogen, C₁-C₃ alkyl or C₃-C₅ cycloalkyl;
R^{3a}, independently in each instance, is hydrogen, halogen, C₁-C₃ alkyl, or C₃-C₆ cycloalkyl;
m is 0, 1, 2, or 3;
n is 0, 1, 2, 3, or 4;
p is 0, 1, 2, 3, 4, 5, or 6;
q1 is 0, 1, or 2; and
q is 0, 1, 2, or 3; or
a pharmaceutically acceptable salt thereof and/or an isomer thereof.

In a further aspect, provided herein is a compound according to any one of the following formulas:

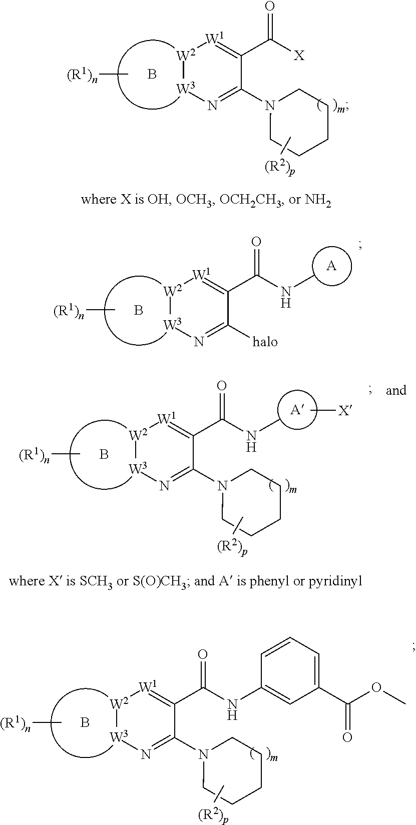

or a salt thereof,
wherein
R¹, independently in each instance, is selected from hydrogen, C₁-C₆ alkyl, C₁-C₆ alkoxy, halogen, C₁-C₆ haloalkyl, halo-C₁-C₆ alkoxy, C₃-C₆-cycloalkyl, and C₃-C₆-cycloalkylC₁-C₃-alkyl; or
wherein two R¹ are attached on adjacent ring carbons in

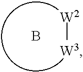

and together with the adjacent carbons to which they are attached form

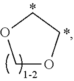

where * indicate the shared carbons in

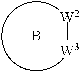

and any remaining R¹ are independently selected from hydrogen, C₁-C₆ alkyl, C₁-C₆ alkoxy, halogen, C₁-C₆ haloalkyl, halo-C₁-C₆ alkoxy, C₃-C₆-cycloalkyl, or C₃-C₆-cycloalkylC₁-C₃-alkyl;
A is C₆-C₁₀ aryl substituted with R³ and optionally substituted with (R^{3a})_{q}; 5- to 10-membered heteroaryl substituted with R³ and optionally substituted with (R^{3a})_{q}; or

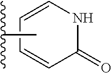

optionally substituted with (R^{3a})_{q};
W¹ is —N═, —C(H)═, —C(halogen)=, —C(C₁-C₆alkyl)=, or —C(cyclopropyl)=;
W² and W³ are each —C—, the dashed bond between W² and W³ is a double bond, and

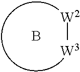

is a partially unsaturated 5 to 7-membered carbocyclic ring, a benzo ring, a partially unsaturated 5 to 7-membered heterocyclic ring, or a 5 or 6-membered heteroaromatic ring; or
one of W² and W³ is —C— and the other is —N—, the dashed bond between W² and W³ is a single bond, and

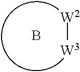

is a 5 or 6-membered heterocyclic ring optionally comprising an additional N and where the remaining ring atoms are C;
R², independently in each instance, is hydrogen, halogen, or C₁-C₆ alkyl; or
two R² are attached on adjacent carbons and together with the carbons to which they are attached form a 4- to 7-membered carbocyclic ring and the remaining R², independently in each instance, is hydrogen, halogen, or C₁-C₆alkyl; or
two R² are attached on the same carbon and together with the carbon to which they are attached form a 4- to 7-membered carbocyclic ring and the remaining R², independently in each instance, is hydrogen, halogen, or C₁-C₆alkyl;
m is 0, 1, 2, or 3;
n is 0, 1, 2, 3, or 4; and
p is 0, 1, 2, 3, 4, 5, or 6.

In an additional aspect, provided herein is a method of preparing a compound of Formula (I) according to any of the following schemes:

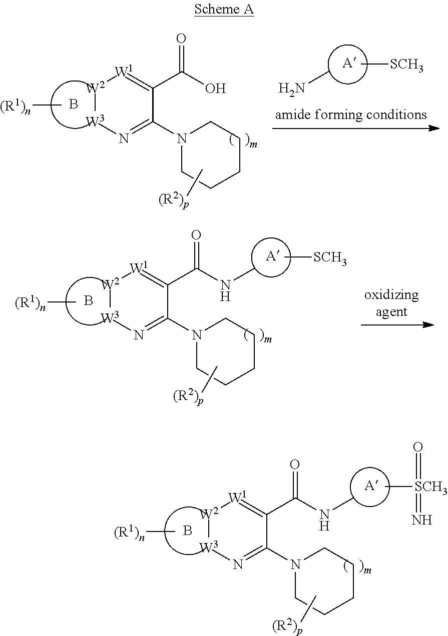

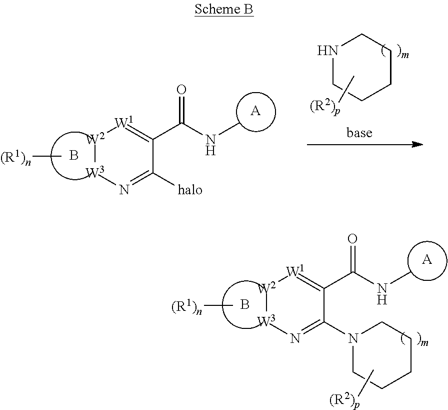

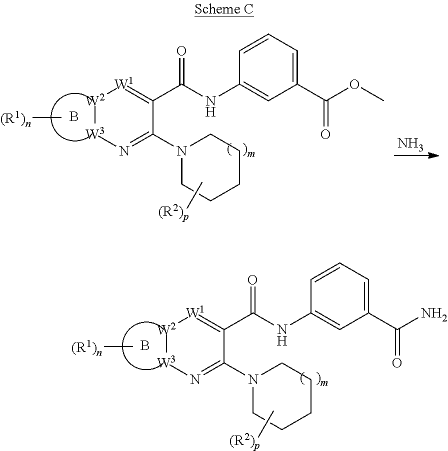

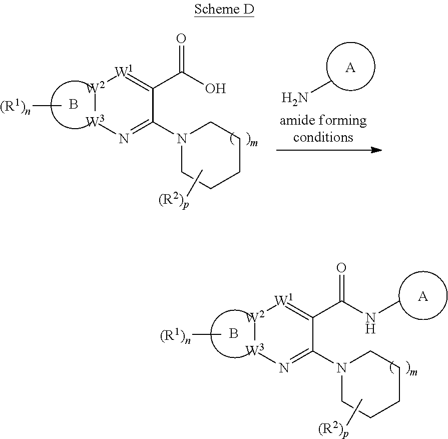

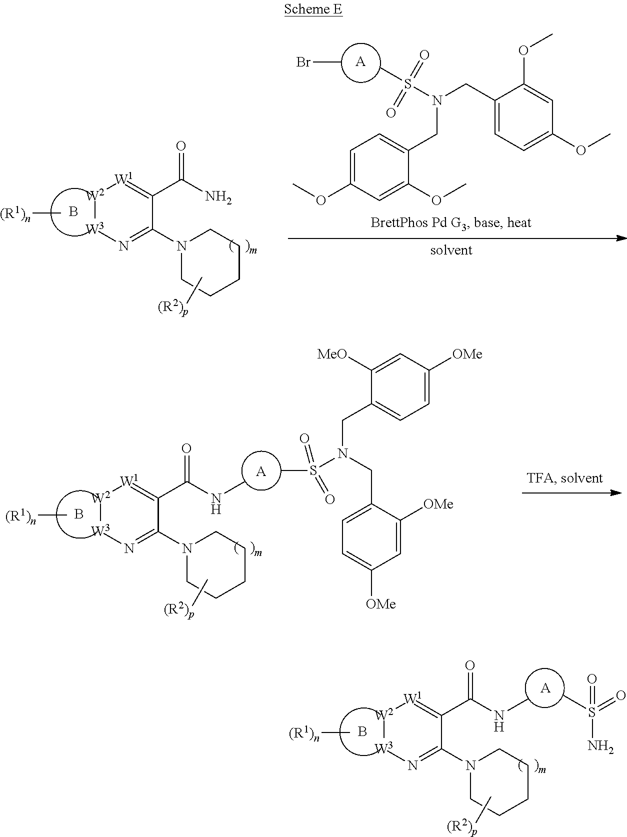

wherein
R¹, independently in each instance, is selected from hydrogen, C₁-C₆ alkyl, C₁-C₆ alkoxy, halogen, C₁-C₆ haloalkyl, halo-C₁-C₆ alkoxy, C₃-C₆-cycloalkyl, and C₃-C₆-cycloalkylC₁-C₃-alkyl; or
wherein two R¹ are attached on adjacent ring carbons in

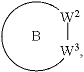

and together with the adjacent carbons to which they are attached form

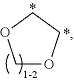

where * indicate the shared carbons in

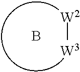

and any remaining R¹ are independently selected from hydrogen, C₁-C₆ alkyl, C₁-C₆ alkoxy, halogen, C₁-C₆ haloalkyl, halo-C₁-C₆ alkoxy, C₃-C₆-cycloalkyl, or C₃-C₆-cycloalkylC₁-C₃-alkyl;
A is C₆-C₁₀ aryl substituted with R³ and optionally substituted with (R^{3a})_{q}; 5- to 10-membered heteroaryl substituted with R³ and optionally substituted with (R^{3a})_{q}; or

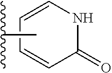

optionally substituted with (R^{3a})_{q1};
W¹ is —N═, —C(H)═, —C(halogen)=, —C(C₁-C₆ alkyl)=, or —C(cyclopropyl)=;
W² and W³ are each —C—, the dashed bond between W² and W³ is a double bond, and

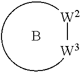

is a partially unsaturated 5 to 7-membered carbocyclic ring, a benzo ring, a partially unsaturated 5 to 7-membered heterocyclic ring, or a 5 or 6-membered heteroaromatic ring; or
one of W² and W³ is —C— and the other is —N—, the dashed bond between W² and W³ is a single bond, and

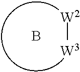

is a 5 or 6-membered heterocyclic ring optionally comprising an additional N and where the remaining ring atoms are C;
R², independently in each instance, is hydrogen, halogen, or C₁-C₆ alkyl; or
two R² are attached on adjacent carbons and together with the carbons to which they are attached form a 4- to 7-membered carbocyclic ring and the remaining R², independently in each instance, is hydrogen, halogen, or C₁-C₆alkyl; or
two R² are attached on the same carbon and together with the carbon to which they are attached form a 4- to 7-membered carbocyclic ring and the remaining R², independently in each instance, is hydrogen, halogen, or C₁-C₆alkyl;
m is 0, 1, 2, or 3;
n is 0, 1, 2, 3, or 4; and
p is 0, 1, 2, 3, 4, 5, or 6;
to yield a compound of Formula I; and optionally isolating the compound of Formula I.

In another aspect provided herein are pharmaceutical compositions, single unit dosage forms, and kits suitable for use in treating pain and/or conditions modulated by voltage-gated sodium channels which comprise a therapeutically or prophylactically effective amount of a compound provided herein, e.g., of some or any of the embodiments, of Formula (I) and compounds 1-97.

In an aspect, a method of treatment of condition associated with voltage-gated sodium channels function, including Naᵥ0.1.8, in a subject is provided comprising administering to an individual in need thereof a therapeutically or prophylactically effective amount of a compound provided herein, e.g., of some or any of the embodiments, of Formula (I) and compounds 1-97.

In another aspect, provided is a therapeutically or prophylactically effective amount of a compound provided herein, e.g., of some or any of the embodiments, of Formula (I) and compounds 1-97, or a therapeutically or prophylactically effective amount of a pharmaceutical composition for use in treating a condition associated with voltage-gated sodium channels function, including Naᵥ0.1.8, in a subject in need thereof.

### BRIEF DESCRIPTION OF FIGURES

None.

### DETAILED DESCRIPTION

Provided herein are compounds, methods of making the compounds, pharmaceutical compositions comprising the compounds, and methods of using the compounds and compositions in the treatment of pain and/or conditions modulated by voltage-gated sodium channels, in particular Na_{V} 1.8. Also provided herein are methods of treating pain in a subject comprising administering a therapeutically or prophylactically effective amount of a compound or composition to a subject. In an embodiment, the subject is a human.

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise. Unless specified otherwise, where a term is defined as being substituted, the groups in the list of substituents are themselves unsubstituted. For example, a substituted alkyl group can be substituted, for example, with a cycloalkyl group, and the cycloalkyl group is not further substituted unless specified otherwise.

Reference to “about” a value or parameter herein includes (and describes) variations that are directed to that value or parameter per se. For example, description referring to “about X” includes description of “X”. As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

The terms “a” or “an,” as used in herein means one or more, unless context clearly dictates otherwise.

The term “alkyl,” as used herein, unless otherwise specified, refers to a saturated straight or branched hydrocarbon. In some or any embodiments, the alkyl group is a primary, secondary, or tertiary hydrocarbon. In some or any embodiments, the alkyl group includes one to ten carbon atoms, i.e., C₁ to C₁₀ alkyl. In some or any embodiments, the alkyl is a C₁₋₆alkyl. In some or any embodiments, the alkyl group is selected from the group consisting of methyl, ethyl, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl.

The term “alkoxy” and “alkyloxy” as used herein, and unless otherwise specified, refer to the group —OR′ where R′ is alkyl. Alkoxy and alkyloxy groups include, in some or any embodiments, methoxy, ethoxy, n-propoxy, isopropoxy, n-butoxy, tert-butoxy, see-butoxy, n-pentoxy, n-hexyloxy, 1,2-dimethylbutoxy, and the like. In some embodiments, alkoxy is C₁₋₆alkoxy.

The term “amino” means —NH₂.

The term “aryl,” as used herein, and unless otherwise specified, refers to a monovalent C₆-C₁₅ carbocyclic ring system which comprises at least one aromatic ring wherein the aryl ring system is mono, di, or tricyclic. The aryl may be attached to the main structure through any of its rings, i.e. any aromatic or nonaromatic ring. In some or any embodiments, the aryl group may be a bridged (where chemically feasible) or non-bridged, spirocyclic (where chemically feasible) or not spirocyclic, and/or fused or not fused multicyclic group. In some or any embodiments, aryl is phenyl, naphthyl, indanyl, fluorenyl, 6,7,8,9-tetrahydro-5H-benzo[7]annulenyl, or tetrahydronaphthyl. When aryl is substituted, it can be substituted on any ring, i.e. on any aromatic or nonaromatic ring comprised by aryl.

The term “C₃-C₁₀-cycloalkyl,” as used herein, refers to a monovalent, saturated, monocyclic hydrocarbon or or bicyclic (fused, bridged, or spirocyclic) ring. In some or any embodiments, the the terms “fused cycloalkyl” and “spirocycloalkyl” are embodiments of the cycloalkyl group. In some or any embodiments, the cycloalkyl group includes three to six carbon atoms, i.e., C₃ to C₆ cycloalkyl. In some or any embodiments, the cycloalkyl has 3, 4, or 5 (C₃₋₅); 3 or 4 (C₃₋₄); 3 (C₃); 4 (C₄); or 5 (C₅) carbon atoms. In some or any embodiments, the cycloalkyl group is cyclopropyl, cyclobutyl, cyclopentyl, or cyclohexyl. In some or any embodiments, the cycloalkyl group is cyclopropyl, cyclobutyl, or cyclopentyl. In some or any embodiments, the cycloalkyl group is cyclopropyl. In some or any embodiments, the cycloalkyl group is cyclobutyl. In some or any embodiments, the cycloalkyl group is cyclopentyl. In some or any embodiments, the cycloalkyl group is bicyclo[3.2.1]octyl, bicyclo[2.2.2]octyl, bicyclo[3.3.1]nonyl, bicyclo[3.3.2]decyl, bicyclo[2.2.2]octyl, or adamantyl.

The term “cycloalkylalkyl” refers to an alkyl as defined herein, which is substituted by one or more than one cycloalkyl groups (which are independently selected) as defined herein. In some embodiments, “cycloalkylalkyl” is C₃₋₈cycloalkylC₁₋₆alkyl. In some embodiments, “cycloalkylalkyl” is alkyl substituted with one cycloalkyl. In some embodiments, cycloalkylalkyl is cyclopropylmethyl.

The term “haloalkyl,” as used herein, and unless otherwise specified, refers to an alkyl group substituted with 1, 2, 3, 4, or 5 halo groups. In some or any embodiments, the haloalkyl is a halo-C₁₋₆alkyl. In some or any embodiments, the haloalkyl is —CF₃, —CH₂F, —CHF₂, or —CH₂CF₃.

The term “haloalkoxy,” as used herein, and unless otherwise specified, refers to an —OR group where R is halo-C₁₋₁₀alkyl as defined herein. In some or any embodiments, the haloalkoxy is a halo-C₁₋₆alkoxy.

The terms “halogen” and “halo,” as used herein, and unless otherwise specified, are synonymous and refer to chloro, bromo, fluoro or iodo.

The term “heteroaryl,” as used herein, and unless otherwise specified, refers to a monocyclic aromatic ring system or multicyclic aromatic ring system wherein one or more (in some or any embodiments, 1, 2, 3, or 4) of the ring atoms is a heteroatom independently selected from O, S(O)₀₋₂, NH, and N, and the remaining ring atoms are carbon atoms, and where the ring may be optionally substituted as described herein. The heteroaryl group is bonded to the rest of the molecule through any atom in the ring system, valency rules permitting. In some or any embodiments, each ring of a heteroaryl group can contain one or two O atoms, one or two S atoms, and/or one to four N atoms, or a combination thereof, provided that the total number of heteroatoms in each ring is four or less and each ring contains at least one carbon atom. In some or any embodiments, the heteroaryl has from 5 to 20, from 5 to 15, from 5 to 6 ring atoms, or from 5 to 10 ring atoms. When heteroaryl is substituted, it can be substituted on any ring. and the like. In some embodiments, a heteroaryl is

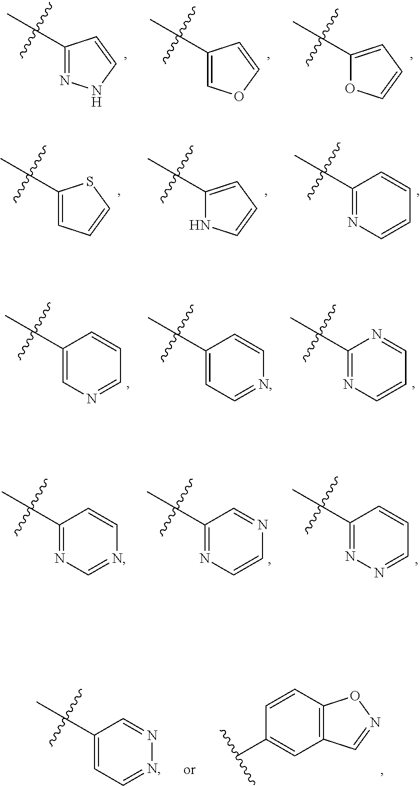

wherein [symbol] indicates the point of attachment of the heteroaryl to the rest of the molecule.

In some or any embodiments, monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl and triazolyl. In some or any embodiments, bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, or quinazolinyl. In some or any embodiments, tricyclic heteroaryl groups include, but are not limited to, acridinyl, benzindolyl, carbazolyl, dibenzofuranyl, perimidinyl, phenanthrolinyl, phenanthridinyl, and phenazinyl. In some or any embodiments, heteroaryl is indolyl, furanyl, pyridinyl, pyrimidinyl, imidazolyl, or pyrazolyl; each of which is optionally substituted with 1, 2, 3, or 4 groups as defined throughout the specification, including in some embodiments with group(s) independently selected from C₁₋₆alkyl, hydroxy, halo, halo-C₁₋₆alkyl, C₁₋₆alkoxy, cyano, or phenyl.

The term “heterocyclic,” as used herein, and unless otherwise specified, refers to a monovalent monocyclic non-aromatic ring system or a monovalent multicyclic ring system that contains at least one non-aromatic ring; wherein one or more (in some or any embodiments, 1, 2, 3, or 4) of the monocyclic non-aromatic ring atoms is a heteroatom independently selected from O, S(O)₀₋₂, and N, and the remaining ring atoms are carbon atoms; and wherein one or more (in some or any embodiments, 1, 2, 3, or 4) of any of the ring atoms in the multicyclic ring system is a heteroatom(s) independently selected from O, S(O)₀₋₂, and N, and the remaining ring atoms are carbon. The term “heterocyclic” does not include fully aromatic ring(s), i.e. does not include imidazole, pyrimidine, pyridine, and the like. In some or any embodiments, the heterocyclic ring comprises one or two heteroatom(s) which are independently selected from nitrogen and oxygen. In some or any embodiments, the heterocyclic ring comprises one or two heteroatom(s) which are oxygen. In some or any embodiments, the heterocyclic ring comprises one or two heteroatom(s) which are nitrogen (where the nitrogen is substituted as described in any aspect or embodiment described herein). In some or any embodiments, heterocyclic is multicyclic and comprises one heteroatom in a non-aromatic ring, or comprises one heteroatom in an aromatic ring, or comprises two heteroatoms in an aromatic ring, or comprises two heteroatoms where one is in an aromatic ring and the other is in a non-aromatic ring. In some or any embodiments, the heterocyclic group has from 3 to 20, 3 to 15, 3 to 10, 3 to 8, 4 to 7, or 5 to 6 ring atoms. In some or any embodiments, the heterocyclic is a monocyclic, bicyclic, tricyclic, or tetracyclic ring system. In some or any embodiments, the heterocyclic group may be a bridged or non-bridged, spirocyclic or not spirocyclic, and/or fused or not fused multicyclic group. One or more of the nitrogen and sulfur atoms may be optionally oxidized, one or more of the nitrogen atoms may be optionally quaternized, one or more of the carbon atoms may be optionally replaced with

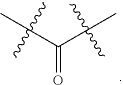 [SMILES: *C(*)=O]

Some rings may be partially or fully saturated, or aromatic provided that heterocyclic is not fully aromatic. The monocyclic and multicyclic heterocyclic rings may be attached to the main structure at any heteroatom or carbon atom which results in a stable compound. The multicyclic heterocyclic may be attached to the main structure through any of its rings, including any aromatic or nonaromatic ring, regardless of whether the ring contains a heteroatom. In some or any embodiments, heterocyclic is “heterocycloalkyl” which is 1) a saturated monovalent monocyclic group which contains at least one ring heteroatom, as described herein, or 2) a saturated monovalent bi- or tri-cyclic group in which at least one ring contains at least one heteroatom as described herein. When heterocyclic and heterocycloalkyl are substituted, they can be substituted on any ring, i.e. on any aromatic or nonaromatic ring comprised by heterocyclic and heterocycloalkyl. In some or any embodiments, such heterocyclic includes, but are not limited to, azepinyl, benzodioxanyl, benzodioxolyl, 3,4-dihydro-2H-benzo[b][1,4]oxazinyl, 3,4-dihydro-2H-benzo[b][1,4]dioxepinyl, 1,3-dihydroisobenzofuranyl, benzofuranonyl, benzopyranonyl, benzopyranyl, dihydrobenzofuranyl, benzotetrahydrothienyl, 2,2-dioxo-1,3-dihydrobenzo[c]thienyl, benzothiopyranyl, benzoxazinyl, β-carbolinyl, chromanyl, chromonyl, cinnolinyl, coumarinyl, decahydroquinolinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, 2,4-dioxo-imidazolidinyl, imidazolinyl, indolinyl, 2-oxo-indolinyl, isobenzotetrahydrofuranyl, isobenzotetrahydrothienyl, isochromanyl, isocoumarinyl, isoindolinyl, 1-oxo-isoindolinyl, 1,3-dioxo-isoindolinyl, isothiazolidinyl, isoxazolidinyl, 3-oxo-isoxazolidinyl, morpholinyl, 3,5-dioxo-morpholinyl, octahydroindolyl, octahydroisoindolyl, 1-oxo-octahydroisoindolyl, 1,3-dioxo-hexahydroisoindolyl, oxazolidinonyl, oxazolidinyl, oxiranyl, piperazinyl, 2,6-dioxo-piperazinyl, piperidinyl, 2,6-dioxo-piperidinyl, 4-piperidonyl, pyrazolidinyl, pyrazolinyl, pyrrolidinyl, pyrrolinyl, 2-oxopyrrolidinyl, 2,5-dioxopyrrolidinyl, quinuclidinyl, tetrahydrofuryl, tetrahydroisoquinolinyl, tetrahydropyranyl, tetrahydrothienyl, thiamorpholinyl, thiomorpholinyl, 3,5-dioxo-thiomorpholinyl, thiazolidinyl, 2,4-dioxo-thiazolidinyl, tetrahydroquinolinyl, phenothiazinyl, phenoxazinyl, xanthenyl, and 1,3,5-trithianyl. In some or any embodiments, heterocyclic is benzo-1,4-dioxanyl, benzodioxolyl, indolinyl, 2-oxo-indolinyl, pyrrolidinyl, piperidinyl, 2,3-dihydrobenzofuranyl, or decahydroquinolinyl; each of which is optionally substituted with 1, 2, 3, or 4 groups as defined throughout the specification, including in some or any embodiments with group(s) independently selected from halo, alkyl, and phenyl. In some embodiments, heterocycloalkyl is pyrrolidinyl. In some embodiments, heterocycloalkyl is an N-linked heterocycloalkyl.

The term “oxo” as used herein and unless otherwise specified, refers to a keto group (C═O). An oxo group that is a substituent of a nonaromatic carbon results in a conversion of a —CH₂— to —C═O. An oxo group that is a substituent of an aromatic carbon results in a conversion of —CH— to —C═O. When a substituent is oxo, then two hydrogens on the atom are replaced. When an oxo group substitutes aromatic moieties, the corresponding partially unsaturated ring replaces the aromatic ring. For example, a pyridyl group substituted by an oxo group is a pyridone. The person of ordinary skill in the art will appreciate that in some embodiments that such a group, e.g. pyridone and 2,4(1H,3H)-dioxo-pyrimidinyl, can exist in its tautomeric form, e.g. hydroxypyridine and 2,4-dihydroxypyrimidinyl, respectively.

The term “protecting group,” as used herein, and unless otherwise specified, refers to a group that is added to an oxygen, nitrogen or phosphorus atom to prevent its further reaction or for other purposes. A wide variety of oxygen and nitrogen protecting groups are known to those skilled in the art of organic synthesis. (See for example those described in Greene, et al., Protective Groups in Organic Synthesis, John Wiley and Sons, Fourth Edition, 2006, hereby incorporated by reference.)

The term “pharmaceutically acceptable salt,” as used herein, and unless otherwise specified, refers to any salt of a compound provided herein which retains its biological properties and which is not toxic or otherwise desirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; and (2) base addition salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, in some or any embodiments, and without limitation, sodium, potassium, calcium, magnesium, ammonium, tetraalkylammonium salts and the like. When the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “substantially free of” or “substantially in the absence of” stereoisomers with respect to a composition refers to a composition that includes at least 85 or 90% by weight, in some or any embodiments 95%, 98%, 99% or 100% by weight, of a designated stereoisomer of a compound in the composition. In some or any embodiments, in the methods and compounds provided herein, the compounds are substantially free of stereoisomers.

Similarly, the term “isolated” with respect to a composition refers to a composition that includes at least 85, 90%, 95%, 98%, 99% to 100% by weight, of a specified compound, the remainder comprising other chemical species or stereoisomers.

The term “isotopic composition,” as used herein, and unless otherwise specified, refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopic enrichment,” as used herein, and unless otherwise specified, refers to the percentage of incorporation of an amount of a specific isotope at a given atom in a molecule in the place of that atom's natural isotopic abundance. In some or any embodiments, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

The term “isotopically enriched,” as used herein, and unless otherwise specified, refers to an atom having an isotopic composition other than the natural isotopic composition of that atom. “Isotopically enriched” may also refer to a compound containing at least one atom having an isotopic composition other than the natural isotopic composition of that atom.

As used herein, the term “local anesthetic” means a drug which provides local numbness or pain relief. In some or any embodiments, local anesthetic includes aminoacylanilide compounds (in some or any embodiments, lidocaine, prilocaine, bupivacaine, ropivacaine, and mepivacaine) and related local anesthetic compounds having various substituents on the ring system or amine nitrogen; aminoalkyl benzoate compounds (in some or any embodiments, procaine, chloroprocaine, propoxycaine, hexylcaine, tetracaine, cyclomethycaine, benoxinate, butacaine, and proparacaine) and related local anesthetic compounds; cocaine; amino carbonate compounds (in some or any embodiments, diperodon); N-phenylamidine compounds (in some or any embodiments, phenacaine); N-aminoalkyl amide compounds (in some or any embodiments, dibucaine); aminoketone compounds (in some or any embodiments, falicaine and dyclonine); and amino ether compounds (in some or any embodiments, pramoxine and dimethisoquien).

As used herein, “alkyl,” “carbocyclic,” “cycloalkyl,” “aryl,” “alkoxy,” “heterocycloalkyl,” and “heterocyclic” groups optionally comprise deuterium at one or more positions where hydrogen atoms are present, and wherein the deuterium composition of the atom or atoms is other than the natural isotopic composition.

Also as used herein, “alkyl,” “carbocyclic,” “cycloalkyl,” “aryl,” “alkoxy,” “heterocycloalkyl,” “heterocyclic” groups optionally comprise carbon-13 at an amount other than the natural isotopic composition.

As used herein, and unless otherwise specified, the term “IC₅₀” refers to an amount, concentration or dosage of a particular test compound that achieves a 50% inhibition of a maximal response in an assay that measures such response.

As used herein, the terms “subject” and “patient” are used interchangeably. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and in some or any embodiments, a human. In some or any embodiments, the subject is a farm animal (e.g., a horse, a cow, a pig, etc.) or a pet (e.g., a dog or a cat). In some or any embodiments, the subject is a human.

As used herein, the terms “therapeutic agent” and “therapeutic agents” refer to any agent(s) which can be used in the treatment or prevention of a disorder or one or more symptoms thereof. In some or any embodiments, the term “therapeutic agent” includes a compound provided herein. In some or any embodiments, a therapeutic agent is an agent which is known to be useful for, or has been or is currently being used for the treatment or prevention of a disorder or one or more symptoms thereof.

“Therapeutically effective amount” refers to an amount of a compound or composition that, when administered to a subject for treating a condition, is sufficient to effect such treatment for the condition. A “therapeutically effective amount” can vary depending on, inter alia, the compound, the condition and its severity, and the age, weight, etc., of the subject to be treated.

“Treating” or “treatment” of any condition or disorder refers, in some or any embodiments, to ameliorating a condition or disorder that exists in a subject, including prophylactically. In another embodiment, “treating” or “treatment” includes ameliorating at least one physical parameter, which may be indiscernible by the subject. In yet another embodiment, “treating” or “treatment” includes modulating the condition or disorder, either physically (e.g., stabilization of a discernible symptom) or physiologically (e.g., stabilization of a physical parameter) or both. In yet another embodiment, “treating” or “treatment” includes delaying the onset of the condition or disorder. In yet another embodiment, “treating” or “treatment” includes the reduction or elimination of either the condition (e.g. pain) or one or more symptoms (e.g. pain) of the condition (e.g. sciatica), or to retard the progression of the condition (e.g. pain) or of one or more symptoms (e.g. pain) of the condition (e.g. sciatica), or to reduce the severity of the condition (e.g. pain) or of one or more symptoms (e.g. pain) of the condition (e.g. sciatica). In yet another embodiment, “treating” or “treatment” includes administering a compound described herein prophylactically.

As used herein, the terms “prophylactic agent” and “prophylactic agents” refer to any agent(s) which can be used in the prevention of a condition or one or more symptoms thereof and/or which prevents or impedes the onset, development, progression and/or severity of a condition. In some or any embodiments, the term “prophylactic agent” includes a compound provided herein. In some or any other embodiments, the term “prophylactic agent” does not refer a compound provided herein. In some or any embodiments, the agent is administered prophylactically, for example before surgery to prevent or impede the onset, duration, progression and/or severity of pain (e.g., post surgical pain).

As used herein, the phrase “prophylactically effective amount” refers to the amount of a therapy (e.g., prophylactic agent) which is sufficient to result in the prevention or reduction of the development, recurrence or onset of one or more symptoms associated with a condition, or to enhance or improve the prophylactic effect(s) of another therapy (e.g., another prophylactic agent).

### Compounds

Provided herein are compounds that can modulate the activity of voltage-gated ion channels (e.g., voltage-gated sodium channels). The compounds can be formed as described herein and used for the treatment of conditions associated with voltage-gated sodium channel function. In some or any embodiments, the condition associated with voltage-gated sodium channel function is pain or a condition associated with pain. In some or any embodiments, the condition associated with voltage-gated sodium channel function is a condition associated with pain. In some or any embodiments, the condition associated with voltage-gated sodium channel function is pain, itch, cough, epilepsy, Parkinson's disease, a mood disorder, psychosis, amyotrophic lateral sclerosis, glaucoma, ischemia, spasticity disorders and obsessive compulsive disorder. In some or any embodiments, the condition associated with voltage-gated sodium channel function is pain (in some embodiments, subacute or chronic pain). In some embodiments, the pain associated with voltage-gated sodium channel function includes pain and/or discomfort associated with dry eye syndrome, pain associated with (acute) corneal injuries or abrasions, acute ocular pain, chronic ocular pain, pain associated with corneal infections, pain associated with Parkinson's disease, pain associated with ALS, and pain associated with surgery (in some embodiments, ocular surgery).

The aspects and embodiments described herein include the recited compounds as well as a pharmaceutically acceptable salt thereof and/or an isomer thereof.

Included herein, if chemically possible, are all stereoisomers of the compounds, including diastereomers and enantiomers. Also included are mixtures of possible stereoisomers in any ratio, including, but not limited to, racemic mixtures. Unless stereochemistry is explicitly indicated in a structure at a particular atom, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

It will be apparent that certain structures recite specific stereochemistry at particular atoms.

Certain multicyclic structures provided herein are drawn with one or more floating substituents. Unless provided otherwise or otherwise clear from the context, the substituent(s) may be present on any atom of the multicyclic ring, where chemically feasible and valency rules permitting.

In some or any embodiments, provided herein is a compound of Formula (I) or a pharmaceutically acceptable salt thereof and/or an isomer thereof, wherein
A is phenyl substituted with R³, pyridinyl substituted with R³, benzoisoxazolyl substituted with R³, unsubstituted pyrazolyl, pyrazolyl substituted with R³, or

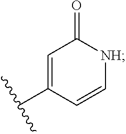 [SMILES: *c1cc[nH]c(=O)c1]

W¹ is —N═ or —C(H)═;

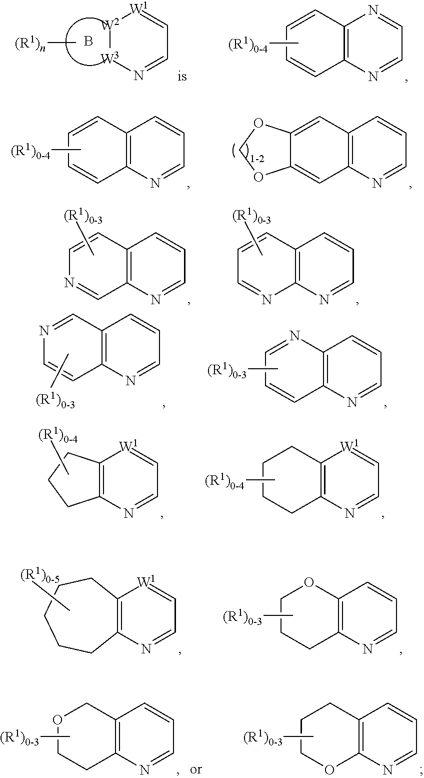

each R¹ is independently hydrogen, C₁-C₆ alkyl, C₁-C₆ alkoxy, C₁-C₆ haloalkyl, halo-C₁-C₆ alkoxy, or halogen;
each R² is independently hydrogen, C₁-C₆ alkyl, or halo;
R³ is —C(O)NH₂, —NH₂, —S(O)₂NH₂, —S(O)₂CH₃, or

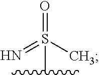 [SMILES: *S(C)(=N)=O]

m is 1 or 2;
n is 0, 1, or 2; and
p is 0, 1, 2, or 3.

Embodiment 1: In some or any embodiments, provided herein is a compound of Formula (I) wherein R¹ is independently in each instance selected from hydrogen, C₁-C₆ alkyl, C₁-C₆ alkoxy, C₁-C₆ haloalkyl, halo-C₁-C₆ alkoxy, halogen, C₃-C₆-cycloalkyl, and C₃-C₆-cycloalkylC₁-C₃-alkyl. In some or any embodiments, R¹ is independently in each instance selected from hydrogen, C₁-C₆ alkyl, C₁-C₆ alkoxy, C₁-C₆ haloalkyl, halo-C₁-C₆ alkoxy, and halogen. In some or any embodiments, each R¹ is hydrogen. In some or any embodiments, each R¹ is C₁-C₆ alkyl. In some or any embodiments, each R¹ is C₁-C₆ alkoxy. In some or any embodiments, each R¹ is C₁-C₆ haloalkyl. In some or any embodiments, each R¹ is halogen. In some or any embodiments, each R¹ is C₃-C₆-cycloalkyl. In some or any embodiments, each R¹ is C₃-C₆-cycloalkyl, C₁-C₃-alkyl.

Embodiment 1a: In some or any embodiments, one R¹ is present and is other than hydrogen. In some or any embodiments, two R¹ are present and each is independently other than hydrogen. In some or any embodiments, three R¹ are present and each is independently other than hydrogen. In some or any embodiments, at least one R¹ is C₁-C₆ alkyl. In some or any embodiments, at least one R¹ is —CH₃. In some or any embodiments, at least one R¹ is C₁-C₆alkoxy. In some or any embodiments, at least one R¹ is —OCH₃. In some or any embodiments, at least one R¹ is C₁-C₆ haloalkyl. In some or any embodiments, at least one R¹ is —CF₃. In some or any embodiments, at least one R¹ is halo-C₁-C₆ alkoxy. In some or any embodiments, at least one R¹ is —OCF₃. In some or any embodiments, at least one R¹ is halogen. In some or any embodiments, at least one R¹ is —Cl. In some or any embodiments embodiments, at least one R¹ is —F. In some or any embodiments, R¹ is C₃-C₆-cycloalkyl. In some or any embodiments, R¹ is C₃-C₆-cycloalkylC₁-C₃-alkyl.

Embodiment 1b: In some or any embodiments, each R¹ is independently selected from C₁-C₆ alkyl, C₁-C₆ alkoxy, halo-C₁-C₆ alkoxy, and halogen. In some or any embodiments, each R¹ is hydrogen.

Embodiment 1c: In some or any embodiments, two R are attached on adjacent ring carbons in

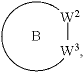

and together with the adjacent carbons to which they are attached form

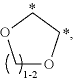

where * indicate the shared carbons in

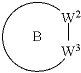

and any remaining R¹ are independently selected from hydrogen, C₁-C₆ alkyl, C₁-C₆ alkoxy, halogen, C₁-C₆ haloalkyl, halo-C₁-C₆ alkoxy, C₃-C₆-cycloalkyl, or C₃-C₆-cycloalkylC₁-C₃-alkyl.

Embodiment 2: In some or any embodiments of Formula (I) including embodiments 1-1b, A is C₆-C₁₀ aryl substituted with R³ and optionally substituted with (R^{3a})_{q}; 5- to 10-membered heteroaryl substituted with R³ and optionally substituted with (R^{3a})_{q}; or

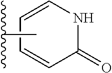

optionally substituted with (R^{3a})_{q1}. In some or any embodiments of Formula (I) including embodiments 1-1b, A is phenyl substituted with R³, pyridinyl substituted with R³, benzoisoxazolyl substituted with R³, unsubstituted pyrazolyl, pyrazolyl substituted with R³, or

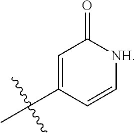 [SMILES: *c1cc[nH]c(=O)c1]

Embodiment 2a: In some or any embodiments including embodiments 1-1b, A is C₆-C₁₀ aryl substituted with R³ and optionally substituted with (R^{3a})_{q}. In some or any embodiments of Formula (I) including embodiments 1-14, A is C₆-C₁₀ aryl substituted with R³. In some or any embodiments of Formula (I) including embodiments 1-14, A is phenyl substituted with R³. In some or any embodiments of Formula (I) including embodiments 1-14, A is

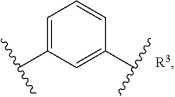

wherein [symbol] designates attachment to R³.

Embodiment 2b: In some or any embodiments including embodiments 1-1b, A is 5- to 10-membered heteroaryl substituted with R³ and optionally substituted with (R^{3a})_{q}. In some or any embodiments including embodiments 1-1b, A is 9- or 10-membered heteroaryl substituted with R³ and optionally substituted with (R^{3a})_{q}. In some or any embodiments including embodiments 1-1b, A is 9-membered heteroaryl substituted with R³ and optionally substituted with (R 3)_{q}. In some or any embodiments including embodiments 1-1b, A is 10-membered heteroaryl substituted with R³ and optionally substituted with (R^{3a})_{q}.

Embodiment 2c: In some or any embodiments of Formula (I) including embodiments 1-1b, A is 5- or 6-membered heteroaryl. In some or any embodiments including embodiments 1-1b, A is selected from the group consisting of:

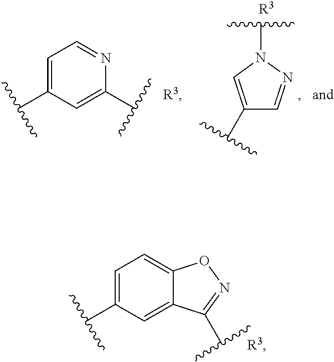

wherein [symbol] designates attachment to R³. In some or any embodiments including embodiments 1-1b, A is

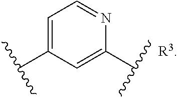

In some or any embodiments including embodiments 1-1b, A is

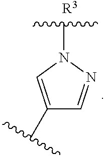

In some or any embodiments including embodiments 1-1b, A is

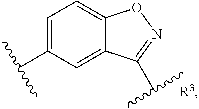 [SMILES: *c1ccc2onc(*)c2c1]

wherein [symbol] designates attachment to R³.

Embodiment 2d: In some or any embodiments including embodiments 1-1b, A is

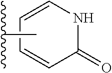

optionally substituted with (R^{3a})_{q1}. In some or any embodiments including embodiments 1-1b, A is

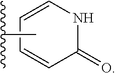

Embodiment 3: In some or any embodiments of Formula (I) including embodiments 1-2d, W¹ is —N═, —C(H)═, —C(halogen)=, —C(C₁-C₆ alkyl)=, or —C(cyclopropyl)=. In some or any embodiments, W¹ is —N═ or —C(H)═. In some or any embodiments including embodiments 1-2d, W¹ is —N═. In some or any embodiments including embodiments 1-2a, W¹ is —C(H)═. In some or any embodiments including embodiments 1-2d, W¹ is —C(halogen)=. In some or any embodiments including embodiments 1-2d, W¹ is —C(C₁-C₆ alkyl)=. In some or any embodiments including embodiments 1-2d, W¹ is —C(cyclopropyl)=. In some or any embodiments including embodiments 1-2d, W¹ is —N═. In some or any embodiments including embodiments 1-2d, W¹ is —C(H)═. In some or any embodiments including embodiments 1-2d, W¹ is —C(halogen)=. In some or any embodiments including embodiments 1-2d, W¹ is —C(C₁-C₆ alkyl)=. In some or any embodiments including embodiments 1-2d, W¹ is —C(cyclopropyl)=.

Embodiment 4: In some or any embodiments of Formula (I) including embodiments 1-3, W² and W³ are each —C—, the dashed bond between W² and W³ is a double bond, and

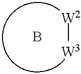

is a partially unsaturated 5 to 7-membered carbocyclic ring, a benzo ring, a partially unsaturated 5 to 7-membered heterocyclic ring, or a 5 or 6-membered heteroaromatic ring; or one of W² and W³ is —C— and the other is —N—, the dashed bond between W² and W³ is a single bond, and

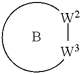

is a 5 or 6-membered heterocyclic ring optionally comprising an additional N and where the remaining ring atoms are C.

Embodiment 5: In some or any embodiments including embodiments 1-4, W² and W³ are each —C—; the dashed bond between W² and W³ is a double bond, and

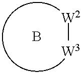

is a partially unsaturated 5 to 7-membered carbocyclic ring, a benzo ring, a partially unsaturated 5 to 7-membered heterocyclic ring, or a 5 or 6-membered heteroaromatic ring. In some or any embodiments including embodiments 1-4, W² and W³ are each —C—; the dashed bond between W² and W³ is a double bond, and

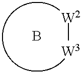

is a partially unsaturated 5 to 7-membered carbocyclic ring. In some or any embodiments including embodiments 1-4, W² and W³ are each —C—; the dashed bond between W² and W³ is a double bond, and

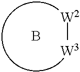

is benzo ring. In some or any embodiments including embodiments 1-4, W² and W³ are each —C—; the dashed bond between W² and W³ is a double bond, and

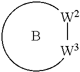

is a partially unsaturated 5 to 7-membered heterocyclic ring. In some or any embodiments including embodiments 1-4, W² and W³ are each —C—; the dashed bond between W² and W³ is a double bond, and

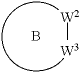

is a 5 or 6-membered heteroaromatic ring.

Embodiment 6: In some or any embodiments including embodiments 1-4, one of W² and W³ is —C— and the other is —N—; the dashed bond between W² and W³ is a single bond, and

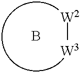

is a 5 or 6-membered heterocyclic ring optionally comprising an additional N and where the remaining ring atoms are C. In some or any embodiments including embodiments 1-4, W² is —C— and W³ is —N—. In some or any embodiments including embodiments 1-4, W² is —N— and W³ is —C—. In some or any embodiments including embodiments 1-4,

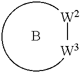

is a 5-membered heterocyclic ring optionally comprising an additional N and where the remaining ring atoms are C. In some or any embodiments including embodiments 1-4,

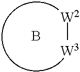

is a 6-membered heterocyclic ring optionally comprising an additional N and where the remaining ring atoms are C.

Embodiment 7: In some or any embodiments of Formula (I) including embodiments 1-6, R², independently in each instance, is hydrogen, halogen, or C₁-C₆ alkyl; or two R² are attached on adjacent carbons and together with the carbons to which they are attached form a 4- to 7-membered carbocyclic ring and the remaining R², independently in each instance, is hydrogen, halogen, or C₁-C₆ alkyl; or two R² are attached on the same carbon and together with the carbon to which they are attached form a 4- to 7-membered carbocyclic ring and the remaining R², independently in each instance, is hydrogen, halogen, or C₁-C₆ alkyl. In some or any embodiments of Formula (I) including embodiments 1-6, R², independently in each instance, is hydrogen, halogen, or C₁-C₆ alkyl. In some or any embodiments including embodiments 1-6, R² is hydrogen. In some or any embodiments including embodiments 1-6, R² is halogen. In some or any embodiments including embodiments 1-6, R² is C₁-C₆ alkyl. In some or any embodiments of Formula (I) including embodiments 1-6, two R² are attached on adjacent carbons and together with the carbons to which they are attached form a 4- to 7-membered carbocyclic ring and the remaining R², independently in each instance, is hydrogen, halogen, or C₁-C₆ alkyl. In some or any embodiments of Formula (I) including embodiments 1-6, two R² are attached on the same carbon and together with the carbon to which they are attached form a 4- to 7-membered carbocyclic ring and the remaining R² independently in each instance, is hydrogen, halogen, or C₁-C₆ alkyl.

Embodiment 7a: In some or any embodiments including embodiments 1-6, one R² is present and is other than hydrogen. In some or any embodiments including embodiments 1-6, two R² are present and each is independently other than hydrogen. In some or any embodiments including embodiments 1-6, three R² are present and each is independently other than hydrogen. In some or any embodiments including embodiments 1-6, at least one R² is halogen. In some or any embodiments including embodiments 1-6, at least one R² is —F. In some or any embodiments including embodiments 1-6, at least one R² is C₁-C₆ alkyl. In some or any embodiments including embodiments 1-6, at least one R² is —CH₃.

Embodiment 7b: In some or any embodiments including embodiments 1-6, each R² is independently selected from C₁-C₆ alkyl and halogen, wherein, in some embodiments, 1, 2, or 3 R² are present, wherein, in some embodiments, 2 or 3 R² are present. In some or any embodiments including embodiments 1-6, each R² is hydrogen.

Embodiment 8: In some or any embodiments of Formula (I) including embodiments 1-7b, R³ is —C(O)NH₂, —NH₂, —S(O)₂NHR, —S(O)₂C₁-C₆-alkyl, or

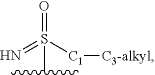

wherein R is hydrogen, C₁-C₃ alkyl or C₃-C₅ cycloalkyl. In some or any embodiments including embodiments 1-7b, R³ is —C(O)NH₂. In some or any embodiments including embodiments 1-7b, R³ is —NH₂. In some or any embodiments including embodiments 1-7b, R³ is —S(O)₂NHR. In some or any embodiments including embodiments 1-7b, R³ is —S(O)₂C₁-C₆-alkyl. In some or any embodiments including embodiments 1-7b, R³ is

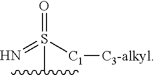

In some or any embodiments including embodiments 1-7b, R is hydrogen, C₁-C₃ alkyl or C₃-C₅ cycloalkyl. In some or any embodiments including embodiments 1-7b, R is hydrogen. In some or any embodiments including embodiments 1-7b. R is C₁-C₃ alkyl. In some or any embodiments including embodiments 1-7b, R is C₃-C₅ cycloalkyl.

Embodiment 8a: In some or any embodiments including embodiments 1-7b, R³ is —C(O)NH₂, —NH₂, —S(O)₂NHR, —S(O)₂C₁-C₆-alkyl, or

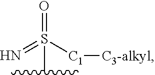

wherein R is hydrogen, C₁-C₃ alkyl or C₃-C₅ cycloalkyl. In some or any embodiments including embodiments 1-7b, R³ is —C(O)NH₂. In some or any embodiments including embodiments 1-7b, R³ is —NH₂. In some or any embodiments including embodiments 1-7b, R³ is —S(O)₂NHR. In some or any embodiments including embodiments 1-7b, R³ is —S(O)₂C₁-C₆-alkyl. In some or any embodiments including embodiments 1-7b, R³ is

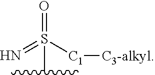

In some or any embodiments including embodiments 1-7b, R is hydrogen, C₁-C₃ alkyl or C₃-C₅ cycloalkyl. In some or any embodiments including embodiments 1-7b, R is hydrogen. In some or any embodiments including embodiments 1-7b, R is C₁-C₃ alkyl. In some or any embodiments including embodiments 1-7b, R is C₃-C₅ cycloalkyl. In some or any embodiments including embodiments 1-7b, R³ is —S(O)₂NH₂. In some or any embodiments including embodiments 1-7b, R³ is —S(O)₂CH₃. In some or any embodiments including embodiments 1-7b, R³ is

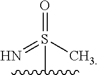 [SMILES: *S(C)(=N)=O]

Embodiment 9: In some or any embodiments of Formula (I) including embodiments 1-8, R^{3a}, independently in each instance, is hydrogen, halogen, C₁-C₃ alkyl, or C₃-C₆ cycloalkyl. In some or any embodiments including embodiments 1-8, R^{3a} is hydrogen. In some or any embodiments including embodiments 1-8, R^{3a} is halogen. In some or any embodiments including embodiments 1-8, R^{3a} is C₁-C₃ alkyl. In some or any embodiments including embodiments 1-8, R^{3a} is C₃-C₆ cycloalkyl.

Embodiment 10: In some or any embodiments of Formula (I) including embodiments 1-9, m is 0, 1, 2, or 3. In some or any embodiments, m is 1 or 2. In some or any embodiments including embodiments 1-9, m is 0. In some or any embodiments including embodiments 1-9, m is 1. In some or any embodiments including embodiments 1-9, m is 2. In some or any embodiments, m is 3.

Embodiment 11: In some or any embodiments of Formula (I) including embodiments 1-10, n is 0, 1, 2, 3, or 4. In some or any embodiments of Formula (I) including embodiments 1-10, n is 0, 1, or 2. In some or any embodiments including embodiments 1-10, n is 0. In some or any embodiments including embodiments 1-10, n is 1. In some or any embodiments, n is 2. In some or any embodiments including embodiments 1-10, n is 3. In some or any embodiments including embodiments 1-10, n is 4.

Embodiment 12: In some or any embodiments of Formula (I) including embodiments 1-11, p is 0, 1, 2, 3, 4, 5, or 6. In some or any embodiments of Formula (I) including embodiments 1-11, p is 0, 1, 2, or 3. In some or any embodiments including embodiments 1-11, p is 0. In some or any embodiments including embodiments 1-11, p is 1. In some or any embodiments including embodiments 1-11, p is 2. In some or any embodiments including embodiments 1-11, p is 3. In some or any embodiments including embodiments 1-11, p is 4. In some or any embodiments including embodiments 1-11, p is 5. In some or any embodiments including embodiments 1-11, p is 6.

Embodiment 13: In some or any embodiments of Formula (I) including embodiments 1-12, q is 0, 1, 2, or 3. In some or any embodiments including embodiments 1-12, q is 0. In some or any embodiments including embodiments 1-12, q is 1. In some or any embodiments including embodiments 1-12, q is 2. In some or any embodiments including embodiments 1-12, q is 3.

Embodiment 13a: In some or any embodiments of Formula (I) including embodiments 1-12, q1 is 0, 1, or 2. In some or any embodiments including embodiments 1-12, q1 is 0. In some or any embodiments including embodiments 1-12, q1 is 1. In some or any embodiments including embodiments 1-12, q1 is 2.

Embodiment 14: In some or any embodiments of Formula (I) including embodiments 1-13, r is 0 or 1. In some or any embodiments including embodiments 1-13, r is 0. In some or any embodiments including embodiments 1-13, r is 1.

Embodiment 15: In some or any embodiments of Formula (I) including embodiments 1-14,

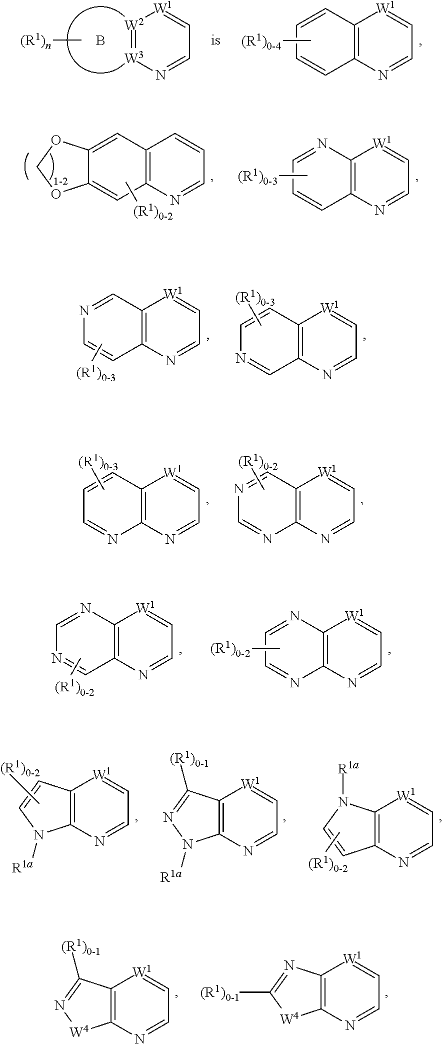

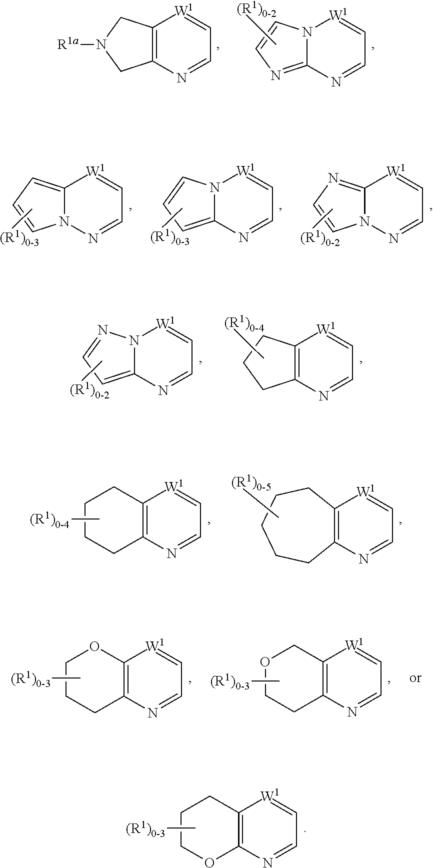

In some or any embodiments of Formula (I) including embodiments 1-14,

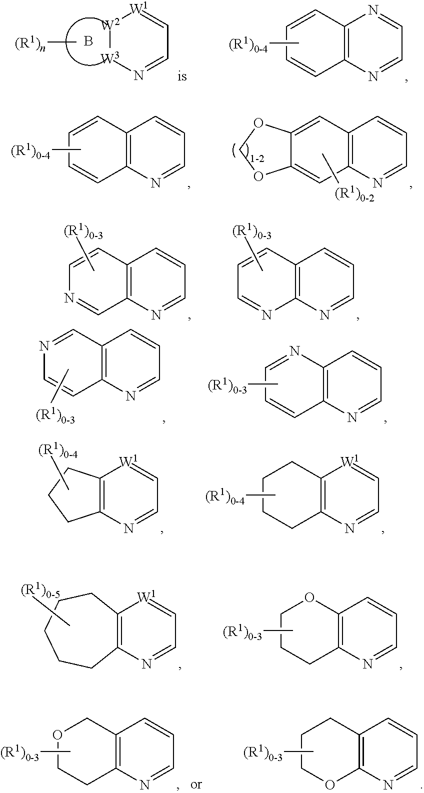

In some or any embodiments of Formula (I) including embodiments 1-14,

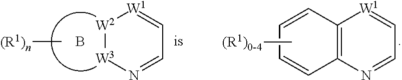

In some or any embodiments of Formula (I) including embodiments 1-14,

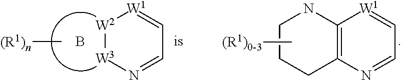

In some or any embodiments of Formula (I) including embodiments 1-14,

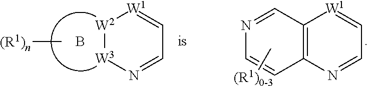

In some or any embodiments of Formula (I) including embodiments 1-14,

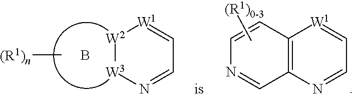

In some or any embodiments of Formula (I) including embodiments 1-14,

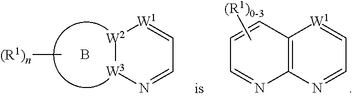

In some or any embodiments of Formula (I) including embodiments 1-14,

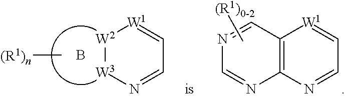

In some or any embodiments of Formula (I) including embodiments 1-14,

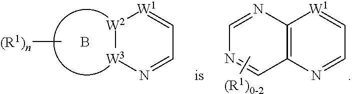

In some or any embodiments of Formula (I) including embodiments 1-14,

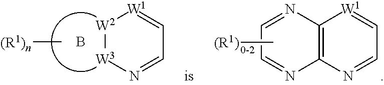

In some or any embodiments of Formula (I) including embodiments 1-14,

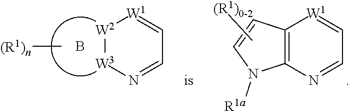

In some or any embodiments of Formula (I) including embodiments 1-14,

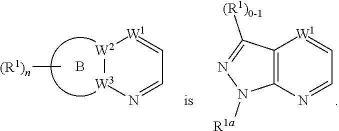

In some or any embodiments of Formula (I) including embodiments 1-14,

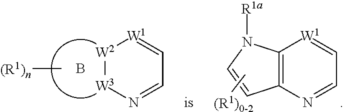

In some or any embodiments of Formula (I) including embodiments 1-14,

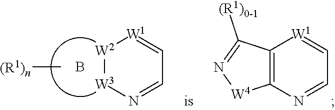

in some or any embodiments of Formula (I) including embodiments 1-14,

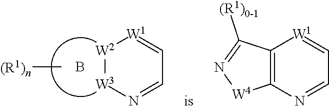

and W⁴ is O; and in some or any embodiments of Formula (I) including embodiments 1-14,

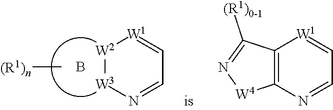

and W⁴ is S. In some or any embodiments of Formula (I) including embodiments 1-14,

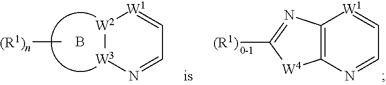

in some or any embodiments of Formula (I) including embodiments 1-14,

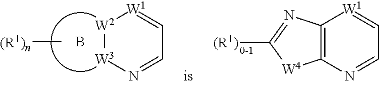

and W⁴ is O; and in some or any embodiments of Formula (I) including embodiments 1-14,

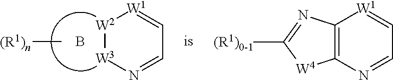

and W⁴ is S. In some or any embodiments of Formula (I) including embodiments 1-14,

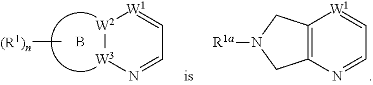

In some or any embodiments of Formula (I) including embodiments 1-14,

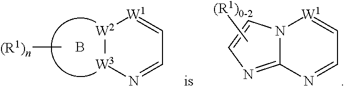

In some or any embodiments of Formula (I) including embodiments 1-14,

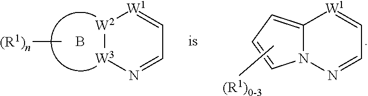

In some or any embodiments of Formula (I) including embodiments 1-14,

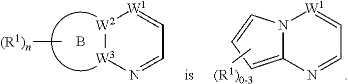

In some or any embodiments of Formula (I) including embodiments 1-14,

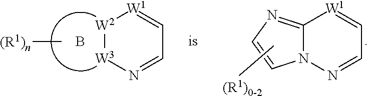

In some or any embodiments of Formula (I) including embodiments 1-14,

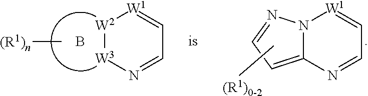

In some or any embodiments of Formula (I) including embodiments 1-14,

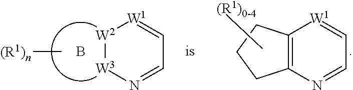

In some or any embodiments of Formula (I) including embodiments 1-14,

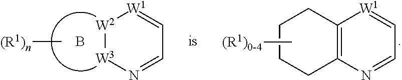

In some or any embodiments of Formula (I) including embodiments 1-14,

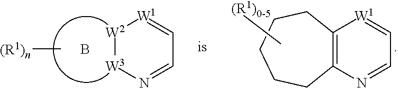

In some or any embodiments of Formula (I) including embodiments 1-14,

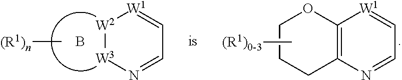

In some or any embodiments of Formula (I) including embodiments 1-14,

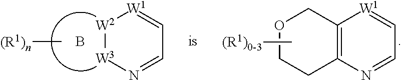

In some or any embodiments of Formula (I) including embodiments 1-14,

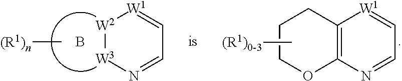

In some or any embodiments of Formula (I) including embodiments 1-14,

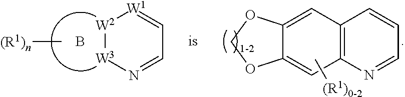

Embodiment 16: In some or any embodiments of Formula (I) including embodiments 1-14,

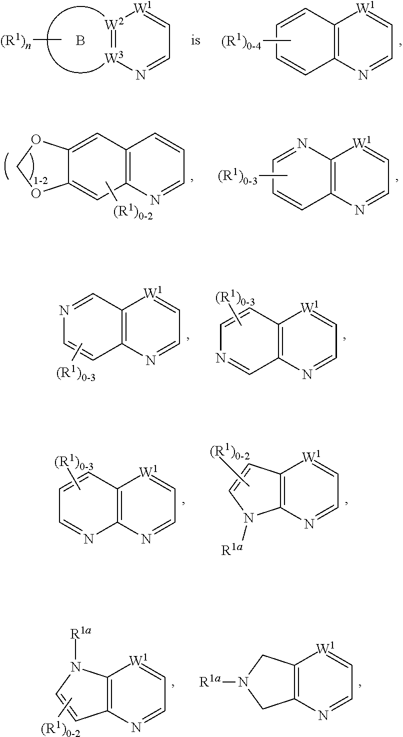

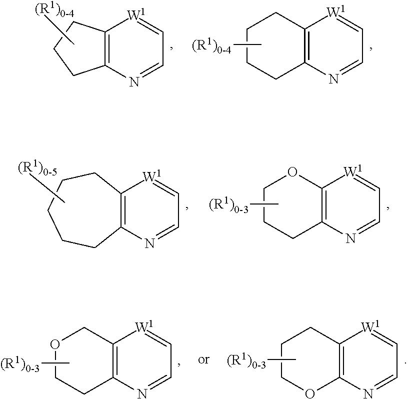

Embodiment 17: In some or any embodiments of Formula (I) including embodiments 1-16, R^{1a} is hydrogen. In some or any embodiments of Formula (I) including embodiments 1-16, R^{1a} is C₁-C₆ alkyl. In some or any embodiments of Formula (I) including embodiments 1-16, R^{1a} is C₃-C₆-cycloalkyl. In some or any embodiments, R^{1a} is C₃-C₆-cycloalkylC₁-C₃-alkyl.

In some or any embodiments of Formula (I), the compound is selected from any of compounds 1-97, or a pharmaceutically acceptable salt and/or an isomer thereof:

TABLE A

| Com- | |
|---|---|
| pound | |
| No. | Structure |
| 1 | 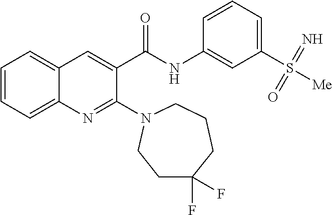 [SMILES: CS(=N)(=O)c1cccc(NC(=O)c2cc3ccccc3nc2N2CCCC(F)(F)CC2)c1] |
| 2 | 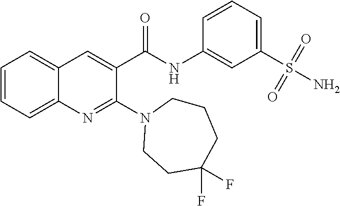 [SMILES: NS(=O)(=O)c1cccc(NC(=O)c2cc3ccccc3nc2N2CCCC(F)(F)CC2)c1] |
| 3 | 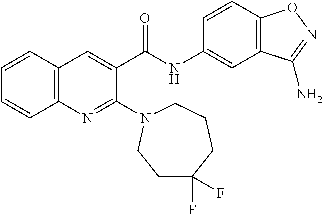 [SMILES: Nc1noc2ccc(NC(=O)c3cc4ccccc4nc3N3CCCC(F)(F)CC3)cc12] |
| 4 | 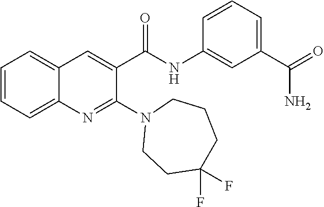 [SMILES: NC(=O)c1cccc(NC(=O)c2cc3ccccc3nc2N2CCCC(F)(F)CC2)c1] |
| 5 | 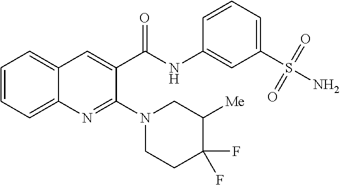 [SMILES: CC1CN(c2nc3ccccc3cc2C(=O)Nc2cccc(S(N)(=O)=O)c2)CCC1(F)F] |
| 6 | 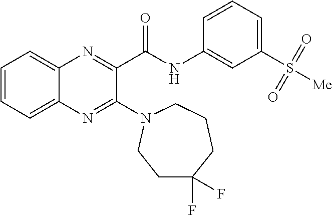 [SMILES: CS(=O)(=O)c1cccc(NC(=O)c2nc3ccccc3nc2N2CCCC(F)(F)CC2)c1] |
| 7 | 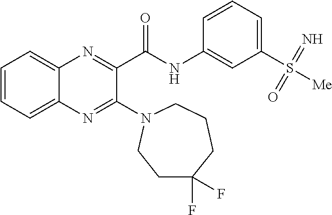 [SMILES: CS(=N)(=O)c1cccc(NC(=O)c2nc3ccccc3nc2N2CCCC(F)(F)CC2)c1] |
| 8 | 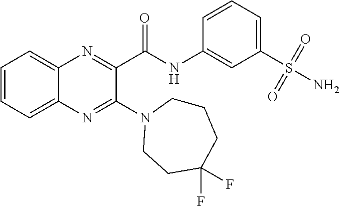 [SMILES: NS(=O)(=O)c1cccc(NC(=O)c2nc3ccccc3nc2N2CCCC(F)(F)CC2)c1] |
| 9 | 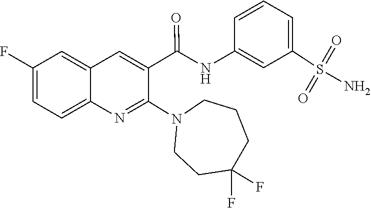 [SMILES: NS(=O)(=O)c1cccc(NC(=O)c2cc3cc(F)ccc3nc2N2CCCC(F)(F)CC2)c1] |
| 10 | 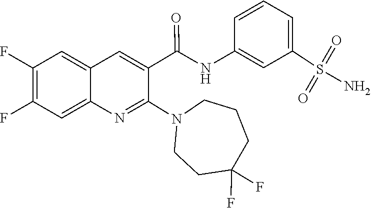 [SMILES: NS(=O)(=O)c1cccc(NC(=O)c2cc3cc(F)c(F)cc3nc2N2CCCC(F)(F)CC2)c1] |
| 11 | 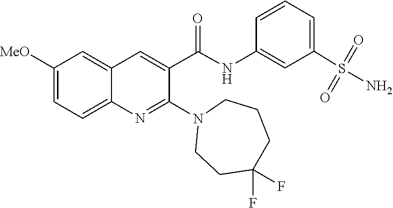 [SMILES: COc1ccc2nc(N3CCCC(F)(F)CC3)c(C(=O)Nc3cccc(S(N)(=O)=O)c3)cc2c1] |
| 12 | 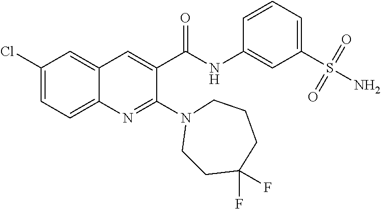 [SMILES: NS(=O)(=O)c1cccc(NC(=O)c2cc3cc(Cl)ccc3nc2N2CCCC(F)(F)CC2)c1] |
| 13 | 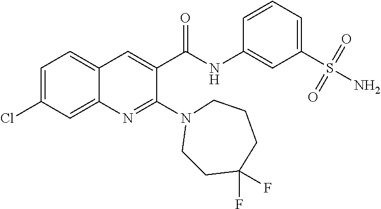 [SMILES: NS(=O)(=O)c1cccc(NC(=O)c2cc3ccc(Cl)cc3nc2N2CCCC(F)(F)CC2)c1] |
| 14 | 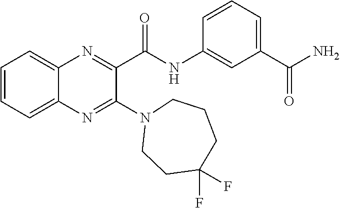 [SMILES: NC(=O)c1cccc(NC(=O)c2nc3ccccc3nc2N2CCCC(F)(F)CC2)c1] |
| 15 | 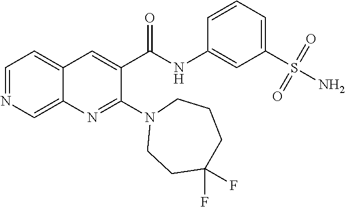 [SMILES: NS(=O)(=O)c1cccc(NC(=O)c2cc3ccncc3nc2N2CCCC(F)(F)CC2)c1] |
| 16 | 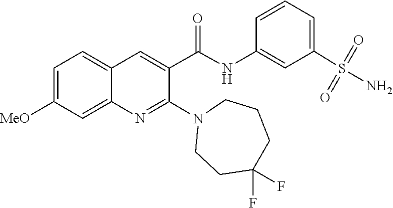 [SMILES: COc1ccc2cc(C(=O)Nc3cccc(S(N)(=O)=O)c3)c(N3CCCC(F)(F)CC3)nc2c1] |
| 17 | 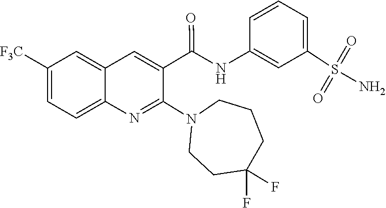 [SMILES: NS(=O)(=O)c1cccc(NC(=O)c2cc3cc(C(F)(F)F)ccc3nc2N2CCCC(F)(F)CC2)c1] |
| 18 | 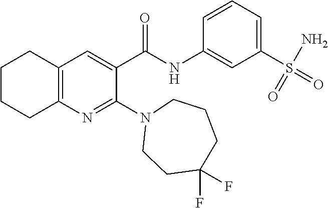 [SMILES: NS(=O)(=O)c1cccc(NC(=O)c2cc3c(nc2N2CCCC(F)(F)CC2)CCCC3)c1] |
| 19 | 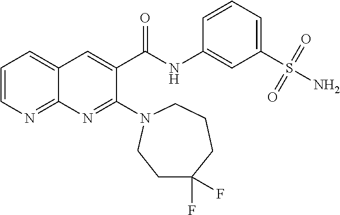 [SMILES: NS(=O)(=O)c1cccc(NC(=O)c2cc3cccnc3nc2N2CCCC(F)(F)CC2)c1] |
| 20 | 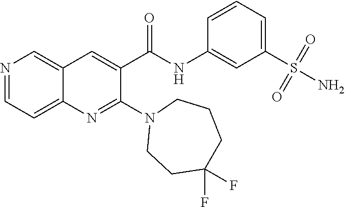 [SMILES: NS(=O)(=O)c1cccc(NC(=O)c2cc3cnccc3nc2N2CCCC(F)(F)CC2)c1] |
| 21 | 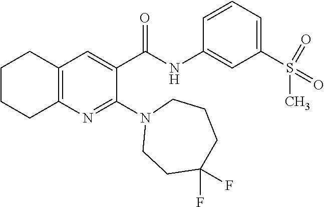 [SMILES: CS(=O)(=O)c1cccc(NC(=O)c2cc3c(nc2N2CCCC(F)(F)CC2)CCCC3)c1] |
| 22 | 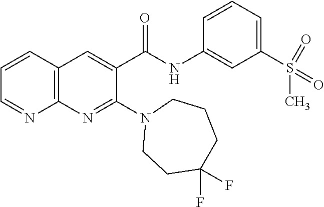 [SMILES: CS(=O)(=O)c1cccc(NC(=O)c2cc3cccnc3nc2N2CCCC(F)(F)CC2)c1] |
| 23 | 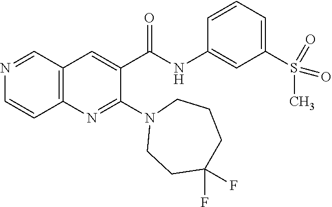 [SMILES: CS(=O)(=O)c1cccc(NC(=O)c2cc3cnccc3nc2N2CCCC(F)(F)CC2)c1] |
| 24 | 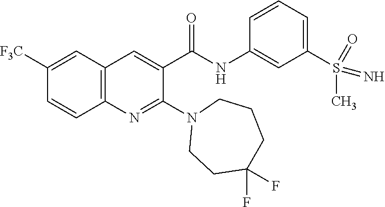 [SMILES: CS(=N)(=O)c1cccc(NC(=O)c2cc3cc(C(F)(F)F)ccc3nc2N2CCCC(F)(F)CC2)c1] |
| 25 | 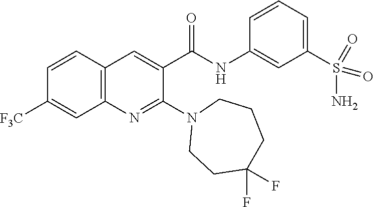 [SMILES: NS(=O)(=O)c1cccc(NC(=O)c2cc3ccc(C(F)(F)F)cc3nc2N2CCCC(F)(F)CC2)c1] |
| 26 | 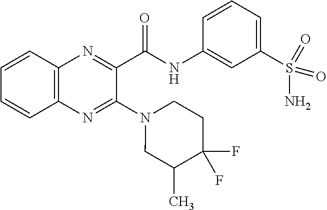 [SMILES: CC1CN(c2nc3ccccc3nc2C(=O)Nc2cccc(S(N)(=O)=O)c2)CCC1(F)F] |
| 27 | 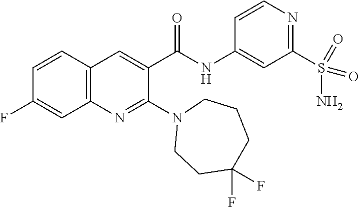 [SMILES: NS(=O)(=O)c1cc(NC(=O)c2cc3ccc(F)cc3nc2N2CCCC(F)(F)CC2)ccn1] |
| 28 | 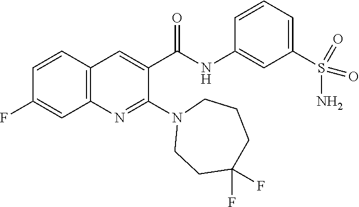 [SMILES: NS(=O)(=O)c1cccc(NC(=O)c2cc3ccc(F)cc3nc2N2CCCC(F)(F)CC2)c1] |
| 29 | 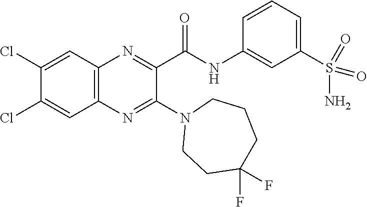 [SMILES: NS(=O)(=O)c1cccc(NC(=O)c2nc3cc(Cl)c(Cl)cc3nc2N2CCCC(F)(F)CC2)c1] |
| 30 | 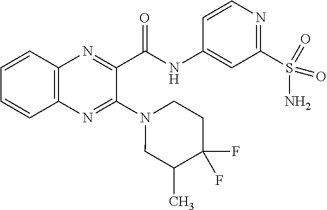 [SMILES: CC1CN(c2nc3ccccc3nc2C(=O)Nc2ccnc(S(N)(=O)=O)c2)CCC1(F)F] |
| 31 | 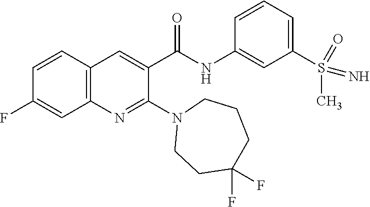 [SMILES: CS(=N)(=O)c1cccc(NC(=O)c2cc3ccc(F)cc3nc2N2CCCC(F)(F)CC2)c1] |
| 32 | 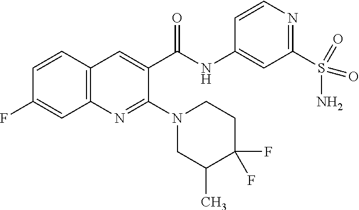 [SMILES: CC1CN(c2nc3cc(F)ccc3cc2C(=O)Nc2ccnc(S(N)(=O)=O)c2)CCC1(F)F] |
| 33 | 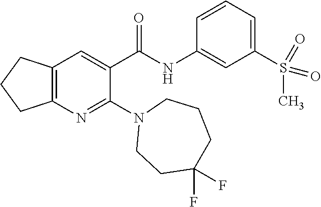 [SMILES: CS(=O)(=O)c1cccc(NC(=O)c2cc3c(nc2N2CCCC(F)(F)CC2)CCC3)c1] |
| 34 | 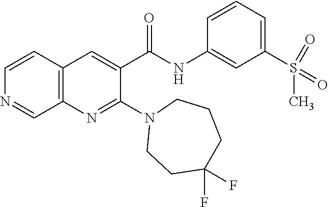 [SMILES: CS(=O)(=O)c1cccc(NC(=O)c2cc3ccncc3nc2N2CCCC(F)(F)CC2)c1] |
| 35 | 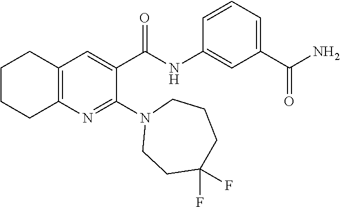 [SMILES: NC(=O)c1cccc(NC(=O)c2cc3c(nc2N2CCCC(F)(F)CC2)CCCC3)c1] |
| 36 | 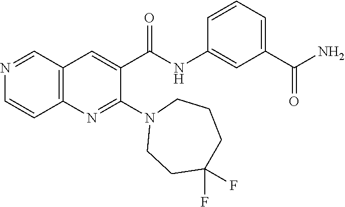 [SMILES: NC(=O)c1cccc(NC(=O)c2cc3cnccc3nc2N2CCCC(F)(F)CC2)c1] |
| 37 | 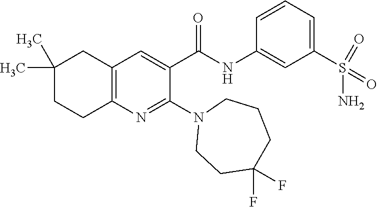 [SMILES: CC1(C)CCc2nc(N3CCCC(F)(F)CC3)c(C(=O)Nc3cccc(S(N)(=O)=O)c3)cc2C1] |
| 38 | 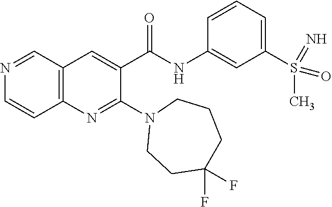 [SMILES: CS(=N)(=O)c1cccc(NC(=O)c2cc3cnccc3nc2N2CCCC(F)(F)CC2)c1] |
| 39 | 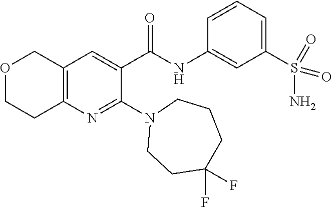 [SMILES: NS(=O)(=O)c1cccc(NC(=O)c2cc3c(nc2N2CCCC(F)(F)CC2)CCOC3)c1] |
| 40 | 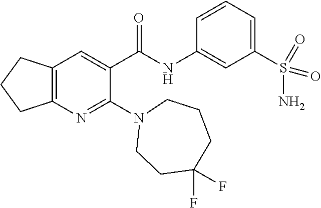 [SMILES: NS(=O)(=O)c1cccc(NC(=O)c2cc3c(nc2N2CCCC(F)(F)CC2)CCC3)c1] |
| 41 | 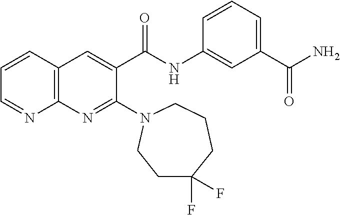 [SMILES: NC(=O)c1cccc(NC(=O)c2cc3cccnc3nc2N2CCCC(F)(F)CC2)c1] |
| 42 | 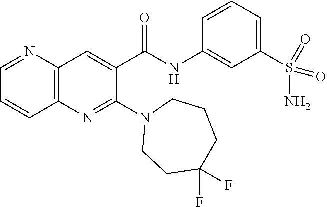 [SMILES: NS(=O)(=O)c1cccc(NC(=O)c2cc3ncccc3nc2N2CCCC(F)(F)CC2)c1] |
| 43 | 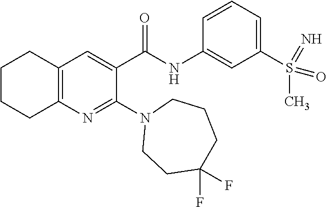 [SMILES: CS(=N)(=O)c1cccc(NC(=O)c2cc3c(nc2N2CCCC(F)(F)CC2)CCCC3)c1] |
| 44 | 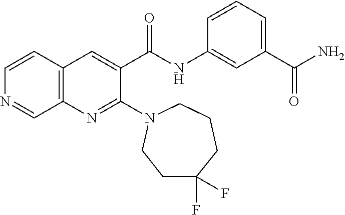 [SMILES: NC(=O)c1cccc(NC(=O)c2cc3ccncc3nc2N2CCCC(F)(F)CC2)c1] |
| 45 | 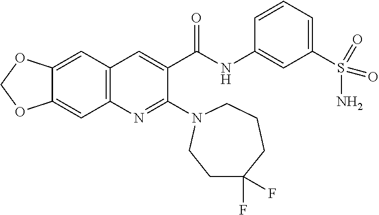 [SMILES: NS(=O)(=O)c1cccc(NC(=O)c2cc3cc4c(cc3nc2N2CCCC(F)(F)CC2)OCO4)c1] |
| 46 | 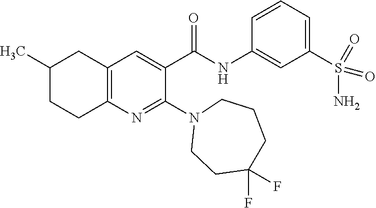 [SMILES: CC1CCc2nc(N3CCCC(F)(F)CC3)c(C(=O)Nc3cccc(S(N)(=O)=O)c3)cc2C1] |
| 47 | 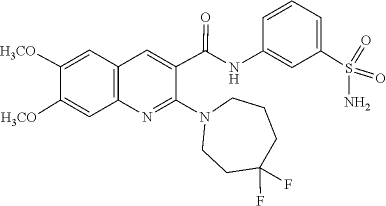 [SMILES: COc1cc2cc(C(=O)Nc3cccc(S(N)(=O)=O)c3)c(N3CCCC(F)(F)CC3)nc2cc1OC] |
| 48 | 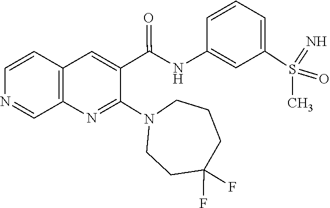 [SMILES: CS(=N)(=O)c1cccc(NC(=O)c2cc3ccncc3nc2N2CCCC(F)(F)CC2)c1] |
| 49 | 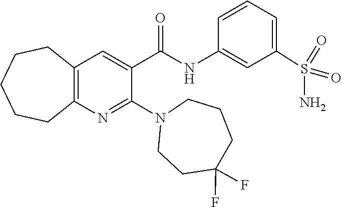 [SMILES: NS(=O)(=O)c1cccc(NC(=O)c2cc3c(nc2N2CCCC(F)(F)CC2)CCCCC3)c1] |
| 50 | 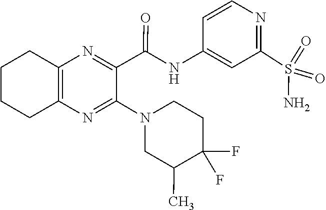 [SMILES: CC1CN(c2nc3c(nc2C(=O)Nc2ccnc(S(N)(=O)=O)c2)CCCC3)CCC1(F)F] |
| 51 | 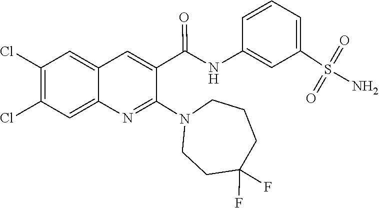 [SMILES: NS(=O)(=O)c1cccc(NC(=O)c2cc3cc(Cl)c(Cl)cc3nc2N2CCCC(F)(F)CC2)c1] |
| 52 | 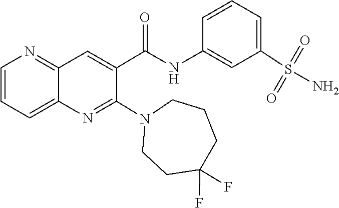 [SMILES: NS(=O)(=O)c1cccc(NC(=O)c2cc3ncccc3nc2N2CCCC(F)(F)CC2)c1] |
| 53 | 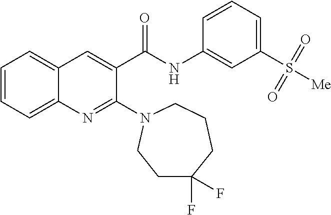 [SMILES: CS(=O)(=O)c1cccc(NC(=O)c2cc3ccccc3nc2N2CCCC(F)(F)CC2)c1] |
| 54 | 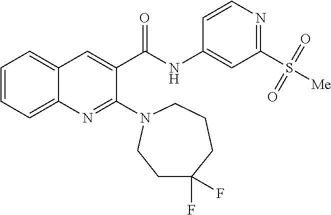 [SMILES: CS(=O)(=O)c1cc(NC(=O)c2cc3ccccc3nc2N2CCCC(F)(F)CC2)ccn1] |
| 55 | 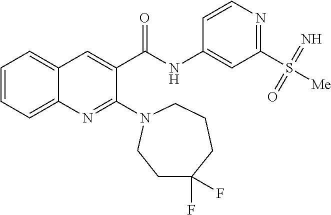 [SMILES: CS(=N)(=O)c1cc(NC(=O)c2cc3ccccc3nc2N2CCCC(F)(F)CC2)ccn1] |
| 56 | 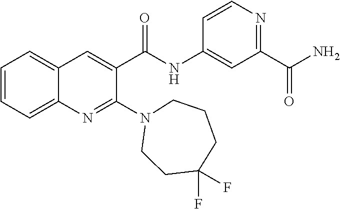 [SMILES: NC(=O)c1cc(NC(=O)c2cc3ccccc3nc2N2CCCC(F)(F)CC2)ccn1] |
| 57 | 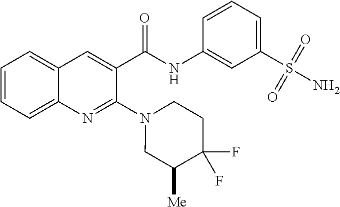 [SMILES: C[C@H]1CN(c2nc3ccccc3cc2C(=O)Nc2cccc(S(N)(=O)=O)c2)CCC1(F)F] |
| 58 | 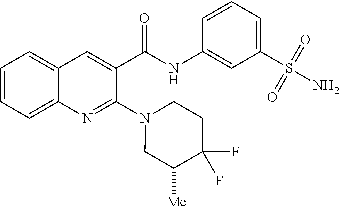 [SMILES: C[C@@H]1CN(c2nc3ccccc3cc2C(=O)Nc2cccc(S(N)(=O)=O)c2)CCC1(F)F] |
| 59 | 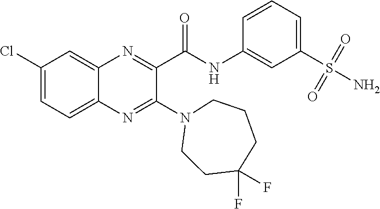 [SMILES: NS(=O)(=O)c1cccc(NC(=O)c2nc3cc(Cl)ccc3nc2N2CCCC(F)(F)CC2)c1] |
| 60 | 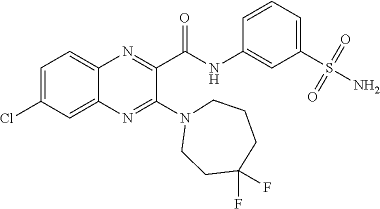 [SMILES: NS(=O)(=O)c1cccc(NC(=O)c2nc3ccc(Cl)cc3nc2N2CCCC(F)(F)CC2)c1] |
| 61 | 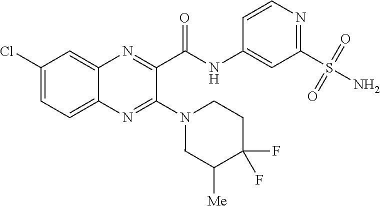 [SMILES: CC1CN(c2nc3ccc(Cl)cc3nc2C(=O)Nc2ccnc(S(N)(=O)=O)c2)CCC1(F)F] |
| 62 | 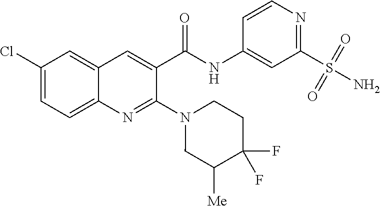 [SMILES: CC1CN(c2nc3ccc(Cl)cc3cc2C(=O)Nc2ccnc(S(N)(=O)=O)c2)CCC1(F)F] |
| 63 | 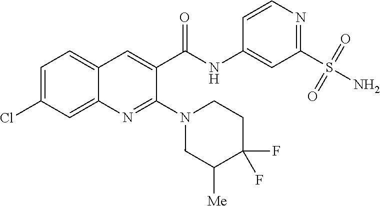 [SMILES: CC1CN(c2nc3cc(Cl)ccc3cc2C(=O)Nc2ccnc(S(N)(=O)=O)c2)CCC1(F)F] |
| 64 | 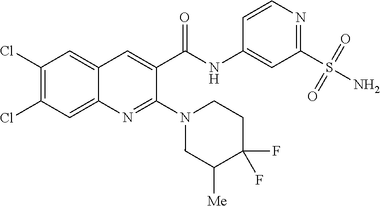 [SMILES: CC1CN(c2nc3cc(Cl)c(Cl)cc3cc2C(=O)Nc2ccnc(S(N)(=O)=O)c2)CCC1(F)F] |
| 65 | 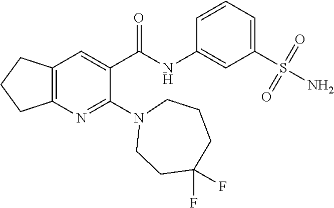 [SMILES: NS(=O)(=O)c1cccc(NC(=O)c2cc3c(nc2N2CCCC(F)(F)CC2)CCC3)c1] |
| 66 | 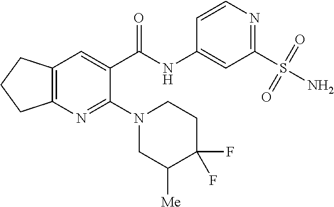 [SMILES: CC1CN(c2nc3c(cc2C(=O)Nc2ccnc(S(N)(=O)=O)c2)CCC3)CCC1(F)F] |
| 67 | 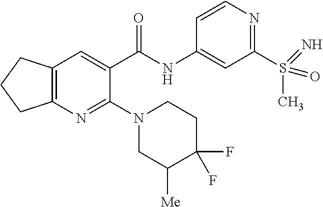 [SMILES: CC1CN(c2nc3c(cc2C(=O)Nc2ccnc(S(C)(=N)=O)c2)CCC3)CCC1(F)F] |
| 68 | 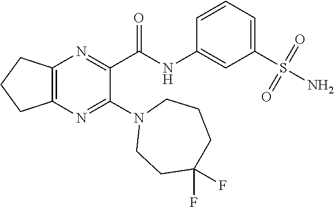 [SMILES: NS(=O)(=O)c1cccc(NC(=O)c2nc3c(nc2N2CCCC(F)(F)CC2)CCC3)c1] |
| 69 | 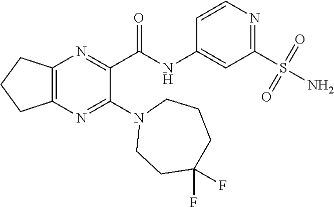 [SMILES: NS(=O)(=O)c1cc(NC(=O)c2nc3c(nc2N2CCCC(F)(F)CC2)CCC3)ccn1] |
| 70 | 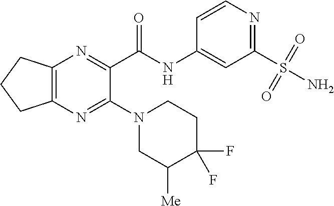 [SMILES: CC1CN(c2nc3c(nc2C(=O)Nc2ccnc(S(N)(=O)=O)c2)CCC3)CCC1(F)F] |
| 71 | 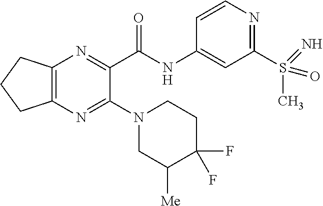 [SMILES: CC1CN(c2nc3c(nc2C(=O)Nc2ccnc(S(C)(=N)=O)c2)CCC3)CCC1(F)F] |
| 72 | 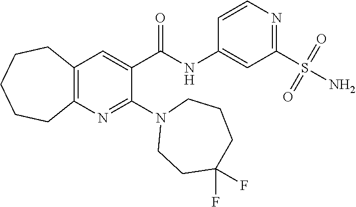 [SMILES: NS(=O)(=O)c1cc(NC(=O)c2cc3c(nc2N2CCCC(F)(F)CC2)CCCCC3)ccn1] |
| 73 | 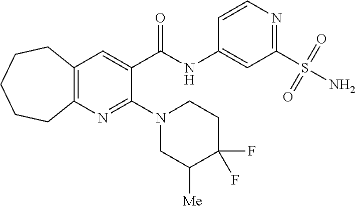 [SMILES: CC1CN(c2nc3c(cc2C(=O)Nc2ccnc(S(N)(=O)=O)c2)CCCCC3)CCC1(F)F] |
| 74 | 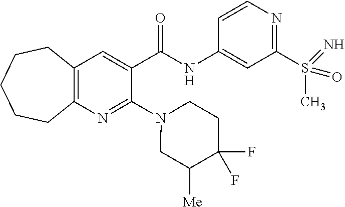 [SMILES: CC1CN(c2nc3c(cc2C(=O)Nc2ccnc(S(C)(=N)=O)c2)CCCCC3)CCC1(F)F] |
| 75 | 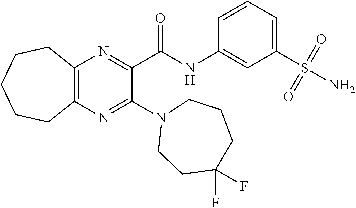 [SMILES: NS(=O)(=O)c1cccc(NC(=O)c2nc3c(nc2N2CCCC(F)(F)CC2)CCCCC3)c1] |
| 76 | 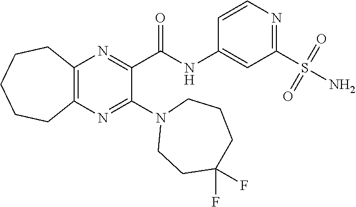 [SMILES: NS(=O)(=O)c1cc(NC(=O)c2nc3c(nc2N2CCCC(F)(F)CC2)CCCCC3)ccn1] |
| 77 | 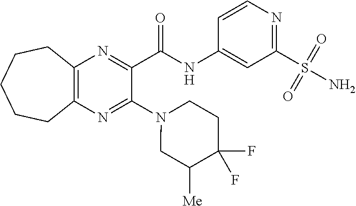 [SMILES: CC1CN(c2nc3c(nc2C(=O)Nc2ccnc(S(N)(=O)=O)c2)CCCCC3)CCC1(F)F] |
| 78 | 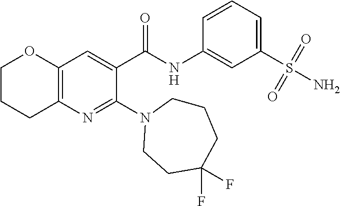 [SMILES: NS(=O)(=O)c1cccc(NC(=O)c2cc3c(nc2N2CCCC(F)(F)CC2)CCCO3)c1] |
| 79 | 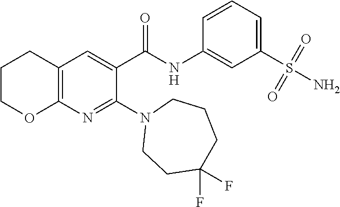 [SMILES: NS(=O)(=O)c1cccc(NC(=O)c2cc3c(nc2N2CCCC(F)(F)CC2)OCCC3)c1] |
| 80 | 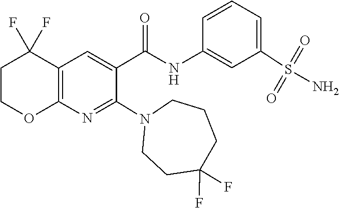 [SMILES: NS(=O)(=O)c1cccc(NC(=O)c2cc3c(nc2N2CCCC(F)(F)CC2)OCCC3(F)F)c1] |
| 81 | 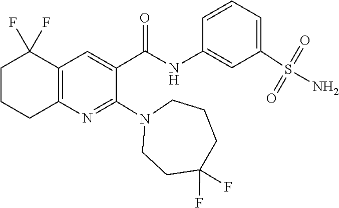 [SMILES: NS(=O)(=O)c1cccc(NC(=O)c2cc3c(nc2N2CCCC(F)(F)CC2)CCCC3(F)F)c1] |
| 82 | 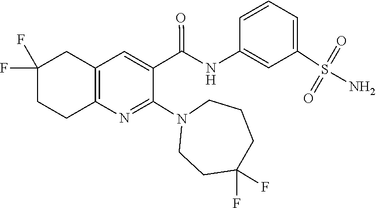 [SMILES: NS(=O)(=O)c1cccc(NC(=O)c2cc3c(nc2N2CCCC(F)(F)CC2)CCC(F)(F)C3)c1] |
| 83 | 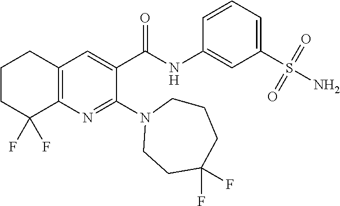 [SMILES: NS(=O)(=O)c1cccc(NC(=O)c2cc3c(nc2N2CCCC(F)(F)CC2)C(F)(F)CCC3)c1] |
| 84 | 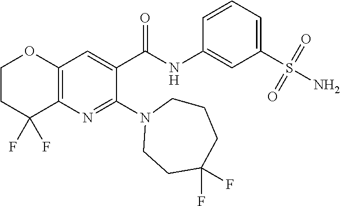 [SMILES: NS(=O)(=O)c1cccc(NC(=O)c2cc3c(nc2N2CCCC(F)(F)CC2)C(F)(F)CCO3)c1] |
| 85 | 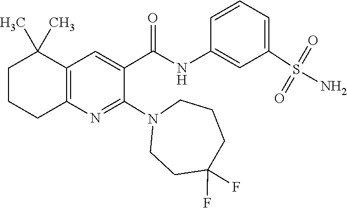 [SMILES: CC1(C)CCCc2nc(N3CCCC(F)(F)CC3)c(C(=O)Nc3cccc(S(N)(=O)=O)c3)cc21] |
| 86 | 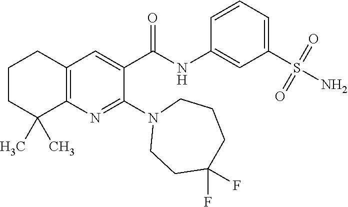 [SMILES: CC1(C)CCCc2cc(C(=O)Nc3cccc(S(N)(=O)=O)c3)c(N3CCCC(F)(F)CC3)nc21] |
| 87 | 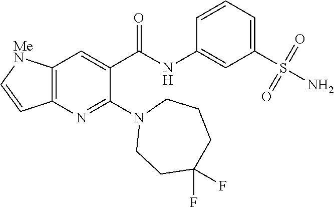 [SMILES: Cn1ccc2nc(N3CCCC(F)(F)CC3)c(C(=O)Nc3cccc(S(N)(=O)=O)c3)cc21] |
| 88 | 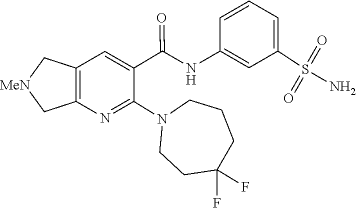 [SMILES: CN1Cc2cc(C(=O)Nc3cccc(S(N)(=O)=O)c3)c(N3CCCC(F)(F)CC3)nc2C1] |
| 89 | 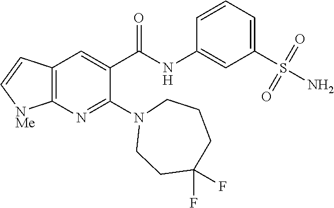 [SMILES: Cn1ccc2cc(C(=O)Nc3cccc(S(N)(=O)=O)c3)c(N3CCCC(F)(F)CC3)nc21] |
| 90 | 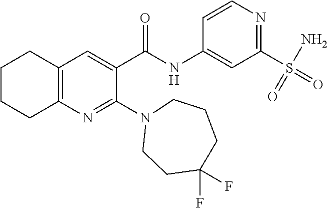 [SMILES: NS(=O)(=O)c1cc(NC(=O)c2cc3c(nc2N2CCCC(F)(F)CC2)CCCC3)ccn1] |
| 91 | 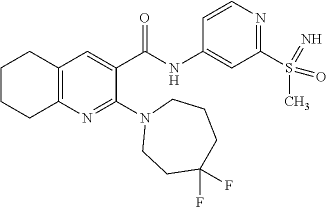 [SMILES: CS(=N)(=O)c1cc(NC(=O)c2cc3c(nc2N2CCCC(F)(F)CC2)CCCC3)ccn1] |
| 92 | 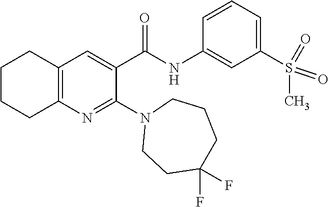 [SMILES: CS(=O)(=O)c1cccc(NC(=O)c2cc3c(nc2N2CCCC(F)(F)CC2)CCCC3)c1] |
| 93 | 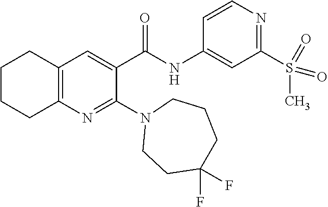 [SMILES: CS(=O)(=O)c1cc(NC(=O)c2cc3c(nc2N2CCCC(F)(F)CC2)CCCC3)ccn1] |
| 94 | 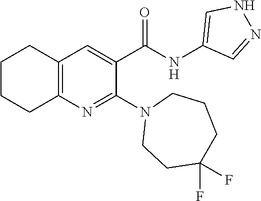 [SMILES: O=C(Nc1cn[nH]c1)c1cc2c(nc1N1CCCC(F)(F)CC1)CCCC2] |
| 95 | 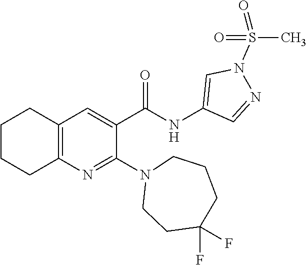 [SMILES: CS(=O)(=O)n1cc(NC(=O)c2cc3c(nc2N2CCCC(F)(F)CC2)CCCC3)cn1] |
| 96 | 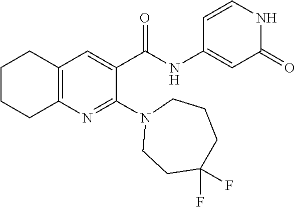 [SMILES: O=C(Nc1cc[nH]c(=O)c1)c1cc2c(nc1N1CCCC(F)(F)CC1)CCCC2] |
| 97 | 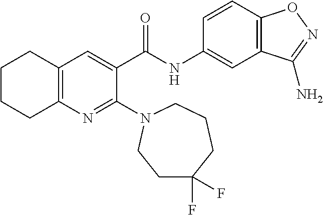 [SMILES: Nc1noc2ccc(NC(=O)c3cc4c(nc3N3CCCC(F)(F)CC3)CCCC4)cc12] |
| | and |
| 98 | 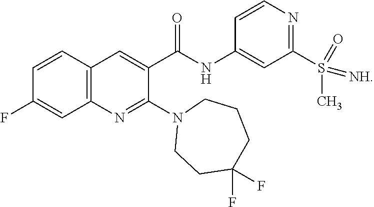 [SMILES: CS(=N)(=O)c1cc(NC(=O)c2cc3ccc(F)cc3nc2N2CCCC(F)(F)CC2)ccn1] |

In some of any embodiments provided herein is a compound according to any one of the following formulas:

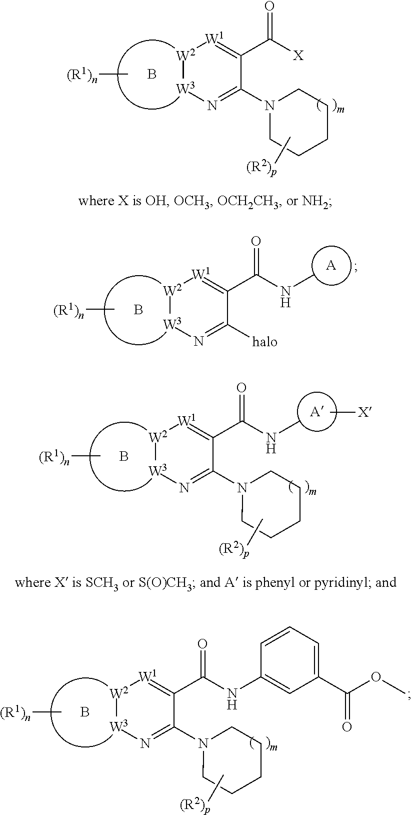

or a salt thereof,
wherein
A is phenyl substituted with R³, pyridinyl substituted with R³, benzoisoxazolyl substituted with R³, unsubstituted pyrazolyl, pyrazolyl substituted with R³, or

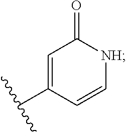 [SMILES: *c1cc[nH]c(=O)c1]

W¹ is —N═ or —C(H)═;

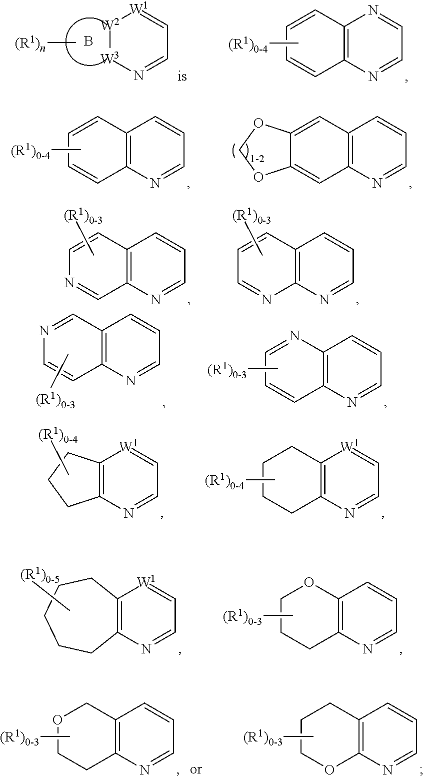

each R¹ is independently hydrogen, C₁-C₆ alkyl, C₁-C₆ alkoxy, C₁-C₆ haloalkyl, halo-C₁-C₆ alkoxy, or halogen;
each R² is independently hydrogen, C₁-C₆ alkyl, or halo;
R³ is —C(O)NH₂, —NH₂, —S(O)₂NH₂, —S(O)₂CH₃, or

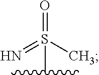 [SMILES: *S(C)(=N)=O]

m is 1 or 2;
n is 0, 1, or 2; and
p is 0, 1, 2, or 3.

### ADDITIONAL EMBODIMENTS

1. A compound of Formula (I):

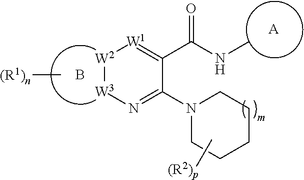

wherein:
R¹, independently in each instance, is selected from hydrogen, C₁-C₆ alkyl, C₁-C₆ alkoxy, halogen, C₁-C₆ haloalkyl, halo-C₁-C₆ alkoxy, C₃-C₆-cycloalkyl, and C₃-C₆-cycloalkylC₁-C₃-alkyl; or wherein two R¹ are attached on adjacent ring carbons in

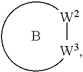

and together with the adjacent carbons to which they are attached form

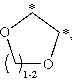

where * indicate the shared carbons in

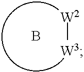

A is C₆-C₁₀ aryl substituted with R³ and optionally substituted with (R^{3a})_{q}; 5- to 10-membered heteroaryl substituted with R³ and optionally substituted with (R^{3a})_{q}; or

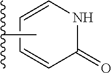

optionally substituted with (R^{3a})_{q1};
W¹ is —N═, —C(H)═, —C(halogen)=, —C(C₁-C₆ alkyl)=, or —C(cyclopropyl)=;
W² and W³ are each —C—, the dashed bond between W² and W³ is a double bond, and

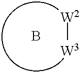

is a partially unsaturated 5 to 7-membered carbocyclic ring, a benzo ring, a partially unsaturated 5 to 7-membered heterocyclic ring, or a 5 or 6-membered heteroaromatic ring; or
one of W^{z} and W³ is —C— and the other is —N—, the dashed bond between W² and W³ is a single bond, and

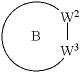

is a 5 or 6-membered heterocyclic ring optionally comprising an additional N and where the remaining ring atoms are C;
R², independently in each instance, is hydrogen, halogen, or C₁-C₆ alkyl; or
two R² are attached on adjacent carbons and together with the carbons to which they are attached form a 4- to 7-membered carbocyclic ring and the remaining R², independently in each instance, is hydrogen, halogen, or C₁-C₆ alkyl; or
two R² are attached on the same carbon and together with the carbon to which they are attached form a 4- to 7-membered carbocyclic ring and the remaining R², independently in each instance, is hydrogen, halogen, or C₁-C₆alkyl; R³ is hydrogen, —C(O)NH₂, —NH₂, —S(O)₂NHR, —S(O)₂C₁-C₆-alkyl, or

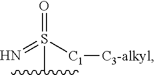

wherein R is hydrogen, C₁-C₃ alkyl or C₃-C₅ cycloalkyl;
R^{3a}, independently in each instance, is hydrogen, halogen, C₁-C₃ alkyl, or C₃-C₆ cycloalkyl;
m is 0, 1, 2, or 3;
n is 0, 1, 2, 3, or 4;
p is 0, 1, 2, 3, 4, 5, or 6;
q1 is 0, 1, or 2; and
q is 0, 1, 2, or 3;
or a pharmaceutically acceptable salt thereof and/or an isomer thereof.

2. The compound of claim 1, wherein A is C₆-C₁₀ aryl substituted with R³ and optionally substituted with (R^{3a})_{q}, optionally wherein A is phenyl substituted with R³ and optionally substituted with (R^{3a})_{q}, or a pharmaceutically acceptable salt thereof and/or an isomer thereof.

3. The compound of claim 1 or 2, wherein A is phenyl substituted with R³, or a pharmaceutically acceptable salt thereof and/or an isomer thereof.

4. The compound of any one of claims 1-3, wherein A is

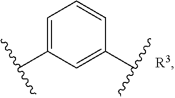

wherein [symbol] designates attachment to R³; or a pharmaceutically acceptable salt thereof and/or an isomer thereof

5. The compound of claim 1, wherein A is 5- to 10-membered heteroaryl substituted with R³ and optionally substituted with (R^{3a})_{q}, optionally wherein A is 9-membered heteroaryl substituted with R³ and optionally substituted with (R^{3a})_{q}, optionally wherein A is 10-membered heteroaryl substituted with R³ and optionally substituted with (R^{3a})_{q}, or a pharmaceutically acceptable salt thereof and/or an isomer thereof.

6. The compound of claim 1, wherein A is 5- or 6-membered heteroaryl substituted with R³ and optionally substituted with (R^{3a})_{q}, wherein A is 5-membered heteroaryl substituted with R³ and optionally substituted with (R^{3a})_{q}, or wherein A is 6-membered heteroaryl substituted with R³ and optionally substituted with (R^{3a})_{q}, or a pharmaceutically acceptable salt thereof and/or an isomer thereof.

7. The compound of claim 1, wherein A is selected from the group consisting of

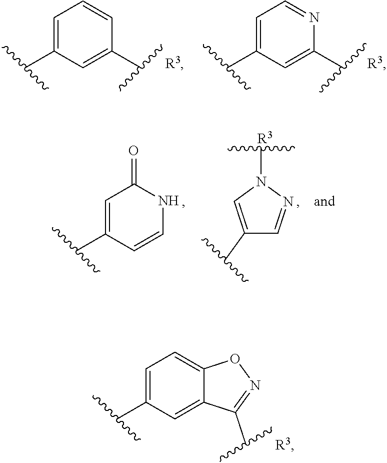

wherein [symbol] designates attachment to R³; or a pharmaceutically acceptable salt thereof and/or an isomer thereof.

8. The compound of any one of claims 1-7, wherein R³ is —C(O)NH₂, —NH₂, —S(O)₂NHR, —S(O)₂C₁-C₆-alkyl, or

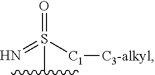

wherein R is hydrogen, C₁-C₃ alkyl or C₃-C₅ cycloalkyl, or a pharmaceutically acceptable salt thereof and/or an isomer thereof.

9. The compound of any one of claims 1-7, wherein A is pyrazolyl and R³ is hydrogen, or a pharmaceutically acceptable salt thereof and/or an isomer thereof.

10. The compound of any one of claims 1-8, wherein R³ is —C(O)NH₂, or a pharmaceutically acceptable salt thereof and/or an isomer thereof.

11. The compound of any one of claims 1-8, wherein R³ is —NH₂, or a pharmaceutically acceptable salt thereof and/or an isomer thereof.

12. The compound of any one of claims 1-8, wherein R³ is —S(O)₂NHR, or a pharmaceutically acceptable salt thereof and/or an isomer thereof.

13. The Compound of claims 1-9, wherein R is C₁-C₃ alkyl, or a pharmaceutically acceptable salt thereof and/or an isomer thereof.

14. The compound of claims 1-9, wherein R is C₃-C₅ cycloalkyl, or a pharmaceutically acceptable salt thereof and/or an isomer thereof.

15. The compound of any one of claims 1-8, wherein R³ is —S(O)₂NH₂, or a pharmaceutically acceptable salt thereof and/or an isomer thereof.

16. The compound of any one of claims 1-8, wherein R³ is —S(O)₂C₁-C₆-alkyl, or a pharmaceutically acceptable salt thereof and/or an isomer thereof.

17. The Compound of any one of claims 1-8 and 16, wherein R³ is —S(O)₂CH₃, or a pharmaceutically acceptable salt thereof and/or an isomer thereof.

18. The compound of any one of claims 1-8, wherein R³ is

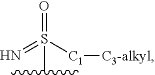

or a pharmaceutically acceptable salt thereof and/or an isomer thereof.

19. The Compound of claims 1-8 and 18, wherein R³ is

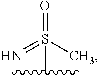 [SMILES: *S(C)(=N)=O]

or a pharmaceutically acceptable salt thereof and/or an isomer thereof.

20. The compound of any one of claims 1-19, wherein

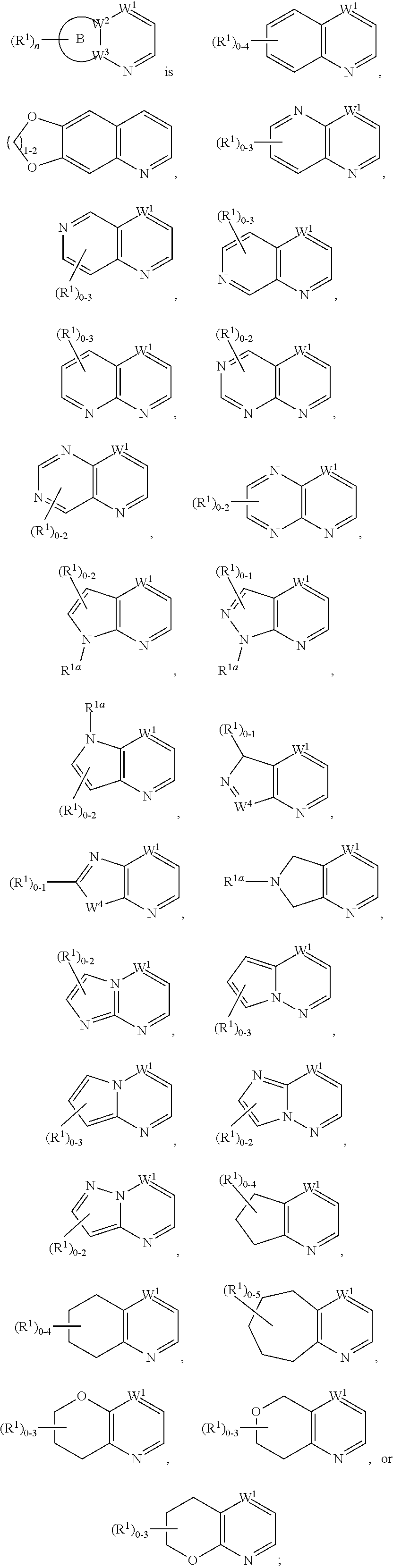

wherein W⁴ is O or S;
one R¹ can be R^{1a} as indicated in the above rings; and
R^{1a} is hydrogen, C₁-C₆-alkyl, C₃-C₆-cycloalkyl, or C₃-C₆-cycloalkylC₁-C₃-alkyl;
or a pharmaceutically acceptable salt thereof and/or an isomer thereof.

21. The compound of any one of claims 1-20, wherein

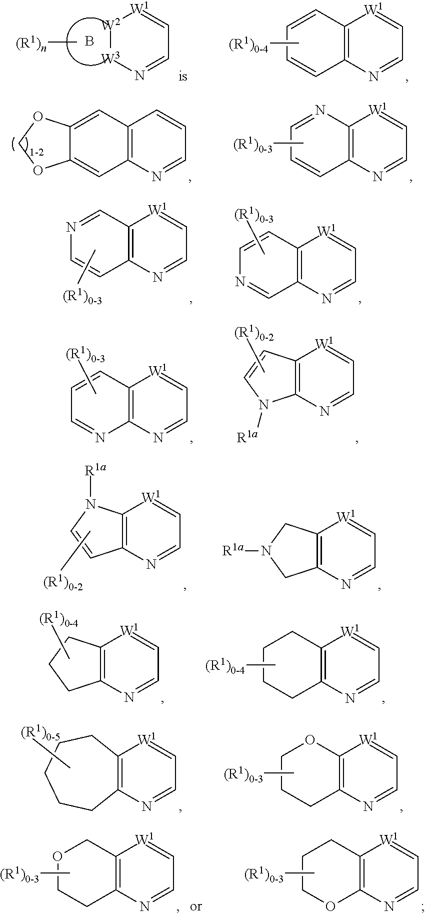

or a pharmaceutically acceptable salt thereof and/or an isomer thereof.

22. The compound of any one of claims 20 and 21, wherein R^{1a} is hydrogen, or a pharmaceutically acceptable salt thereof and/or an isomer thereof.

23. The compound of any one of claims 20 and 21, wherein R^{1a} is C₁-C₆-alkyl, or a pharmaceutically acceptable salt thereof and/or an isomer thereof.

24. The compound of any one of claims 20 and 21, wherein R^{1a} is C₃-C₆-cycloalkyl, or a pharmaceutically acceptable salt thereof and/or an isomer thereof.

25. The compound of any one of claims 20 and 21, wherein R^{1a} is C₃-C₆-cycloalkylC₁-C₃-alkyl, or a pharmaceutically acceptable salt thereof and/or an isomer thereof.

26. The compound of any one of claims 1-25, wherein one R¹ is present and is other than hydrogen, or a pharmaceutically acceptable salt thereof and/or an isomer thereof.

27. The compound of any one of claims 1-25, wherein two R¹ are present and each is independently other than hydrogen, or a pharmaceutically acceptable salt thereof and/or an isomer thereof.

28. The compound of any one of claims 1-25, wherein three R¹ are present and each is independently other than hydrogen, or a pharmaceutically acceptable salt thereof and/or an isomer thereof.

29. The compound of any one of claims 1-28, wherein at least one R¹ is C₁-C₆ alkyl, or a pharmaceutically acceptable salt thereof and/or an isomer thereof.

30. The compound of any one of claims 1-29, wherein at least one R¹ is —CH₃, or a pharmaceutically acceptable salt thereof and/or an isomer thereof.

31. The compound of any one of claims 1-28, wherein at least one R¹ is C₁-C₆ alkoxy, or a pharmaceutically acceptable salt thereof and/or an isomer thereof.

32. The compound of any one of claims 1-28 and 31, wherein at least one R¹ is —OCH₃, or a pharmaceutically acceptable salt thereof and/or an isomer thereof.

33. The compound of any one of claims 1-28, wherein at least one R¹ is C₁-C₆ haloalkyl, or a pharmaceutically acceptable salt thereof and/or an isomer thereof.

34. The compound of any one of claims 1-28 and 33, wherein at least one R¹ is —CF₃, or a pharmaceutically acceptable salt thereof and/or an isomer thereof.

35. The compound of any one of claims 1-28, wherein at least one R¹ is halogen, or a pharmaceutically acceptable salt thereof and/or an isomer thereof.

36. The compound of any one of claims 1-28 and 35, wherein at least one R¹ is —Cl, or a pharmaceutically acceptable salt thereof and/or an isomer thereof.

37. The compound of any one of claims 1-28 and 35, wherein at least one R¹ is —F, or a pharmaceutically acceptable salt thereof and/or an isomer thereof.

38. The compound of any one of claims 1-28, wherein at least one R¹ is halo-C₁-C₆ alkoxy, or a pharmaceutically acceptable salt thereof and/or an isomer thereof.

39. The compound of any one of claims 1-28 and 38, wherein at least one R¹ is —OCF₃, or a pharmaceutically acceptable salt thereof and/or an isomer thereof.

40. The compound of any one of claims 1-28, wherein each R¹ is independently selected from C₁-C₆ alkyl, C₁-C₆ alkoxy, halo-C₁-C₆ alkoxy, and halogen, or a pharmaceutically acceptable salt thereof and/or an isomer thereof.

41. The compound of any one of claims 1-25, wherein each R¹ is hydrogen, or a pharmaceutically acceptable salt thereof and/or an isomer thereof.

41a. The compound of any one of claims 1-28, wherein two R¹ are attached on adjacent ring carbons in

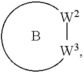

and together with the adjacent carbons to which they are attached form

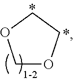

where * indicate the shared carbons in

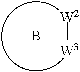

and any remaining R¹ are independently selected from hydrogen, C₁-C₆ alkyl, C₁-C₆ alkoxy, halogen, C₁-C₆ haloalkyl, halo-C₁-C₆ alkoxy, C₃-C₆-cycloalkyl, or C₃-C₆-cycloalkylC₁-C₃-alkyl.

42. The compound of any one of claims 1-41, wherein one R² is present and is other than hydrogen, or a pharmaceutically acceptable salt thereof and/or an isomer thereof.

43. The compound of any one of claims 1-41, wherein two R² are present and each is independently other than hydrogen, or a pharmaceutically acceptable salt thereof and/or an isomer thereof.

44. The compound of any one of claims 1-41, wherein three R² are present and each is independently other than hydrogen, or a pharmaceutically acceptable salt thereof and/or an isomer thereof.

45. The compound of any one of claims 1-44, wherein at least one R² is halogen, or a pharmaceutically acceptable salt thereof and/or an isomer thereof.

46. The compound of any one of claims 1-45, wherein at least one R² is —F, or a pharmaceutically acceptable salt thereof and/or an isomer thereof.

47. The compound of any one of claims 1-44, wherein at least one R² is C₁-C₆ alkyl, or a pharmaceutically acceptable salt thereof and/or an isomer thereof.

48. The compound of any one of claims 1-44 and 47, wherein at least one R² is —CH₃, or a pharmaceutically acceptable salt thereof and/or an isomer thereof.

49. The compound of any one of claims 1-41, 43, and 44, wherein two R² are attached on adjacent carbons and together with the carbons to which they are attached form a 4- to 7-membered carbocyclic ring and the remaining R², independently in each instance, is hydrogen, halogen, or C₁-C₆ alkyl, or a pharmaceutically acceptable salt thereof and/or an isomer thereof.

50. The compound of any one of claims 1-41, 43, and 44, wherein two R² are attached on the same carbon and together with the carbon to which they are attached form a 4- to 7-membered carbocyclic ring and the remaining R², independently in each instance, is hydrogen, halogen, or C₁-C₆ alkyl, or a pharmaceutically acceptable salt thereof and/or an isomer thereof.

51. The compound of any one of claims 1-44 wherein each R² is independently selected from C₁-C₆ alkyl and halogen, or a pharmaceutically acceptable salt thereof and/or an isomer thereof.

52. The compound of any one of claims 1-41, wherein each R² is hydrogen, or a pharmaceutically acceptable salt thereof and/or an isomer thereof.

53. The compound of any one of claims 1-52, wherein m is 1 or 2, or a pharmaceutically acceptable salt thereof and/or an isomer thereof.

54. The compound of any one of claims 1-53, wherein m is 1, or a pharmaceutically acceptable salt thereof and/or an isomer thereof.

55. The compound of any one of claims 1-53, wherein m is 2, or a pharmaceutically acceptable salt thereof and/or an isomer thereof.

56. The compound of any one of claims 1-55, wherein W¹ is —N═, or a pharmaceutically acceptable salt thereof and/or an isomer thereof.

57. The compound of any one of claims 1-55, wherein W¹ is —C(H)═, or a pharmaceutically acceptable salt thereof and/or an isomer thereof.

58. The compound of any one of claims 1-55, wherein W¹ is —C(halogen)=, or a pharmaceutically acceptable salt thereof and/or an isomer thereof.

59. The compound of any one of claims 1-55, wherein W¹ is —C(C₁-C₆ alkyl)=, or a pharmaceutically acceptable salt thereof and/or an isomer thereof.

60. The compound of any one of claims 1-55, wherein W¹ is —C(cyclopropyl)=, or a pharmaceutically acceptable salt thereof and/or an isomer thereof.

61. The Compound of claim 1, or a pharmaceutically acceptable salt thereof and/or an isomer thereof, selected from the group consisting of the compounds provided in Table A.

62. A pharmaceutical composition comprising the compound of any one of claims 1-61, or a pharmaceutically acceptable salt thereof and/or an isomer thereof, and a pharmaceutically acceptable carrier.

63. The pharmaceutical composition of claim 62, wherein the composition is an oral or injectable composition.

64. The pharmaceutical composition of claim 63, wherein the injectable composition is a subcutaneously injectable composition.

65. A method for the treatment of a condition associated with voltage-gated sodium channel function, including Naᵥ1.8, in a subject, comprising the administration of a therapeutically or prophylactically effective amount of the compound of any one of claims 1-61 or the pharmaceutical composition of any one of claims 62-64.

66. The method of claim 65, wherein the subject is a human.

67. The method of claim 65 or 66, wherein the condition is pain or wherein the condition is associated with pain.

68. The method of claim 65 or 66, wherein the condition is pain.

69. The method of claim 65 or 66, wherein the condition is associated with pain.

70. The method of any one of claims 65-69, wherein the condition is selected from the group consisting of pain associated with erythromelalgia, pain associated with diabetic peripheral neuropathy, paroxysmal extreme pain disorder, complex regional pain syndrome, pain associated with trigeminal neuralgia, pain associated with multiple sclerosis, pain associated with arthritis (including osteoarthritis), pain associated with postherpetic neuralgia, cancer pain, pain associated with cluster headache, pain associated with migraine, pain associated with sciatica, pain associated with endometriosis, pain associated with fibromyalgia, postsurgical pain, subacute pain, chronic pain, pain and/or discomfort associated with dry eye syndrome, pain associated with (acute) corneal injuries or abrasions, acute ocular pain, chronic ocular pain, pain associated with corneal infections, pain associated with Parkinson's disease, pain associated with ALS, pain associated with ocular surgery, pain associated with epilepsy, pain associated with Parkinson's disease, a mood disorder, psychosis, pain associated with amyotropic lateral sclerosis, glaucoma, ischemia, a spasticity disorder, and obsessive compulsive disorder.

71. The method of claim 65 or 66, wherein the condition is selected from the group consisting of acute pain, subacute pain, post-surgical pain, and ocular pain.

72. A method of inhibiting NAᵥ1.8 comprising contacting NAᵥ1.8 with a compound of any one of claims 1-61.

In some or any embodiments, provided herein are:
(a) compounds as described herein, e.g., of Formula (I) and Compounds 1-98 and pharmaceutically acceptable salts and compositions thereof;
(b) compounds as described herein, e.g., of Formula (I), and Compounds 1-98 and pharmaceutically acceptable salts and compositions thereof for use in the treatment of pain and/or conditions modulated by voltage-gated sodium channels;
(c) processes for the preparation of compounds as described herein, e.g., of Formula (I) and Compounds 1-98 as described in more detail elsewhere herein;
(d) pharmaceutical formulations comprising a compound as described herein, e.g., of Formula (I) and Compounds 1-98 or a pharmaceutically acceptable salt thereof together with a pharmaceutically acceptable carrier or diluent;
(e) a method for the treatment of a condition associated with voltage-gated sodium channel function in a subject that includes the administration of a therapeutically or prophylactically effective amount of a compound as described herein, e.g., of Formula (I) and Compounds 1-98 or its pharmaceutically acceptable salt or composition;
(f) a method for the treatment of pain in a subject that includes the administration of a therapeutically or prophylactically effective amount of a compound as described herein, e.g., of Formula (T) and Compounds 1-98 or its pharmaceutically acceptable salt or composition;
(g) pharmaceutical formulations comprising a compound as described herein, e.g., of Formula (I) and Compounds 1-98 or a pharmaceutically acceptable salt thereof together with one or more other effective agents for treating pain and/or conditions modulated by voltage-gated sodium channels, optionally in a pharmaceutically acceptable carrier or diluent;
(h) a method for the treatment of pain in a subject that includes the administration of a therapeutically or prophylactically effective amount of a compound as described herein, e.g., of Formula (I) and 1-Compounds 1-98 or its pharmaceutically acceptable salt or composition in combination and/or alternation with one or more agent for the treatment of pain and/or conditions modulated by voltage-gated sodium channels; and
(i) a method for the treatment of a condition associated with voltage-gated sodium channel function in a subject that includes the administration of a therapeutically or prophylactically effective amount of a compound as described herein, e.g., of Formula (I) and Compounds 1-98 or its pharmaceutically acceptable salt or composition in combination and/or alternation with one or more agent for the treatment of pain.
(j) use of any compound described herein, e.g., of Formula (I) and Compounds 1-98 or a composition comprising any compound described herein, e.g., of Formula (I) and Compounds 1-98 or a pharmaceutically acceptable salt or composition for the treatment of a condition associated with voltage-gated sodium channel function described herein (e.g., pain), optionally in combination and/or alternation with one or more agent for the treatment of pain.

### Optically Active Compounds

It is appreciated that compounds provided herein have several chiral centers and may exist in and be isolated in optically active and racemic forms. It is to be understood that any racemic, optically-active, diasteromeric, tautomeric, or stereoisomeric form, mixture, or combination thereof, of a compound provided herein, which possess the useful properties described herein is within the scope of the invention. It being well known in the art how to prepare optically active forms (in some or any embodiments, by resolution of the racemic form by recrystallization techniques, by synthesis from optically-active starting materials, by chiral synthesis, or by chromatographic separation using a chiral stationary phase).

In some or any embodiments, methods to obtain optically active materials are known in the art, and include at least the following.
i) physical separation of crystals—a technique whereby macroscopic crystals of the individual stereoisomers are manually separated. This technique can be used if crystals of the separate stereoisomers exist, i.e., the material is a conglomerate, and the crystals are visually distinct;
ii) simultaneous crystallization—a technique whereby the individual stereoisomers are separately crystallized from a solution of the racemate, possible only if the latter is a conglomerate in the solid state;
iii) enzymatic resolutions—a technique whereby partial or complete separation of a racemate by virtue of differing rates of reaction for the stereoisomers with an enzyme;
iv) enzymatic asymmetric synthesis—a synthetic technique whereby at least one step of the synthesis uses an enzymatic reaction to obtain an stereoisomerically pure or enriched synthetic precursor of the desired stereoisomer;
v) chemical asymmetric synthesis—a synthetic technique whereby the desired stereoisomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the stereoisomers with a chiral, non-racemic reagent or catalyst under kinetic conditions;
ix) stereospecific synthesis from non-racemic precursors—a synthetic technique whereby the desired stereoisomer is obtained from non-chiral starting materials and where the stereochemical integrity is not or is only minimally compromised over the course of the synthesis;
x) chiral liquid chromatography—a technique whereby the stereoisomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and stereoisomers are separated by virtue of their differing interactions in the gaseous mobile phase with a column containing a fixed non-racemic chiral adsorbent phase;
xii) extraction with chiral solvents—a technique whereby the stereoisomers are separated by virtue of preferential dissolution of one stereoisomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one stereoisomer of the racemate to pass through.

In some or any embodiments, provided is a composition of a compound that comprises a substantially pure designated stereoisomer of the compound. In some or any embodiments, in the methods and compounds, the compounds are substantially free of other stereoisomer. In some or any embodiments, a composition includes a compound that is at least 85%, 90%, 95%, 98%, 99% or 100% by weight, of the designated stereoisomer, the remainder comprising other chemical species or stereoisomers.

### Isotopically Enriched Compounds

Also provided herein are isotopically enriched compounds.

Isotopic enrichment (in some or any embodiments, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al, Mutation Res. 308: 33 (1994); Gordon et. al, Drug Metab. Dispos., 15: 589 (1987); Zello et. al, Metabolism, 43: 487 (1994); Gately et. al., J. Nucl. Med., 27: 388 (1986); Wade D, Chem. Biol. Interact. 117: 191 (1999).

Isotopic enrichment of a drug can be used, in some or any embodiments, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrees the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). See, e.g., Foster et alt, Adv. Drug Res., vol. 14, pp. 1-36 (1985); Kushner et al, Can. J. Physiol. Pharmacol., vol. 77, pp. 79-88 (1999).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity.

In some embodiments, the compounds described herein may be used as radiopharmaceuticals such as, for example, imaging agents. In one instance, radiopharmaceuticals are positron emission tomography (PET) imaging agents. In such embodiments, substitution of radionuclides (e.g., positron emitting isotopes) for atoms in the compounds allows for the syntheses of radiopharmaceuticals that can function as imaging agents. In some embodiments, radionuclides which can be substituted in the compounds described herein include, and are not limited to, ¹⁸F, ¹¹C, ¹³N, ¹⁵O, ⁷⁶Br, and ¹²⁴. In some embodiments, the compound is isotopically enriched at one or more atoms, one atom, two atoms, or three atoms. In some embodiments, the compound is administered as an isotopic composition.

The animal body expresses a variety of enzymes for the purpose of eliminating foreign substances, such as therapeutic agents, from its circulation system. In some or any embodiments, such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein will produce a detectable KTE that will affect the pharmacokinetic, pharmacologic, and/or toxicological profiles of a compound provided herein in comparison with a similar compound having a natural isotopic composition.

### Preparation of Compounds

The compounds provided herein can be prepared, isolated or obtained by any method apparent to those of skill in the art. Compounds provided herein can be prepared according to the Exemplary Preparation Schemes provided below. Reaction conditions, steps and reactants not provided in the Exemplary Preparation Schemes would be apparent to, and known by, those skilled in the art.

Additional steps and reagents not provided in the Exemplary Preparation Scheme would be known to those of skill in the art. For example, intermediates and compounds could be prepared using the procedures known by one of ordinary skill in the art or as disclosed in International Patent Application Nos. PCT/US2019/058251 and PCT/US2019/058999 (wherein the synthetic methods disclosed therein are herein incorporated by reference in their entirety). Exemplary methods of preparation are described in detail in the Examples herein.

In another embodiment, provided herein is a method of preparing a compound of Formula (I) according to any of the following schemes:

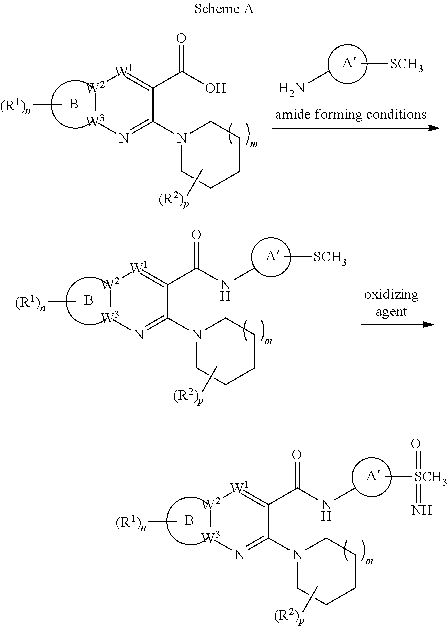

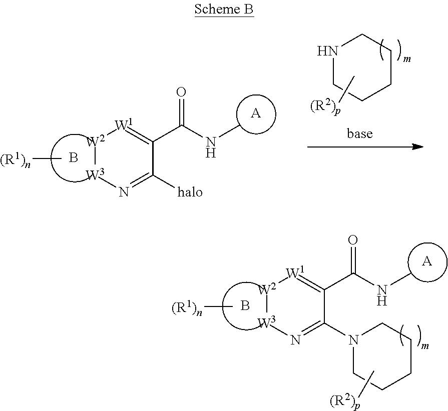

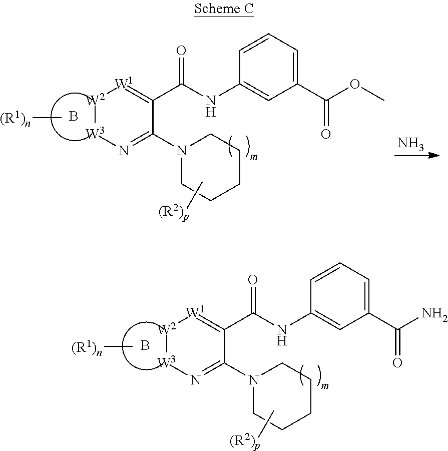

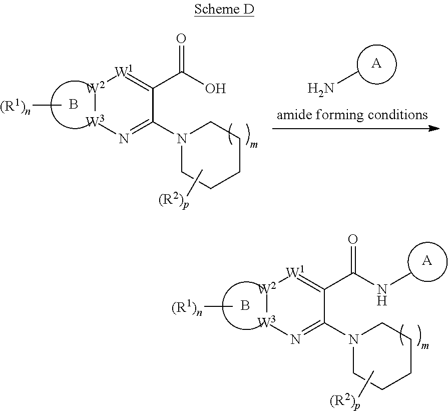

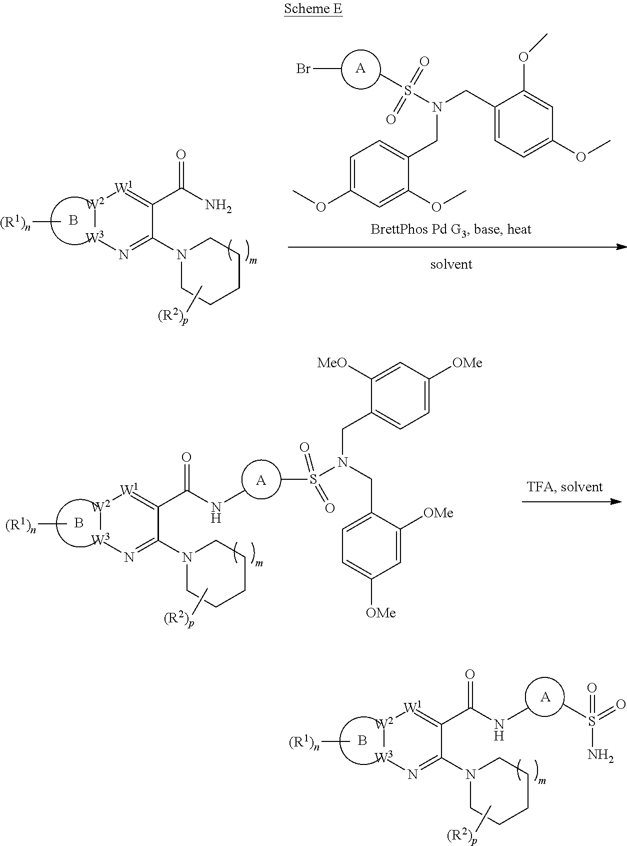

wherein
A is phenyl substituted with R³, pyridinyl substituted with R³, benzoisoxazolyl substituted with R³, unsubstituted pyrazolyl, pyrazolyl substituted with R³, or
W¹ is —N═ or —C(H)═;

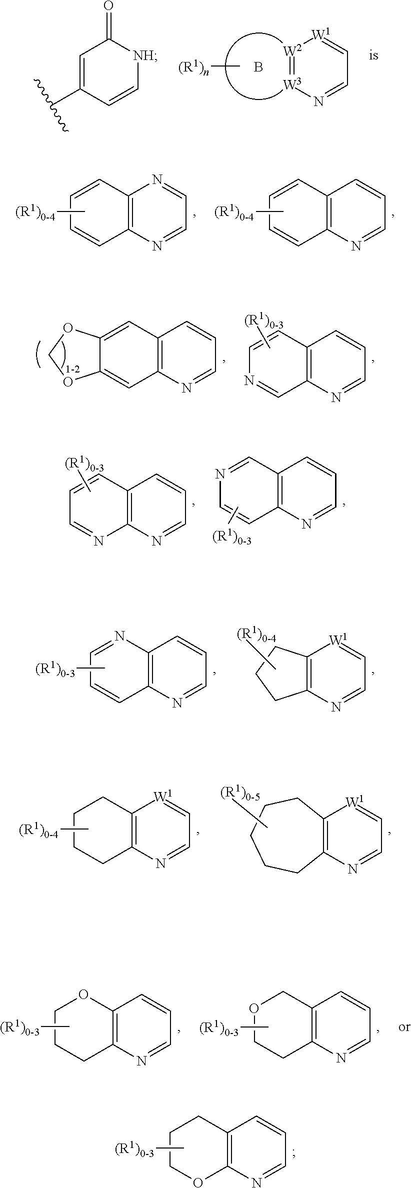

each R is independently hydrogen, C₁-C₆ alkyl, C₁-C₆ alkoxy, C₁-C₆ haloalkyl, halo-C₁-C₆ alkoxy, or halogen;
each R² is independently hydrogen, C₁-C₆ alkyl, or halo;
R³ is —C(O)NH₂, —NH₂, —S(O)₂NH₂, —S(O)₂CH₃, or

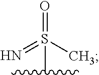 [SMILES: *S(C)(=N)=O]

m is 1 or 2;
n is 0, 1, or 2; and
p is 0, 1, 2, or 3;
   to yield a compound of Formula I; and
   b) optionally isolating the compound of Formula T.

One of skill will understand that the order of steps for any process described herein may be changed. Other variations will be apparent to one of skill in the art and all such variations are contemplated within the scope of embodiments presented herein.

### Pharmaceutical Compositions and Methods of Administration

The compounds provided herein can be formulated into pharmaceutical compositions using methods available in the art and those disclosed herein. Any of the compounds disclosed herein can be provided in the appropriate pharmaceutical composition and be administered by a suitable route of administration. Provided herein are pharmaceutical compositions comprising a compound of Formula (I), as described herein in some and any embodiments, and a pharmaceutically acceptable carrier. In some embodiments, the composition is an oral or injectable composition. In some of such embodiments, the injectable composition is a subcutaneously injectable composition.

The methods provided herein encompass administering pharmaceutical compositions containing at least one compound as described herein, including a compound of Formula (I) if appropriate in a salt form, either used alone or in the form of a combination with one or more compatible and pharmaceutically acceptable carriers, such as diluents or adjuvants, or with another agent for the treatment of pain and/or conditions modulated by voltage-gated sodium channels.

In some or any embodiments, the second agent can be formulated or packaged with the compound provided herein. Of course, the second agent will only be formulated with the compound provided herein when, according to the judgment of those of skill in the art, such co-formulation should not interfere with the activity of either agent or the method of administration. In some or any embodiments, the compound provided herein and the second agent are formulated separately. They can be packaged together, or packaged separately, for the convenience of the practitioner of skill in the art.

In clinical practice the active agents provided herein may be administered by any conventional route, in particular orally, parenterally, rectally or by inhalation (e.g. in the form of aerosols). In some or any embodiments, the compound provided herein is administered orally.

Use may be made, as solid compositions for oral administration, of tablets, pills, hard gelatin capsules, powders or granules. In these compositions, the active product is mixed with one or more inert diluents or adjuvants, such as sucrose, lactose or starch.

These compositions can comprise substances other than diluents, for example a lubricant, such as magnesium stearate, or a coating intended for controlled release.

Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, in some or any embodiments, wetting, sweetening or flavoring products.

The compositions for parenteral administration can be emulsions or sterile solutions. Use may be made, as solvent or vehicle, of propylene glycol, a polyethylene glycol, vegetable oils, in particular olive oil, or injectable organic esters, in some or any embodiments, ethyl oleate. These compositions can also contain adjuvants, in particular wetting, isotonizing, emulsifying, dispersing and stabilizing agents. Sterilization can be carried out in several ways, in some or any embodiments, using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in a pyrogenic sterile water, in saline or any other pharmaceutically acceptable vehicle. For use in the form of dry aerosols intended to be directly inhaled, the active principle is finely divided and combined with a water-soluble solid diluent or vehicle, in some or any embodiments, dextran, mannitol or lactose.

In some or any embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents (e.g., a compound provided herein, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Remington: The Science and Practice of Pharmacy; Pharmaceutical Press; 22 edition (Sep. 15, 2012).

Typical pharmaceutical compositions and dosage forms comprise one or more excipients. Suitable excipients are well-known to those skilled in the art of pharmacy, and in some or any embodiments, suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, in some or any embodiments, in the U.S. Pharmacopeia (USP 36-NF 31 S2). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, New York, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. In some or any embodiments, suitable packaging include, but are not limited to, hermetically scaled foils, plastics, unit dose containers (e.g., vials), blister packs, and strip packs.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in some or any embodiments, in purified form, together with a suitable amount of carrier so as to provide the form for proper administration to the subject. The formulation should suit the mode of administration. In a some or any embodiment, the pharmaceutical compositions or single unit dosage forms are sterile and in suitable form for administration to a subject, in some or any embodiments, an animal subject, such as a mammalian subject, in some or any embodiments, a human subject.

A pharmaceutical composition is formulated to be compatible with its intended route of administration. In some or any embodiments, routes of administration include, but are not limited to, parenteral, e.g., intrathecal, epidural, local or regional for peripheral nerve block, intravenous, intradermal, subcutaneous, intramuscular, subcutaneous, oral, buccal, sublingual, inhalation, intranasal, transdermal, topical (including administration to the eye, and in some embodiments to the cornea), transmucosal, intra-tumoral, intra-synovial, and rectal administration. In a specific embodiment, the composition is formulated in accordance with routine procedures as a pharmaceutical composition adapted for intravenous, subcutaneous, intramuscular, oral, intranasal, or topical (including administration to the eye, and in some embodiments to the cornea) administration to human beings. In a specific embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

In some or any embodiments, dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non-aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms provided herein will typically vary depending on their use. In some or any embodiments, a dosage form used in the initial treatment of pain may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms encompassed herein will vary from one another will be readily apparent to those skilled in the art. See, e.g., Remington: The Science and Practice of Pharmacy; Pharmaceutical Press; 22 edition (Sep. 15, 2012).

Generally, the ingredients of compositions are supplied either separately or mixed together in unit dosage form, in some or any embodiments, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms comprise a compound provided herein, or a pharmaceutically acceptable salt, solvate or hydrate thereof lie within the range of from about 0.1 mg to about 1000 mg per day, given as a single once-a-day dose in the morning or as divided doses throughout the day taken with food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active compound.

### Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington: The Science and Practice of Pharmacy; Pharmaceutical Press; 22 edition (Sep. 15, 2012).

In some or any embodiments, the oral dosage forms are solid and prepared under anhydrous conditions with anhydrous ingredients, as described in detail herein. However, the scope of the compositions provided herein extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. In some or any embodiments, excipients suitable for use in oral liquid or aerosol dosage forms include, but are not limited to, water, glycols, oils, alcohols, flavoring agents, preservatives, and coloring agents. In some or any embodiments, excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

In some or any embodiments, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

In some or any embodiments, excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

In some or any embodiments, fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions is typically present in from about 50 to about 99 weight percent of the pharmaceutical composition or dosage form.

In some or any embodiments, suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, PA), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, in some or any embodiments, a syloid silica gel (AEROSIL 200, manufactured by W.R. Grace Co. of Baltimore, MD), a coagulated aerosol of synthetic silica (marketed by Degussa Co. of Plano, TX), CAB O SIL (a pyrogenic silicon dioxide product sold by Cabot Co. of Boston, MA), and mixtures thereof. If used at all, lubricants are typically used in an amount of less than about 1 weight percent of the pharmaceutical compositions or dosage forms into which they are incorporated.

### Delayed Release Dosage Forms

Active ingredients such as the compounds provided herein can be administered by controlled release means or by delivery devices that are well known to those of ordinary skill in the art. In some or any embodiments, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113.943; 6,197,350; 6,248,363; 6,264,970; 6,267,981; 6,376,461; 6,419,961; 6,589,548; 6,613,358; and 6,699,500; each of which is incorporated herein by reference in its entirety. Such dosage forms can be used to provide slow or controlled release of one or more active ingredients using, in some or any embodiments, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Thus encompassed herein are unit dosage forms suitable for oral administration such as, but not limited to, tablets, capsules, gel caps, and caplets that are adapted for controlled release.

All controlled release pharmaceutical products have a common goal of improving drug therapy over that achieved by their non-controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In some or any embodiments, the drug may be administered using intravenous infusion, an implantable osmotic pump, a transdermal patch, liposomes, or other modes of administration. In some or any embodiments, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in a subject at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

### Parenteral Dosage Forms

In some or any embodiments, provided are parenteral dosage forms. Parenteral dosage forms can be administered to subjects by various routes including, but not limited to, subcutaneous, intravenous (including bolus injection), intramuscular, and intra-arterial. Because their administration typically bypasses subjects' natural defenses against contaminants, parenteral dosage forms are typically, sterile or capable of being sterilized prior to administration to a subject. In some or any embodiments, parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. In some or any embodiments, suitable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms.

### Transdermal, Topical & Mucosal Dosage Forms

Also provided are transdermal, topical, and mucosal dosage forms. Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington: The Science and Practice of Pharmacy; Pharmaceutical Press; 22 edition (Sep. 15, 2012); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are nontoxic and pharmaceutically acceptable. Moisturizers or humectants can also be added to pharmaceutical compositions and dosage forms if desired. Examples of such additional ingredients are well known in the art. See, e.g., Remington: The Science and Practice of Pharmacy; Pharmaceutical Press; 22 edition (Sep. 15, 2012).

Depending on the specific tissue to be treated, additional components may be used prior to, in conjunction with, or subsequent to treatment with active ingredients provided. In some or any embodiments, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

### Dosage and Unit Dosage Forms

In human therapeutics, the doctor will determine the posology which the doctor considers most appropriate according to a preventive or curative treatment and according to the age, weight, stage of the infection and other factors specific to the subject to be treated. In some or any embodiments, doses are from about 1 to about 1000 mg per day for an adult, or from about 5 to about 250 mg per day or from about 10 to 50 mg per day for an adult. In some or any embodiments, doses are from about 5 to about 400 mg per day or 25 to 200 mg per day per adult. In some or any embodiments, dose rates of from about 50 to about 500 mg per day are also contemplated. In some or any embodiments, doses for subcutaneous administration are from about 1 to about 50 mg per day, or from about 1 to about 25 mg per day, or from about 1 to about 10 mg per day, or from about 1 to about 20 mg per day, or from about 5 to about 25 mg per day, or from about 5 mg to about 20 mg per day, or from about 10 to about 20 mg per day. In some or any embodiments, doses for oral administration are from about 5 to about 250 mg per day, from about 5 to 200 mg per day, or from about 50 mg to about 100 mg per day, or from about 75 mg to about 1125 mg per day, or from about 10 mg to about 200 mg per day. In some embodiments, the mg/day amounts are for an adult. In further aspects, provided are methods of treating a condition associated with voltage-gated sodium channel function and/or pain in a subject by administering, to a subject in need thereof, a therapeutically or prophylactically effective amount of a compound provided herein, or a pharmaceutically acceptable salt thereof. The amount of the compound or composition which will be therapeutically or prophylactically effective in the treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In some or any embodiments, exemplary doses of a composition include milligram or microgram amounts of the active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in some or any embodiments, the dosage administered to a subject is 0.01 mg/kg to 3 mg/kg of the subject's body weight, or 0.10 mg/kg to 3 mg/kg of the subject's body weight, based on weight of the active compound. In some or any embodiments, the dosage administered to a subject is between 0.20 mg/kg and 2.00 mg/kg, or between 0.30 mg/kg and 1.50 mg/kg of the subject's body weight. In some embodiments, the dosage is administered subcutaneously to a subject and is between about 0.01 mg/kg to 1 mg/kg (inclusive), or between about 0.03 mg/kg to 0.5 mg/kg (inclusive) of the subject's body weight, based on weight of the active compound. In some embodiments, the dosage is administered orally to a subject and is between about 0.10 mg/kg to 5 mg/kg (inclusive) of the subject's body weight, or between about 0.10 mg/kg to 2 mg/kg (inclusive) of the subject's body weight, based on weight of the active compound.

In some or any embodiments, the recommended daily dose range of a composition provided herein for the conditions described herein lie within the range of from about 0.1 mg to about 1000 mg per day, given as a single once-a-day dose or as divided doses throughout a day. In some or any embodiments, the daily dose is administered twice daily in equally divided doses. In some or any embodiments, the daily dose is administered thrice daily in equally divided doses. In some or any embodiments, the daily dose is administered four times daily in equally divided doses. In some or any embodiments, a daily dose range should be from about 0.01 mg to about 400 mg per day, from about 0.1 mg to about 250 mg per day, from about 10 mg to about 200 mg per day, in other embodiments, or from about 10 mg and about 150 mg per day, in further embodiments, between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the herein described dosage amounts and dose frequency schedules. Further, when a subject is administered multiple dosages of a composition provided herein, not all of the dosages need be the same. In some or any embodiments, the dosage administered to the subject may be increased to improve the prophylactic or therapeutic effect of the composition or it may be decreased to reduce one or more side effects that a particular subject is experiencing.

In some or any embodiment, the dosage of the composition provided herein, based on weight of the active compound, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 201 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In some or any embodiments, treatment or prevention can be initiated with one or more loading doses of a compound or composition provided herein followed by one or more maintenance doses. In such embodiments, the loading dose can be, for instance, about 6 to about 40 mg per day, or about 10 to about 20 mg per day for one day to five weeks. The loading dose can be followed by one or more maintenance doses. In some or any embodiments, each maintenance does is, independently, about from about 1 mg to about 20 mg per day, between about 2.5 mg and about 15 mg per day, or between about 2.5 and about 8 mg per day. Maintenance doses can be administered daily and can be administered as single doses, or as divided doses.

In some or any embodiments, a dose of a compound or composition provided herein can be administered to achieve a steady-state concentration of the active ingredient in blood or serum of the subject. The steady-state concentration can be determined by measurement according to techniques available to those of skill or can be based on the physical characteristics of the subject such as height, weight and age. In some or any embodiments, a sufficient amount of a compound or composition provided herein is administered to achieve a steady-state concentration in blood or serum of the subject of from about 100 to about 1000 ng/mL, from about 150 to about 800 ng/mL, or from about 300 to about 600 ng/mL. In some or any embodiments, loading doses can be administered to achieve steady-state blood or serum concentrations of about 300 to about 2000 ng/mL, or about 400 to about 800 ng/mL for one to five days. In some or any embodiments, maintenance doses can be administered to achieve a steady-state concentration in blood or serum of the subject of from about 100 to about 1000 ng/mL, from about 150 to about 800 ng/mL, or from about 300 to about 600 ng/mL. In some or any embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In some or any embodiments, provided herein are unit dosages comprising a compound, or a pharmaceutically acceptable salt thereof, in a form suitable for administration. Such forms are described in detail herein. In some or any embodiments, the unit dosage comprises 1 to 1000 mg, 1 to 100 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art.

In some or any embodiments, dosages of the second agents to be used in a combination therapy are provided herein. In some or any embodiments, dosages lower than those which have been or are currently being used to treat pain are used in the combination therapies provided herein. The recommended dosages of second agents can be obtained from the knowledge of those of skill in the art. For those second agents that are approved for clinical use, recommended dosages are described in, for example, Hardman et al., eds., 1996, Goodman & Gilman's The Pharmacological Basis Of Therapeutics 9^{th} Ed, Mc-Graw-Hill, New York; Physician's Desk Reference (PDR) 57^{th} Ed., 2003, Medical Economics Co., Inc., Montvale, NJ; which are incorporated herein by reference in their entirety.

In various embodiments, the therapies (e.g., a compound provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. In various embodiments, the therapies are administered no more than 24 hours apart or no more than 48 hours apart. In some or any embodiments, two or more therapies are administered within the same patient visit. In other embodiments, the compound provided herein and the second agent are administered concurrently.

In other embodiments, the compound provided herein and the second agent are administered at about 2 to 4 days apart, at about 4 to 6 days apart, at about 1 week part, at about 1 to 2 weeks apart, or more than 2 weeks apart.

In some or any embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In some or any embodiments, a compound provided herein and a second agent are administered to a patient, in some or any embodiments, a subject, such as a human, in a sequence and within a time interval such that the compound provided herein can act together with the other agent to provide an increased benefit than if they were administered otherwise. In some or any embodiments, the second active agent can be administered at the same time or sequentially in any order at different points in time; however, if not administered at the same time, they should be administered sufficiently close in time so as to provide the desired therapeutic or prophylactic effect. In some or any embodiments, the compound provided herein and the second active agent exert their effect at times which overlap. Each second active agent can be administered separately, in any appropriate form and by any suitable route. In other embodiments, the compound provided herein is administered before, concurrently or after administration of the second active agent.

In some or any embodiments, the compound provided herein and the second agent are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some or any embodiments, the compound provided herein and the second active agent are administered in a cycle of less than about 3 weeks, about once every two weeks, about once every 10 days or about once every week. One cycle can comprise the administration of a compound provided herein and the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the second agent are administered separately yet within a time interval such that the compound provided herein can work together with the second active agent. In some or any embodiments, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the compound provided herein. In some or any embodiments, the compound provided herein is administered concurrently with one or more second agents in the same pharmaceutical composition. In another embodiment, a compound provided herein is administered concurrently with one or more second agents in separate pharmaceutical compositions. In still another embodiment, a compound provided herein is administered prior to or subsequent to administration of a second agent. Also contemplated are administration of a compound provided herein and a second agent by the same or different routes of administration, e.g., oral and parenteral. In some or any embodiments, when the compound provided herein is administered concurrently with a second agent that potentially produces adverse side effects including, but not limited to, toxicity, the second active agent can advantageously be administered at a dose that falls below the threshold that the adverse side effect is elicited.

### Kits

Also provided are kits for use in methods of treatment of pain and/or a condition associated with voltage-gated sodium channel function or a pain-related disorder. The kits can include a compound or composition provided herein, a second agent or composition, and instructions providing information to a health care provider regarding usage for treating the pain or a pain-related disorder. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 day. In some or any embodiments, a compound or composition can be included as a sterile aqueous pharmaceutical composition or dry powder (e.g., lyophilized) composition.

In some or any embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### Methods of Use

Provided herein is a method for treating a condition associated with voltage-gated sodium channel function and/or pain in a subject, which comprises contacting the subject with a therapeutically or prophylactically effective amount of a compound of Formula (I) including a single enantiomer, a mixture of an enantiomeric pair, an individual diastereomer, a mixture of diastereomers, an individual stereoisomer, a mixture of stereoisomers; or a pharmaceutically acceptable salt thereof.

Provided herein is a method for the treatment of a condition associated with voltage-gated sodium channel function in a subject, comprising the administration of a therapeutically or prophylactically effective amount of a compound of Formula (I) described herein or a pharmaceutical composition described herein.

In some embodiments, the subject is a human. In a group of embodiments, the condition is pain or the condition is associated with pain. In some embodiments, the condition is pain. In some embodiments, the condition is associated with pain. In some embodiments, the pain is nociceptive pain. In some embodiments, the pain is neuropathic pain. In some embodiments, the pain is inflammatory pain. In some embodiments, the pain is refractory to other forms of pain medications.

In a group of embodiments, the condition is selected from the group consisting of erythromelalgia, diabetic peripheral neuropathy, paroxysmal extreme pain disorder, complex regional pain syndrome, trigeminal neuralgia, multiple sclerosis, osteoarthritis, postherpetic neuralgia, cancer pain, cluster headache, migraine, sciatica, endometriosis, fibromyalgia, and postsurgical pain. In a group of embodiments, the condition is selected from the group consisting of epilepsy, Parkinson's disease, a mood disorder, psychosis, amyotropic lateral sclerosis, glaucoma, ischemia, a spasticity disorder, and obsessive compulsive disorder.

In some or any embodiments, provided herein are methods for treating pain and/or a condition associated with voltage-gated sodium channel function in a subject. In some or any embodiments, the methods encompass the step of administering to the subject in need thereof an amount of a compound effective for the treatment pain and/or a condition associated with voltage-gated sodium channel function in combination with a second agent effective for the treatment or prevention of pain and/or a condition associated with voltage-gated sodium channel function. The compound can be any compound as described herein, and the second agent can be any second agent described in the art or herein. In some or any embodiments, the compound is in the form of a pharmaceutical composition or dosage form, as described elsewhere herein.

In some or any embodiments, provided herein are methods for treating a condition associated with voltage-gated sodium channel function in a subject. In some or any embodiments, the methods encompass the step of administering to the subject in need thereof a therapeutically or prophylactically effective amount of a compound effective for the treatment of a condition associated with voltage-gated sodium channel function in combination with a second agent effective for the treatment of a condition associated with voltage-gated sodium channel function. The compound can be any compound as described herein, and the second agent can be any second agent described in the art or herein. In some or any embodiments, the compound is in the form of a pharmaceutical composition or dosage form, as described elsewhere herein.

In some or any embodiments, provided herein is of inhibiting NAᵥ1.8 comprising contacting NAᵥ1.8 with a compound of Formula (I) and Compounds 1-97.

In some or any embodiments, the pain to be reduced, ameliorated, treated, or prevented is associated with a condition or is a condition selected from acute pain, anal fissures, back pain, chronic pain, dental pain, joint pain, neck pain, neuropathic pain, obstetric pain, post-herpetic neuralgia, shingles, tension headaches, trigeminal blepharospasm, pain associated with cardiac arrythmia, focal dystonia, hyperhidrosis, muscle spasms, urinary bladder relaxation, visceral pain, sympathetically maintained pain, myositis pain, musculoskeletal pain, lower back pain, pain from sprains and strains, pain associated with functional bowel disorders, non-cardiac chest pain, pain associated with irritable bowel syndrome, pain associated with myocardial ischemia, toothache pain, pain from dysmenorrhea, erythromelalgia, diabetic peripheral neuropathy, paroxysmal extreme pain disorder, complex regional pain syndrome, trigeminal neuralgia, multiple sclerosis, osteoarthritis, postherpetic neuralgia, cancer, cluster headache, migraine, sciatica, endometriosis, fibromyalgia, dry eye syndrome, (acute) corneal injures or abrasions, conical infections, pain associated with Parkinson's disease, pain associated with ALS, and surgery (in some embodiments, post-surgery; in some embodiments, ocular surgery). In some or any embodiments, the pain to be reduced, ameliorated, treated, or prevented is pain in an acute care setting, including post-surgery. In some or any embodiments, the pain to be reduced, ameliorated, treated, or prevented is pain in an acute care setting, including post-surgery and the compound is administered intraveneously. In some or any embodiments, the pain to be reduced, ameliorated, treated, or prevented is ocular pain. In some or any embodiments, the pain to be reduced, ameliorated, treated, or prevented is ocular pain and the compound is administered topically. In some or any embodiments, the pain to be reduced, ameliorated, treated, or prevented is subacute or chronic pain. In some or any embodiments, the pain to be reduced, ameliorated, treated, or prevented is subacute or chronic pain and the compound is administered subcutaneously. In some or any embodiments, the pain to be reduced, ameliorated, treated, or prevented is subacute or chronic pain and the compound is administered orally.

In some or any embodiments, the condition associated with voltage-gated sodium channel function is selected from itch, cough, epilepsy, Parkinson's disease, a mood disorder, psychosis, amyotrophic lateral sclerosis (ALS), cardiac arrhythmia, glaucoma, ischemia, a spasticity disorder, and obsessive compulsive disorder. In some or any embodiments, the condition associated with voltage-gated sodium channel function is selected from pain, itch, cough, glaucoma, and ischemia. In some or any embodiments, the condition associated with voltage-gated sodium channel function is selected from pain, itch, and cough. In some or any embodiments, the condition associated with voltage-gated sodium channel function is pain.

In some or any embodiments, the compounds described herein are used for delaying the onset of pain, or reducing the severity or duration of pain. In some or any embodiments, the compounds described herein are used for the reduction of the severity or duration of pain associated with voltage-gated sodium channel function. In some embodiments, the compounds described herein are used for delaying or preventing onset of pain.

In some or any embodiments, the compounds described herein are used for prevention of pain or of a condition associated with voltage-gated sodium channel function.

In some or any embodiments, the compounds described herein are used for treatment of pain or of a condition associated with voltage-gated sodium channel function.

### Assay Methods

Compounds can be assayed for efficacy in treating pain and/or a condition associated with voltage-gated sodium channel function according to any assay known to those of skill in the art. Exemplary assay methods are provided elsewhere herein.

### Second Therapeutic Agents

In some or any embodiments, the compounds and compositions provided herein are useful in methods of treatment of pain and/or a condition associated with voltage-gated sodium channel function, that comprise further administration of a second agent effective for the treatment of pain and/or a pain-related disorder and/or a condition associated with voltage-gated sodium channel function. The second agent can be any agent known to those of skill in the art to be effective for the treatment of pain and/or a pain-related disorder and/or a condition associated with voltage-gated sodium channel function, including those currently approved by the United States Food and Drug Administration, or other similar body of a country foreign to the United States. In some or any embodiments, the second agent is a local anesthetic (in some or any embodiments, a steroid), an opioid, a vasoconstrictor, a glucocorticoid, adrenergic drugs (in some or any embodiments, alpha agonists or mixed central-peripheral alpha-2-agonists), vanilloids, an anti-inflammatory agent (e.g. NSAID, or an anti-inflammatory agent associated with ocular conditions, including cyclosporine and lifitegrast) or a chemical permeation enhancer. In some embodiments, the second agent is an inhibitor of Na_{V} 1.8. In some or any embodiments, chemical permeation enhancers include anionic surfactants, cationic surfactants, nonionic surfactants. In some or any embodiments, the second agent is bupivacaine, levobupivicaine, tetracaine, ropivacaine, epinephrine, phenylephrine, clonidine, sodium lauryl sulfate, sodium octyl sulfate, dodecyltrimethylammonium bromide, octyltrimethylammonium bromide, polyoxyethylene (20) sorbitan monolaurate, and/or polyoxyethylene (20) sorbitan monooleate.

In some or any embodiments, a compound provided herein is administered in combination with one second agent. In further embodiments, a compound provided herein is administered in combination with two second agents. In still further embodiments, a compound provided herein is administered in combination with two or more second agents.

As used herein, the term “in combination” includes the use of more than one therapy (e.g., one or more prophylactic and/or therapeutic agents). The use of the term “in combination” does not restrict the order in which therapies (e.g., prophylactic and/or therapeutic agents) are administered to a subject with a disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disorder.

As used herein, the term “synergistic” includes a combination of a compound provided herein and another therapy (e.g., a prophylactic or therapeutic agent) which has been or is currently being used to prevent, manage or treat a disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The active compounds provided herein can be administered in combination or alternation with another therapeutic agent, in particular an agent effective in the treatment of pain and/or a pain-related disorder and/or a condition associated with voltage-gated sodium channel function. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the pain or a pain-related disorder to be alleviated. It is to be further understood that for any particular subject, specific dosage regimens and schedules should be adjusted over time according to the individual need and the professional judgment of the person administering or supervising the administration of the compositions.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); M (micromolar); Hz (Hertz); MHz (megahertz); mmol (millimoles); hr or hrs (hours); min (minutes); MS (mass spectrometry); ESI (electrospray ionization); Ph (phenyl); TLC (thin layer chromatography); HPLC (high pressure liquid chromatography); THF (tetrahydrofuran); CDCl₃ (deuterated chloroform); AcOH (acetic acid); DCM (dichloromethane); DMSO (dimethylsulfoxide); DMSO-d₆ (deuterated dimethylsulfoxide); EtOAc (ethyl acetate); MeOH (methanol); Tces (2,2,2-trichloroethoxysulfonyl); —Si(tert-Bu)(Ph)₂ and —Si^{t}BuPh₂ (tert-butyl-diphenylsilyl); and BOC (t-butyloxycarbonyl). AZADO refers to

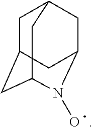

CDI refers to

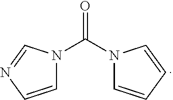 [SMILES: O=C(n1cccc1)n1ccnc1]

For all of the following examples, standard work-up and purification methods known to those skilled in the art can be utilized. Unless otherwise indicated, all temperatures are expressed in ° C. (degrees Celsius). All reactions are conducted at room temperature unless otherwise noted. Synthetic methodologies illustrated herein are intended to exemplify the applicable chemistry through the use of specific examples and are not indicative of the scope of the disclosure.

### SYNTHETIC EXAMPLES

### Compound 1

### Synthesis of 2-(4,4-difluoroazepan-1-yl)-N-(3-(S-methylsulfonimidoyl)phenyl)quinoline-3-carboxamide

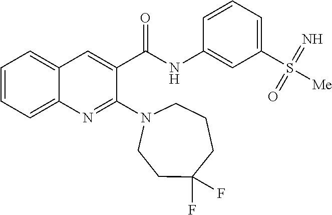 [SMILES: CS(=N)(=O)c1cccc(NC(=O)c2cc3ccccc3nc2N2CCCC(F)(F)CC2)c1]

### Synthesis of 2-(4,4-difluoroazepan-1-yl)quinoline-3-carboxylic acid

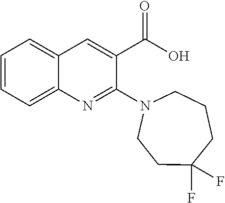 [SMILES: O=C(O)c1cc2ccccc2nc1N1CCCC(F)(F)CC1]

To a mixture of 2-chloroquinoline-3-carboxylic acid (104 mg, 0.5 mmol) and 4,4-difluoroazepane hydrogen chloride (85.5 mg, 0.5 mmol) in N,N-dimethylformamide (5 mL), potassium carbonate (345 mg, 2.5 mmol) was added at room temperature. The reaction mixture was stirred at 60° C. for 16 hours. The progress of the reaction was monitored by TLC. After completion, the reaction mixture was diluted with ice cold water and extracted with ethyl acetate. The organic layer was dried over anhydrous sodium sulphate and filtered. The filtrate was concentrated under reduced pressure and the crude residue was purified by silica gel column chromatography with a gradient of 0-100% ethyl acetate in hexanes to afford 2-(4,4-difluoroazepan-1-yl)quinoline-3-carboxylic acid as a yellow oil.

### Synthesis of 2-(4,4-difluoroazepan-1-yl)-N-(3-(methylthio)phenyl)quinoline-3-carboxamide

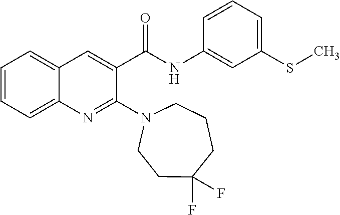 [SMILES: CSc1cccc(NC(=O)c2cc3ccccc3nc2N2CCCC(F)(F)CC2)c1]

### Synthesis of 2-(4,4-difluoroazepan-1-yl)-N-(3-(S-methylsulfonimidoyl)phenyl)quinoline-3-carboxamide

To a stirred solution of 2-(4,4-difluoroazepan-1-yl)quinoline-3-carboxylic acid (18.5 mg, 0.06 mmol) in dichloromethane (2 mL) was added (1-[Bis(dimethylamino)methylene]-1H-1,2,3-triazolo[4,5-b]pyridinium 3-oxide hexafluorophosphate (HATU) (0.344 mg, 0.09 mmol) and 4-dimethylaminopyridine (DMAP) (22.1 mg. 0.18 mmol). The reaction mixture was stirred at room temperature for 15 minutes. Then, 3-(methylthio)aniline (7.4 uL, 0.06 mmol) was added to the reaction mixture and stirred at room temperature for 18 hours. The progress of the reaction was monitored by LCMS. After the completion of reaction, dichloromethane was removed in vacuo and the resulting mixture was diluted with water and acetonitrile and purified using reversed-phase HPLC with a gradient of 5-95% acetonitrile in water to afford 2-(4,4-difluoroazepan-1-yl)-N-(3-(methylthio)phenyl)quinoline-3-carboxamide was a white powder.

### Synthesis of 2-(4,4-difluoroazepan-1-yl)-N-(3-(S-methylsulfonimidoyl)phenyl)quinoline-3-carboxamide

To a stirred solution of 2-(4,4-difluoroazepan-1-yl)-N-(3-(methylthio)phenyl)quinoline-3-carboxamide (25.8 mg, 0.056 mmol) in MeOH (3 mL) was added ammonium carbonate (10.7 mg, 0.11 mmol) then (diacetoxyiodo)benzene (44.8 mg, 0.14 mmol). The reaction mixture was stirred at room temperature for 90 minutes. The progress of the reaction was monitored by LCMS. Methanol was removed in vacuo and the resulting crude mass was diluted with water and acetonitrile and purified using reversed-phase HPLC with a gradient of 5-95% acetonitrile in water to afford 2-(4,4-difluoroazepan-1-yl)-N-(3-(S-methylsulfonimidoyl)phenyl)quinoline-3-carboxamide as a white powder. LRMS (ESI): Calcd [M+H]⁺: 459.17, found: 458.8.

¹H NMR (400 MHz, CD₃CN) δ 8.52 (s, 1H), 8.43 (s, 1H), 7.96 (dd, J=8.0, 1.8 Hz, 1H), 7.93-7.85 (m, 2H), 7.82-7.75 (m, 2H), 7.66 (t, J=8.0 Hz, 1H), 7.52-7.45 (m, 1H), 3.91-3.83 (m, 2H), 3.73 (t, J=5.8 Hz, 2H), 3.23 (s, 3H), 2.17-2.07 (m, 2H), 2.03-1.97 (m, 2H).

### Compound 2

### Synthesis of 2-(4,4-difluoroazepan-1-yl)-N-(3-sulfamoylphenyl)quinoline-3-carboxamide

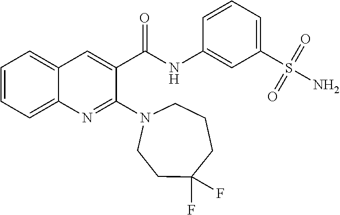 [SMILES: NS(=O)(=O)c1cccc(NC(=O)c2cc3ccccc3nc2N2CCCC(F)(F)CC2)c1]

### Synthesis of 2-chloro-N-(3-sulfamoylphenyl)quinoline-3-carboxamide

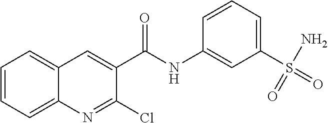 [SMILES: NS(=O)(=O)c1cccc(NC(=O)c2cc3ccccc3nc2Cl)c1]

To a mixture of 2-chloroquinoline-3-carboxylic acid (52 mg, 0.25 nmol) and oxalyl chloride (39.4 μL, 0.45 mmol) in dichloromethane (2 mL) was added one drop of N,N-dimethylformamide. The reaction mixture was stirred for 15 minutes at room temperature. Then, a solution of 3-aminobenzenesulfonamide (43 mg, 0.25 mmol) and diisopropylethylamine (131 μL, 0.75 mmol) in dichloromethane (2 mL) and N,N-dimethylformamide (0.5 mL) was added to the stirred solution. The reaction mixture was stirred for 1 hour at room temperature. Dichloromethane was removed in vacuo and the crude mixture was purified by silica gel column chromatography with a gradient of ethyl acetate and hexanes to afford 2-chloro-N-(3-sulfamoylphenyl)quinoline-3-carboxamide as a yellow oil.

### Synthesis of 2-(4,4-difluoroazepan-1-yl)-N-(3-sulfamoylphenyl)quinoline-3-carboxamide

To a solution of 2-chloro-N-(3-sulfamoylphenyl)quinoline-3-carboxamide (14 mg, 0.04 mmol) and 4,4-difluoroazepane hydrogen chloride (7 mg, 0.04 mmol) in N,N-dimethylformamide (1 mL), potassium carbonate (22 mg, 0.16 mmol) was added at room temperature. The reaction mixture was stirred at 70° C. for 16 hours then 80° C. for 18 hours. The progress of the reaction was monitored by TLC and LCMS. After completion, the reaction mixture was diluted with water and hydrochloric acid 1 N then extracted with dichloromethane. The organic layer was dried over anhydrous sodium sulphate and filtered. The filtrate was concentrated under reduced pressure and the crude residue was purified by reverse phase HPLC to afford 2-(4,4-difluoroazepan-1-yl)-N-(3-sulfamoylphenyl)quinoline-3-carboxamide as white solid. LRMS (ESI): Calcd [M+H]⁺: 461.15, found: 460.9.

¹H NMR (400 MHz, CD₃CN) δ 8.44 (s, 1H), 8.38-8.32 (m, 1H), 7.92-7.87 (m, 1H), 7.83-7.80 (m, 2H), 7.69-7.63 (m, 1H), 7.57 (t, J=8.0 Hz, 1H), 7.44-7.37 (m, 1H), 7.00-6.93 (m, 1H), 3.87-3.79 (m, 2H), 3.72-3.65 (m, 1H), 2.49-2.40 (m, 4H), 2.15-2.06 (m, 2H).

### Compound 3

### Synthesis of N-(3-aminobenzo[d]isoxazol-S-yl)-2-(4,4-difluoroazepan-1-yl)quinoline-3-carboxamide

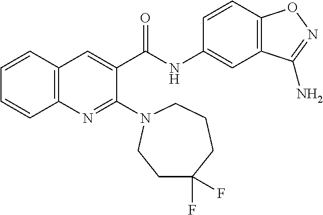 [SMILES: Nc1noc2ccc(NC(=O)c3cc4ccccc4nc3N3CCCC(F)(F)CC3)cc12]

### Synthesis of tert-butyl (5-(2-(4,4-difluoroazepan-1-yl)quinoline-3-carboxamido)benzo[d]isoxazol-3-yl)carbamate

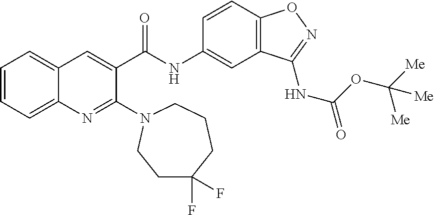 [SMILES: CC(C)(C)OC(=O)Nc1noc2ccc(NC(=O)c3cc4ccccc4nc3N3CCCC(F)(F)CC3)cc12]

To a mixture of 2-chloroquinoline-3-carboxylic acid (15 mg, 0.05 mmol) and oxalyl chloride (8 μL, 0.09 mmol) in dichloromethane (1 mL) was added one drop of N,N-dimethylformamide. The reaction mixture was stirred for 15 minutes at room temperature. Then, a solution of tert-butyl (5-aminobenzo[d]isoxazol-3-yl)carbamate (14 mg, 0.05 mmol) and diisopropylethylamine (26 μL, 0.15 mmol) in dichloromethane (1 mL) was added to the stirred solution. The reaction mixture was stirred for 18 hour at room temperature. Dichloromethane was removed in vacuo and the crude mixture was purified by reverse phase HPLC to afford tert-butyl (5-aminobenzo[d]isoxazol-3-yl)carbamate as white solid.

### Synthesis of N-(3-aminobenzo[d]isoxazol-5-yl)-2-(4,4-difluoroazepan-1-yl)quinoline-3-carboxamide

To a solution of tert-butyl (5-(2-(4,4-difluoroazepan-1-yl)quinoline-3-carboxamido)benzo[d]isoxazol-3-yl)carbamate (26 mg, 0.05 mmol) in dichloromethane (5 mL) was added trifluoroacetic acid (300 μL). The reaction mixture was stirred for 2 hours then the volatiles were removed in vacuo. The crude mixture was purified with Combiflash using a gradient of 0 to 100% ethyl acetate in hexane to afford tert-butyl (5-(2-(4,4-difluoroazepan-1-yl)quinoline-3-carboxamido)benzo[d]isoxazol-3-yl)carbamate as a light yellow oil. LRMS (ESI): Calcd [M+H]⁺: 438.17, found: 437.9.

¹H NMR (400 MHz, CD₃CN) δ 8.31 (s, 1H), 8.29-8.26 (m, 1H), 7.78 (d, J=8.1 Hz, 1H), 7.72-7.57 (m, 3H), 7.41 (d, J=9.0 Hz, TH), 7.37-7.28 (m, 1H), 3.84-3.76 (m, 2H), 3.65 (t, J=6.0 Hz, 2H), 2.51-2.37 (m, 2H), 2.34-2.31 (m, 2H), 2.13-2.02 (m, 2H).

### Compound 4

### Synthesis of N-(3-carbamoylphenyl)-2-(4,4-difluoroazepan-1-yl)quinoline-3-carboxamide

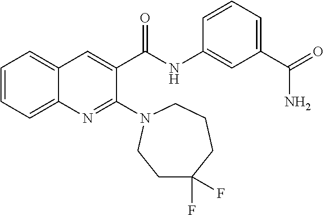 [SMILES: NC(=O)c1cccc(NC(=O)c2cc3ccccc3nc2N2CCCC(F)(F)CC2)c1]

### Synthesis of methyl 3-(2-(4,4-difluoroazepan-1-yl)quinoline-3-carboxamido)benzoate

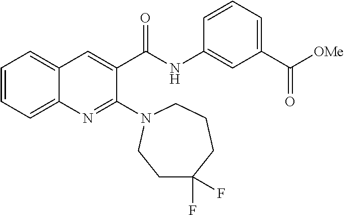 [SMILES: COC(=O)c1cccc(NC(=O)c2cc3ccccc3nc2N2CCCC(F)(F)CC2)c1]

To a mixture of 2-chloroquinoline-3-carboxylic acid (15 mg, 0.05 nmol) and oxalyl chloride (8 μL, 0.09 mmol) in dichloromethane (1 mL) was added one drop of N,N-dimethylformamide. The reaction mixture was stirred for 15 minutes at room temperature. Then, a solution of 3-aminobenzamide (8 mg, 0.05 mmol) and diisopropylethylamine (26 μL, 0.15 mmol) in dichloromethane (1 mL) was added to the stirred solution. The reaction mixture was stirred for 18 hour at room temperature. Dichloromethane was removed in vacuo and the crude mixture was purified by reverse phase HPLC to afford methyl 3-(2-(4,4-difluoroazepan-1-yl)quinoline-3-carboxamido)benzoate as white solid.

### Synthesis of N-(3-carbamoylphenyl)-2-(4,4-difluoroazepan-1-yl)quinoline-3-carboxamide

Methyl 3-(2-(4,4-difluoroazepan-1-yl)quinoline-3-carboxamido)benzoate (19 mg, 4.3 mmol) was mixed with 7 N ammonia in methanol (5 mL) in a sealed vial at 50° C. for 18 hours. The volatiles were removed in vacuo and the crude mixture was purified by reverse phase HPLC to afford N-(3-carbamoylphenyl)-2-(4,4-difluoroazepan-1-yl)quinoline-3-carboxamide as white solid. LRMS (ESI): Calcd [M+H]⁺: 425.18, found: 424.9.

¹H NMR (400 MHz, CDCl₃) δ 8.17 (s, 1H), 7.88-7.78 (m, 2H), 7.65-7.57 (m, 2H), 7.54-7.46 (m, 2H), 7.33 (t, J=7.9 Hz, 1H), 7.19 (t, J=7.5 Hz, 1H), 3.73-3.64 (m, 2H), 3.56-3.47 (m, 2H), 2.39-2.26 (m, 2H), 2.00-1.86 (m, 2H), 1.86-1.77 (m, 2H).

### Compound 5

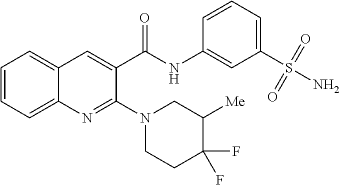 [SMILES: CC1CN(c2nc3ccccc3cc2C(=O)Nc2cccc(S(N)(=O)=O)c2)CCC1(F)F]

### Synthesis of 2-(4,4-difluoro-3-methylpiperidin-1-yl)-N-(3-sulfamoylphenyl)quinoline-3-carboxamide

To a solution of 2-chloro-N-(3-sulfamoylphenyl)quinoline-3-carboxamide (14 mg, 0.04 mmol) and 4,4-difluoro-3-methylpiperidine hydrogen chloride (7 mg, 0.04 mmol) in N,N-dimethylformamide (1 mL), potassium carbonate (22 mg, 0.16 mmol) was added at room temperature. The reaction mixture was stirred at 70° C. for 16 hours then 80° C. for 18 hours. The progress of the reaction was monitored by TLC and LCMS. After completion, the reaction mixture was diluted with water and hydrochloric acid 1 N then extracted with dichloromethane. The organic layer was dried over anhydrous sodium sulphate and filtered. The filtrate was concentrated under reduced pressure and the crude residue was purified by reverse phase HPLC to afford 2-(4,4-difluoro-3-methylpiperidin-1-yl)-N-(3-sulfamoylphenyl)quinoline-3-carboxamide as white solid. LRMS (ESI): Calcd [M+H]⁺: 461.15, found: 460.9.

¹H NMR (400 MHz, CDCl₃) δ 10.70 (s, 1H), 8.85 (s, 1H), 8.49-8.40 (m, 1H), 7.93 (d, J=8.4 Hz, 1H), 7.87 (d, J=8.0 Hz, 1H), 7.81-7.70 (m, 3H), 7.58-7.48 (m, 2H), 3.78-3.61 (m, 2H), 3.46-3.33 (m, 1H), 3.19-3.09 (m, 1H), 2.40-2.33 (m, 1H), 2.29-2.17 (m, 2H), 1.09 (d, J=6.8 Hz, 3H).

### Compound 6

### Synthesis of 3-(4,4-difluoroazepan-1-yl)-N-(3-methanesulfonylphenyl)quinoxaline-2-carboxamide

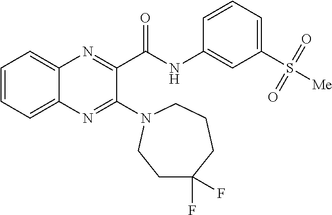 [SMILES: CS(=O)(=O)c1cccc(NC(=O)c2nc3ccccc3nc2N2CCCC(F)(F)CC2)c1]

To a solution of 3-(4,4-difluoroazepan-1-yl)quinoxaline-2-carboxylic acid (0.2 g, 0.65 mmol) in N,N-dimethylformamide (5 mL) was added (1-[Bis(dimethylamino)methylene]-1H-1,2,3-triazolo[4,5-b]pyridinium 3-oxide hexafluorophosphate (0.37 g, 0.98 mmol) and N,N-diisopropylethylamine (0.57 mL, 3.25 mmol) at 0° C. and the resulting mixture was stirred at the same temperature for 15 minutes. Afterwards, 3-methanesulfonylaniline (0.13 g, 0.78 mmol) was added and the reaction mixture was stirred for 12 hours at room temperature. Progress of the reaction was monitored by TLC. After completion of reaction, the reaction mixture was diluted with water and extracted with ethyl acetate. The organic layer was washed with brine, dried over sodium sulphate and evaporated under vacuum to obtain the crude product. The crude product was purified by flash column chromatography with a gradient of 40-50% ethyl acetate in hexanes to afford 3-(4,4-difluoroazepan-1-yl)-N-(3-methanesulfonylphenyl)quinoxaline-2-carboxamide as a yellow solid. MS (ESI): Calcd [M+H]⁺: 461.15, found: 461.20.

¹H NMR (400 MHz, DMSO-d₆): δ 11.40 (s, 1H), 8.39 (s, 1H), 8.08-8.06 (m, 1H), 7.95-7.93 (d, J=8 Hz, 1H), 7.72-7.67 (m, 4H), 7.52-7.48 (m, 1H), 3.80-3.78 (m, 2H), 3.67-3.64 (m, 2H), 3.24 (s, 3H), 2.55-2.32 (m, 2H), 2.04-2.02 (m, 2H), 1.93-1.92 (m, 2H).

### Compound 7

### 3-(4,4-difluoroazepan-1-yl)-N-(3-(S-methylsulfonimidoyl)phenyl)quinoxaline-2-carboxamide

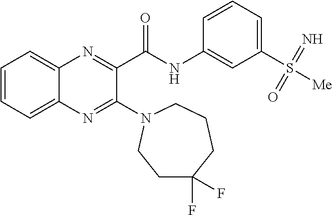 [SMILES: CS(=N)(=O)c1cccc(NC(=O)c2nc3ccccc3nc2N2CCCC(F)(F)CC2)c1]

To a solution of 3-(4,4-difluoroazepan-1-yl)quinoxaline-2-carboxylic acid (0.35 g, 1.15 mmol) in N,N-dimethylformamide (5 mL) was added N,N-diisopropylethyl amine (0.77 g, 5.7 mmol) and (1-[Bis(dimethylamino)methylene]-1H-1,2,3-triazolo[4,5-b]pyridinium 3-oxide hexafluoro phosphate (0.65 g, 1.7 mmol) at 0° C. and stirred for 15 min at room temperature. Afterwards, (3-aminophenyl)(imino)methyl-λ [symbol] -sulfanone (0.23 g, 1.4 mmol) was added and reaction mixture was stirred at room temperature for 12 hours. The progress of reaction was monitored by TLC and LCMS. After completion of reaction, the reaction mixture was diluted with water and extracted with ethyl acetate. The organic layer was dried over sodium sulphate and concentrated to obtain the crude. The crude mass was purified by reverse phase HPLC to afford 3-(4,4-difluoroazepan-1-yl)-N-(3-(S-methylsulfonimidoyl) phenyl)quinoxaline-2-carboxamide as white solid. MS (ESI): Calcd [M+H]⁺: 460.16, found: 460.25.

¹H NMR (400 MHz, DMSO-d₆) δ 11.31 (s, 1H), 8.38 (s, 1H), 8.031 (d, J=8 Hz, 1H), 7.95 (d, J=8 Hz, 1H), 7.71 (d, J=8 Hz, 3H), 7.65 (t, J=8 Hz, 1H), 7.52-7.49 (m, 1H), 4.34 (s, 1H), 3.80-3.78 (m, 2H), 3.67-3.64 (m, 2H), 3.085 (s, 3H), 2.40-2.37 (m, 2H), 2.07-1.99 (m, 2H), 1.89-1.88 (m, 2H).

### Compound 8

### Synthesis of 3-(4,4-difluoroazepan-1-yl)-N-(3-sulfamoylphenyl)quinoxaline-2-carboxamide

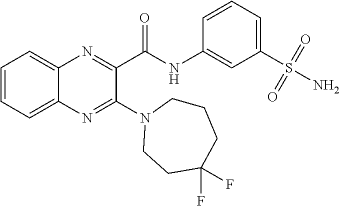 [SMILES: NS(=O)(=O)c1cccc(NC(=O)c2nc3ccccc3nc2N2CCCC(F)(F)CC2)c1]

### Synthesis of methyl 3-hydroxyquinoxaline-2-carboxylate

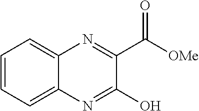 [SMILES: COC(=O)c1nc2ccccc2nc1O]

A stirred solution of 2-hydroxyquinoline-3-carboxylic acid (10 g, 52.6 nmol) in methanol (100 mL) was cooled to 0° C. Then sulfuric acid (20 mL) was added dropwise. The reaction mixture was heated at 70° C. for 12 hours. The progress of the reaction was monitored by TLC. After completion, the methanol was removed under vacuum. The residue thus obtained was dissolved into ethyl acetate and washed with sodium bicarbonate solution. The organic layer was separated, washed with brine, dried over anhydrous sodium sulfate and concentrated in vacuo to afford the crude material. The crude material was triturated with diethyl ether and pentane to afford the desire product methyl 3-hydroxyquinoxaline-2-carboxylate as a light orange solid.

### Synthesis of methyl 3-chloroquinoxaline-2-carboxylate

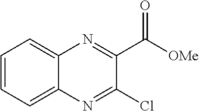 [SMILES: COC(=O)c1nc2ccccc2nc1Cl]

A mixture of methyl 3-hydroxyquinoxaline-2-carboxylate (5 g, 24.5 mmol) and phosphorus oxychloride (75 mL) was refluxed at 130° C. for 12 hours. The progress of the reaction was monitored by TLC. After completion, the reaction mixture was then cooled to room temperature and excess of phosphorus oxychloride distilled out. The residue thus obtained was dissolved into ethyl acetate and neutralized with sodium bicarbonate solution. The organic layer was separated, washed with brine, dried over anhydrous sodium sulfate and concentrated in vacuo to afford the crude material. The crude material was triturated with diethyl ether and pentane to afford the desire product methyl 3-chloroquinoxaline-2-carboxylate as a light brown solid.

### Synthesis of methyl 3-(4,4-difluoroazepan-1-yl)quinoxaline-2-carboxylate

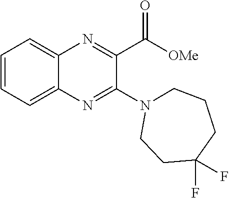 [SMILES: COC(=O)c1nc2ccccc2nc1N1CCCC(F)(F)CC1]

To a solution of methyl 3-chloroquinoxaline-2-carboxylate (5 g, 22.5 mmol) in N,N-dimethylformamide (50 mL) was added 4,4-difluoroazepane hydrochloride (5.8 g, 33.7 mmol) and potassium carbonate (15.5 g, 112 mmol). The mixture was heated at 70° C. for 16 hours under N₂ atmosphere. The progress of reaction was monitored by TLC. After completion, the reaction mixture was diluted with water and extracted with ethyl acetate. The organic layer was washed with brine solution and dried over anhydrous sodium sulfate and concentrated under reduced pressure to give the crude residue which was purified by silica gel column chromatography with a gradient of 20-30% ethyl acetate in hexanes to afford the desired compound methyl 3-(4,4-difluoroazepan-1-yl)quinoxaline-2-carboxylate as a brown solid.

### Synthesis of 3-(4,4-Difluoroazepan-1-yl)quinoxaline-2-carboxylic acid

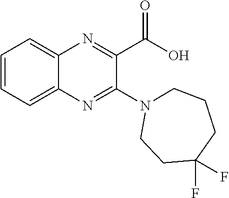 [SMILES: O=C(O)c1nc2ccccc2nc1N1CCCC(F)(F)CC1]

To a solution of methyl 3-(4,4-difluoroazepan-1-yl) quinoxaline-2-carboxylate (5 g, 15.6 mmol) in tetrahydrofuran (20 mL) and methanol (20 mL), lithium hydroxide (1.5 g, 62 mmol) and water (20 mL) was added. The reaction mixture was stirred at room temperature for 12 hours. After completion of reaction, the solvent was evaporated under vacuum and residue was diluted with water, acidified with 1N hydrochloric acid solution and extracted with ethyl acetate. The organic layer was washed with brine solution and dried over anhydrous sodium sulfate and concentrated under reduced pressure to give the crude material which was purified by triturated with diethyl ether and pentane to afford 3-(4,4-difluoroazepan-1-yl) quinoxaline-2-carboxylic acid as a light brown solid.

### Synthesis of 3-(4,4-Difluoroazepan-1-yl)-N-(3-sulfamoylphenyl)quinoxaline-2-carboxamide

To a solution of 3-(4,4-difluoroazepan-1-yl)quinoxaline-2-carboxylic acid (0.3 g, 0.9 mmol) in dichloromethane (6 mL) was added N,N′-dicyclohexylmethanediimine (0.24 mg, 1.17 mmol) and N,N-dimethylpyridin-4-amine (5.96 mg, 5 mol %) at 0° C. Then 3-aminobenzene-1-sulfonamide (0.2 mg, 1.17 mmol) was added slowly and the resulting mixture was stirred at room temperature for overnight. After completion of reaction, the reaction mixture was passed through celite pad and filtrate was washed with brine water and dried over sodium sulfate and concentrated under reduced pressure to get crude material. The crude residue was purified by flash column chromatography with a gradient of 50-60% ethyl acetate in hexanes to afford 3-(4,4-difluoroazepan-1-yl)-N-(3-sulfamoylphenyl)quinoxaline-2-carboxamide as yellow solid. MS (ESI): Calcd [M+H]⁺: 462.14, found: 462.20.

¹H NMR (400 MHz, DMSO-d₆): δ 11.27 (s, 1H), 8.33 (s, 1H), 7.94 (d, J=8.4 Hz, 1H), 7.91-7.88 (m, 1H), 7.10 (d, J=3.6 Hz, 2H), 7.61-7.59 (m, 2H), 7.52-7.48 (m, 1H), 7.43 (brs, 2H), 3.80-3.78 (m, 2H), 3.67 (t, J=7.5 Hz, 2H), 2.44-2.40 (m, 2H), 2.09-2.04 (m, 2H), 1.99-1.92 (m, 2H).

### Compound 9

### Synthesis of 2-(4,4-difluoroazepan-1-yl)-6-fluoro-N-(3-sulfamoylphenyl)quinoline-3-carboxamide

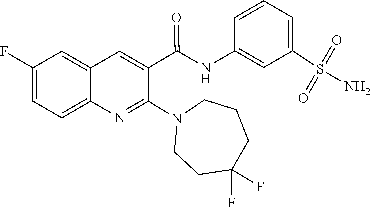 [SMILES: NS(=O)(=O)c1cccc(NC(=O)c2cc3cc(F)ccc3nc2N2CCCC(F)(F)CC2)c1]

### Synthesis of 2-chloro-6-fluoroquinoline-3-carboxylic acid

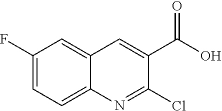 [SMILES: O=C(O)c1cc2cc(F)ccc2nc1Cl]

A stirred solution of 2-chloro-6-fluoroquinoline-3-carbaldehyde (=1 g, 4.8 mmol) in ethanol (30 mL), silver nitrate (1.3 g, 7.6 mmol) was added and the resulting reaction mixture was stirred for 1 hours at room temperature. Afterwards, to this, a solution of sodium hydroxide (0.96 g, 24 mmol) in 80% aqueous ethanol was added dropwise. The reaction mixture was stirred at room temperature for 5 hours. After completion of reaction, the reaction mixture was filtered through celite and solvent was evaporated under rotary and diluted with water, acidified with 1N hydrochloric acid solution and extracted with ethyl acetate. The organic layer was dried over anhydrous sodium sulphate and filtered. The filtrate was concentrated under reduced pressure to afford 2-chloro-6-fluoroquinoline-3-carboxylic acid as an off-white solid.

### Synthesis of methyl 2-chloro-6-fluoroquinoline-3-carboxylate

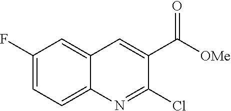 [SMILES: COC(=O)c1cc2cc(F)ccc2nc1Cl]

To a solution of 2-chloro-6-fluoroquinoline-3-carboxylic acid (0.75 g, 3.3 mmol) in N,N-dimethylformamide (7.5 mL) was added potassium carbonate (1.4 g, 10 mmol) and methyl iodide (0.5 ml, 8.3 mmol) at room temperature and stirred at 50° C. for 12 hours. After completion of reaction, the reaction mixture was quenched with ice-cold water and extracted with ethyl acetate. The organic layer was dried over anhydrous sodium sulphate and filtered. The filtrate was concentrated under reduced pressure to afford methyl 2-chloro-6-fluoroquinoline-3-carboxylate as a light yellow solid.

### Synthesis of methyl 2-(4,4-difluoroazepan-1-yl)-6-fluoroquinoline-3-carboxylate

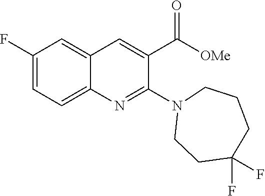 [SMILES: COC(=O)c1cc2cc(F)ccc2nc1N1CCCC(F)(F)CC1]

To s stirred solution of methyl 2-chloro-6-fluoroquinoline-3-carboxylate (0.55 g, 2.3 mmol) in N,N-dimethylformamide (5.5 mL), 4, 4-difluoroazepane hydrogen chloride (0.59 g, 3.45 mmol) and potassium carbonate (1.3 g, 9.2 mmol) was added at room temperature. The reaction mixture was stirred at 80° C. for 16 hours. The progress of the reaction was monitored by TLC. After completion of reaction, the reaction mixture was diluted with ice-cold water and extracted with ethyl acetate, dried over anhydrous sodium sulphate and filtered. The filtrate was concentrated under reduced pressure and the crude residue was purified by flash column chromatography with a gradient of 20-30% ethyl acetate in hexanes to afford the methyl 2-(4,4-difluoroazepan-1-yl)-6-fluoroquinoline-3-carboxylate as a yellow solid.

### Synthesis of 2-(4,4-difluoroazepan-1-yl)-6-fluoroquinoline-3-carboxylic acid

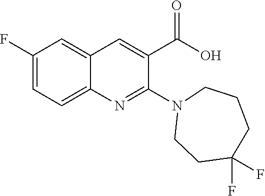 [SMILES: O=C(O)c1cc2cc(F)ccc2nc1N1CCCC(F)(F)CC1]

To a stirred solution of methyl 2-(4,4-difluoroazepan-1-yl)-6-fluoroquinoline-3-carboxylate (0.55 g, 1.6 mmol) in a mixture of methanol (5.5 mL), tetrahydrofuran (5.5 mL) and water (5.5 mL) (1:1:1) was added lithium hydroxide (0.14 g, 3.2 mmol) at room temperature. The reaction mixture was stirred at room temperature for 16 hours. The progress of reaction was monitored by TLC and LCMS. After completion of reaction, solvent was removed under rotatory and diluted with water, acidified with 1N hydrochloric solution and extracted with ethyl acetate. The organic layer was dried over anhydrous sodium sulphate and filtered. The filtrate was concentrated under reduced pressure to afford 2-(4,4-difluoroazepan-1-yl)-6-fluoroquinoline-3-carboxylic acid as an off-white solid.

### Synthesis of 2-(4,4-difluoroazepan-1-yl)-6-fluoro-N-(3-sulfamoylphenyl)quinoline-3-carboxamide

To a stirred solution of 2-(4,4-difluoroazepan-1-yl)-6-methoxyquinoline-3-carboxylic acid (0.4 g, 1.2 mmol) in dichloromethane (4 mL), oxalyl chloride (0.2 mL, 2.5 mmol) and one drop of N,N-dimethylfomamide was added at 0° C. The reaction mixture was stirred at room temperature for 2 hours. After completion of reaction, solvent was evaporated under nitrogen atmosphere and diluted with dry dichloromethane (4 mL) and added to the mixture of 3-aminobenzenesulfonamide (0.25 g, 1.5 mmol) and N,N-diisopropyl ethyl amine (1.1 mL, 6.2 mmol) in dry dichloromethane (4 mL) at 0° C. and allowed to stirred at room temperature for 5 hours. The progress of the reaction was monitored by TLC. After completion of reaction, the reaction mixture was diluted with water and extracted with dichloromethane. The organic layer was dried over anhydrous sodium sulphate and filtered. The filtrate was concentrated under reduced pressure and the crude residue was purified by flash column chromatography with a gradient of 40-60% ethyl acetate in hexanes to afford 2-(4,4-difluoroazepan-1-yl)-6-fluoro-N-(3-sulfamoylphenyl)quinoline-3-carboxamide as an off-white solid. MS (ESI): Calcd [M+H]⁺: 479.14, found: 479.25.

¹H NMR (400 MHz, DMSO-d₆): δ 10.99 (brs, 1H), 8.33 (s, 1H), 8.31 (s, 1H), 7.86-7.83 (m, 1H), 7.70-7.67 (m, 1H), 7.67-7.65 (m, 1H), 7.58.751 (m, 3H), 7.43 (brs, 2H), 3.75-3.73 (m, 2H), 3.61-3.58 (t, J=6.0 Hz, 2H), 2.54-2.38 (m, 2H), 2.07-1.96 (m, 2H), 1.89-1.88 (m, 2H).

### Compound 10

### Synthesis of 2-(4,4-difluoroazepan-1-yl)-6,7-difluoro-N-(3-sulfamoylphenyl)quinoline-3-carboxamide

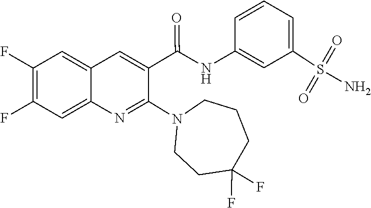 [SMILES: NS(=O)(=O)c1cccc(NC(=O)c2cc3cc(F)c(F)cc3nc2N2CCCC(F)(F)CC2)c1]

### Synthesis of 2-chloro-6,7-difluoroquinoline-3-carboxylic acid

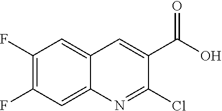 [SMILES: O=C(O)c1cc2cc(F)c(F)cc2nc1Cl]

To a stirred solution of 2-chloro-6,7-difluoroquinoline-3-carbaldehyde (1 g, 4.4 mmol) in ethanol (50 mL) was added silver nitrate (1.1 g, 6.6 mmol) and reaction mixture was stirred for 30 min at room temperature. To this, a solution of sodium hydroxide (0.88 g, 22 mmol) in 80% aqueous ethanol was added dropwise. The reaction mixture was stirred at room temperature for 5 hours. The progress of reaction was monitored by TLC. After completion of reaction, the reaction mixture was filtered through Celite and solvent was evaporated by rotary and diluted with water and acidified with 1N hydrochloric acid solution and extracted with ethyl acetate. The organic layer was combined, dried over sodium sulphate, filtered and concentrated to afford 2-chloro-6,7-difluoroquinoline-3-carboxylic acid as light yellow solid.

### Synthesis of methyl 2-chloro-6,7-difluoroquinoline-3-carboxylate

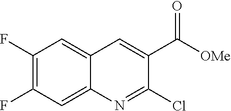 [SMILES: COC(=O)c1cc2cc(F)c(F)cc2nc1Cl]

To a stirred solution of 2-chloro-6,7-difluoroquinoline-3-carboxylic acid (1 g, 4.1 mmol) in N,N-dimethylformamide (10 mL) was added potassium carbonate (2.3 g, 16. mmol) and iodomethane (0.62 mL, 12.3 mmol). The reaction mixture was stirred for 12 hours at 50° C. The progress of reaction was monitored by TLC. After completion of reaction, the reaction mixture was quenched with water and extracted with ethyl acetate. The organic layer was combined, dried with sodium sulphate, filtered and concentrated to afford methyl 2-chloro-6,7-difluoroquinoline-3-carboxylate as a white solid.

### Synthesis of methyl 2-(4,4-difluoroazepan-1-yl)-6,7-difluoroquinoline-3-carboxylate

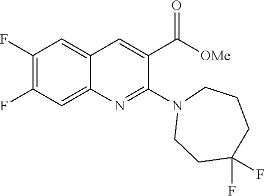 [SMILES: COC(=O)c1cc2cc(F)c(F)cc2nc1N1CCCC(F)(F)CC1]

To a solution of methyl 2-chloro-6,7-difluoroquinoline-3-carboxylate (1 g, 3.9 mmol) in N,N-dimethylformamide (10 mL) was added potassium carbonate (2.2 g, 15.5 mmol) and 4,4-difluoroazepane hydrogen chloride (1.0 g, 5.8 mmol). The resulting reaction mixture was heated to 70° C. and stirred for 16 hours. The progress of reaction was monitor by TLC and LCMS. After completion of reaction, the reaction mixture was quenched with water and extracted with ethyl acetate. The organic layer was combined, dried over sodium sulphate, filtered and concentrated to afford the crude material. The crude material was purified by combi-flash with gradient 30-50% ethyl acetate in heptane to afford methyl 2-(4,4-difluoroazepan-1-yl)-6,7-difluoroquinoline-3-carboxylate as a white solid.

### Synthesis of 2-(4,4-difluoroazepan-1-yl)-6,7-difluoroquinoline-3-carboxylic acid

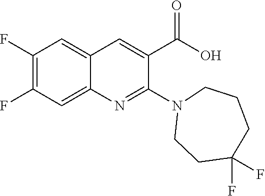 [SMILES: O=C(O)c1cc2cc(F)c(F)cc2nc1N1CCCC(F)(F)CC1]

To a solution of methyl 2-(4,4-difluoroazepan-1-yl)-6,7-difluoroquinoline-3-carboxylate (0.75 g, 2.1 mmol) in 1:1 methanol (5.48 mL) and tetrahydrofuran (5.48 mL) was added lithium hydroxide (0.18 g, 4.2 mmol). The reaction mixture was stirred 16 hours at room temperature. The progress of reaction was monitored by TLC and LCMS. After completion of reaction, the reaction mixture was concentrated to remove solvent. The crude residue was diluted with water, acidified with 1 N hydrochloric acid solution and extracted with ethyl acetate, dried over sodium sulphate and concentrated to afford 2-(4,4-difluoroazepan-1-yl)-6,7-difluoroquinoline-3-carboxylic acid as a yellow solid.

### Synthesis of 2-(4,4-difluoroazepan-1-yl)-6,7-difluoro-N-(3-sulfamoylphenyl)quinoline-3-carboxamide

To a solution of 2-(4,4-difluoroazepan-1-yl)-6,7-difluoroquinoline-3-carboxylic acid (0.5 g, 1.5 mmol) in anhydrous dichloromethane (10 mL) was added oxalyl chloride (0.25 mL, 2.9 mmol) and a drop of anhydrous N,N-dimethylformamide at 0° C. The reaction mixture was stirred at 0° C. for 30 minutes. After completion of reaction, the reaction mixture was evaporated in vacuum. The crude residue was redissolved into dry dichloromethane and added to the solution of 3-aminobenzene-1-sulfonamide (0.3 g, 1.75 mmol) and N,N-diisopropyl ethyl amine (0.76 g, 5.8 mmol) in dichloromethane (70 mL) at 0° C. The resulting reaction mixture was stirred at room temperature for 1 hour. After completion of reaction, the reaction mixture was diluted with dichloromethane and wash with water, brine solution. The combined organic layer was dried over sodium sulphate, filtered and concentrated to afford the crude product. The crude material was purified by using flash chromatography with gradient 20-50% ethyl acetate in hexanes to afford 2-(4,4-difluoroazepan-1-yl)-6,7-difluoro-N-(3-sulfamoylphenyl)quinoline-3-carboxamide as a white solid. MS (ESI): Calcd [M+H]⁺: 497.13, found: 497.15.

¹H NMR (400 MHz, DMSO-d₆): δ 10.99 (s, 1H), 8.33 (s, 1H), 8.30 (s, 1H), 7.94 (t, J=9.6 Hz, 1H), 7.85 (d, J=8 Hz, 1H), 7.61 (s, 1H), 7.58 (d, J=8 Hz, 2H), 7.42 (s, 2H), 3.74 (s, 2H), 3.60-3.58 (m, 2H), 2.40-2.37 (m, 2H), 2.07-1.99 (m, 2H), 1.89-1.88 (m, 2H).

### Compound 11

### Synthesis of 2-(4,4-difluoroazepan-1-yl)-6-methoxy-N-(3-sulfamoylphenyl)quinoline-3-carboxamide

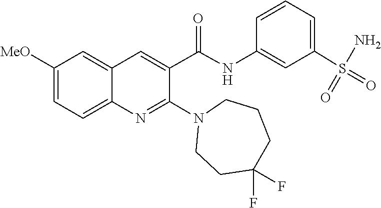 [SMILES: COc1ccc2nc(N3CCCC(F)(F)CC3)c(C(=O)Nc3cccc(S(N)(=O)=O)c3)cc2c1]

### Synthesis of 2-chloro-6-methoxyquinoline-3-carboxylic acid

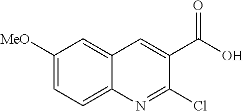 [SMILES: COc1ccc2nc(Cl)c(C(=O)O)cc2c1]

To a stirred solution of 2-chloro-6-methoxyquinoline-3-carbaldehyde (1.4 g, 6.3 mmol) in ethanol (28 mL), silver nitrate (1.7 g, 10 mmol) was added and reaction mixture was stirred at room temperature for 1 hour. Afterwards, a solution of sodium hydroxide (1.3 g, 32 mmol) in 80% aqueous ethanol was added dropwise. The reaction mixture was stirred at room temperature for 5 hours. After completion of reaction, the reaction mixture was filtered through celite and solvent was evaporated under rotary and diluted with water, acidified with 1N hydrochloric acid solution and extracted with ethyl acetate. The organic layer was dried over anhydrous sodium sulphate and filtered. The filtrate was concentrated under reduced pressure to afford 2-chloro-6-methoxyquinoline-3-carboxylic acid as an off-white solid.

### Synthesis methyl 2-chloro-6-methoxyquinoline-3-carboxylate

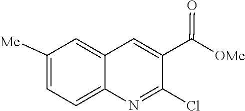 [SMILES: COC(=O)c1cc2cc(C)ccc2nc1Cl]

To a solution of 2-chloro-6-methoxyquinoline-3-carboxylic acid (0.6 g, 2.5 mmol) in N,N-dimethylformamide (6 mL) was added potassium carbonate (1.0 g, 7.6 mmol) and methyl iodide (0.4 ml, 6.3 mmol) at room temperature and stirred for 12 hours at 50° C. After completion of reaction, the reaction mixture was quenched with ice-cold water and extracted with ethyl acetate. The organic layer was dried over anhydrous Na₂SO₄ and filtered. The filtrate was concentrated under reduced pressure concentrated to afford methyl 2-chloro-6-methoxyquinoline-3-carboxylate as a light yellow solid.

### Synthesis of methyl 2-(4,4-difluoroazepan-1-yl)-6-methoxyquinoline-3-carboxylate

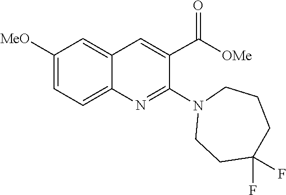 [SMILES: COC(=O)c1cc2cc(OC)ccc2nc1N1CCCC(F)(F)CC1]

To a stirred solution of methyl 2-chloro-6-methoxyquinoline-3-carboxylate (0.5 g, 2 mmol) in NN-dimethylformamide (10 mL) was added 4, 4-difluoroazepane hydrogen chloride (0.50 g, 3.0 mmol) and potassium carbonate (1 g, 8 mmol) was added at room temperature. The reaction mixture was stirred at 80° C. for 16 hours. The progress of the reaction was monitored by TLC. After completion of reaction, the reaction mixture was diluted with ice-cold water and extracted with ethyl acetate, dried over anhydrous sodium sulphate and filtered. The filtrate was concentrated under reduced pressure and the crude residue was purified by flash column chromatography with a gradient of 30-50% ethyl acetate in hexanes to afford methyl 2-(4,4-difluoroazepan-1-yl)-6-methoxyquinoline-3-carboxylate as a yellow solid.

### Synthesis of 2-(4,4-difluoroazepan-1-yl)-6-methoxyquinoline-3-carboxylic acid

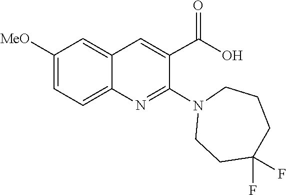 [SMILES: COc1ccc2nc(N3CCCC(F)(F)CC3)c(C(=O)O)cc2c1]

To a stirred solution of methyl 2-(4,4-difluoroazepan-1-yl)-6-methoxyquinoline-3-carboxylate (0.45 g, 1.3 mmol) in a mixture of methanol (4.5 mL), tetrahydrofuran (4.5 mL) and water (4.5 mL) was added lithium hydroxide (0.11 g, 2.6 mmol) at room temperature and stirred for 16 hours. After completion of reaction, solvent was evaporated under rotary and diluted with water, acidified with 1 N hydrochloric solution and extracted with ethyl acetate. The organic layer was combined, dried over sodium sulphate, filtered and concentrated to afford 2-(4,4-difluoroazepan-1-yl)-6-methoxyquinoline-3-carboxylic acid as a yellow solid.

### Synthesis of 2-(4,4-difluoroazepan-1-yl)-6-methoxy-N-(3-sulfamoylphenyl)quinoline-3-carboxamide

To a stirred solution of 2-(4,4-difluoroazepan-1-yl)-6-methoxyquinoline-3-carboxylic acid (0.24 g, 0.71 mmol) in dichloromethane (3 mL) was added (1-[Bis(dimethylamino)methylene]-1H-1,2,3-triazolo[4,5-b]pyridinium 3-oxide hexafluorophosphate (HATU) (0.41 g, 1.1 mmol) and 4-dimethylaminopyridine (DMAP) (0.043 g, 0.36 mmol) at 0° C. The reaction mixture was stirred at room temperature for 8 hours. After completion of reaction, solvent was evaporated and diluted with acetonitrile (3 mL) and 3-aminobenzenesulfonamide (122 mg, 0.71 mmol) was added to the above solution and stirred at 70° C. for 16 hours. The progress of the reaction was monitored by TLC. After completion of reaction, the reaction mixture was diluted with water and extracted with ethyl acetate. The organic layer was combined, dried over anhydrous sodium sulphate, filtered and concentrated to afford crude material. The crude residue was purified by flash column chromatography with a gradient of 50-70% ethyl acetate in hexanes to afford 2-(4,4-difluoroazepan-1-yl)-6-methoxy-N-(3-sulfamoylphenyl)quinoline-3-carboxamide as a yellow solid. MS (ESI): Calcd [M+H]⁺: 491.16, found: 491.20.

¹H NMR (400 MHz, DMSO-d₆): δ 10.91 (s, 1H), 8.34 (brs, 1H), 8.24 (s, 1H), 7.86-7.83 (m, 1H), 7.60-7.54 (m, 3H), 7.42 (s, 2H), 7.32-7.30 (m, 2H), 3.25 (s, 3H), 3.72-3.70 (m, 2H), 3.56 (t, J=6.0 Hz, 2H), 2.43-2.36 (m, 2H), 2.02-1.96 (m, 2H), 1.88-1.86 (m, 2H).

### Compound 12

### Synthesis of 6-chloro-2-(4,4-difluoroazepan-1-yl)-N-(3-sulfamoylphenyl)quinoline-3-carboxamide

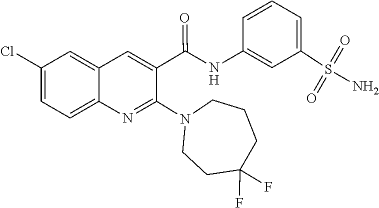 [SMILES: NS(=O)(=O)c1cccc(NC(=O)c2cc3cc(Cl)ccc3nc2N2CCCC(F)(F)CC2)c1]

### Synthesis of 2,6-dichloroquinoline-3-carboxylic acid

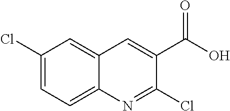 [SMILES: O=C(O)c1cc2cc(Cl)ccc2nc1Cl]

To a stirred solution of 2,6-dichloroquinoline-3-carbaldehyde (1.0 g, 4.4 mmol) in ethanol (30 mL) was added silver nitrate (1.2 g, 7.1 mmol) and reaction mixture was stirred for 1 hour at room temperature. Afterwards, a solution of sodium hydroxide (0.88 g, 22 mmol) in 80% aqueous ethanol was added drop wise and allowed to stirrer at room temperature for 5 hours. After completion of reaction, the reaction mixture was filtered through Celite and solvent was evaporated under rotary and diluted with water, acidified with 1 N hydrochloric acid solution and extracted with ethyl acetate. The organic layer was dried over anhydrous sodium sulphate and filtered. The filtrate was concentrated under reduced pressure to afford 2,6-dichloroquinoline-3-carboxylic acid as an off-white solid.

### Synthesis of methyl 2,6-dichloroquinoline-3-carboxylate

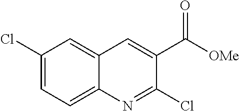 [SMILES: COC(=O)c1cc2cc(Cl)ccc2nc1Cl]

To a solution of 2,6-dichloroquinoline-3-carboxylic acid (0.9 g, 3.7 mmol) in N,N-dimethylformamide (9 mL) was added dipotassium carbonate (1.5 g, 11 mmol) and methyl iodide (0.6 ml, 9.3 mmol) at room temperature and stirred at 50° C. for 12 hours. After completion of reaction, reaction mixture was quenched with ice-cold water and extracted with ethyl acetate. The organic layer was dried over anhydrous sodium sulphate and filtered. The filtrate was concentrated under reduced pressure to afford methyl 2,6-dichloroquinoline-3-carboxylate as a light yellow solid.

### Synthesis of methyl 6-chloro-2-(4,4-difluoroazepan-1-yl) quinoline-3-carboxylate

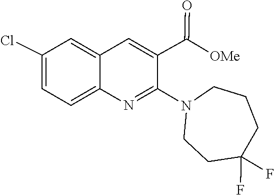 [SMILES: COC(=O)c1cc2cc(Cl)ccc2nc1N1CCCC(F)(F)CC1]

To a stirred solution of methyl 2,6-dichloroquinoline-3-carboxylate (0.8 g, 3.1 mmol) in N,N-dimethylformamide (8 mL) was added 4, 4-difluoroazepane hydrogen chloride (0.63 g, 4.7 mmol) and potassium carbonate (1.7 g, 12.5 mmol) at room temperature. The reaction mixture was stirred at 80° C. for 16 hours. The progress of the reaction was monitored by TLC. After completion of reaction, the reaction mixture was diluted with ice-cold water, extracted with ethyl acetate, dried over anhydrous sodium sulphate and filtered. The filtrate was concentrated under reduced pressure and the resulting crude residue was purified by flash column chromatography with a gradient of 50-70% ethyl acetate in hexanes to afford methyl 6-chloro-2-(4, 4-difluoroazepan-1-yl) quinoline-3-carboxylate as a yellow solid.

### Synthesis of 6-chloro-2-(4,4-difluoroazepan-1-yl) quinoline-3-carboxylic acid

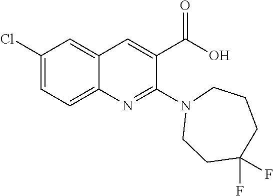 [SMILES: O=C(O)c1cc2cc(Cl)ccc2nc1N1CCCC(F)(F)CC1]

To a stirred solution of methyl 6-chloro-2-(4, 4-difluoroazepan-1-yl) quinoline-3-carboxylate (0.8 g, 2.3 mmol) in a mixture of methanol (8 mL), tetrahydrofuran (8 mL), and water (8 mL) was added lithium hydroxide (0.19 g, 4.5 mmol) and the reaction mixture was stirred at room temperature for 16 hours. After completion of reaction, solvent was evaporated under rotatory and diluted with water, acidified with 1N hydrochloric acid and extracted with ethyl acetate. The organic layer was dried over anhydrous sodium sulphate and filtered. The filtrate was concentrated under reduced pressure to afford 6-chloro-2-(4,4-difluoroazepan-1-yl) quinoline-3-carboxylic acid as a yellow solid.

### Synthesis of 6-chloro-2-(4,4-difluoroazepan-1-yl)-N-(3-sulfamoylphenyl) quinoline-3-carboxamide

To a stirred solution of 6-chloro-2-(4,4-difluoroazepan-1-yl) quinoline-3-carboxylic acid (0.33 g, 0.88 mmol) in dichloromethane (3 mL), was added (1-[Bis(dimethylamino)methylene]-1H-1,2,3-triazolo[4,5-b]pyridinium 3-oxide hexafluorophosphate (HATU) (0.50 g, 1.3 mmol) and 4-dimethylaminopyridine (DMAP) (0.054 g, 0.44 mmol) at 0° C. The reaction mixture was stirred at room temperature for 12 hours. After completion of reaction, solvent was evaporated in rotary and diluted with acetonitrile (3 mL) and to this, 3-aminobenzenesulfonamide (0.18 g, 1.1 mmol) was added and reflux for 16 hours. The progress of the reaction was monitored by TLC. After completion of reaction, the reaction mixture was diluted with water and extracted with ethyl acetate. The organic layer was dried over anhydrous sodium sulphate and filtered. The filtrate was concentrated under reduced pressure and the crude residue was purified by flash column chromatography with a gradient of 50-70% ethyl acetate in hexanes to afford 6-chloro-2-(4,4-difluoroazepan-1-yl)-N-(3-sulfamoylphenyl) quinoline-3-carboxamide as a yellow solid. MS (ESI): Calcd [M+H]⁺: 495.11, found: 495.20.

¹H NMR (400 MHz, DMSO-d₆): δ 10.99 (s, 1H), 8.31 (d, J=6.0 Hz, 2H), 7.95 (s, 1H), 7.85-7.83 (m, 1H), 7.65-7.62 (m, 2H), 7.60-7.55 (m, 2H), 7.42 (brs, 2H), 3.76-3.74 (m, 2H), 3.61 (t, J=6.0 Hz, 2H), 2.40-2.38 (m, 2H), 2.06-1.99 (m, 2H), 1.96-1.88 (m, 2H).

### Compound 13

### Synthesis of 7-chloro-2-(4,4-difluoroazepan-1-yl)-N-(3-sulfamoylphenyl)quinoline-3-carboxamide

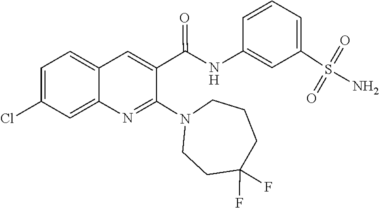 [SMILES: NS(=O)(=O)c1cccc(NC(=O)c2cc3ccc(Cl)cc3nc2N2CCCC(F)(F)CC2)c1]

### Synthesis of 2,7-dichloroquinoline-3-carboxylic acid

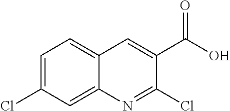 [SMILES: O=C(O)c1cc2ccc(Cl)cc2nc1Cl]

To a stirred solution of 2,7-dichloroquinoline-3-carbaldehyde (1.0 g, 4.4 mmol) in ethanol (20 mL) was added silver nitrate (1.2 g, 7.1 mmol). To this mixture, a solution of sodium hydroxide (0.9 g, 22 mmol) in water (3 mL) was added dropwise. The reaction mixture was stirred at room temperature for 5 hours. After completion of reaction, the reaction mixture was filtered through a Celite pad and solvent was removed under rotatory evaporation to obtain crude mixture. The crude mixture was diluted with water and acidified with 1 N hydrochloric acid solution and extracted with ethyl acetate. The organic layer was separated, washed with brine, dried over anhydrous sodium sulfate and concentrated in vacuo to afford the crude material. The crude material was triturated with diethyl ether and pentane to afford 2,7-dichloroquinoline-3-carboxylic acid as an off-white solid.

### Synthesis of 7-Chloro-2-(4,4-difluoroazepan-1-yl)quinoline-3-carboxylic acid

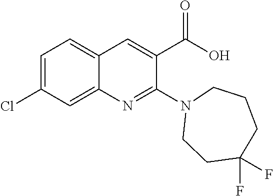 [SMILES: O=C(O)c1cc2ccc(Cl)cc2nc1N1CCCC(F)(F)CC1]

To a solution of 2,7-dichloroquinoline-3-carboxylic acid (0.65 g, 2.7 mmol) in N,N-dimethylformamide (20 mL) was added 4,4-difluoroazepane hydrogen chloride (0.67 g, 34 mmol) and potassium carbonate (1.5 g, 11 mmol). The mixture was heated at 70° C. for 12 hours under nitrogen atmosphere. The progress of reaction was monitored by TLC. After completion, the crude material was diluted with water followed by treatment with 1N hydrochloric acid solution to pH 6.0 and extracted with ethyl acetate. The organic layer was washed with brine solution and dried over anhydrous sodium sulphate. The crude material was triturated with diethyl ether and pentane to afford the product 7-chloro-2-(4,4-difluoroazepan-1-yl)quinoline-3-carboxylic acid as light brown solid.

### Synthesis of 7-Chloro-2-(4,4-difluoroazepan-1-yl)-N-(3-sulfamoylphenyl)quinoline-3-carboxamide

To a solution of 7-chloro-2-(4,4-difluoroazepan-1-yl)quinoline-3-carboxylic acid (0.30 g, 0.90 mmol) in dichloromethane (6 mL) was added oxalyl chloride (0.15 g, 1.0 mmol) under nitrogen atmosphere followed by 1-2 drops of N,N-dimethylformamide at 0° C. and continue the reaction for 1 hour at room temperature. After completion of reaction, solvent was removed under nitrogen atmosphere. The crude mixture was dissolved into dichloromethane (6 mL) and added to the solution of 3-aminobenzene-1-sulfonamide (0.18 g, 1.2 mmol) and N,N-diisopropylethyl amine (0.4 mL, 4.5 mmol) in dichloromethane (6 mL) solution at 0° C. and stirred the mixture for additional 1 hour at room temperature. The progress of reaction was monitor by TLC. After completion of reaction, the mixture was quenched with water then extracted with dichloromethane. The organic layer was combined, dried over sodium sulphate, filtered and concentrated to afford crude material. The crude residue was purified by flash column chromatography with a gradient of 40-50% ethyl acetate in hexanes to afford 7-chloro-2-(4,4-difluoroazepan-1-yl)-N-(3-sulfamoylphenyl)quinoline-3-carboxamide as light brown solid. MS (ESI): Calcd [M+H]⁺: 495.11, found: 495.15.

¹H NMR (400 MHz, DMSO-d₆): δ 10.98 (s, 1H), 8.37 (s, 1H), 8.30 (s, 1H), 7.88-7.83 (m, 2H), 7.64 (d, J=1.6 Hz, 1H), 7.58-7.54 (m, 2H), 7.42 (s, 2H), 7.32 (dd, J=2.4, 8.8 Hz, 1H), 3.77-3.74 (m, 2H), 3.60 (t, J=5.6 Hz, 2H), 2.44-2.37 (m, 2H), 2.00-1.99 (m, 2H), 1.95-1.88 (m, 2H).

### Compound 14

### Synthesis of N-(3-carbamoylphenyl)-3-(4,4-difluoroazepan-1-yl)quinoxaline-2-carboxamide

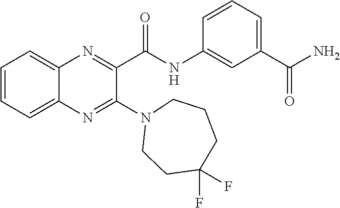 [SMILES: NC(=O)c1cccc(NC(=O)c2nc3ccccc3nc2N2CCCC(F)(F)CC2)c1]

### Synthesis of 3-chloroquinoxaline-2-carboxylic acid

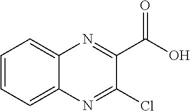 [SMILES: O=C(O)c1nc2ccccc2nc1Cl]

A mixture of 3-hydroxyquinoxaline-2-carboxylic acid (2 g, 9.6 mmol) and phosphorous oxychloride (20 mL) was slowly heated to 120° C. for 5 hours. After completion of the reaction, the reaction mixture was quenched with ice-cold water, extracted with ethyl acetate. The organic layer was dried over anhydrous sodium sulphate and filtered. The filtrate was concentrated under reduced pressure and resultant crude residue was triturated with n-pentane and diethyl ether to afford 3-chloroquinoxaline-2-carboxylic acid as a yellow solid.

### Synthesis of 3-(4,4-difluoroazepan-1-yl)quinoxaline-2-carboxylic acid

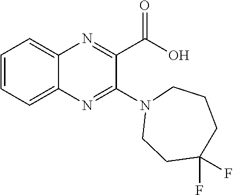 [SMILES: O=C(O)c1nc2ccccc2nc1N1CCCC(F)(F)CC1]

To a solution of 3-chloroquinoxaline-2-carboxylic acid (1.0 g, 1.65 mmol) in N,N-dimethylformamide (10 mL) was added 4,4-difluoroazepane hydrogen chloride (0.43 g, 2.5 mmol) and potassium carbonate (0.9 g, 6.6 mmol) at room temperature. The reaction mixture was heated at 78° C. for 12 hours. After completion of reaction, the crude material was diluted with cold-water followed by treatment with 1N hydrochloric acid solution and extracted with ethyl acetate. The organic layer was washed with brine solution, dried over anhydrous sodium sulphate and filtered. The filtrate was concentrated under reduced pressure. The crude residue was purified by flash column chromatography with a gradient of 50-70% ethyl acetate in hexanes to afford the desired compound 3-(4,4-difluoroazepan-1-yl)quinoxaline-2-carboxylic acid as a yellow solid.

### Synthesis of methyl 3-(3-(4,4-difluoroazepan-1-yl)quinoxaline-2-carboxamido)benzoate

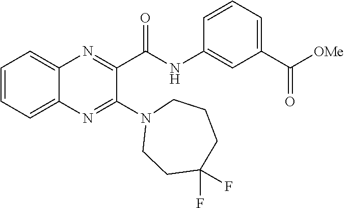 [SMILES: COC(=O)c1cccc(NC(=O)c2nc3ccccc3nc2N2CCCC(F)(F)CC2)c1]

To a solution of 3-(4,4-difluoroazepan-1-yl)quinoxaline-2-carboxylic acid (0.4 g, 1.4 mmol) in dichloromethane (10 mL) was added oxalyl chloride (0.15 g, 2.7 mmol) and a drop of N,N-dimethylformamide at 0° C. under nitrogen atmosphere and stirred for 1 hour at room temperature. After completion of reaction, solvent was distilled out under nitrogen atmosphere. The crude mixture was dissolved into dichloromethane (10 mL) and added to the solution of 3-aminomethylbenzoate (0.29 g, 1.7 mmol) and N,N-diisopropylethylamine (0.40 mL, 4.5 mmol) in dichloromethane (10 mL) at 0° C. and stirred the mixture for 12 hours at room temperature. The progress of reaction was monitored by TLC. After completion of reaction, the mixture was quenched with water, extracted with dichloromethane. The combined organic layer was dried over sodium sulphate, filtered and concentrated to afford crude residue which was purified by flash column chromatography with a gradient of 40-50% ethyl acetate in hexanes to afford methyl 3-(3-(4,4-difluoroazepan-1-yl)quinoxaline-2-carboxamido)benzoate as yellow solid.

### Synthesis of N-(3-carbamoylphenyl)-3-(4,4-difluoroazepan-1-yl)quinoxaline-2-carboxamide

To a stirred solution of methyl 3-(3-(4,4-difluoroazepan-1-yl)quinoxaline-2-carboxamido)benzoate (0.28 g, 0.75 mmol) in ammonia solution (7 N ammonia in methanol, 10 mL) was added at room temperature. The reaction mixture was stirred at 100° C. in an autoclave for 28 hours. The progress of the reaction was monitored by TLC and LCMS. After completion of reaction, the solvent was removed in vacuo. The reaction mixture was diluted with water and extracted with ethyl acetate. The organic layer was dried over anhydrous sulphate and filtered. The filtrate was concentrated under reduced pressure and the crude residue was purified by flash column chromatography with a gradient of 50-70% ethyl acetate in hexanes to afford N-(3-carbamoylphenyl)-3-(4,4-difluoroazepan-1-yl) quinoxaline-2-carboxamide as yellow solid. MS (ESI): Calcd [M+H]⁺: 426.17, found: 426.20.

¹H NMR (400 MHz, DMSO-d₆): δ 11.07 (s, 1H), 8.21 (t, J=16.0 Hz, 1H), 7.95 (s, 1H), 7.93 (d, J=8.4 Hz, 1H), 7.89 (dd, J=8 Hz, 1.6 Hz, 1H), 7.70-7.71 (m, 2H), 7.64 (d, J=8 Hz, 1H), 7.45-7.52 (m, 2H), 7.40 (s, 1H), 3.79-3.81 (m, 2H), 3.68 (t, J=6 Hz, 2H), 2.34-2.43 (m, 2H), 2.03-2.08 (m, 2H), 1.91-1.99 (m, 2H)

### Compound 15

### Synthesis of 2-(4,4-difluoroazepan-1-yl)-N-(3-sulfamoylphenyl)-1,7-naphthyridine-3-carboxamide

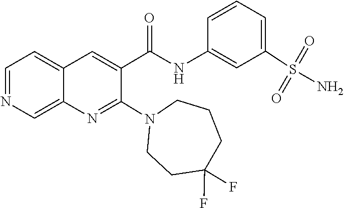 [SMILES: NS(=O)(=O)c1cccc(NC(=O)c2cc3ccncc3nc2N2CCCC(F)(F)CC2)c1]

### Synthesis of ethyl 2-hydroxy-1,7-naphthyridine-3-carboxylate

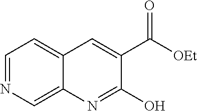 [SMILES: CCOC(=O)c1cc2ccncc2nc1O]

To a stirred solution of 3-aminoisonicotinaldehyde (2.5 g, 20.4 mmol) in ethanol (25 mL) was added diethyl malonate (9.4 mL, 61.5 mmol) and piperidine (0.65 mL, 6.2 mmol). The reaction mixture was refluxed and stirred for 18 hours. After completion of reaction, the reaction mixture was concentrated under reduced pressure to afford the crude material which was triturated with diethyl ether and pentane to furnish ethyl 2-hydroxy-1,7-naphthyridine-3-carboxylate as a yellow solid.

### Synthesis of ethyl 2-chloro-1,7-naphthyridine-3-carboxylate

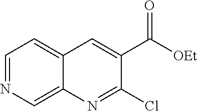 [SMILES: CCOC(=O)c1cc2ccncc2nc1Cl]

A mixture of ethyl 2-hydroxy-1,7-naphthyridine-3-carboxylate (2.8 g, 12.8 mmol) and phosphorus oxychloride (14 mL) was heated at 130° C. for 3 hours. The progress of reaction was monitored by TLC. After completion of reaction, the reaction mixture was concentrated under reduced pressure. The reaction mixture was diluted with cold water and basified with aq. sodium bicarbonate solution and extracted with ethyl acetate. The organic layer was dried over anhydrous sodium sulphate. The filtrate was concentrated under reduced pressure to afford crude material. The crude residue was purified by flash column chromatography with a gradient of 20-30% ethyl acetate in hexanes to afford ethyl 2-chloro-1,7-naphthyridine-3-carboxylate as a yellow solid.

### Synthesis of ethyl 2-(4,4-difluoroazepan-1-yl)-1,7-naphthyridine-3-carboxylate

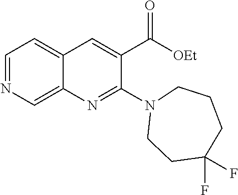 [SMILES: CCOC(=O)c1cc2ccncc2nc1N1CCCC(F)(F)CC1]

To a stirred solution of ethyl 2-chloro-1,7-naphthyridine-3-carboxylate (1.0 g, 4.2 mmol) in N,N-dimethylformamide (10 mL), 4, 4-difluoroazepane hydrogen chloride (1.0 g, 6.4 mmol) and dipotassium carbonate (2.3 g, 17 mmol) was added at room temperature and slowly heated to 70° C. and stirred for 12 hours. After completion of reaction, the reaction mixture was cool to room temperature, diluted with ice cold water and extracted with ethyl acetate. The organic layer was combined, dried over anhydrous sodium sulphate, filtered and concentrated. The crude residue was purified by flash column chromatography with a gradient of 30-40% ethyl acetate in hexanes to afford ethyl 2-(4,4-difluoroazepan-1-yl)-1,7-naphthyridine-3-carboxylate as a light yellow solid.

### Synthesis of 2-(4,4-difluoroazepan-1-yl)-1,7-naphthyridine-3-carboxylic acid

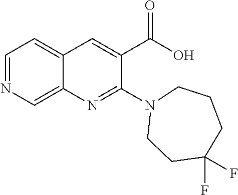 [SMILES: O=C(O)c1cc2ccncc2nc1N1CCCC(F)(F)CC1]

To a stirred solution of ethyl 2-(4,4-difluoroazepan-1-yl)-1,7-naphthyridine-3-carboxylate (0.8 g, 2.4 mmol) in a mixture of methanol (8 mL), tetrahydrofuran (8 mL), and water (8 mL) was added lithium hydroxide (0.7 g, 4.8 mmol). The reaction mixture was stirred at room temperature for 12 hours. The progress of reaction was monitored by TLC. After completion of reaction, solvent was evaporated under rotary evaporation and diluted with water, acidified with 1N hydrochloric acid solution and extracted with ethyl acetate. The organic layer was combined, dried over anhydrous sodium sulphate, filtrated and concentrated in vacuo to afford the crude material. The crude material was triturated with diethyl ether and pentane to afford 2-(4,4-difluoroazepan-1-yl)-1,7-naphthyridine-3-carboxylic acid as a yellow solid.

### Synthesis of 2-(4,4-difluoroazepan-1-yl)-N-(3-sulfamoylphenyl)-1,7-naphthyridine-3-carboxamide

To a stirred solution of 2-(4,4-difluoroazepan-1-yl)-1,7-naphthyridine-3-carboxylic acid (0.4 g, 1.3 mmol) in dichloromethane (4 mL), (1-[Bis(dimethylamino)methylene]-1H-1,2,3-triazolo[4,5-b]pyridinium 3-oxide hexafluorophosphate (HATU) (0.74 g, 2 mmol) and 4-dimethylaminopyridine (DMAP) (0.08 g, 0.65 mmol) was added at room temperature and the reaction mixture was stirred at room temperature for 16 hours. After completion of reaction, solvent was evaporated under rotary and residue was diluted with acetonitrile (4 mL) and to this solution, 3-aminobenzenesulfonamide (0.27 g, 1.6 mmol) was added in one portion. The reaction mixture was heated to 70° C. for 18 hours. The progress of the reaction was monitored by TLC. After completion of reaction, the reaction mixture was diluted with water and extracted with ethyl acetate. The organic layer was combined, dried over anhydrous sodium sulphate, filtered and concentrated to afford crude material. The crude residue was purified by flash column chromatography with a gradient of 30-50% ethyl acetate in hexanes to afford 2-(4,4-difluoroazepan-1-yl)-N-(3-sulfamoylphenyl)-1,7-naphthyridine-3-carboxamide as an off-white solid. MS (ESI): Calcd [M+H]⁺: 462.14, found: 462.20.

¹H NMR (400 MHz, DMSO-d₆): δ 11.07 (s, 1H), 9.01 (s, 1H), 8.40 (s, 1H), 8.35 (d, J=6.5 Hz, 1H), 8.30 (brs, 1H), 7.87-7.84 (m, 1H), 7.73 (d, J=6.5 Hz, 1H), 7.60-7.56 (m, 2H), 7.43 (brs, 2H), 3.80-3.78 (m, 2H), 3.64 (t, J=6.0 Hz, 2H), 2.42-2.39 (m, 2H), 2.07-1.98 (m, 2H), 1.92-1.90 (m, 2H)

### Compound 16

### Synthesis of 2-(4,4-difluoroazepan-1-yl)-7-methoxy-N-(3-sulfamoylphenyl)quinoline-3-carboxamide

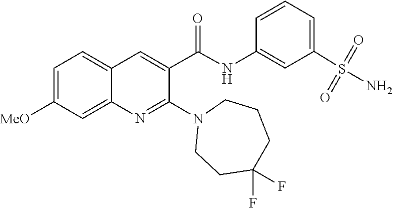 [SMILES: COc1ccc2cc(C(=O)Nc3cccc(S(N)(=O)=O)c3)c(N3CCCC(F)(F)CC3)nc2c1]

### Synthesis of 2-chloro-7-methoxyquinoline-3-carboxylic acid (2)

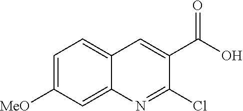 [SMILES: COc1ccc2cc(C(=O)O)c(Cl)nc2c1]

To a stirred solution of 2-chloro-7-methoxyquinoline-3-carbaldehyde (2.0 g, 9.0 mmol) in ethanol (40 mL) was added silver nitrate (2.45 g, 14.4 mmol). To this mixture, a solution of sodium hydroxide (1.8 g, 45 mmol) in water (10 mL) was added dropwise. The reaction mixture was stirred at room temperature for 5 hours. After completion of reaction, the reaction mixture was filtered through Celite and concentrated in vacuo to obtain the crude mixture. The crude mixture was diluted with water and acidified with 1N hydrochloric acid solution and extracted with ethyl acetate. The combined organic layer was washed with brine solution, dried over anhydrous sodium sulfate and filtered. The filtrate was concentrated in vacuo to afford the crude material which purified by was trituration with diethyl ether and n-pentane to afford 2-chloro-7-methoxyquinoline-3-carboxylic acid as a brown solid.

### Synthesis of 2-(4,4-difluoroazepan-1-yl)-7-methoxyquinoline-3-carboxylic acid

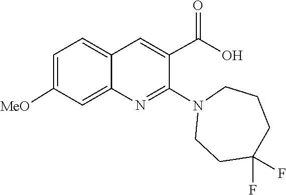 [SMILES: COc1ccc2cc(C(=O)O)c(N3CCCC(F)(F)CC3)nc2c1]

To a solution of 2-chloro-7-methoxyquinoline-3-carboxylic acid (1.0 g, 4.2 mmol) in N,N-dimethylformamide (20 mL) was added 4,4-difluoroazepane hydrogen chloride (0.85 g, 6.3 mmol) and potassium carbonate (2.3 g, 16.8 mmol). The mixture was heated at 70° C. for 12 hours under nitrogen atmosphere. The progress of reaction was monitored by TLC. After completion of reaction, the crude material was diluted with cold water followed by treatment with 1N hydrochloric acid solution to pH 3-4.0 and extracted with ethyl acetate. The combined organic layer was washed with brine solution and dried over anhydrous sodium sulphate and filtered. The filtrate was concentrated under reduced pressure to obtain the crude material which was purified by trituration with diethyl ether and n-pentane to afford 2-(4,4-difluoroazepan-1-yl)-7-methoxyquinoline-3-carboxylic acid as a light brown solid.

### Synthesis of 2-(4,4-difluoroazepan-1-yl)-7-methoxy-N-(3-sulfamoylphenyl)quinoline-3-carboxamide

To a stirred solution of 2-(4,4-difluoroazepan-1-yl)-7-methoxyquinoline-3-carboxylic acid (0.2 g, 0.6 mmol) in dichloromethane (4 mL) was added bis(dimethylamino)methylene]-1H-1,2,3-triazolo[4,5-b]pyridinium 3-oxide hexafluorophosphate (HATU) (0.34 g, 0.9 mmol) and N,N-dimethylpyridin-4-amine (DMAP) (0.037 g, 0.30 mmol) was added at 0° C. The reaction mixture was stirred at room temperature for 5 hours. After completion of reaction, solvent was removed under rotatory and diluted with acetonitrile (4 mL) and added to the solution of 3-aminobenzenesulfonamide (0.1 g, 0.7 mmol) at room temperature. The reaction mixture was reflux for 18 hours. The progress of the reaction was monitored by TLC. After completion of the reaction, the mixture was diluted with water and extracted with ethyl acetate. The combined organic layer was dried over anhydrous sodium sulphate and filtered. The filtrate was concentrated under reduced pressure and the resultant crude residue was purified by flash column chromatography with a gradient of 50-70% ethyl acetate in hexanes to afford 2-(4,4-difluoroazepan-1-yl)-7-methoxy-N-(3-sulfamoylphenyl)quinoline-3-carboxamide as white solid. MS (ESI): Calcd [M+H]⁺: 491.16, found: 491.20.

¹H NMR (400 MHz, DMSO-d₆): δ 10.98 (s, 1H), 8.32 (s, 1H), 8.24 (s, 1H), 7.86 (d, J=8 Hz, 1H), 7.74 (d, J=8 Hz, 1H), 7.56 (s, 1H), 7.55 (d, J=8 Hz, 1H), 7.40 (s, 2H), 7.02 (d, J=4 Hz, 1H), 7.95 (dd, J=2.4 Hz, J=8 Hz, 1H), 3.88 (s, 3H), 3.74-3.73 (m, 2H), 3.59 (t, J=8 Hz. 2H), 2.41-2.38 (m, 2H), 2.00-1.98 (m, 2H), 1.89-1.88 (m, 2H).

### Compound 18

### Synthesis of 2-(4,4-difluoroazepan-1-yl)-N-(3-sulfamoylphenyl)-5,6,7,8-tetrahydroquinoline-3-carboxamide

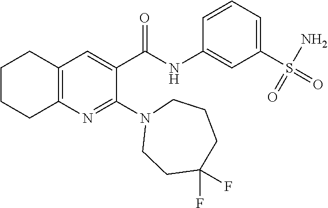 [SMILES: NS(=O)(=O)c1cccc(NC(=O)c2cc3c(nc2N2CCCC(F)(F)CC2)CCCC3)c1]

### Synthesis of (E)-2-((dimethylamino)methylene)cyclohexan-1-one (2)

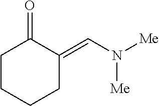 [SMILES: CN(C)/C=C1\CCCCC1=O]

A mixture of cyclohexanone (10.0 g, 100 mmol) and 1-tert-butoxy-N,N,N′,N′-tetramethylmethanediamine (18 g, 100 mmol) was stirred at room temperature overnight. Afterwards, heated the reaction mixture at 110° C. for 12 hours. The progress of reaction was monitored by TLC and LCMS. After completion, the solvent was removed under rotatory evaporation to obtain crude (E)-2-((dimethylamino)methylene)cyclohexan-1-one as a brown liquid which was used as such into next step without any purification.

### Synthesis of methyl 2-hydroxy-5,6,7,8-tetrahydroquinoline-3-carboxylate

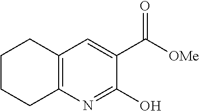 [SMILES: COC(=O)c1cc2c(nc1O)CCCC2]

To the crude (E)-2-((dimethylamino)methylene)cyclohexan-1-one (15.0 g, 98 mmol) in methanol (75 mL) was added methyl 2-cyanoacetate (11 g, 107 mmol) and the reaction mixture was heated at 80° C. for 12 hours. After completion of reaction, the solvent was removed under rotatory evaporation and the crude mass which was purified by flash column chromatography with a gradient of 70-80% ethyl acetate in hexane to afford methyl 2-hydroxy-5,6,7,8-tetrahydroquinoline-3-carboxylate as white solid.

### Synthesis of methyl 2-chloro-5,6,7,8-tetrahydroquinoline-3-carboxylate

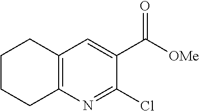 [SMILES: COC(=O)c1cc2c(nc1Cl)CCCC2]

Methyl 2-hydroxy-5,6,7,8-tetrahydroquinoline-3-carboxylate (3.3 g, 15 mmol) was dissolved into phosphorus oxychloride (33 mL) at room temperature and slowly heated to reflux for 12 hours. The reaction mixture was then cooled to room temperature and excess of phosphorus oxychloride was distilled out in vacuo. The residue obtained was dissolved into ethyl acetate and washed with aq. sodium bicarbonate solution. The organic layer was separated, washed with brine solution, dried over anhydrous sodium sulphate and concentrated in vacuum to afford the crude material. The crude material was triturated with diethyl ether and pentane to afford methyl 2-chloro-5,6,7,8-tetrahydroquinoline-3-carboxylate as a white solid.

### Synthesis of methyl 2-(4,4-difluoroazepan-1-yl)-5,6,7,8-tetrahydroquinoline-3-carboxylate

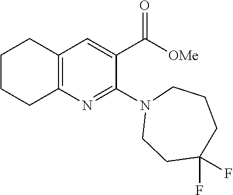 [SMILES: COC(=O)c1cc2c(nc1N1CCCC(F)(F)CC1)CCCC2]

To a solution of methyl 2-chloro-5,6,7,8-tetrahydroquinoline-3-carboxylate (3.0 g, 13 mmol) in N,N-dimethylformamide (30 mL) was added 4,4-difluoroazepane hydrogen chloride (2.7 g, 15.6 mmol) and potassium carbonate (7.3 g, 5.2 mmol). The reaction mixture was heated at 100° C. for 48 hours under nitrogen atmosphere. The progress of reaction was monitored by TLC. After completion of reaction, the crude material was diluted with ice-cold water and extracted with ethyl acetate. The organic layer was washed with brine solution and dried over anhydrous sodium sulphate. The crude material was purified by flash column chromatography with a gradient of 20-30% ethyl acetate in hexanes to afford methyl 2-(4,4-difluoroazepan-1-yl)-5,6,7,8-tetrahydroquinoline-3-carboxylate as a yellow liquid.

### Synthesis of 2-(4,4-difluoroazepan-1-yl)-5,6,7,8-tetrahydroquinoline-3-carboxylic acid

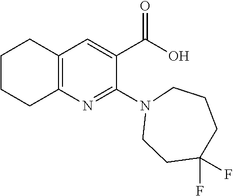 [SMILES: O=C(O)c1cc2c(nc1N1CCCC(F)(F)CC1)CCCC2]

To a solution of methyl 2-(4,4-difluoroazepan-1-yl)-5,6,7,8-tetrahydroquinoline-3-carboxylate (1.5 g, 4.6 mmol) in tetrahydrofuran (20 mL) and methanol (20 mL) was added lithium hydroxide (0.97 g, 20 mmol) in water (20 mL) and stirred at room temperature for 12 h. After completion of reaction, solvent was removed under rotatory evaporation and diluted with water, acidified with 1N hydrochloric solution and extracted with ethyl acetate. The organic layer was dried over sodium sulphate and filtered. The filtrate was concentrated under reduced pressure and the residue was purified by trituration with diethyl ether and n-pentane to afford 2-(4,4-difluoroazepan-1-yl)-5,6,7,8-tetrahydroquinoline-3-carboxylic acid as a white solid.

### Synthesis of 2-(4,4-difluoroazepan-1-yl)-N-(3-sulfamoylphenyl)-5,6,7,8-tetrahydroquinoline-3-carboxamide

To a solution of 2-(4,4-difluoroazepan-1-yl)-5,6,7,8-tetrahydroquinoline-3-carboxylic acid (0.31 g, 1 mmol) in dichloromethane (10 mL) was added (1-[Bis(dimethylamino)methylene]-1H-1,2,3-triazolo[4,5-b]pyridinium3-oxide hexafluorophosphate (HATU) (0.57 g, 1.5 mmol) and 4-(dimethylamino)pyridine (0.006 g, 0.05 mmol) at 0° C. and stirred the mixture at room temperature for 12 hours. After completion of reaction, solvent was evaporated under nitrogen atmosphere and diluted with acetonitrile and to this solution, 3-aminobenzenesulfonamide (0.18 g, 1.1 mmol) was added and the mixture was heated to 80° C. for 12 hours. The progress of reaction was monitored by TLC. After completion of reaction, acetonitrile was removed under rotatory evaporation. The crude mass was diluted with water and extracted with ethyl acetate. The organic layer was washed with brine solution, dried over anhydrous sodium sulphate and concentrated. The crude material was purified by flash column chromatography with a gradient of 40-50% ethyl acetate in hexanes to afford 2-(4,4-difluoroazepan-1-yl)-N-(3-sulfamoylphenyl)-5,6,7,8-tetrahydroquinoline-3-carboxamide as white solid. MS (ESI): Calcd [M+H]⁺: 465.18, found: 465.25.

¹H NMR (400 MHz, DMSO-d₆): δ 10.58 (s, 1H), 8.29 (s, 1H), 7.8 (d, J=3.6 Hz, 1H), 7.52-7.51 (m, 2H), 7.42 (s, 1H), 7.37 (s, 2H), 3.57-3.56 (m, 2H), 3.38 (t, J=5.6 Hz, 2H), 2.70-2.67 (m, 2H), 2.65-2.63 (m, 2H), 2.32-2.29 (m, 2H), 1.94-1.90 (m, 2H), 1.80-1.79 (m, 4H), 1.73-1.72 (m, 2H).

### Compound 19

### Synthesis of 2-(4,4-difluoroazepan-1-yl)-N-(3-sulfamoylphenyl)-1,8-naphthyridine-3-carboxamide

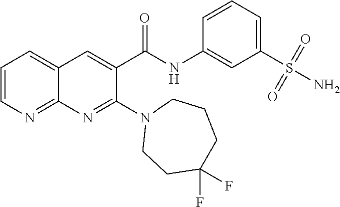 [SMILES: NS(=O)(=O)c1cccc(NC(=O)c2cc3cccnc3nc2N2CCCC(F)(F)CC2)c1]

### Synthesis of methyl 2-hydroxy-1,8-naphthyridine-3-carboxylate

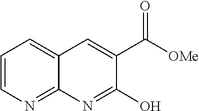 [SMILES: COC(=O)c1cc2cccnc2nc1O]

To a stirred solution of 2-hydroxy-1,8-naphthyridine-3-carboxylic acid (5.0 g, 26 mmol) in methanol (50 mL), thionyl chloride (50 ml) was added dropwise at 0° C. under nitrogen atmosphere. After addition of thionyl chloride, the reaction mixture was slowly warmed to room temperature and then heated at 70° C. for 16 hours. After completion of reaction, the reaction mixture was cooled to room temperature, the solid precipitate formed was filtered and washed with water and dried under vacuum to afford methyl 2-hydroxy-1,8-naphthyridine-3-carboxylate as an off-white solid.

### Synthesis of methyl 2-chloro-1,8-naphthyridine-3-carboxylate

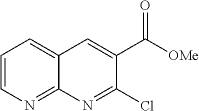 [SMILES: COC(=O)c1cc2cccnc2nc1Cl]

A solution of methyl 2-hydroxy-1,8-naphthyridine-3-carboxylate (4.5 g, 22 mmol) in phosphorus oxychloride (50 mL) was heated at 130° C. for 16 hours under nitrogen atmosphere. Progress of the reaction was monitored by TLC. After completion of reaction, the reaction mixture was concentrated under reduced pressure and the crude residue was poured into crushed ice and basified with aqueous sodium bicarbonate and then extracted with ethyl acetate. The organic layer was washed with brine solution, dried over anhydrous sodium sulfate and evaporated under reduced pressure to obtain the crude product. The crude material was purified by flash column chromatography with a gradient of 50% ethyl acetate in hexanes to afford methyl 2-chloro-1,8-naphthyridine-3-carboxylate as a yellow oil.

### Synthesis of methyl 2-(4,4-difluoroazepan-1-yl)-1,8-naphthyridine-3-carboxylate

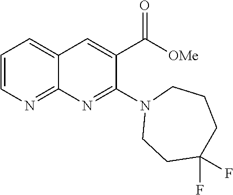 [SMILES: COC(=O)c1cc2cccnc2nc1N1CCCC(F)(F)CC1]

To a stirred solution of methyl 2-chloro-1,8-naphthyridine-3-carboxylate (1.2 g, 5.4 mmol) in N,N-dimethylformamide (12 mL), 4,4-difluoroazepane hydrochloride (1.4 g, 8.1 mmol) and potassium carbonate (3 g, 21.6 mmol) were added at room temperature. The resulting mixture was heated at 100° C. for 16 hours. Progress of the reaction was monitored by TLC. After completion, the reaction mixture was diluted with water and extracted with ethyl acetate. The organic layer was washed with brine, dried over anhydrous sodium sulfate and evaporated under reduced pressure to afford the crude product. The crude product was purified by flash column chromatography with a gradient of 40-50% ethyl acetate in heptane to afford methyl 2-(4,4-difluoroazepan-1-yl)-1,8-naphthyridine-3-carboxylate as a light yellow oil.

Synthesis of 2-(4,4-difluoroazepan-1-yl)-1,8-naphthyridine-3-carboxylic acid

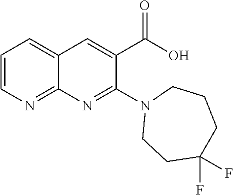 [SMILES: O=C(O)c1cc2cccnc2nc1N1CCCC(F)(F)CC1]

To a stirred solution of methyl 2-(4,4-difluoroazepan-1-yl)-1,8-naphthyridine-3-carboxylate (1.2 g, 3.7 mmol) in methanol (20 mL) and tetrahydrofuran (20 mL), aqueous solution of lithium hydroxide (0.36 g, 15 mmol) in water (20 mL) was added and the resulting reaction mixture was stirred at room temperature for 24 hours. Progress of the reaction was monitored by TLC. The organic solvents were removed under reduced pressure and diluted with water (50 mL), acidified with hydrochloric acid 1N and extracted with 20% isopropyl alcohol in chloroform. The organic layer was washed with brine, dried over anhydrous sodium sulfate and evaporated under reduced pressure. The crude material was purified through trituration with diethyl ether and n-pentane to afford 2-(4,4-difluoroazepan-1-yl)-1,8-naphthyridine-3-carboxylic acid as a white solid.

### Synthesis of 2-(4,4-difluoroazepan-1-yl)-N-(3-sulfamoylphenyl)-1,8-naphthyridine-3-carboxamide

To a stirred suspension of 2-(4,4-difluoroazepan-1-yl)-1,8-naphthyridine-3-carboxylic acid (0.2 g, 0.65 mmol) in dry dichloromethane (20 mL), [Bis(dimethylamino)methylene]-1H-1,2,3-triazolo[4,5-b]pyridinium 3-oxid hexafluorophosphate (HATU) (0.37 g, 0.98 mmol) and N,N-dimethylpyridin-4-amine (0.039 g, 0.3 mmol) were added sequentially at 0° C. The resulting mixture was stirred at room temperature for 18 hours. Then, the reaction mixture was evaporated under vacuum to remove the dichloromethane and the resulting residue was dissolved in acetonitrile (10 mL). To the resulting mixture, 3-aminobenzene-1-sulfonamide (0.13 g, 0.78 mmol) was added and reaction mixture was heated at 80° C. for 18 hours. After completion of reaction, the reaction mixture was cooled to room temperature, diluted with water and extracted with ethyl acetate. The organic layer was washed with brine, dried over anhydrous sodium sulfate and evaporated under reduced pressure to afford the crude. The crude product was purified by flash column chromatography with a gradient of 100% ethyl acetate in hexanes to afford 2-(4,4-difluoroazepan-1-yl)-N-(3-sulfamoylphenyl)-1,8-naphthyridine-3-carboxamide as a light yellow solid. MS (ESI): Calcd [M+H]⁺: 462.14, found: 462.30.

¹H NMR (400 MHz, DMSO-d₆): δ 11.01 (s, 1H), 8.84-8.83 (m, 1H), 8.41 (s, 1H), 8.30-8.26 (m, 2H), 7.86-7.84 (m, 1H), 7.58-7.57 (m, 2H), 7.42 (s, 2H), 7.33-7.30 (m, 1H), 3.82-3.80 (m, 2H), 3.63-3.62 (m, 2H), 2.15-2.01 (m, 3H), 1.92-1.90 (m, 3H).

### Compound 20

### 2-(4,4-difluoroazepan-1-yl)-N-(3-sulfamoylphenyl)-1,6-naphthyridine-3-carboxamide

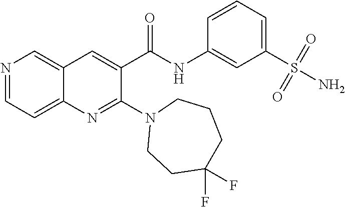 [SMILES: NS(=O)(=O)c1cccc(NC(=O)c2cc3cnccc3nc2N2CCCC(F)(F)CC2)c1]

### Synthesis of ethyl 2-hydroxy-1,6-naphthyridine-3-carboxylate

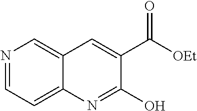 [SMILES: CCOC(=O)c1cc2cnccc2nc1O]

To a stirred solution of 4-aminonicotinaldehyde (5 g, 41 mmol) in ethanol (80 mL) was added 1,3-diethyl propanedioate (13 g, 82 mmol) and piperidine (0.87 g, 10 mmol) was added dropwise. The reaction mixture was stirred at 80° C. for 18 hours. After completion of reaction, the reaction mixture was cooled to room temperature and filtered. The solid mass was dried under vacuum to afford ethyl 2-hydroxy-1,6-naphthyridine-3-carboxylate as an off-white solid.

### Synthesis of ethyl 2-chloro-1,6-naphthyridine-3-carboxylate

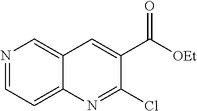 [SMILES: CCOC(=O)c1cc2cnccc2nc1Cl]

Mixture of ethyl 2-hydroxy-1,6-naphthyridine-3-carboxylate (2 g, 9.2 mmol) and phosphorous(V) oxychloride (20 mL) was heated at 130° C. for 18 hours under nitrogen atmosphere. After completion of reaction, the crude material was concentrated under reduced pressure. The reaction mixture was quenched with aqueous sodium carbonate and extracted with ethyl acetate. The organic layer was washed with brine solution and dried over anhydrous sodium sulphate and concentrated under reduced pressure. The crude material was purified by flash column chromatography with a gradient of 30-40% ethyl acetate in hexanes to afford ethyl 2-chloro-1,6-naphthyridine-3-carboxylate as a light yellow solid.

### Synthesis of ethyl 2-(4,4-difluoroazepan-1-yl)-1,6-naphthyridine-3-carboxylate

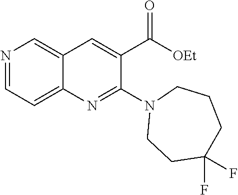 [SMILES: CCOC(=O)c1cc2cnccc2nc1N1CCCC(F)(F)CC1]

To a solution of ethyl 2-chloro-1,6-naphthyridine-3-carboxylate (0.8 g, 3.4 mmol) in N,N-dimethylformamide (10 mL) was added 4,4-difluoroazepane hydrogen chloride (0.9 g, 5.1 mmol) and potassium carbonate (1.9 g, 13.5 mmol). The resulting reaction mixture was heated at 80° C. for 16 hours under nitrogen atmosphere. The progress of reaction was monitored by TLC. After completion of reaction, the reaction mixture was diluted with water and extracted with ethyl acetate. The organic layer was washed with brine and dried over anhydrous sodium sulphate, filtered and concentrated. The crude residue was purified by flash column chromatography with a gradient of 50-70% ethyl acetate in hexanes to afford ethyl 2-(4,4-difluoroazepan-1-yl)-1,6-naphthyridine-3-carboxylate as light yellow solid.

### Synthesis of 2-(4,4-difluoroazepan-1-yl)-1,6-naphthyridine-3-carboxylic acid

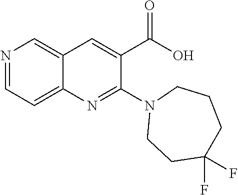 [SMILES: O=C(O)c1cc2cnccc2nc1N1CCCC(F)(F)CC1]

To a solution of ethyl 2-(4,4-difluoroazepan-1-yl)-1,6-naphthyridine-3-carboxylate (0.75 g, 2.3 mmol) in a mixture of methanol (25 mL) and tetrahydrofuran (25 mL) was added a solution of lithium hydroxide monohydrate (0.4 g, 9.2 mmol) in water (25 mL). The mixture was stirred at room temperature for 12 hours. The progress of reaction was monitored by TLC. After completion of reaction, the crude material was diluted with water followed by treatment with 1 N hydrochloric acid solution and extracted with 10% isopropanol in chloroform. The organic layer was washed with brine solution and dried over anhydrous sodium sulphate and concentrated under reduced pressure. The crude material was triturated with diethyl ether and n-pentane to afford 2-(4,4-difluoroazepan-1-yl)-1,6-naphthyridine-3-carboxylic acid as a light yellow solid.

### Synthesis of 2-(4,4-difluoroazepan-1-yl)-N-(3-sulfamoylphenyl)-1,6-naphthyridine-3-carboxamide

To a stirred solution of 2-(4,4-difluoroazepan-1-yl)-1,6-naphthyridine-3-carboxylic acid (0.25 g, 0.8 mmol) in dichloromethane (20 mL) was added bis(dimethylamino)methylene]-1H-1,2,3-triazolo[4,5-b]pyridinium-3-oxide hexafluorophosphate (HATU) (0.46 g, 1.2 mmol) and N,N-dimethylpyridin-4-amine (DMAP) (0.049 g, 0.41 mmol) was added at 0° C. The reaction mixture was stirred at room temperature for 18 hours. The progress of reaction was monitored by TLC and LCMS. After completion of reaction, dichloromethane was removed under reduced pressure. The resulting residue was diluted with acetonitrile (20 mL) and to this mixture, 3-aminobenzenesulfonamide (0.17 g, 1 mmol) was added and stirred at 70° C. for 18 hours. The progress of the reaction was monitored by TLC. After completion of the reaction, the mixture was diluted with water and extracted with ethyl acetate. The organic layer was dried over anhydrous sodium sulphate and filtered. The filtrate was concentrated under reduced pressure and the crude residue was purified by flash column chromatography with a gradient of 50-10% methanol in dichloromethane to afford 2-(4,4-difluoroazepan-1-yl)-N-(3-sulfamoylphenyl)-1,6-naphthyridine-3-carboxamide as a light yellow solid. MS (ESI): Calcd [M+H]⁺: 462.14, found: 462.30.

¹H NMR (400 MHz, DMSO-d₆): δ 11.02 (s, 1H), 9.07 (s, 1H), 8.52-8.49 (m, 2H), 8.30 (m, 1H), 7.86-7.84 (m, 1H), 7.59-7.55 (m, 2H), 7.46-7.42 (m, 3H), 7.95 (s, 1H), 3.85-3.75 (m, 2H), 3.64 (m, 2H), 2.1-2.0 (m, 1H), 1.90 (s, 3H), 1.75 (s, 1H)

### Compound 21

### Synthesis of 2-(4,4-difluoroazepan-1-yl)-N-(3-(methylsulfonyl)phenyl)-5,6,7,8-tetrahydroquinoline-3-carboxamide

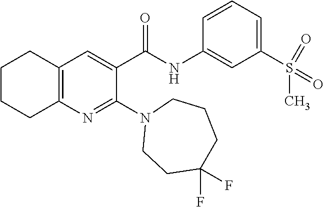 [SMILES: CS(=O)(=O)c1cccc(NC(=O)c2cc3c(nc2N2CCCC(F)(F)CC2)CCCC3)c1]

To a solution of 2-(4,4-difluoroazepan-1-yl)-5,6,7,8-tetrahydroquinoline-3-carboxylic acid (0.20 g, 0.64 mmol) in dichloromethane (4 mL) was added (1-[Bis(dimethylamino)methylene]-1H-1,2,3-triazolo[4,5-b]pyridinium 3-oxide hexafluorophosphate (HATU) (0.38 g, 1 mmol) and 4-dimethylaminopyridine (0.004 g, 0.03 mmol) at 0° C. and stirred the mixture at room temperature for 12 hours. After completion of reaction, dichloromethane was evaporated under reduced pressure and the crude residue was dissolved into acetonitrile (4 mL) and to this mixture, 3-(methylsulfonyl)aniline (0.19 g, 1.1 mmol) was added and heated to reflux for 12 hours. After completion of reaction, acetonitrile was removed under rotatory evaporation and the crude mass was diluted with water and extracted with ethyl acetate. The organic layer was washed with brine solution and dried over anhydrous sodium sulphate, filtered and concentrated. The crude material was purified by preparative HPLC to afford 2-(4,4-difluoroazepan-1-yl)-N-(3-(methylsulfonyl)phenyl)-5,6,7,8-tetrahydroquinoline-3-carboxamide as an off-white solid. MS (ESI): Calcd [M+H]⁺: 464.18, found: 464.30.

¹H NMR (400 MHz, DMSO-d₆) δ 10.67 (s, 1H), 8.34 (s, 1H), 7.96-7.95 (m, 1H), 7.63-7.59 (m, 2H), 7.45 (s, 1H), 3.57-3.56 (m, 2H), 3.38 (t, J=5.6 Hz, 2H), 3.2 (s, 3H), 2.70-2.67 (m, 2H), 2.65-2.62 (m, 2H), 2.35-2.26 (m, 2H), 2.00-1.93 (m, 2H), 1.83-1.79 (m, 4H), 1.77-1.73 (m, 2H).

### Compound 22

### Synthesis of 2-(4,4-difluoroazepan-1-yl)-N-(3-(methylsulfonyl)phenyl)-1,8-naphthyridine-3-carboxamide

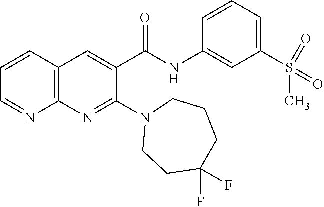 [SMILES: CS(=O)(=O)c1cccc(NC(=O)c2cc3cccnc3nc2N2CCCC(F)(F)CC2)c1]

To a stirred suspension of 2-(4,4-difluoroazepan-1-yl)-1,8-naphthyridine-3-carboxylic acid (0.2 g, 0.65 mmol) in dry dichloromethane (10 mL), [Bis(dimethylamino)methylene]-1H-1,2,3-triazolo[4,5-b]pyridinium 3-oxid hexafluorophosphate (HATU) (0.37 g, 0.98 mmol) and N,N-dimethylpyridin-4-amine (0.039 g, 0.325 mmol) were added sequentially at 0° C. The resulting mixture was stirred at room temperature for 18 hours. Afterwards, reaction mixture was evaporated under vacuum to remove the dichloromethane and the resulting residue was dissolved in acetonitrile (10 mL) and this solution, 3-(methylsulfonyl)aniline (0.13 g, 0.78 mmol) was added and heated at 80° C. for 18 hours. After completion of reaction, the reaction mixture was cooled to room temperature, diluted with water and extracted with ethyl acetate. The organic layer was washed with brine, dried over anhydrous sodium sulfate and evaporated under reduced pressure to afford the crude. The crude product was purified by flash column chromatography with a gradient of 100% ethyl acetate in hexanes to afford 2-(4,4-difluoroazepan-1-yl)-N-(3-(methylsulfonyl)phenyl)-1,8-naphthyridine-3-carboxamide as a light yellow solid. MS (ESI): Calcd [M+H]⁺: 461.15, found: 461.30.

¹H NMR (400 MHz, DMSO-d₆): δ 11.09 (s, 1H), 8.85-8.83 (m, 1H), 8.43 (s, 1H), 8.36-8.34 (m, 1H), 8.27-8.25 (m, 1H), 8.02-8.00 (m, 1H), 7.70-7.65 (m, 2H), 7.33-7.30 (m, 1H), 3.85-3.75 (m, 2H), 3.64-3.63 (m, 2H), 3.23 (s, 3H), 2.44-2.40 (m, 2H), 2.07-2.00 (m, 2H), 1.96-1.91 (m, 2H)

### Compound 23

### Synthesis of 2-(4,4-difluoroazepan-1-yl)-N-(3-(methylsulfonyl)phenyl)-1,6-naphthyridine-3-carboxamide

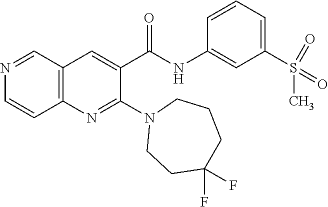 [SMILES: CS(=O)(=O)c1cccc(NC(=O)c2cc3cnccc3nc2N2CCCC(F)(F)CC2)c1]

To a stirred solution of 2-(4,4-difluoroazepan-1-yl)-1,6-naphthyridine-3-carboxylic acid (0.25 g, 0.81 mmol) in dichloromethane (5 mL) was added Bis(dimethylamino)methylene]-1H-1,2,3-triazolo[4,5-b]pyridinium 3-oxide hexafluorophosphate (HATU) (0.46 g, 1.2 mmol) and N,N-dimethylpyridin-4-amine (0.05 g, 0.41 mmol) was added at 0° C. The reaction mixture was stirred at room temperature for 5 hours. After completion of reaction, solvent was removed under reduced pressure and diluted with acetonitrile (5 mL) and to this, 3-methanesulfonylaniline (0.14 g, 0.81 mmol) was added at room temperature and slowly heated to 70° C. for 18 hours. The progress of the reaction was monitored by TLC. After completion of reaction, the reaction mixture was diluted with water and extracted with ethyl acetate. The organic layer was dried over anhydrous sodium sulfate and filtered. The filtrate was concentrated under reduced pressure and the crude residue was purified by flash column chromatography with a gradient of 50-70% ethyl acetate in hexanes to afford 2-(4,4-difluoroazepan-1-yl)-N-(3-(methylsulfonyl)phenyl)-1,6-naphthyridine-3-carboxamide as an off-white solid. MS (ESI): Calcd [M+H]⁺: 461.15, found: 461.3.

¹H NMR (400 MHz, DMSO-d₆): δ 11.15 (s, 1H), 9.00 (s, 1H), 8.43 (s, 1H), 8.36 (d, J=4 Hz, 2H), 7.74 (m, 1H), 7.75 (d, J=4 Hz, 1H), 7.72 (d, J=8 Hz, 1H), 7.95 (s, 1H), 3.80-3.77 (m, 2H), 3.64 (t, J=8 Hz, 2H), 3.23 (s, 3H), 2.42-2.39 (m, 2H), 2.00-1.97 (m, 2H), 1.93-1.90 (m, 2H).

### Compound 24

### Synthesis of 2-(4,4-difluoroazepan-1-yl)-N-(3-(S-methylsulfonimidoyl)phenyl)-6-(trifluoromethyl)quinoline-3-carboxamide

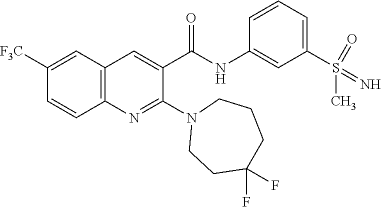 [SMILES: CS(=N)(=O)c1cccc(NC(=O)c2cc3cc(C(F)(F)F)ccc3nc2N2CCCC(F)(F)CC2)c1]

To a solution of 2-(4,4-difluoroazepan-1-yl)-6-(trifluoromethyl)quinoline-3-carboxylic acid (0.2 g, 0.53 mmol) in dry dichloromethane (6.0 mL) was added oxalyl chloride (0.10 mL, 1.16 mmol) and catalytic amount of N,N-dimethylformamide (2 drops) under nitrogen atmosphere at 0° C. and the reaction mixture was stirred for 2 hours at room temperature. After completion of reaction, the solvent was removed under nitrogen atmosphere and the crude mixture was dissolved into dry dichloromethane (6 mL) and added to the solution of (3-aminophenyl)(imino)(methyl)-λ⁶-sulfanone (0.11 g. 0.64 mmol) and N,N-diisopropylethyl amine (0.50 mL, 2.65 mmol) in dichloromethane (6 mL) at 0° C. and stirred the mixture for 2 hours at room temperature. The progress of reaction was monitor by TLC. After completion of reaction, the mixture was quenched with water, extracted with dichloromethane. The organic layer was dried over sodium sulfate, filtered and concentrated to afford crude material. The crude residue was purified through reversed phase prep-HPLC to afford 2-(4,4-difluoroazepan-1-yl)-N-(3-(S-methylsulfonimidoyl)phenyl)-6-(trifluoromethyl)quinoline-3-carboxamide as a light brown solid. MS (ESI): Calcd [M+H]⁺: 527.15, found: 527.20.

¹H NMR (400 MHz, DMSO-d₆): δ 11.04 (s, 1H), 8.50 (s, 1H), 8.34 (t, J=2.0 Hz, 1H), 8.30 (s, 1H), 7.96 (d, J=8.8 Hz, 1H), 7.85 (dd. J=2.0, 8.8 Hz, 1H), 7.76 (d, J=8.0 Hz, 1H), 7.69 (d, J=8.0 Hz, 1H), 7.61 (t, J=7.6 Hz, 1H), 4.23 (s, 1H), 3.97-3.74 (m, 2H), 3.64 (t, J=5.6 Hz, 2H), 3.07 (s, 3H), 2.45-2.37 (m, 2H), 2.01-1.96 (m, 2H), 1.91-1.90 (m, 2H).

### Compound 25

### Synthesis of 2-(4,4-difluoroazepan-1-yl)-N-(3-sulfamoylphenyl)-7-(trifluoromethyl)quinoline-3-carboxamide

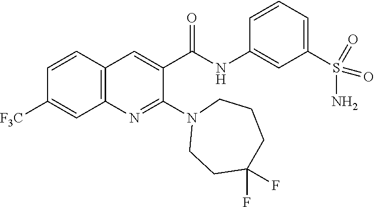 [SMILES: NS(=O)(=O)c1cccc(NC(=O)c2cc3ccc(C(F)(F)F)cc3nc2N2CCCC(F)(F)CC2)c1]

### Synthesis of 2-amino-4-(trifluoromethyl)benzaldehyde

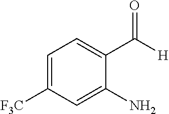 [SMILES: Nc1cc(C(F)(F)F)ccc1C=O]

To a stirred solution of 2-nitro-4-(trifluoromethyl)benzaldehyde (5 g, 23 mmol) in ethanol (50 mL) was added iron powder (13.2 g, 22.8 mmol) and conc. HCl (0.6 mL) at room temperature and slowly refluxed for 2 hours. After completion of reaction, the reaction mixture was filtered through Celite and solvent was removed by rotary evaporation and diluted with water and then extracted with ethyl acetate. The organic layer was dried over anhydrous sodium sulphate and filtered. The filtrate was concentrated under reduced pressure and the resultant crude material was triturated with n-pentane to afford 2-amino-4-(trifluoromethyl)benzaldehyde as pale yellow solid.

### Synthesis of ethyl 2-hydroxy-7-(trifluoromethyl)quinoline-3-carboxylate

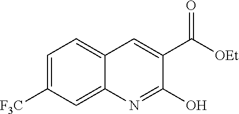 [SMILES: CCOC(=O)c1cc2ccc(C(F)(F)F)cc2nc1O]

To a stirred solution of 2-amino-4-(trifluoromethyl)benzaldehyde (2 g, 11 mmol) in ethanol (20 mL) was added diethyl malonate (13.8 g, 105.7 mmol) and piperidine (5.3 mL, 52.9 mmol) at room temperature. The reaction mixture was stirred at 80° C. for 12 hours. After completion of reaction, the reaction mixture was quenched with ice-cold water and extracted with ethyl acetate. The organic layer was dried over anhydrous sodium sulphate and filtered. The filtrate was concentrated under reduced pressure and triturated with diethyl ether to afford ethyl 2-hydroxy-7-(trifluoromethyl)quinoline-3-carboxylate as an off-white solid.

### Synthesis of ethyl 2-chloro-7-(trifluoromethyl)quinoline-3-carboxylate

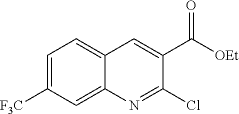 [SMILES: CCOC(=O)c1cc2ccc(C(F)(F)F)cc2nc1Cl]

A mixture of ethyl 2-hydroxy-7-(trifluoromethyl)quinoline-3-carboxylate (1 g, 11 mmol) and phosphorous oxychloride (10 mL) was slowly heated to 120° C. for 5 hours. After completion of the reaction, the reaction mixture was quenched with ice-water followed by saturated sodium bicarbonate solution, extracted with ethyl acetate. The organic layer was dried over anhydrous sodium sulphate and filtered. The filtrate was concentrated under reduced pressure to obtain the crude material which was triturated with n-pentane to afford ethyl 2-chloro-7-(trifluoromethyl)quinoline-3-carboxylate as a brown oil.

### Synthesis of ethyl 2-(4,4-difluoroazepan-1-yl)-7-(trifluoromethyl)quinoline-3-carboxylate

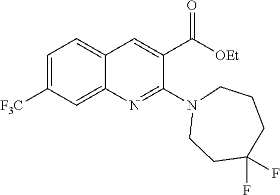 [SMILES: CCOC(=O)c1cc2ccc(C(F)(F)F)cc2nc1N1CCCC(F)(F)CC1]

To a solution of ethyl 2-chloro-7-(trifluoromethyl)quinoline-3-carboxylate (0.5 g, 1.65 mmol) in N,N-dimethylformamide (5 mL) was added 4,4-difluoroazepane hydrogen chloride (0.42 g, 2.5 mmol) and potassium carbonate (0.9 g, 6.6 mmol) at room temperature. The reaction mixture was heated at 80° C. for 16 hours under nitrogen atmosphere. The progress of reaction was monitored by TLC. After completion, the crude material was diluted with water, extracted with ethyl acetate. The combined organic layer was washed with brine solution and dried over anhydrous sodium sulphate and filtered. The filtrate was concentrated under reduced pressure to obtain the crude material which was purified by flash column chromatography with a gradient of 50-70% EtOAc in hexanes to afford ethyl 2-(4,4-difluoroazepan-1-yl)-7-(trifluoromethyl)quinoline-3-carboxylate as a yellow solid.

### Synthesis of 2-(4,4-difluoroazepan-1-yl)-7-(trifluoromethyl)quinoline-3-carboxylic acid

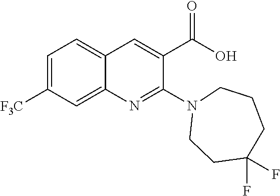 [SMILES: O=C(O)c1cc2ccc(C(F)(F)F)cc2nc1N1CCCC(F)(F)CC1]

To a stirred solution of ethyl 2-(4,4-difluoroazepan-1-yl)-7-(trifluoromethyl)quinoline-3-carboxylate (0.5 g, 1.2 mmol) in a mixture of methanol (8 mL) and THF (8 mL) was added a solution of lithium hydroxide (0.12 g, 5 mmol) in water (8 mL) at 0° C. and stirred at room temperature for 24 hours. The progress of reaction mixture was monitored by TLC and LCMS. After completion of reaction, solvent was removed by rotary and diluted with ice-water followed by treatment with 1 N hydrochloric acid solution and extracted with ethyl acetate. The organic layer was dried over anhydrous sodium sulphate and filtered. The filtrate was concentrated under reduced pressure and resultant crude was triturated with n-pentane and diethyl ether to afford 2-(4,4-difluoroazepan-1-yl)-7-(trifluoromethyl)quinoline-3-carboxylic acid as a brownish white solid.

### Synthesis of 2-(4,4-difluoroazepan-1-yl)-N-(3-sulfamoylphenyl)-7-(trifluoromethyl)quinoline-3-carboxamide

To a stirred solution of 2-(4,4-difluoroazepan-1-yl)-7-(trifluoromethyl)quinoline-3-carboxylic acid (0.28 g, 0.75 mmol) in dichloromethane (8 mL), (1-[Bis(dimethylamino)methylene]-1H-1,2,3-triazolo[4,5-b]pyridinium 3-oxide hexafluorophosphate (HATU) (0.43 g, 1.1 mmol) and 4-dimethylamino pyridine (DMAP) (0.048 g, 0.37 mmol) was added at 0° C. and stirred at room temperature for 12 hours. The progress of reaction mixture was monitored by TLC and LCMS. After completion of reaction, solvent was removed in vacuo and diluted with acetonitrile (3 mL) and added to the solution of 3-aminobenzenesulfonamide (0.16 g, 0.9 mmol) in acetonitrile (8 mL). The reaction mixture was heated at 80° C. for 12 hours. Then progress of the reaction was monitored by TLC. After completion of reaction, the reaction mixture was diluted with water and extracted with ethyl acetate, dried over anhydrous sodium sulphate and filtered. The filtrate was concentrated under reduced pressure and the crude residue was purified by flash column chromatography with a gradient of 50-70% ethyl acetate in hexanes to afford 2-(4,4-difluoroazepan-1-yl)-N-(3-sulfamoylphenyl)-7-(trifluoromethyl)quinoline-3-carboxamide as an off-white solid. MS (ESI): Calcd [M+H]⁺: 529.13, found: 529.25.

¹H NMR (400 MHz, DMSO-d₆): δ 11.05 (s, 1H), 8.48 (s, 1H), 8.31 (s, 1H), 8.07 (d, J=8.4 Hz, 1H), 7.92 (s, 1H), 7.85-7.87 (m, 1H), 7.54-7.60- (m, 3H), 7.43 (brs, 2H), 3.80-3.79 (m, 2H), 3.64 (t, J=6 Hz, 2H), 2.33-2.43 (m, 2H), 1.91-2.0 (m, 4H).

### Compound 26

### Synthesis of 3-(4,4-difluoro-3-methylpiperidin-1-yl)-N-(3-sulfamoylphenyl)quinoxaline-2-carboxamide

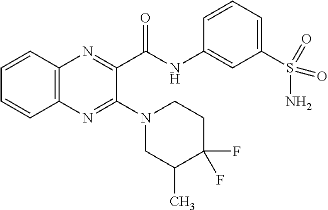 [SMILES: CC1CN(c2nc3ccccc3nc2C(=O)Nc2cccc(S(N)(=O)=O)c2)CCC1(F)F]

### Synthesis of 3-(4,4-difluoro-3-methylpiperidin-1-yl)quinoxaline-2-carboxylic acid

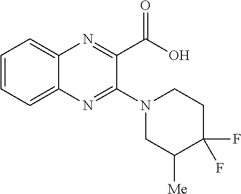 [SMILES: CC1CN(c2nc3ccccc3nc2C(=O)O)CCC1(F)F]

To a mixture of 3-chloroquinoxaline-2-carboxylic acid (180 mg, 0.87 mmol) and 4,4-difluoro-3-methylpiperidine hydrogen chloride (160 mg, 0.87 mmol) in N,N-dimethylformamide (4 mL), potassium carbonate (600 mg, 4.3 mmol) was added at room temperature. The reaction mixture was stirred at 70° C. for 18 hours. The progress of the reaction was monitored by TLC. After completion, the reaction mixture was diluted with ice cold water and extracted with ethyl acetate. The organic layers were combined and washed with brine. The organic layer then was dried over anhydrous sodium sulphate and filtered. The filtrate was concentrated under reduced pressure and the crude residue was purified by silica gel column chromatography with a gradient of 0-50% methanol in dichloromethane to afford 3-(4,4-difluoro-3-methylpiperidin-1-yl)quinoxaline-2-carboxylic acid as a yellow oil.

### Synthesis of 3-(4,4-difluoro-3-methylpiperidin-1-yl)-N-(3-sulfamoylphenyl)quinoxaline-2-carboxamide

To a stirred solution of 3-(4,4-difluoro-3-methylpiperidin-1-yl)quinoxaline-2-carboxylic acid (44 mg, 0.14 mmol) in dichloromethane (2 mL), (1-[Bis(dimethylamino)methylene]-1H-1,2,3-triazolo[4,5-b]pyridinium 3-oxide hexafluorophosphate (HATU) (81 mg, 1.4 mmol) and 4-dimethylamino pyridine (DMAP) (52 mg, 0.43 mmol) was added at room temperature and stirred at room temperature for 18 hours. The progress of reaction mixture was monitored by TLC and LCMS. Then progress of the reaction was monitored by TLC. After completion of reaction, the reaction mixture was concentrated under reduced pressure and the crude residue was purified using reversed-phase HPLC with a gradient of 5-95% acetonitrile in water to afford 3-(4,4-difluoro-3-methylpiperidin-1-yl)-N-(3-sulfamoylphenyl)quinoxaline-2-carboxamide as a white solid. LRMS (ESI): Calcd [M+H]⁺: 462.14, found: 461.9.

¹H NMR (400 MHz, CDCl₃) δ 10.01 (s, 1H), 8.32 (t, J=2.0 Hz, 1H), 8.11-8.06 (m, 1H), 7.99-7.94 (m, 1H), 7.83-7.79 (m, 1H), 7.76-7.71 (m, 2H), 7.60-7.54 (m, 2H), 4.90 (s, 2H), 4.09-3.95 (m, 2H), 3.40-3.31 (m, 1H), 3.16-3.07 (m, 1H), 2.33-2.19 (m, 3H), 1.11 (d, J=6.8 Hz, 3H).

### Compound 27

### Synthesis of 2-(4,4-difluoroazepan-1-yl)-7-fluoro-N-(2-sulfamoylpyridin-4-yl)quinoline-3-carboxamide

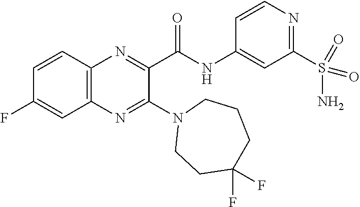 [SMILES: NS(=O)(=O)c1cc(NC(=O)c2nc3ccc(F)cc3nc2N2CCCC(F)(F)CC2)ccn1]

### Synthesis of 2-(4,4-difluoroazepan-1-yl)-7-fluoroquinoline-3-carboxylic acid

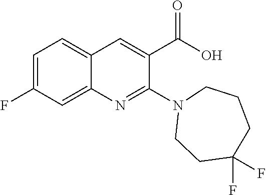 [SMILES: O=C(O)c1cc2ccc(F)cc2nc1N1CCCC(F)(F)CC1]

To a mixture of 2-chloro-7-fluoroquinoline-3-carboxylic acid (115 mg, 0.51 mmol) and 4,4-difluoroazepane hydrogen chloride (88 mg, 0.51 mmol) in N,N-dimethylformamide (4 mL), potassium carbonate (352 mg, 2.55 mmol) was added at room temperature. The reaction mixture was stirred at 60° C. for 18 hours. After completion, the reaction mixture was diluted with hydrochloric acid 1 N and extracted with dichloromethane. The organic layer then was dried over anhydrous sodium sulphate and filtered. The filtrate was concentrated under reduced pressure and the crude residue was purified by silica gel column chromatography with a gradient 0 to 20% methanol in dichloromethane to afford 2-(4,4-difluoro-3-methylpiperidin-1-yl)-7-fluoroquinoline-3-carboxylic acid as a yellow oil.

### Synthesis of 2-(4,4-difluoroazepan-1-yl)-7-fluoroquinoline-3-carboxamide

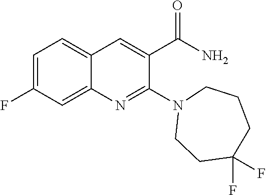 [SMILES: NC(=O)c1cc2ccc(F)cc2nc1N1CCCC(F)(F)CC1]

To a stirred solution of 2-(4,4-difluoro-3-methylpiperidin-1-yl)-7-fluoroquinoline-3-carboxamide (81 mg, 0.25 mmol) in dichloromethane (2 mL) was added (1-[Bis(dimethylamino)methylene]-1H-1,2,3-triazolo[4,5-b]pyridinium 3-oxide hexafluorophosphate (HATU) (0.14 g, 0.37 mmol) and ammonium carbonate (120 mg, 1.25 mmol) at room temperature. The reaction mixture was stirred at room temperature for 72 hours. After completion of reaction, the reaction mixture was diluted with water and extracted dichloromethane. The organic layers were combined, dried over anhydrous sodium sulphate, filtered and concentrated to afford crude material. The crude residue was purified by flash column chromatography using a Combiflash with a gradient of 0 to 100% ethyl acetate in hexanes to afford 2-(4,4-difluoroazepan-1-yl)-7-fluoroquinoline-3-carboxamide as a yellow powder.

### Synthesis of N-(2-(N,N-bis(2,4-dimethoxybenzyl)sulfamoyl)pyridin-4-yl)-2-(4,4-difluoroazepan-1-yl)-7-fluoroquinoline-3-carboxamide

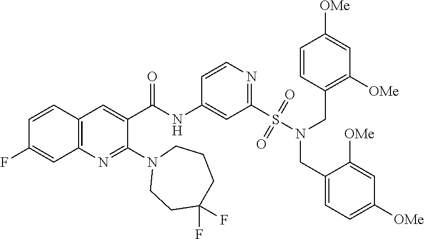 [SMILES: COc1ccc(CN(Cc2ccc(OC)cc2OC)S(=O)(=O)c2cc(NC(=O)c3cc4ccc(F)cc4nc3N3CCCC(F)(F)CC3)ccn2)c(OC)c1]

2-(4,4-difluoroazepan-1-yl)-7-fluoroquinoline-3-carboxamide (13.5 mg, 0.042 mmol), 4-bromo-N,N-bis(2,4-dimethoxybenzyl)pyridine-2-sulfonamide (22.4 mg, 0.042 mmol), cesium carbonate (27 mg, 0.084 mmol), and BrettPhos Pd G3 (4 mg, 0.0042 mmol) were added to a flame dried round bottom flask and capped. The reaction vessel was evacuated and the atmosphere was replace with nitrogen. Then, degassed anhydrous dioxane (2 mL) was added to the reaction vessel and the mixture was stirred at 100° C. for 18 hours. Dioxane was removed in vacuo and the crude mixture was purified by flash column chromatography with a gradient of ethyl acetate in hexanes to afford N-(2-(N,N-bis(2,4-dimethoxybenzyl)sulfamoyl)pyridin-4-yl)-2-(4,4-difluoroazepan-1-yl)-7-fluoroquinoline-3-carboxamide as a light yellow oil.

### Synthesis of 2-(4,4-difluoroazepan-1-yl)-7-fluoro-N-(2-sulfamoylpyridin-4-yl)quinoline-3-carboxamide

To a solution of N-(2-(N,N-bis(2,4-dimethoxybenzyl)sulfamoyl)pyridin-4-yl)-2-(4,4-difluoroazepan-1-yl)-7-fluoroquinoline-3-carboxamide (9 mg, 0.0115 mmol) in dichloromethane (2 mL) was added trifluoroacetic acid (500 μL). The reaction mixture was stirred for 18 hours then the volatiles were removed in vacuo. The resulting crude mixture was diluted with water and acetonitrile then purified using reversed-phase HPLC with a gradient of 5-95% acetonitrile in water to afford 2-(4,4-difluoroazepan-1-yl)-7-fluoro-N-(2-sulfamoylpyridin-4-yl)quinoline-3-carboxamide as a white powder. LRMS (ESI): Calcd [M+H]⁺: 480.13, found: 479.8.

¹H NMR (400 MHz, CDCl₃) δ 8.39-8.31 (m, 1H), 8.03-7.96 (m, 1H), 7.85-7.78 (m, 1H), 7.55-7.43 (m, 1H), 7.25-7.23 (m, 1H), 7.15-7.06 (m, 1H), 6.92-6.80 (m, 1H), 3.67-3.51 (m, 2H), 3.44-3.26 (m, 2H), 2.34-2.12 (m, 2H), 1.95-1.61 (m, 4H).

### Compound 28

### Synthesis of 2-(4,4-difluoroazepan-1-yl)-7-fluoro-N-(3-sulfamoylphenyl)quinoline-3-carboxamide

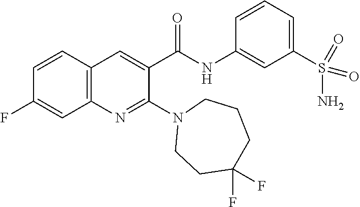 [SMILES: NS(=O)(=O)c1cccc(NC(=O)c2cc3ccc(F)cc3nc2N2CCCC(F)(F)CC2)c1]

### Synthesis of 2-chloro-7-fluoroquinoline-3-carboxylic acid

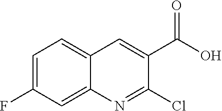 [SMILES: O=C(O)c1cc2ccc(F)cc2nc1Cl]

A stirred solution of 2-chloro-7-fluoroquinoline-3-carbaldehyde (1, 1.0 g, 4.77 mmol) in ethanol (30 mL), silver nitrate (1.3 g, 7.63 mmol) was added. The reaction mixture was stirred for 1 hours at room temperature. Afterwards, a solution of sodium hydroxide (0.95 g, 23.9 mmol) in 80% aqueous ethanol was added dropwise. The reaction mixture was stirred at room temperature for 5 hours. After completion of reaction, the reaction mixture was filtered through celite bed and solvent was removed by rotary evaporation and diluted with water, acidified with 1N hydrochloric acid solution and extracted with ethyl acetate. The organic layer was dried over anhydrous sulphate and filtered. The filtrate was concentrated under reduced pressure to afford 2-chloro-7-fluoroquinoline-3-carboxylic acid as an off-white solid.

### Synthesis of methyl 2-chloro-7-fluoroquinoline-3-carboxylate

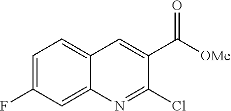 [SMILES: COC(=O)c1cc2ccc(F)cc2nc1Cl]

To a solution of 2-chloro-7-fluoroquinoline-3-carboxylic acid (0.9 g, 3.99 mmol) in N,N-dimethylformamide (10 mL) was added potassium carbonate (2.21 g, 12 mmol) and (0.60 ml, 9.97 mmol) at room temperature and stirred for 12 hours at 50° C. After completion of reaction, the reaction mixture was quenched with ice cold water and extracted with ethyl acetate. The organic layer was dried over anhydrous sodium sulphate and filtered. The filtrate was concentrated under reduced pressure to afford methyl 2-chloro-7-fluoroquinoline-3-carboxylate as a light yellow solid.

### Synthesis of methyl 2-(4,4-difluoroazepan-1-yl)-7-fluoroquinoline-3-carboxylate

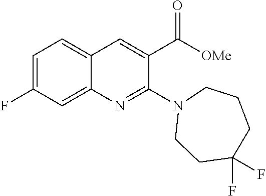 [SMILES: COC(=O)c1cc2ccc(F)cc2nc1N1CCCC(F)(F)CC1]

To a mixture of methyl 2-chloro-7-fluoroquinoline-3-carboxylate (550 mg, 2.3 mmol) and 4,4-difluoroazepane hydrogen chloride (0.59 g, 3.44 mmol) in N,N-dimethylformamide (5 mL), potassium carbonate (1.27 g, 12 mmol) was added at room temperature. The reaction mixture was stirred at 80° C. for 16 hours. The progress of the reaction was monitored by TLC. After completion, the reaction mixture was diluted with ice cold water and extracted with ethyl acetate. The organic layer was dried over anhydrous sodium sulphate and filtered. The filtrate was concentrated under reduced pressure and the crude residue was purified by silica gel column chromatography with a gradient of 40-50% ethyl acetate in hexanes to afford methyl 2-(4,4-difluoroazepan-1-yl)-7-fluoroquinoline-3-carboxylate as a yellow solid.

### Synthesis of 2-(4,4-difluoroazepan-1-yl)-7-fluoroquinoline-3-carboxylic acid

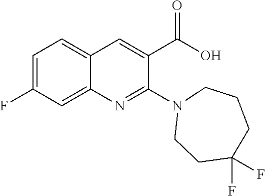 [SMILES: O=C(O)c1cc2ccc(F)cc2nc1N1CCCC(F)(F)CC1]

To a stirred solution of methyl 2-(4,4-difluoroazepan-1-yl)-7-fluoroquinoline-3-carboxylate (0.65 g, 1.92 mmol) in methanol (8 mL), tetrahydrofuran (8 mL) and water (8 mL) was added lithium hydroxide (0.162 g, 3.84 mmol). The reaction mixture was stirred at room temperature for 16 hours. The progress of reaction was monitored by TLC and LCMS. After completion of reaction, solvent was removed under rotatory and diluted with water, then acidified with 1N hydrochloric acid solution and extracted with ethyl acetate. The organic layer was combined, dried with sodium sulphate, filtered and concentrated to afford 2-(4,4-difluoroazepan-1-yl)-7-fluoroquinoline-3-carboxylic acid as a yellow solid.

### Synthesis of 2-(4,4-difluoroazepan-1-yl)-7-fluoro-N-(3-sulfamoylphenyl)quinoline-3-carboxamide

To a solution of 2-(4,4-difluoroazepan-1-yl)-7-fluoroquinoline-3-carboxylic acid (0.2 g, 0.62 mmol) in anhydrous dichloromethane (2 mL), was added oxalyl chloride (0.16 g, 1.2 mmol) and one drop of anhydrous N,N-dimethylformamide at 0° C. The Reaction mixture was stirred at 0° C. for 30 minutes. The progress of reaction was monitored by TLC. After completion, the reaction mixture was evaporated on vacuum under nitrogen atmosphere. The crude residue was dissolved into dry dichloromethane (6 mL) and added to the solution of 3-aminobenzene-1-sulfonamide (7, 0.13 g, 0.74 mmol) and N,N-diisopropylethylamine (0.32 g, 2.5 mmol) in dichloromethane (5 mL) at 0° C. The reaction mixture was stirred at room temperature for 1 hour. After completion, the reaction mixture was diluted with dichloromethane and washed with water and brine solution. The combined organic layer was dried with sodium sulphate, filtered and concentrated to afford a crude product. The crude residue was purified by silica gel column chromatography with a gradient of 20-50% ethyl acetate in hexanes to afford 2-(4,4-difluoroazepan-1-yl)-7-fluoro-N-(3-sulfamoylphenyl)quinoline-3-carboxamide as a white solid. MS (ESI): Calcd [M+H]⁺: 479.14, found: 479.21.

¹H NMR (400 MHz, DMSO-d₆): δ 10.95 (s, 1H), 8.37 (s, 1H), 8.31 (s, 1H), 7.94 (t, J=8 Hz, 1H), 7.86-7.84 (m, 1H), 7.58-7.56 (m, 2H), 7.42 (s, 2H), 7.34-7.31 (m, 1H), 7.23-7.18 (m, 1H), 4.04 (d, J=3.2 Hz, 2H), 3.60 (t, J=5.6 Hz, 2H), 2.40-2.35 (m, 2H), 2.06-1.98 (m, 2H), 1.90-1.88 (m, 2H).

### Compound 29

### Synthesis of 6,7-dichloro-2-(4,4-difluoroazepan-1-yl)-N-(3-sulfamoylphenyl)quinoxaline-3-carboxamide

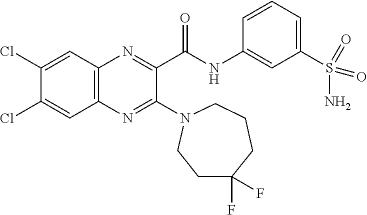 [SMILES: NS(=O)(=O)c1cccc(NC(=O)c2nc3cc(Cl)c(Cl)cc3nc2N2CCCC(F)(F)CC2)c1]

To a stirred solution of 6,7-dichloro-2-(4,4-difluoroazepan-1-yl)quinoxaline-3-carboxylic acid (15 mg, 0.04 mmol) in dichloromethane (2 mL) and N,N-dimethylformamide (0.5 mL), (1-[Bis(dimethylamino)methylene]-1H-1,2,3-triazolo[4,5-b]pyridinium 3-oxide hexafluoro phosphate (HATU) (23 mg, 0.12 mmol), 4-dimethylamino pyridine (DMAP) (15 mg, 0.12 mmol) was added at room temperature and stirred at room temperature for 15 minutes. Then 3-aminobenzenesulfonamide (7 mg, 0.04 mmol) was added to the solution. The reaction mixture was stirred at room temperature for 18 hours then at 60° C. for 18 hours. Dichloromethane was removed in vacuo and the crude residue was purified by reversed-phase HPLC with a gradient of 30-80% acetonitrile in water to afford 6,7-dichloro-2-(4,4-difluoroazepan-1-yl)-N-(3-sulfamoylphenyl)quinoline-3-carboxamide an off-white solid. LRMS (ESI): Calcd [M+H]⁺: 530.06, found: 529.7.

### Compound 30

### Synthesis of 3-(4,4-difluoro-3-methylpiperidin-1-yl)-N-(2-sulfamoylpyridin-4-yl)quinoxaline-2-carboxamide

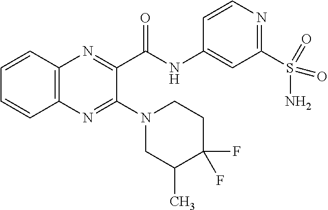 [SMILES: CC1CN(c2nc3ccccc3nc2C(=O)Nc2ccnc(S(N)(=O)=O)c2)CCC1(F)F]

### Synthesis of 3-(4,4-difluoro-3-methylpiperidin-1-yl)quinoxaline-2-carboxamide

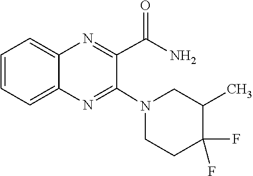 [SMILES: CC1CN(c2nc3ccccc3nc2C(N)=O)CCC1(F)F]

To a stirred solution of 3-(4,4-difluoro-3-methylpiperidin-1-yl)quinoxaline-2-carboxylic acid (215 mg, 0.7 mmol) in dichloromethane (5 mL) was added (1-[Bis(dimethylamino)methylene]-1H-1,2,3-triazolo[4,5-b]pyridinium 3-oxide hexafluorophosphate (HATU) (0.4 g, 1.05 mmol) and ammonium carbonate (330 mg, 3.5 mmol) at room temperature. The reaction mixture was stirred at room temperature for 18 hours. The progress of the reaction was monitored by TLC and LCMS. After completion of reaction, the reaction mixture was diluted with water and extracted dichloromethane. The organic layers were combined, dried over anhydrous sodium sulphate, filtered and concentrated to afford crude material. The crude residue was purified by flash column chromatography using a Combiflash with a gradient of 0 to 50% ethyl acetate in hexanes to afford 3-(4,4-difluoro-3-methylpiperidin-1-yl)quinoxaline-2-carboxamide as a light yellow powder.

### Synthesis of N-(2-(N,N-bis(2,4-dimethoxybenzyl)sulfamoyl)pyridin-4-yl)-3-(4,4-difluoro-3-methylpiperidin-1-yl)quinoxaline-2-carboxamide

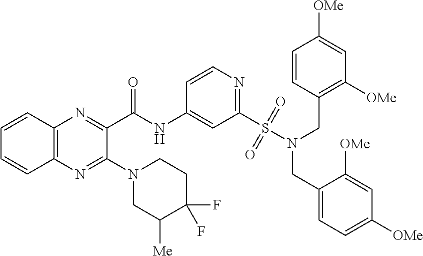 [SMILES: COc1ccc(CN(Cc2ccc(OC)cc2OC)S(=O)(=O)c2cc(NC(=O)c3nc4ccccc4nc3N3CCC(F)(F)C(C)C3)ccn2)c(OC)c1]

3-(4,4-difluoro-3-methylpiperidin-1-yl)quinoxaline-2-carboxamide (31 mg, 0.1 mmol), 4-bromo-N,N-bis(2,4-dimethoxybenzyl)pyridine-2-sulfonamide (54 mg, 0.1 mmol), cesium carbonate (65 mg, 0.2 mmol), and BrettPhos Pd G3 (18 mg, 0.01 mmol) were added to a flame dried round bottom flask and capped. The reaction vessel was evacuated and the atmosphere was replace with nitrogen. Then, degassed anhydrous dioxane (2 mL) was added to the reaction vessel and the mixture was stirred at 100° C. for 18 hours. Dioxane was removed in vacuo and the crude mixture was purified by flash column chromatography with a gradient of ethyl acetate in hexanes to afford N-(2-(N,N-bis(2,4-dimethoxybenzyl)sulfamoyl)pyridin-4-yl)-3-(4,4-difluoro-3-methylpiperidin-1-yl)quinoxaline-2-carboxamide as a yellow foam.

### Synthesis of 3-(4,4-difluoro-3-methylpiperidin-1-yl)-N-(2-sulfamoylpyridin-4-yl)quinoxaline-2-carboxamide

To a solution of N-(2-(N,N-bis(2,4-dimethoxybenzyl)sulfamoyl)pyridin-4-yl)-3-(4,4-difluoro-3-methylpiperidin-1-yl)quinoxaline-2-carboxamide (70 mg, 0.091 mmol) in dichloromethane (10 mL) was added trifluoroacetic acid (140 μL). The reaction mixture was stirred for 18 hours then the volatiles were removed in vacuo. The resulting crude mixture was diluted with water and acetonitrile then purified using reversed-phase HPLC with a gradient of 5-95% acetonitrile in water to afford 3-(4,4-difluoro-3-methylpiperidin-1-yl)-N-(2-sulfamoylpyridin-4-yl)quinoxaline-2-carboxamide as a white powder. LRMS (ESI): Calcd [M+H]⁺: 463.14, found: 462.9.

¹H NMR (400 MHz, CDCl₃) δ 8.56-8.45 (m, 1H), 8.22-8.07 (m, 2H), 7.94-7.84 (m, 1H), 7.78-7.60 (m, 2H), 7.56-7.41 (m, 1H), 4.20-3.49 (m, 3H), 3.01 (s, 2H). 2.13 (s, 2H), 0.99 (s, 3H).

### Compound 31

### Synthesis of 2-(4,4-difluoroazepan-1-yl)-7-fluoro-N-(3-(S-methylsulfonimidoyl)phenyl)quinoline-3-carboxamide

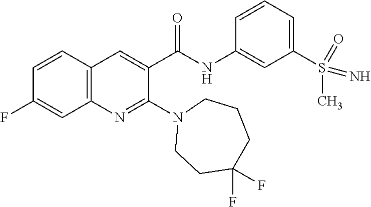 [SMILES: CS(=N)(=O)c1cccc(NC(=O)c2cc3ccc(F)cc3nc2N2CCCC(F)(F)CC2)c1]

### Synthesis of 2-(4,4-difluoroazepan-1-yl)-7-fluoro-N-(3-(methylthio)phenyl)quinoline-3-carboxamide

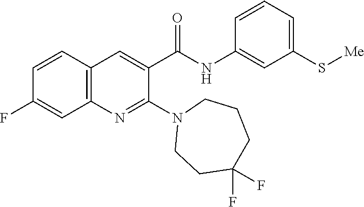 [SMILES: CSc1cccc(NC(=O)c2cc3ccc(F)cc3nc2N2CCCC(F)(F)CC2)c1]

To a stirred solution of 2-(4,4-difluoroazepan-1-yl)-7-fluoroquinoline-3-carboxylic acid (41.4 mg, 0.13 mmol) in dichloromethane (2 mL) and N,N-dimethylformamide (2 mL) was added (1-[Bis(dimethylamino)methylene]-1H-1,2,3-triazolo[4,5-b]pyridinium 3-oxide hexafluorophosphate (HATU) (73 mg, 0.19 mmol) and 4-dimethylaminopyridine (DMAP) (47 mg, 0.38 mmol). The reaction mixture was stirred at room temperature for 15 minutes. Then, 3-(methylthio)aniline (15.7 uL, 0.13 mmol) was added to the reaction mixture and stirred at room temperature for 18 hours. Then the reaction mixture was stirred at 60° C. for 72 hours. After the completion of reaction, dichloromethane was removed in vacuo and the resulting mixture was purified by flash column chromatography with a gradient of 0 to 50% ethyl acetate in hexanes to afford 2-(4,4-difluoroazepan-1-yl)-7-fluoro-N-(3-(methylthio)phenyl)quinoline-3-carboxamide as a yellow oil.

### Synthesis of 2-(4,4-difluoroazepan-1-yl)-7-fluoro-N-(3-(S-methylsulfonimidoyl)phenyl)quinoline-3-carboxamide

To a stirred solution of 2-(4,4-difluoroazepan-1-yl)-7-fluoro-N-(3-(methylthio)phenyl)quinoline-3-carboxamide (57 mg, 0.13 mmol) in MeOH (10 mL) was added ammonium carbonate (43 mg, 0.45 mmol) then (diacetoxyiodo)benzene (125 mg, 0.384 mmol). The reaction mixture was stirred at 60° C. for 18 hours. The progress of the reaction was monitored by LCMS. Methanol was removed in vacuo and the resulting crude mass was diluted with water and acetonitrile and purified using reversed-phase HPLC with a gradient of 0-50% acetonitrile in water to afford 2-(4,4-difluoroazepan-1-yl)-N-(3-(S-methylsulfonimidoyl)phenyl)quinoline-3-carboxamide as a white powder. LRMS (ESI): Calcd [M+H]⁺: 477.16, found: 476.9.

¹H NMR (400 MHz, CDCl₃) δ 8.19-8.06 (m, 2H), 8.02-7.94 (m, 1H), 7.69-7.56 (m, 2H), 7.54-7.45 (m, 1H), 7.24-7.23 (m, 1H), 7.02-6.94 (m, 1H), 3.70-3.67 (m, 2H), 3.58-3.48 (m, 2H), 3.12-3.00 (s, 3H), 2.44-2.26 (m, 2H), 2.04-1.81 (m, 4H).

### Compound 32

### Synthesis of 2-(4,4-difluoro-3-methylpiperidin-1-yl)-7-fluoro-N-(2-sulfamoylpyridin-4-yl)quinoline-3-carboxamide

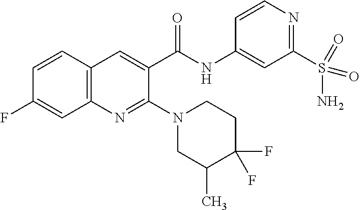 [SMILES: CC1CN(c2nc3cc(F)ccc3cc2C(=O)Nc2ccnc(S(N)(=O)=O)c2)CCC1(F)F]

### Synthesis of 2-(4,4-difluoro-3-methylpiperidin-1-yl)-7-fluoroquinoline-3-carboxylic acid

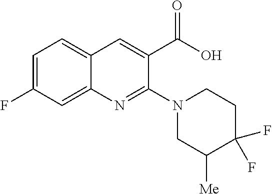 [SMILES: CC1CN(c2nc3cc(F)ccc3cc2C(=O)O)CCC1(F)F]

To a mixture of 2-chloro-7-fluoroquinoline-3-carboxylic acid (113 mg, 0.5 mmol) and 4,4-difluoro-3-methylpiperidine hydrogen chloride (86 mg, 0.5 mmol) in N,N-dimethylformamide (2 mL), potassium carbonate (345 mg, 2.5 mmol) was added at room temperature. The reaction mixture was stirred at 60° C. for 72 hours. After completion, the reaction mixture was diluted with hydrochloric acid 1 N and extracted with dichloromethane. The organic layer then was dried over anhydrous sodium sulphate and filtered. The filtrate was concentrated under reduced pressure and the crude residue was purified by silica gel column chromatography with a gradient methanol in dichloromethane to afford 2-(4,4-difluoro-3-methylpiperidin-1-yl)-7-fluoroquinoline-3-carboxylic acid as an orange solid.

### Synthesis of 2-(4,4-difluoro-3-methylpiperidin-1-yl)-7-fluoroquinoline-3-carboxamide

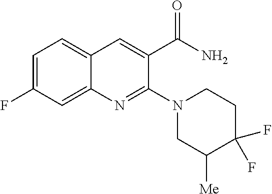 [SMILES: CC1CN(c2nc3cc(F)ccc3cc2C(N)=O)CCC1(F)F]

To a stirred solution of 2-(4,4-difluoro-3-methylpiperidin-1-yl)-7-fluoroquinoline-3-carboxamide (65 mg. 0.2 mmol) in dichloromethane (2 mL) was added (1-[Bis(dimethylamino)methylene]-1H-1,2,3-triazolo[4,5-b]pyridinium 3-oxide hexafluorophosphate (HATU) (0.114 g, 0.3 mmol) and ammonium carbonate (96 mg, 1 mmol) at room temperature. The reaction mixture was stirred at room temperature for 18 hours. The progress of the reaction was monitored by TLC and LCMS. After completion of reaction, the reaction mixture was diluted with water and extracted dichloromethane. The organic layers were combined, dried over anhydrous sodium sulphate, filtered and concentrated to afford crude material. The crude residue was purified by flash column chromatography using a Combiflash with a gradient of ethyl acetate in hexanes to afford 2-(4,4-difluoro-3-methylpiperidin-1-yl)-7-fluoroquinoline-3-carboxamide as a light yellow oil.

### Synthesis of N-(2-(N,N-bis(2,4-dimethoxybenzyl)sulfamoyl)pyridin-4-yl)-2-(4,4-difluoro-3-methylpiperidin-1-yl)-7-fluoroquinoline-3-carboxamide

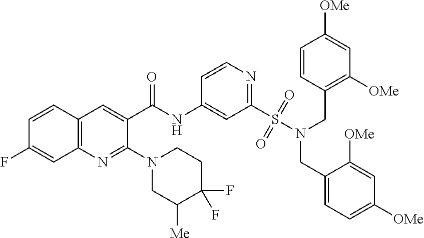 [SMILES: COc1ccc(CN(Cc2ccc(OC)cc2OC)S(=O)(=O)c2cc(NC(=O)c3cc4ccc(F)cc4nc3N3CCC(F)(F)C(C)C3)ccn2)c(OC)c1]

2-(4,4-difluoro-3-methylpiperidin-1-yl)-7-fluoroquinoline-3-carboxamide (20.6 mg, 0.064 mmol), 4-bromo-N,N-bis(2,4-dimethoxybenzyl)pyridine-2-sulfonamide (34 mg, 0.064 mmol), cesium carbonate (41.5 mg, 0.127 mmol), and BrettPhos Pd G3 (12 mg, 0.013 mmol) were added to a flame dried round bottom flask and capped. The reaction vessel was evacuated and the atmosphere was replace with nitrogen. Then, degassed anhydrous dioxane (2 mL) was added to the reaction vessel and the mixture was stirred at 100° C. for 18 hours. Dioxane was removed in vacuo and the crude mixture was purified by flash column chromatography with a gradient of ethyl acetate in hexanes to afford N-(2-(N,N-bis(2,4-dimethoxybenzyl)sulfamoyl)pyridin-4-yl)-2-(4,4-difluoro-3-methylpiperidin-1-yl)-7-fluoroquinoline-3-carboxamide as a yellow oil.

### Synthesis of 2-(4,4-difluoro-3-methylpiperidin-1-yl)-7-fluoro-N-(2-sulfamoylpyridin-4-yl)quinoline-3-carboxamide

To a solution of N-(2-(N,N-bis(2,4-dimethoxybenzyl)sulfamoyl)pyridin-4-yl)-2-(4,4-difluoro-3-methylpiperidin-1-yl)-7-fluoroquinoline-3-carboxamide (40 mg, 0.052 mmol) in dichloromethane (2 mL) was added trifluoroacetic acid (80 μL). The reaction mixture was stirred for 18 hours then the volatiles were removed in vacuo. The resulting crude mixture was diluted with water and acetonitrile then purified using reversed-phase HPLC with a gradient of 5-95% acetonitrile in water to afford 2-(4,4-difluoro-3-methylpiperidin-1-yl)-7-fluoro-N-(2-sulfamoylpyridin-4-yl)quinoline-3-carboxamide as a white powder. LRMS (ESI): Calcd [M+H]⁺: 480.13, found: 479.8.

¹H NMR (400 MHz, CDCl₃) δ 8.36 (t, J=4.7 Hz, 1H), 8.19 (d, J=4.2 Hz, 1H), 8.10-7.97 (m, 1H), 7.86-7.73 (m, 1H), 7.65-7.49 (m, 1H), 7.03-6.90 (m, 1H), 3.74-3.53 (m, 2H), 2.89-2.77 (m, 1H), 2.13-1.71 (m, 4H), 0.87-0.70 (m, 3H).

### Compound 33

### Synthesis of 2-(4,4-difluoroazepan-1-yl)-N-(3-(methylsulfonyl) phenyl)-6,7-dihydro-5H-cyclopenta[b]pyridine-3-carboxamide

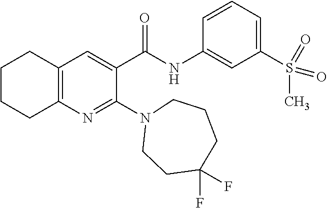 [SMILES: CS(=O)(=O)c1cccc(NC(=O)c2cc3c(nc2N2CCCC(F)(F)CC2)CCCC3)c1]

To a stirred solution of 2-(4,4-difluoroazepan-1-yl)-6,7-dihydro-5H-cyclopenta[b]pyridine-3-carboxylic acid (0.15 g, 0.5 mmol) in dichloromethane (6 mL) was added (1-[Bis(dimethylamino)methylene]-1H-1,2,3-triazolo[4,5-b]pyridinium-3-oxide hexafluorophosphate (HATU) (0.38 g, 1 mmol) and 4-(dimethylamino)pyridine (0.03 g, 0.25 mmol) at 0° C. and stirred the mixture at room temperature for 12 hours. After completion of reaction, solvent was evaporated under nitrogen atmosphere and diluted with acetonitrile (6 mL) and to this, 3-(methyl sulfonyl) aniline (0.1 g, 0.6 mmol) was added and heated to 80° C. for 12 hours. The progress of reaction was monitored by TLC. After completion of reaction, acetonitrile was removed under rotatory evaporation. The crude mass was diluted with water and extracted with ethyl acetate. The organic layer was washed with brine solution, dried over anhydrous sodium sulfate and concentrated. The crude material was purified by flash column chromatography with a gradient of 40-50% ethyl acetate in hexanes to afford 2-(4,4-difluoroazepan-1-yl)-N-(3-(methylsulfonyl)phenyl)-6,7-dihydro-5H-cyclopenta[b]pyridine-3-carboxamide as an off-white solid. MS (ESI): Calcd [M+H]⁺: 450.17, found: 450.25.

¹H NMR (400 MHz, DMSO-d₆): δ 10.65 (s, 1H), 8.35 (s, 1H), 7.95 (d, J=6.0 Hz, 1H), 7.62-7.59 (m, 3H), 3.58 (s, 2H), 3.38 (t, J=5.6 Hz, 2H), 3.20 (s, 3H), 2.83-2.79 (m, 4H), 2.36-2.27 (m, 2H), 2.08-2.03 (m, 2H), 2.01-1.91 (m, 2H), 1.83-1.81 (m, 2H).

### Compound 34

### Synthesis of 2-(4,4-difluoroazepan-1-yl)-N-(3-(methylsulfonyl)phenyl)-1,7-naphthyridine-3-carboxamide

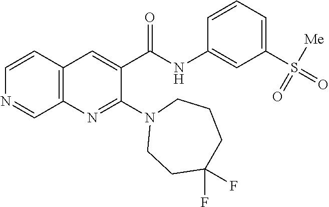 [SMILES: CS(=O)(=O)c1cccc(NC(=O)c2cc3ccncc3nc2N2CCCC(F)(F)CC2)c1]

To a stirred solution of 2-(4,4-difluoroazepan-1-yl)-1,7-naphthyridine-3-carboxylic acid (0.25 g, 0.8 mmol) in dry dichloromethane (5 mL) was added Bis(dimethylamino)methylene]-1H-1,2,3-triazolo[4,5-b]pyridinium 3-oxide hexafluorophosphate (HATU) (0.46 g, 1.2 mmol) and N,N-dimethylpyridin-4-amine (0.05 g, 0.4 mmol) at 0° C. and reaction mixture was stirred at room temperature for 12 hours. The progress of reaction mixture was monitored by TLC and LCMS. After completion of reaction, solvent was evaporated under rotatory evaporation and diluted with acetonitrile (5 mL) and to this, 3-methanesulfonylaniline (0.17 g, 0.97 mmol) was added at room temperature. The reaction mixture was stirred at 70° C. for 16 hours. The progress of the reaction was monitored by TLC. After completion the reaction, the reaction mixture was diluted with water and extracted with ethyl acetate. The organic layer was dried over anhydrous sodium sulfate and filtered. The filtrate was concentrated under reduced pressure and the crude residue was purified by flash column chromatography with a gradient of 50-70% ethyl acetate in hexanes to afford 2-(4,4-difluoroazepan-1-yl)-N-(3-(methylsulfonyl)phenyl)-1,7-naphthyridine-3-carboxamide as a white solid. MS (ESI): Calcd [M+H]⁺: 461.15, found: 461.30.

¹H NMR (400 MHz, DMSO-d₆): δ 11.15 (s, 1H), 9.00 (s, 1H), 8.43 (s, 1H), 8.36 (d, J=4 Hz, 2H), 8.02-7.99 (m, 1H), 7.75 (d, J=4 Hz, 1H), 7.72-7.66 (m, 2H), 3.80-3.77 (m, 2H), 3.64 (t, J=4 Hz, 2H), 3.23 (s, 3H), 2.42-2.39 (m, 2H), 2.06-1.97 (m, 2H), 1.93-1.90 (m, 2H).

### Compound 35

### Synthesis of N-(3-carbamoylphenyl)-2-(4,4-difluoroazepan-1-yl)-5,6,7,8-tetrahydroquinoline-3-carboxamide

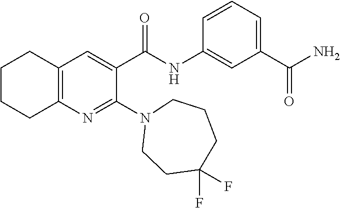 [SMILES: NC(=O)c1cccc(NC(=O)c2cc3c(nc2N2CCCC(F)(F)CC2)CCCC3)c1]

### Synthesis of methyl 3-(2-(4,4-difluoroazepan-1-yl)-5,6,7,8-tetrahydroquinoline-3-carboxamido) benzoate

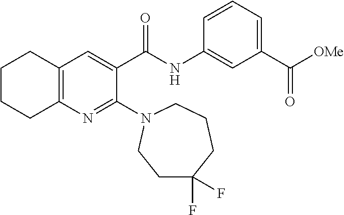 [SMILES: COC(=O)c1cccc(NC(=O)c2cc3c(nc2N2CCCC(F)(F)CC2)CCCC3)c1]

To a solution of 2-(4,4-difluoroazepan-1-yl)-5,6,7,8-tetrahydroquinoline-3-carboxylic acid (0.25 g, 0.8 mmol) in dichloromethane (10 mL) was added (1-[Bis(dimethylamino)methylene]-1H-1,2,3-triazolo[4,5-b]pyridinium 3-oxide hexafluorophosphate (HATU) (0.45 g, 1.2 mmol) and 4-dimethylaminopyridine (DMAP) (0.005 g, 0.04 mmol) at 0° C. and stirred the mixture at room temperature for 12 hours. After completion of reaction, dichloromethane was evaporated under reduced pressure. The resulting mass was dissolved into acetonitrile (5 mL) and to this methyl 3-aminobenzoate (0.14 g, 0.96 mmol) was added at room temperature and heated at 80° C. for 12 hours. After completion of reaction, acetonitrile was removed in vacuo. The crude mass was diluted with water and extracted with ethyl acetate. The organic layer was washed with brine solution and dried over anhydrous sodium sulphate and concentrated under rotatory. The crude material was purified by flash column chromatography with a gradient of 30-40% ethyl acetate in hexanes to afford methyl 3-(2-(4,4-difluoroazepan-1-yl)-5,6,7,8-tetrahydroquinoline-3-carboxamido) benzoate as a white solid.

### Synthesis of N-(3-carbamoylphenyl)-2-(4,4-difluoroazepan-1-yl)-5,6,7,8-tetrahydroquinoline-3-carboxamide

A solution of methyl 3-(2-(4,4-difluoroazepan-1-yl)-5,6,7,8-tetrahydroquinoline-3-carboxamido) benzoate (0.15 g, 0.3 mmol) in 7 N ammonia in methanol (5 mL) was heated at 100° C. for 48 hours. The progress of reaction was monitored by TLC. After completion of reaction, methanol was removed under reduced pressure. The crude material was purified by flash column chromatography with a gradient of 70-80% ethyl acetate in hexanes to afford N-(3-carbamoylphenyl)-2-(4,4-difluoroazepan-1-yl)-5,6,7,8-tetrahydroquinoline-3-carboxamide as white solid. MS (ESI): Calcd [M+H]⁺: 429.21, found: 429.35.

¹H NMR (400 MHz, DMSO-d₆): δ 10.41 (s, 1H), 8.15 (s, 1H), 7.93 (s, 1H), 7.81 (d, J=8.0 Hz, 1H), 7.55 (d, J=7.6 Hz, 1H), 7.41-7.35 (m, 3H), 3.57-3.56 (m, 2H), 3.39 (t, J=5.6 Hz, 2H), 2.69-2.66 (m, 2H), 2.65-2.62 (m, 2H), 2.31-2.24 (m, 2H), 1.98-1.190 (m, 2H), 1.81-1.79 (m, 4H), 1.73-1.72 (m, 2H).

### Compound 36

### Synthesis N-(3-carbamoylphenyl)-2-(4,4-difluoroazepan-1-yl)-1,6-naphthyridine-3-carboxamide

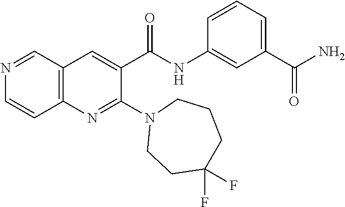 [SMILES: NC(=O)c1cccc(NC(=O)c2cc3cnccc3nc2N2CCCC(F)(F)CC2)c1]

### Synthesis methyl 3-(2-(4,4-difluoroazepan-1-yl)-1,6-naphthyridine-3-carboxamido)benzoate

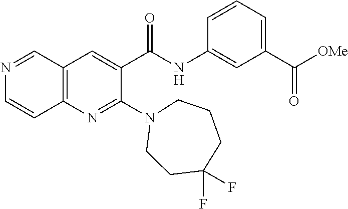 [SMILES: COC(=O)c1cccc(NC(=O)c2cc3cnccc3nc2N2CCCC(F)(F)CC2)c1]

To a stirred solution of 2-(4,4-difluoroazepan-1-yl)-1,7-naphthyridine-3-carboxylic acid (0.50 g, 1.6 mmol) in dry dichloromethane (20 mL) was added Bis(dimethylamino)methylene]-1H-1,2,3-triazolo[4,5-b]pyridinium 3-oxid hexafluorophosphate (HATU) (0.93 g, 2.4 mmol) and N,N-dimethylpyridin-4-amine (0.1 g, 0.82 mmol) was added at 0° C. The reaction mixture was stirred at room temperature for 16 hours. The reaction progress of reaction was monitored by TLC and LCMS. After completion of reaction, solvent was evaporated under rotatory evaporation and diluted with acetonitrile (10 mL) and to this solution, methyl 3-aminobenzoate (0.25 g, 2 mmol) and stirred the reaction mixture at 70° C. for 16 hours. The progress of the reaction was monitored by TLC. After completion of the reaction, the reaction mixture was diluted with water and extracted with ethyl acetate. The organic layer was dried over anhydrous sodium sulfate and filtered. The filtrate was concentrated under reduced pressure and the resultant crude residue was purified by flash column chromatography with a gradient of 50-70% ethyl acetate in heptane to afford methyl 3-(2-(4,4-difluoroazepan-1-yl)-1,6-naphthyridine-3-carboxamido)benzoate as a white solid.

### Synthesis N-(3-carbamoylphenyl)-2-(4,4-difluoroazepan-1-yl)-1,6-naphthyridine-3-carboxamide

Methyl 3-(2-(4,4-difluoroazepan-1-yl)-1,6-naphthyridine-3-carboxamido)benzoate (0.50 g, 1.1 mmol) was dissolved into 7N Ammonia in methanol (10 mL) and heated to 80° C. for 12 hours. The progress of reaction was monitored by TLC and LCMS. After completion of the reaction, mixture was concentrated under reduced pressure and the crude residue was purified by flash column chromatography with a gradient of 0-5% methanol in dichloromethane to afford N-(3-carbamoylphenyl)-2-(4,4-difluoroazepan-1-yl)-1,6-naphthyridine-3-carboxamide as a white solid. MS (ESI): Calcd [M+H]⁻: 426.17, found: 426.30.

¹H NMR (400 MHz, DMSO-d₆): δ 11.84 (s, 1H), 9.07 (s, 1H), 8.51 (d, J=4 Hz, 1H), 8.46 (s, 1H), 8.19 (s, 1H), 7.97 (s, 1H), 7.87 (d, J=8 Hz, 1H), 7.63 (d, J=8 Hz, 1H), 7.468 (s, 1H), 7.46 (t, J=8 Hz, 1H), 7.38 (s, 1H), 3.82-3.80 (m, 2H), 3.67 (t, J=8 Hz, 2H), 2.42-2.39 (m, 2H), 2.02-1.99 (m, 2H), 1.92-1.90 (m, 2H).

### Compound 37

### Synthesis of 2-(4,4-difluoroazepan-1-yl)-6,6-dimethyl-N-(3-sulfamoylphenyl)-5,6,7,8-tetrahydroquinoline-3-carboxamide

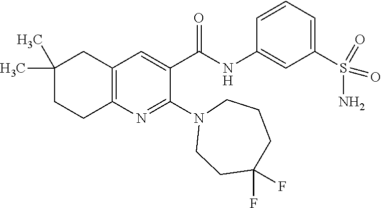 [SMILES: CC1(C)CCc2nc(N3CCCC(F)(F)CC3)c(C(=O)Nc3cccc(S(N)(=O)=O)c3)cc2C1]

### Synthesis of (Z)-2-((dimethylamino)methylene)-4,4-dimethylcyclohexan-1-one

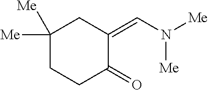 [SMILES: CN(C)/C=C1/CC(C)(C)CCC1=O]

To a stirred solution of 4,4-dimethylcyclohexan-1-one (10 g, 79 mmol) in toluene (50 mL), [(tert-butoxy)(dimethylamino)methyl]dimethylamine (15 g, 87 mmol) was added and the resulting mixture was stirred at 50° C. for 24 hours. Progress of the reaction was monitored by LCMS. After completion of the reaction, the reaction mixture was evaporated under vacuum to afford the crude (Z)-2-((dimethylamino)methylene)-4,4-dimethylcyclohexan-1-one as a brown liquid. The crude product was used as such in the next step without purification.

### Synthesis of methyl 2-hydroxy-6,6-dimethyl-5,6,7,8-tetrahydroquinoline-3-carboxylate

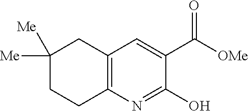 [SMILES: COC(=O)c1cc2c(nc1O)CCC(C)(C)C2]

To a stirred solution of crude (Z)-2-((dimethylamino)methylene)-4,4-dimethylcyclohexan-1-one (15 g, 83 mmol) in methanol (75 mL), methyl 2-cyanoacetate (9.8 g, 99 mmol) was added and the resulting mixture was heated at 70° C. for 24 hours. The progress of the reaction was monitored by TLC and LCMS. After completion of reaction, methanol was removed under reduced pressure and the resulting residue was purified by trituration with acetonitrile to afford methyl 2-hydroxy-6,6-dimethyl-5,6,7,8-tetrahydroquinoline-3-carboxylate as a white solid.

### Synthesis of methyl 2-chloro-6,6-dimethyl-5,6,7,8-tetrahydroquinoline-3-carboxylate

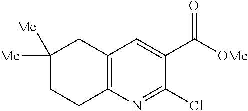 [SMILES: COC(=O)c1cc2c(nc1Cl)CCC(C)(C)C2]

A solution of methyl 2-hydroxy-6,6-dimethyl-5,6,7,8-tetrahydroquinoline-3-carboxylate (6 g, 26 mmol) in phosphoryl oxychloride (60 mL) was heated at 130° C. for 18 hours under nitrogen atmosphere. Progress of the reaction was monitored by TLC. After completion, the reaction mixture was concentrated under reduced pressure and then poured into a beaker containing crushed ice. The mixture was basified with aqueous sodium bicarbonate and then extracted with ethyl acetate. The organic layer was washed with brine solution, dried over anhydrous sodium sulfate and evaporated under reduced pressure to obtain the crude product. The crude material was purified by flash column chromatography with a gradient of 10-15% ethyl acetate in hexanes to afford methyl 2-chloro-6,6-dimethyl-5,6,7,8-tetrahydroquinoline-3-carboxylate as a yellow oil.

### Synthesis of methyl 2-(4,4-difluoroazepan-1-yl)-6,6-dimethyl-5,6,7,8-tetrahydroquinoline-3-carboxylate

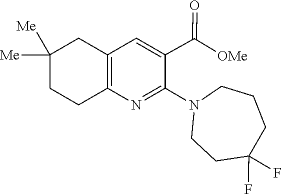 [SMILES: COC(=O)c1cc2c(nc1N1CCCC(F)(F)CC1)CCC(C)(C)C2]

To a stirred solution of methyl 2-chloro-6,6-dimethyl-5,6,7,8-tetrahydroquinoline-3-carboxylate (2.0 g, 7.9 mmol) in N,N-dimethylformamide (10 mL), 4,4-difluoroazepane hydrochloride (2.7 g, 15.8 mmol) and dipotassium carbonate (6.5 g, 47.4 mmol) were added at room temperature. The resulting mixture was heated at 100° C. for 24 hours. Progress of the reaction was monitored by TLC. After completion of reaction, the reaction mixture was diluted with water and extracted with ethyl acetate. The organic layer was washed with brine, dried over anhydrous sodium sulfate and evaporated under reduced pressure to afford the crude product which was purified by flash column chromatography with a gradient of 5-10% ethyl acetate in hexanes to afford methyl 2-(4,4-difluoroazepan-1-yl)-6,6-dimethyl-5,6,7,8-tetrahydroquinoline-3-carboxylate as a yellow viscous oil.

### Synthesis of 2-(4,4-difluoroazepan-1-yl)-6,6-dimethyl-5,6,7,8-tetrahydroquinoline-3-carboxylic acid

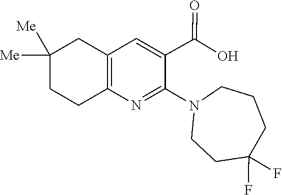 [SMILES: CC1(C)CCc2nc(N3CCCC(F)(F)CC3)c(C(=O)O)cc2C1]

To a stirred solution of methyl 2-(4,4-difluoroazepan-1-yl)-6,6-dimethyl-5,6,7,8-tetrahydroquinoline-3-carboxylate (1 g, 2.8 mmol) in methanol (20 mL) and tetrahydrofuran (20 mL), aqueous solution of lithium hydroxide (0.48 g, 11.4 mmol) in water (20 mL) was added. The resulting mixture was stirred at room temperature for 24 hours. Progress of the reaction was monitored by TLC. The organic solvents were removed under the reduced pressure and the resulting aqueous solution was acidified with 1N hydrochloric acid. The resulting mixture was extracted with ethyl acetate. The organic layer was washed with brine, dried over anhydrous sodium sulfate and evaporated under reduced pressure to afford the crude product. The crude product was purified by flash column chromatography with a gradient of 40-50% ethyl acetate in heptane to afford 2-(4,4-difluoroazepan-1-yl)-6,6-dimethyl-5,6,7,8-tetrahydroquinoline-3-carboxylic acid as a white solid.

### Synthesis of 2-(4,4-difluoroazepan-1-yl)-6,6-dimethyl-N-(3-sulfamoylphenyl)-5,6,7,8-tetrahydroquinoline-3-carboxamide

To a stirred suspension of 2-(4,4-difluoroazepan-1-yl)-6,6-dimethyl-5,6,7,8-tetrahydroquinoline-3-carboxylic acid (0.3 g, 0.9 mmol) in dichloromethane (10 mL) was added [Bis(dimethylamino)methylene]-1H-1,2,3-triazolo[4,5-b]pyridinium 3-oxide hexafluorophosphate (HATU) (0.5 g, 1.3 mmol) and N,N-dimethylpyridin-4-amine (0.054 g, 0.44 mmol) were added sequentially between 0-5° C. The resulting mixture was stirred at room temperature for 16 hours. The progress of reaction was monitored by TLC. After completion of the reaction, mixture was evaporated under vacuum to remove the dichloromethane and the resulting residue was dissolved into acetonitrile (10 mL). To this solution, 3-aminobenzene-1-sulfonamide (0.18 g, 1 mmol) was added and reaction mixture was heated at 80° C. for 16 hours. After the completion of reaction, the reaction mixture was cooled to room temperature, diluted with water and extracted with ethyl acetate. The organic layer was washed with brine, dried over anhydrous sodium sulfate and evaporated under reduced pressure to afford the crude product which was purified by flash column chromatography with a gradient of 20-30% ethyl acetate in heptane to afford 2-(4,4-difluoroazepan-1-yl)-6,6-dimethyl-N-(3-sulfamoylphenyl)-5,6,7,8-tetrahydroquinoline-3-carboxamide as a white solid. MS (ESI): Calcd [M+H]⁻: 493.21, found: 493.00.

¹H NMR (400 MHz, DMSO-d₆): δ 10.61 (s, 1H), 8.30 (s, 1H), 7.80-7.77 (m, 1H), 7.54-7.49 (m, 2H), 7.39-7.37 (m, 3H), 3.58-3.56 (m, 2H), 3.41-3.38 (m, 2H), 2.72-2.68 (m, 2H), 2.43 (s, 2H), 2.29-2.26 (m, 2H), 1.97-1.90 (m, 2H), 1.81-1.80 (m, 2H), 1.61-1.58 (m, 2H), 0.96 (s, 6H).

### Compound 38

### Synthesis of 2-(4,4-difluoroazepan-1-yl)-N-(3-(S-methylsulfonimidoyl)phenyl)-1,6-naphthyridine-3-carboxamide

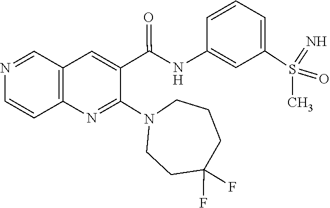 [SMILES: CS(=N)(=O)c1cccc(NC(=O)c2cc3cnccc3nc2N2CCCC(F)(F)CC2)c1]

### Synthesis of 2-(4,4-difluoroazepan-1-yl)-N-(3-(methylsulfinyl)phenyl)-1,6-naphthyridine-3-carboxamide

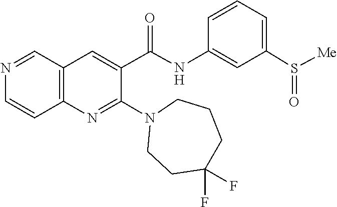 [SMILES: CS(=O)c1cccc(NC(=O)c2cc3cnccc3nc2N2CCCC(F)(F)CC2)c1]

To a stirred solution of 2-(4,4-difluoroazepan-1-yl)-1,6-naphthyridine-3-carboxylic acid (0.10 g, 0.325 mmol) in dry dichloromethane (5 mL) was added Bis(dimethylamino)methylene]-1H-1,2,3-triazolo[4,5-b]pyridinium 3-oxide hexafluorophosphate (0.19 g, 0.5 mmol) and N,N-dimethylpyridin-4-amine (0.02 g, 0.16 mmol) was added at 0° C. The reaction mixture was stirred at room temperature for 16 hours. The reaction mixture was monitored by TLC and LCMS. After completion of reaction, solvent was removed in vacuo and diluted with acetonitrile (5 mL) and to this solution, 3-methanesulfonylaniline (0.06 g, 0.39 mmol) was added at room temperature and the reaction mixture was heated at 70° C. for 16 hours. The progress of the reaction was monitored by TLC. After completion of the reaction, the mixture was diluted with water and extracted with ethyl acetate. The organic layer was dried over anhydrous sodium sulfate and filtered. The filtrate was concentrated under reduced pressure and the crude residue was purified by flash column chromatography with a gradient of 50-70% ethyl acetate in hexanes to afford 2-(4,4-difluoroazepan-1-yl)-N-(3-(methylsulfinyl)phenyl)-1,6-naphthyridine-3-carboxamide as a white solid.

### Synthesis of 2-(4,4-difluoroazepan-1-yl)-N-(3-(S-methylsulfonimidoyl)phenyl)-1,6-naphthyridine-3-carboxamide

To a stirred solution of 2-(4,4-difluoroazepan-1-yl)-N-(3-(methylsulfinyl)phenyl)-1,6-naphthyridine-3-carboxamide (0.100 g, 0.225 mmol) in Eaton's Reagent (1 mL) was added sodium azide (0.03 g, 0.45 mmol) at room temperature. The reaction mixture was stirred at 50° C. for 2 hours. The progress of reaction mixture was monitored by TLC and LCMS. After completion of the reaction, the mixture was diluted with water and basified by using sodium carbonate and extracted with ethyl acetate. The organic layer was dried over anhydrous sodium sulfate. The filtrate was concentrated under reduced pressure and the crude residue was purified by flash column chromatography with a gradient of 0-5% methanol in dichloromethane to afford 2-(4,4-difluoroazepan-1-yl)-N-(3-(S-methylsulfonimidoyl)phenyl)-1,6-naphthyridine-3-carboxamide a white solid. MS (ESI): Calcd [M+H]⁺: 461.15, found: 460.30.

¹H NMR (400 MHz, DMSO-d₆): δ 11.05 (s, 1H), 9.07 (s, 1H), 8.52 (d, J=4 Hz, 1H), 8.49 (s, 1H), 8.337-8.333 (m, 1H), 7.99 (d, J=8 Hz, 1H), 7.70 (d, J=8 Hz, 1H), 7.63 (t, J=8 Hz, 1H), 7.46 (d, J=4 Hz, 1H), 4.22 (s, 1H), 3.82-3.79 (m, 2H), 3.65 (t, J=4 Hz, 2H), 3.07 (s, 3H), 2.42-2.39 (m, 2H), 2.01-2.00 (m, 2H), 1.96-1.90 (m, 2H).

### Compound 39

### Synthesis of 2-(4,4-difluoroazepan-1-yl)-N-(3-sulfamoylphenyl)-7,8-dihydro-5H-pyrano[4,3-b]pyridine-3-carboxamide

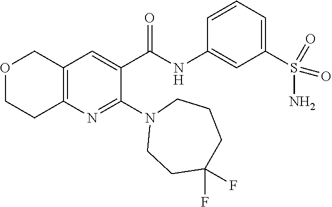 [SMILES: NS(=O)(=O)c1cccc(NC(=O)c2cc3c(nc2N2CCCC(F)(F)CC2)CCOC3)c1]

### Synthesis of (Z)-3-((dimethylamino)methylene)tetrahydro-4H-pyran-4-one

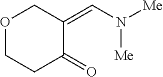 [SMILES: CN(C)/C=C1/COCCC1=O]

A mixture of tetrahydro-4H-pyran-4-one (5 g, 5 mmol) and 1-tert-butoxy-N,N,N′,N′-tetramethylmethanediamine (9.6 g, 55 mmol) was stirred at room temperature for overnight. Afterwards, heated the reaction mixture at 70° C. for 24 hours. The progress of reaction was monitored by TLC and LCMS. After completion of reaction, the solvent was removed under rotatory to obtain crude (Z)-3-((dimethylamino)methylene)tetrahydro-4H-pyran-4-one as a brown liquid which was used as such into next step without any purification.

### Synthesis of methyl 2-hydroxy-7,8-dihydro-5H-pyrano[4,3-b]pyridine-3-carboxylate

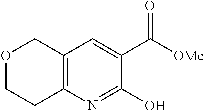 [SMILES: COC(=O)c1cc2c(nc1O)CCOC2]

To the crude (Z)-3-((dimethylamino)methylene)tetrahydro-4H-pyran-4-one (7 g, 45 mmol) in methanol (35 mL) was added methyl 2-cyanoacetate (4.8 mL, 54 mmol) and reflux the reaction mixture for 24 hours. After completion of reaction, the solvent was removed under rotatory and the resultant crude residue was purified by trituration with acetonitrile to afford methyl 2-hydroxy-7,8-dihydro-5H-pyrano[4,3-b]pyridine-3-carboxylate as a light yellow solid.

### Synthesis of methyl 2-chloro-7,8-dihydro-5H-pyrano[4,3-b]pyridine-3-carboxylate

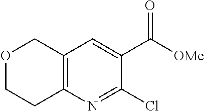 [SMILES: COC(=O)c1cc2c(nc1Cl)CCOC2]

Methyl 2-hydroxy-7,8-dihydro-5H-pyrano[4,3-b]pyridine-3-carboxylate (6 g, 29 mmol) was dissolved into phosphorus oxychloride (60 mL) at room temperature and the reaction mixture was heated at 130° C. for 18 hours. After completion of reaction, the reaction mixture was then cooled to room temperature and excess of phosphorus oxychloride was distilled in vacuo. The residue thus obtained was dissolved into ethyl acetate and washed with sodium bicarbonate solution. The organic layer was separated, washed with brine solution, dried over anhydrous sodium sulfate and concentrated in vacuum to afford the crude material which was triturated with diethyl ether and n-pentane to afford methyl 2-chloro-7,8-dihydro-5H-pyrano[4,3-b]pyridine-3-carboxylate as a white solid.

### Synthesis of methyl 2-(4,4-difluoroazepan-1-yl)-7,8-dihydro-5H-pyrano[4,3-b]pyridine-3-carboxylate

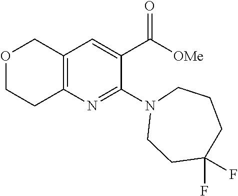 [SMILES: COC(=O)c1cc2c(nc1N1CCCC(F)(F)CC1)CCOC2]

To a solution of methyl 2-chloro-7,8-dihydro-5H-pyrano[4,3-b]pyridine-3-carboxylate (3 g, 13 mmol) in N,N-dimethylformamide (25 mL) was added 4,4-difluoroazepane hydrogen chloride (4.5 g, 26 mmol) and potassium carbonate (11 g, 79 mmol). The reaction mixture was heated at 130° C. for 28 hours under nitrogen atmosphere. The progress of reaction was monitored by TLC. After completion of reaction, the crude material was diluted with ice-cold water and extracted with ethyl acetate. The organic layer was washed with brine solution and dried over anhydrous sodium sulfate. The crude material was purified by flash column chromatography with a gradient of 30-40% ethyl acetate in hexanes to afford methyl 2-(4,4-difluoroazepan-1-yl)-7,8-dihydro-5H-pyrano[4,3-b]pyridine-3-carboxylate as a yellow liquid.

### Synthesis of 2-(4,4-difluoroazepan-1-yl)-7,8-dihydro-5H-pyrano[4,3-b]pyridine-3-carboxylic acid

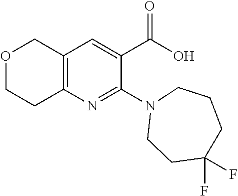 [SMILES: O=C(O)c1cc2c(nc1N1CCCC(F)(F)CC1)CCOC2]

To a solution of methyl 2-(4,4-difluoroazepan-1-yl)-7,8-dihydro-5H-pyrano[4,3-b]pyridine-3-carboxylate (2.2 g, 6.7 mmol) in tetrahydrofuran (22 mL) and methanol (22 mL) was added aqueous solution of lithium hydroxide (0.57 g, 13.6 mmol) in water (22 mL) and stirred at room temperature for 12 hours. After completion of reaction, solvent was removed under rotatory and diluted with water, acidified with 1N hydrochloric solution and extracted with ethyl acetate. The organic layer was dried over anhydrous sodium sulfate and filtered. The filtrate was concentrated under reduced pressure and the resultant crude residue was purified by trituration with diethyl ether and n-pentane to afford 2-(4,4-difluoroazepan-1-yl)-7,8-dihydro-5H-pyrano[4,3-b]pyridine-3-carboxylic acid as an off-white solid.

### Synthesis of 2-(4,4-difluoroazepan-1-yl)-N-(3-sulfamoylphenyl)-7,8-dihydro-5H-pyrano[4,3-b]pyridine-3-carboxamide

To a solution of 2-(4,4-difluoroazepan-1-yl)-7,8-dihydro-5H-pyrano[4,3-b]pyridine-3-carboxylic acid (0.40 g, 1.3 mmol) in dichloromethane (12 mL) was added (1-[Bis(dimethylamino)methylene]-1H-1,2,3-triazolo[4,5-b]pyridinium3-oxide hexafluorophosphate (HATU) (0.730 g, 1.92 mmol) and 4-(dimethylamino) pyridine (0.078 g, 0.64 mmol) at 0° C. and stirred the mixture at room temperature for 12 hours. After completion of reaction, solvent was evaporated under nitrogen atmosphere and diluted with acetonitrile (12 mL) and to this, 3-aminobenzenesulfonamide (0.27 g, 1.5 mmol) was added and heated to 80° C. for 12 hours. The progress of reaction was monitored by TLC. After completion of reaction, acetonitrile was removed under rotatory. The crude mass was diluted with water and extracted with ethyl acetate. The organic layer was washed with brine solution, dried over anhydrous sodium sulfate and concentrated. The crude material was purified by flash column chromatography with a gradient of 60-70% ethyl acetate in hexanes to afford 2-(4,4-difluoroazepan-1-yl)-N-(3-sulfamoylphenyl)-7,8-dihydro-5H-pyrano[4,3-b]pyridine-3-carboxamide as a white solid. MS (ESI): Calcd [M+H]⁺: 467.16, found: 467.25.

¹H NMR (400 MHz, DMSO-d₆): δ 10.63 (s, 1H), 8.29 (brs, 1H), 7.81-7.78 (m, 1H), 7.53-7.50 (m, 2H), 7.46 (s, 1H), 7.39 (brs, 2H), 4.61 (s, 2H), 3.95 (t, J=5.6 Hz, 2H), 3.61-3.58 (m, 2H), 3.40 (t, J=6.0 Hz, 2H), 2.75 (t, J=5.6 Hz, 2H), 2.30-2.27 (m, 2H), 1.97-1.82 (m, 4H).

### Compound 40

### Synthesis of 2-(4,4-difluoroazepan-1-yl)-N-(3-sulfamoylphenyl)-6,7-dihydro-5H-cyclopenta[b]pyridine-3-carboxamide

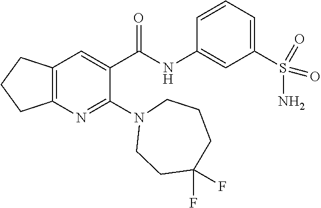 [SMILES: NS(=O)(=O)c1cccc(NC(=O)c2cc3c(nc2N2CCCC(F)(F)CC2)CCC3)c1]

### Synthesis of (Z)-2-((dimethylamino)methylene) cyclopentan-1-one

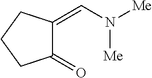

Cyclopentanone (100 g) was dissolved into 1,1-dimethoxy-N,N-dimethylmethanamine (180 mL) and slowly heated to 110° C. for 24 hours. The progress of reaction was monitored by TLC and LCMS. After completion of reaction, the solvent was removed under rotatory to obtain the crude (Z)-2-((dimethylamino)methylene)cyclopentan-1-one as a brown liquid which was used as such into next step without any purification.

### Synthesis of methyl 2-hydroxy-6,7-dihydro-5H-cyclopenta[b]pyridine-3-carboxylate

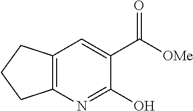 [SMILES: COC(=O)c1cc2c(nc1O)CCC2]

To the crude (Z)-2-((dimethylamino)methylene) cyclopentan-1-one (150 g) in methanol (75 mL) was added methyl 2-cyanoacetate (114 mL) and heated the reaction mixture at 80° C. for 12 hours. After completion of reaction, the solvent was removed in vacuo and the resultant crude residue was purified by trituration with acetonitrile to afford methyl 2-hydroxy-6,7-dihydro-5H-cyclopenta[b]pyridine-3-carboxylate as light yellow solid.

### Synthesis of 2-hydroxy-6,7-dihydro-5H-cyclopenta[b]pyridine-3-carboxylic acid

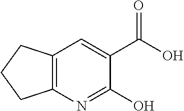 [SMILES: O=C(O)c1cc2c(nc1O)CCC2]

To a solution of methyl 2-hydroxy-6,7-dihydro-5H-cyclopenta[b]pyridine-3-carboxylate (20 g, 104 mmol) in tetrahydrofuran (20 mL), methanol (20 mL) and water (20 mL) was added lithium hydroxide (8.7 g, 207 mmol) at room temperature. The reaction mixture was stirred at room temperature for 12 hours. After completion of reaction, solvent was removed in vacuo and diluted with water, acidified with 1N hydrochloric solution and extracted with 10% methanol in dichloromethane. The combined organic layer was dried over anhydrous sodium sulfate and filtered. The filtrate was concentrated under reduced pressure and the resultant crude residue was purified by trituration with diethyl ether and n-pentane to afford 2-hydroxy-6,7-dihydro-5H-cyclopenta[b]pyridine-3-carboxylic acid as an off-white solid.

### Synthesis of 2-chloro-6,7-dihydro-5H-cyclopenta[b]pyridine-3-carboxylic acid

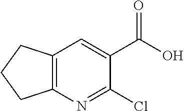 [SMILES: O=C(O)c1cc2c(nc1Cl)CCC2]

To a stirred solution of 2-hydroxy-6,7-dihydro-5H-cyclopenta[b]pyridine-3-carboxylic acid (5 g, 28 mmol) was dissolved into phosphorus oxychloride (50 mL) and heated to 110° C. for 19 hours. Progress of reaction was monitored by TLC. After completion of reaction, mixture was distilled using downward distillation to remove excess phosphorus oxychloride, poured into ice-cold water and extracted with ethyl acetate (2 times). The combined organic layer was washed with cold saturated solution of sodium bicarbonate, brine solution, dried over sodium sulfate, filtered and concentrated to afford the crude material. The crude material was purified through trituration with diethyl ether and n-pentane to afford 2-chloro-6,7-dihydro-5H-cyclopenta[b]pyridine-3-carboxylic acid as an off-white solid.

### Synthesis of 2-(4,4-difluoroazepan-1-yl)-6,7-dihydro-5H-cyclopenta[b]pyridine-3-carboxylic acid

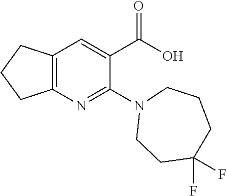 [SMILES: O=C(O)c1cc2c(nc1N1CCCC(F)(F)CC1)CCC2]

To a stirred solution of 2-chloro-6,7-dihydro-5H-cyclopenta[b]pyridine-3-carboxylic acid (2.5 g, 13 mmol) in N,N-dimethylformamide (25 mL) was added 4,4-difluoroazepane hydrogen chloride (4.8 g, 28.0 mmol) and dipotassium carbonate (8.8 g, 63.5 mmol). The reaction mixture was heated at 130° C. for 48 hours under nitrogen atmosphere. The progress of reaction was monitored by TLC. After completion of reaction, the crude material was diluted with ice-cold water and extracted with ethyl acetate. The organic layer was washed with brine solution and dried over anhydrous sodium sulfate. The crude material was purified by flash column chromatography with a gradient of 60-70% ethyl acetate in hexanes to afford 2-(4,4-difluoroazepan-1-yl)-6,7-dihydro-5H-cyclopenta[b]pyridine-3-carboxylic acid as a white solid.

### Synthesis of 2-(4,4-difluoroazepan-1-yl)-N-(3-sulfamoylphenyl)-6,7-dihydro-5H-cyclopenta[b]pyridine-3-carboxamide

To a solution of 2-(4,4-difluoroazepan-1-yl)-6,7-dihydro-5H-cyclopenta[b]pyridine-3-carboxylic acid (0.25 g, 0.84 mmol) in dichloromethane (5 mL) was added (1-[Bis(dimethylamino)methylene]-1H-1,2,3-triazolo[4,5-b]pyridinium-3-oxide hexafluorophosphate (HATU) (0.064 g, 1.69 mmol) and 4-(dimethylamino) pyridine (0.052 g, 0.42 mmol) at 0° C. and stirred the mixture at room temperature for 12 hours. After completion of reaction, solvent was evaporated under nitrogen atmosphere and diluted with acetonitrile (5 mL) and to this, 3-aminobenzenesulfonamide (0.17 g, 1.2 mmol) was added and heated to 80° C. for 12 hours. The progress of reaction was monitored by TLC. After completion of reaction, acetonitrile was removed in vacuo. The crude mass was diluted with water and extracted with ethyl acetate. The organic layer was washed with brine solution, dried over anhydrous sodium sulfate and concentrated. The crude material was purified by flash column chromatography with a gradient of 40-50% ethyl acetate in hexanes to afford 2-(4,4-difluoroazepan-1-yl)-N-(3-sulfamoylphenyl)-6,7-dihydro-5H-cyclopenta[b]pyridine-3-carboxamide as an off-white solid. MS (ESI): Calcd [M+H]⁺: 451.16, found: 451.25.

¹H NMR (400 MHz, DMSO-d₆): δ 10.59 (s, 1H), 8.31 (s, 1H), 7.80-7.77 (m, 1H), 7.57 (s, 1H), 7.52-7.51 (m, 2H), 7.39 (s, 2H), 3.58-3.57 (m, 2H), 3.38 (t, J=6.0 Hz, 2H), 2.81 (m, J=8.0 Hz, 4H), 2.30-2.26 (m, 1H), 2.08-2.04 (m, 1H), 2.02-1.90 (m, 2H), (m, 2H), 1.82-81 (m, 2H)

### Compound 41

### Synthesis of N-(3-carbamoylphenyl)-2-(4,4-difluoroazepan-1-yl)-1,8-naphthyridine-3-carboxamide

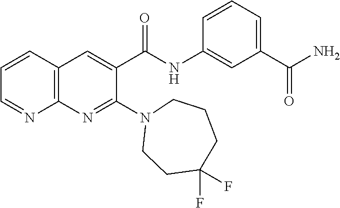 [SMILES: NC(=O)c1cccc(NC(=O)c2cc3cccnc3nc2N2CCCC(F)(F)CC2)c1]

### Synthesis of methyl 3-(2-(4,4-difluoroazepan-1-yl)-1,8-naphthyridine-3-carboxamido)benzoate

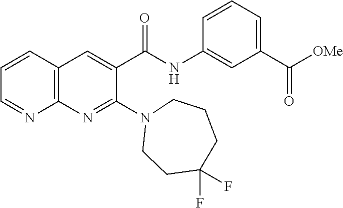 [SMILES: COC(=O)c1cccc(NC(=O)c2cc3cccnc3nc2N2CCCC(F)(F)CC2)c1]

To a solution of 2-(4,4-difluoroazepan-1-yl)-1,8-naphthyridine-3-carboxylic acid (0.15 g, 0.5 mmol) in dichloromethane (5 mL) was added [Bis(dimethylamino)methylene]-1H-1,2,3-triazolo[4,5-b]pyridinium 3-oxide hexafluorophosphate (HATU) (0.28 g, 0.73 mmol) and 4-Dimethylaminopyridine (0.003 g, 0.02 mmol) at 0° C. and stirred the reaction mixture at room temperature for 12 hours. After completion of reaction, dichloromethane was evaporated under reduced pressure and resultant crude mass was dissolved into acetonitrile (5 mL) and to this, methyl-3-aminobenzoate (0.082 g, 0.54 mmol) was added and reflux for 12 hours. After completion of reaction, acetonitrile was removed in vacuo. The crude mass was diluted with water and extracted with ethyl acetate. The organic layer was washed with brine solution and dried over anhydrous sodium sulfate, filtered and concentrated. The crude residue was purified by flash column chromatography with a gradient of 30-40% ethyl acetate in hexanes to afford methyl-3-(2-(4,4-difluoroazepan-1-yl)-1,8-naphthyridine-3-carboxamido)benzoate as a yellow solid.

### Synthesis of 3-(2-(4,4-difluoroazepan-1-yl)-1,8-naphthyridine-3-carboxamido)benzoic acid

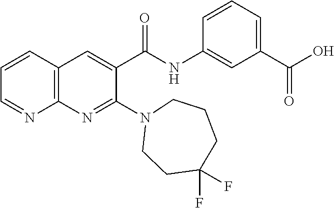 [SMILES: O=C(O)c1cccc(NC(=O)c2cc3cccnc3nc2N2CCCC(F)(F)CC2)c1]

To a solution of methyl-3-(2-(4,4-difluoroazepan-1-yl)-1,8-naphthyridine-3-carboxamido)benzoate (0.15 g, 0.34 mmol) in a mixture of tetrahydrofuran (5 mL) and methanol (5 mL) was added lithium hydroxide (0.029 g, 0.68 mmol) and water (5 mL). The reaction mixture was stirred at room temperature for 12 hours. After completion of reaction, the solvent was evaporated under vacuum. The crude mass was dissolved into water (10 mL), acidified with 1N hydrochloric acid solution and extracted with ethyl acetate. The combined organic layer was washed with brine solution and dried over anhydrous sodium sulfate, filtered and concentrated under reduced pressure. The crude residue was purified through trituration with diethyl ether and n-pentane to obtain 3-(2-(4,4-difluoroazepan-1-yl)-1,8-naphthyridine-3-carboxamido)benzoic acid as a yellow solid.

### Synthesis of N-(3-carbamoylphenyl)-2-(4,4-difluoroazepan-1-yl)-1,8-naphthyridine-3-carboxamide

To a solution of 3-(2-(4,4-difluoroazepan-1-yl)-1,8-naphthyridine-3-carboxamido)benzoic acid (0.10 g, 0.23 mmol) in N,N-dimethylformamide (5 mL) was added 1-[Bis(dimethylamino)methylene]-1H-1,2,3-triazolo[4,5-b]pyridinium 3-Oxide Hexafluorophosphate (HATU) (0.13 g, 0.35 mmol), N,N-diisopropylethylamine (0.2 mL, 1.15 mmol) and ammonium chloride (0.12 g. 2.3 mmol) respectively at 0° C. and stirred the mixture at room temperature for 12 hours. The progress of the reaction was monitor by TLC. After completion of reaction, the reaction mixture was diluted with ice-cold water and extracted with ethyl acetate. The organic layer was washed with brine solution and dried over anhydrous sodium sulfate, filtered and concentrated. The crude material was purified through reversed phase prep-HPLC to afford N-(3-carbamoylphenyl)-2-(4,4-difluoroazepan-1-yl)-1,8-naphthyridine-3-carboxamide as an off-white solid. MS (ESI): Calcd [M+H]⁺: 426.17, found: 426.25.

¹H NMR (400 MHz, DMSO-d₆): δ 10.82 (s, 1H), 8.83 (dd, J=1.6, 4.0 Hz, 1H), 8.37 (s, 1H), 8.26 (dd, J=1.6, 7.6 Hz, 1H), 8.19 (s, 1H), 7.96 (s, 1H), 7.86 (d, J=8.0 Hz, 1H), 7.61 (d, J=7.6, 1H), 7.44 (t, J=8.4, 1H), 7.38 (s, 1H), 7.32-7.29 (m, 1H), 3.82-3.79 (m, 2H), 3.65 (t, J=5.6 Hz, 2H), 2.42-2.38 (m, 2H), 2.05-2.00 (m, 2H), 1.96-1.90 (m, 2H).

### Compound 42

### Synthesis of 2-(4,4-difluoroazepan-1-yl)-N-(3-sulfamoylphenyl)-1,5-naphthyridine-3-carboxamide

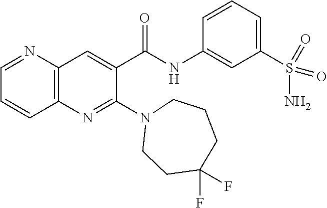 [SMILES: NS(=O)(=O)c1cccc(NC(=O)c2cc3ncccc3nc2N2CCCC(F)(F)CC2)c1]

### Synthesis of tert-butyl (2-bromopyridin-3-yl)carbamate

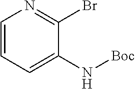 [SMILES: CC(C)(C)OC(=O)Nc1cccnc1Br]

To a solution of 2-bromonicotinic acid (10 g, 49.5 mmol) in tert-butanol (100 mL) was added diphenylphosphoryl azide (10 mL, 49.5 mmol) and triethylamine (7 mL, 49.5 mmol) at room temperature and the reaction mixture was heated at 80° C. for 2 hours. After completion of reaction, the solvent was evaporated under reduced pressure to obtain the crude mass which was purified by flash column chromatography with a gradient of 20-30% ethyl acetate in hexanes to afford tert-butyl (2-bromopyridin-3-yl)carbamate as a white solid.

### Synthesis of tert-butyl (2-formylpyridin-3-yl)carbamate

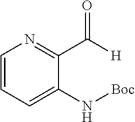 [SMILES: CC(C)(C)OC(=O)Nc1cccnc1C=O]

To a solution of tert-butyl (2-bromopyridin-3-yl)carbamate (8.4 g, 31 mmol) in dry tetrahydrofurane (80 mL) was added 1.6 M solution of n-butyl lithium (48 mL, 77 mmol) dropwise at −78° C. under nitrogen atmosphere. After stirring for 1 hour, a solution of piperidine-1-carbaldehyde (4.2 g, 37 mmol) in tetrahydrofuran was added at −78° C. and the reaction mixture was allowed to stirrer at room temperature for 2 hours. After completion of reaction, the reaction mixture was quenched at 0° C. by the slow addition of 1N hydrochloric acid solution. Afterwards, solid sodium carbonate was then added and the solution was extracted with ethyl acetate. The combined organic layer was washed with brine, dried over anhydrous sodium sulfate, filtered and concentrated in vacuum to afford the crude material which was purified by trituration with diethyl ether and n-pentane to afford tert-butyl (2-formylpyridin-3-yl)carbamate as a yellow solid

### Synthesis of 3-aminopicolinaldehyde

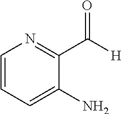

To a solution of tert-butyl (2-formylpyridin-3-yl)carbamate (1 g, 4.5 mmol) in dichloromethane (20 mL) was added trifluoroacetic acid (10 mL) and stirred the reaction mixture at room temperature for 5 hours. The progress of reaction was monitored by TLC. After completion of reaction, solvent was evaporated under rotatory and the residue thus obtained was dissolved into ethyl acetate and washed with saturated sodium bicarbonate solution. The organic layer was separated, washed with brine, dried over anhydrous sodium sulfate, filtered and concentrated under vacuum. The crude material was purified by trituration with diethyl ether and n-pentane to afford 3-aminopicolinaldehyde as a yellow solid.

### Synthesis of ethyl 2-hydroxy-1,5-naphthyridine-3-carboxylate

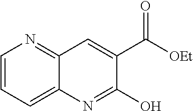 [SMILES: CCOC(=O)c1cc2ncccc2nc1O]

To a solution of 3-aminopicolinaldehyde (0.5 g, 4 mmol) in ethanol (20 mL) was added diethyl malonate (1.3 mL, 8 mmol) and piperidine (1.4 mL, 16.4 mmol) and the reaction mixture was heated to 80° C. for 12 hours. After completion of reaction, the solvent was evaporated and the crude residue was purified by column chromatography with gradient of 5-10% methanol in dichloromethane to afford ethyl 2-hydroxy-1,5-naphthyridine-3-carboxylate as a brown solid.

### Synthesis of ethyl 2-chloro-1,5-naphthyridine-3-carboxylate

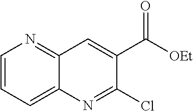 [SMILES: CCOC(=O)c1cc2ncccc2nc1Cl]

A mixture of ethyl 2-hydroxy-1,5-naphthyridine-3-carboxylate (0.5 g, 2.2 mmol) and phosphorus oxychloride (5.0 mL) was heated to 80° C. for 12 hours. The progress of reaction was monitored by TLC. After completion of reaction, the reaction mixture was cooled to room temperature and excess phosphorus oxychloride was distilled out. The crude residue was dissolved into ethyl acetate and washed with saturated sodium bicarbonate solution. The organic layer was separated, washed with brine solution, dried over anhydrous sodium sulfate, filtered and concentrated under vacuum to afford the crude material which was triturated with diethyl ether and n-pentane to obtain ethyl 2-chloro-1,5-naphthyridine-3-carboxylate as a light brown solid.

### Synthesis of ethyl 2-(4,4-difluoroazepan-1-yl)-1,5-naphthyridine-3-carboxylate

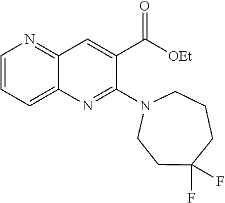 [SMILES: CCOC(=O)c1cc2ncccc2nc1N1CCCC(F)(F)CC1]

To a solution of ethyl 2-chloro-1,5-naphthyridine-3-carboxylate (0.5 g, 2.1 mmol) in N,N-dimethylformamide (10 mL) was added 4,4-difluoroazepane hydrogen chloride (0.54 g, 3.2 mmol) and dipotassium carbonate (1.2 g, 8.4 mmol). The resulting reaction mixture was heated at 100° C. for 72 hours under nitrogen atmosphere. The progress of reaction was monitored by TLC. After completion of reaction, the crude material was diluted with ice cold water and extracted with ethyl acetate. The organic layer was washed with brine solution and dried over anhydrous sodium sulfate, filtered and concentrated. The crude material was purified by flash column chromatography with a gradient of 20-30% ethyl acetate in hexanes to afford ethyl 2-(4,4-difluoroazepan-1-yl)-1,5-naphthyridine-3-carboxylate as a yellow liquid.

### Synthesis of 2-(4,4-difluoroazepan-1-yl)-1,5-naphthyridine-3-carboxylic acid

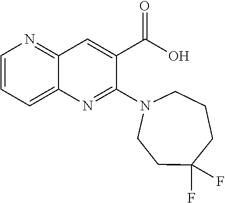 [SMILES: O=C(O)c1cc2ncccc2nc1N1CCCC(F)(F)CC1]

To a solution of ethyl 2-(4,4-difluoroazepan-1-yl)-1,5-naphthyridine-3-carboxylate (0.5 g, 1.5 mmol) in a mixture of tetrahydrofuran (10 mL) and methanol (10 mL) was added lithium hydroxide (0.07 g, 3 mmol) and water (10 mL). The reaction mixture was stirred at room temperature for 12 hours. After completion of reaction, the solvent was evaporated under vacuum. The crude mass was dissolved into water (10 mL), acidified with 1N hydrochloric acid solution and extracted with ethyl acetate. The combined organic layer was washed with brine solution and dried over anhydrous sodium sulfate, filtered and concentrated under reduced pressure. The crude residue was purified by trituration with diethyl ether and n-pentane to afford 2-(4,4-difluoroazepan-1-yl)-1,5-naphthyridine-3-carboxylic acid as a light brown solid.

### Synthesis of 2-(4,4-difluoroazepan-1-yl)-N-(3-sulfamoylphenyl)-1,5-naphthyridine-3-carboxamide

To a solution of 2-(4,4-difluoroazepan-1-yl)-1,5-naphthyridine-3-carboxylic acid (0.2 g, 0.65 mmol) in dichloromethane (10 mL) was added 1-[Bis(dimethylamino)methylene]-1H-1,2,3-triazolo[4,5-b]pyridinium 3-Oxide Hexafluorophosphate (HATU) (0.38 g, 1 mmol) and 4-Dimethylaminopyridine (0.004 g, 0.03 mmol) at 0° C. and stirred the mixture at room temperature for 12 hours. The progress of the reaction was monitor by TLC. After completion of reaction, dichloromethane was evaporated under reduced pressure. The crude mass was dissolved into acetonitrile (5.0 mL) and to this, 3-aminobenzenesulfonamide (0.13 g, 0.78 mmol) was added at room temperature and heated at 80° C. for 12 hours. After completion of reaction, acetonitrile was removed under rotatory and diluted with water, extracted with ethyl acetate. The organic layer was washed with brine solution and dried over anhydrous sodium sulfate, filtered and concentrated. The crude material was purified through reversed phase prep-HPLC to obtain 2-(4,4-difluoroazepan-1-yl)-N-(3-sulfamoylphenyl)-1,5-naphthyridine-3-carboxamide as a white solid. MS (ESI): Calcd [M+H]⁺: 462.14, found: 462.30.

¹H NMR (400 MHz, DMSO-d₆): δ 11.04 (s, 1H), 8.68 (d, J=4.0, 1.2 Hz, 1H), 8.32 (s, 1H), 8.28 (s, 1H), 8.01 (d, J=8.4 Hz, 1H), 7.88-7.86 (m, 1H), 7.65-7.61 (m, 1H), 7.59-7.55 (m, 2H), 7.41 (s, 2H), 3.78-3.77 (m, 2H), 3.62 (t, J=5.6 Hz, 2H), 2.43-2.36 (m, 2H), 2.07-1.97 (m, 2H), 1.96-1.90 (m, 2H).

### Compound 43

### Synthesis of 2-(4,4-difluoroazepan-1-yl)-N-(3-(S-methylsulfonimidoyl)phenyl)-5,6,7,8-tetrahydroquinoline-3-carboxamide

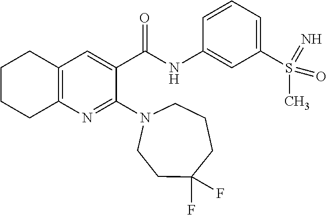 [SMILES: CS(=N)(=O)c1cccc(NC(=O)c2cc3c(nc2N2CCCC(F)(F)CC2)CCCC3)c1]

### Synthesis of 2-(4,4-difluoroazepan-1-yl)-N-(3-(methylsulfinyl)phenyl)-5,6,7,8-tetrahydroquinoline-3-carboxamide

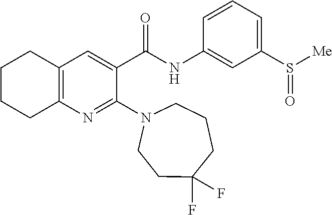 [SMILES: CS(=O)c1cccc(NC(=O)c2cc3c(nc2N2CCCC(F)(F)CC2)CCCC3)c1]

To a solution of 2-(4,4-difluoroazepan-1-yl)-5,6,7,8-tetrahydroquinoline-3-carboxylic acid (0.15 g, 0.5 mmol) in dichloromethane (10 mL) was added (1-[Bis(dimethylamino)methylene]-1H-1,2,3-triazolo[4,5-b]pyridinium 3-oxide hexafluorophosphate (HATU) (0.28 g, 0.75 mmol) and 4-Dimethylaminopyridine (0.003 g, 0.025 mmol) at 0° C. and the mixture was stirred at room temperature for 12 hours. After completion of reaction, dichloromethane was evaporated under reduced pressure. The crude mass was dissolved into acetonitrile (5 mL) and to this, 3-(methylsulfinyl) aniline (0.093 g, 0.6 mmol) was added and heated at 80° C. for 12 hours. After completion of reaction, acetonitrile was removed in vacuo. The crude mass was diluted with water and extracted with ethyl acetate. The organic layer was washed with brine solution and dried over anhydrous sodium sulphate, filtered and concentrated under reduced pressure. The crude material was purified by flash column chromatography with a gradient of 30-40% ethyl acetate in hexanes to afford 2-(4,4-difluoroazepan-1-yl)-N-(3-(methylsulfinyl)phenyl)-5,6,7,8-tetrahydroquinoline-3-carboxamide as white solid.

### Synthesis of 2-(4,4-difluoroazepan-1-yl)-N-(3-(S-methylsulfonimidoyl)phenyl)-5,6,7,8-tetrahydroquinoline-3-carboxamide

To a solution of 2-(4,4-difluoroazepan-1-yl)-N-(3-(methylsulfinyl)phenyl)-5,6,7,8-tetrahydroquinoline-3-carboxamide (0.09 g, 0.2 mmol) in Eaton's reagent (2.0 mL) was added sodium azide (0.026 g, 0.4 mmol) at 0° C., stirred for 15 min at room temperature and heated the mixture at 80° C. for 2 hours. The progress of reaction was monitored by TLC. After completion of reaction, the mixture is quenched with sodium carbonate solution and extracted with ethyl acetate. The organic layer was washed with brine solution and dried over anhydrous sodium sulphate, filtered and concentrated. The crude material was purified by flash column chromatography with a gradient of 70-80% ethyl acetate in hexanes to afford 2-(4,4-difluoroazepan-1-yl)-N-(3-(S-methylsulfonimidoyl)phenyl)-5,6,7,8-tetrahydroquinoline-3-carboxamide as white solid. MS (ESI): Calcd [M+H]⁺: 463.20, found: 463.35.

¹H NMR (400 MHz, DMSO-d₆): δ 10.61 (s, 1H), 8.59 (s, 1H), 7.91 (d, J=8.0 Hz, 1H), 7.61 (d, J=7.6 Hz, 1H), 7.55 (t, J=8.0, 1H), 7.43 (s, 1H), 4.17 (s, 1H), 3.57-3.36 (m, 2H), 3.38 (t, J=5.6 Hz, 2H), 3.04 (s, 3H), 2.70-2.68 (m, 4H), 2.28-2.57 (m, 2H), 1.94-1.90 (m, 2H), 1.80-1.73 (m, 6H).

### Compound 44

### Synthesis of N-(3-carbamoylphenyl)-2-(4,4-difluoroazepan-1-yl)-1,6-naphthyridine-3-carboxamide

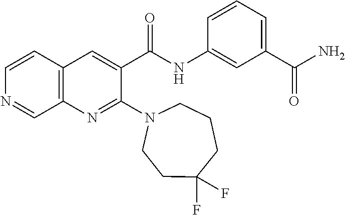 [SMILES: NC(=O)c1cccc(NC(=O)c2cc3ccncc3nc2N2CCCC(F)(F)CC2)c1]

### Synthesis of methyl 3-(2-(4,4-difluoroazepan-1-yl)-1,6-naphthyridine-3-carboxamido)benzoate

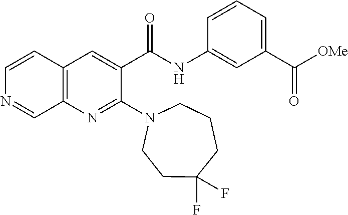 [SMILES: COC(=O)c1cccc(NC(=O)c2cc3ccncc3nc2N2CCCC(F)(F)CC2)c1]

To a stirred solution of 2-(4,4-difluoroazepan-1-yl)-1,7-naphthyridine-3-carboxylic acid (0.25 g, 0.81 mmol) in dry dichloromethane (5 mL) was added Bis(dimethylamino)methylene]-1H-1,2,3-triazolo[4,5-b]pyridinium 3-oxide hexafluorophosphate (HATU) (0.46 g, 1.2 mmol) and N,N-dimethylpyridin-4-amine (0.05 g, 0.41 mmol) was added at 0° C. and reaction mixture was stirred at room temperature for 16 hours. After completion of reaction, solvent was evaporated in vacuo and diluted with acetonitrile (5 mL) and to this, methyl 3-aminobenzoate (0.15 g, 0.98 mmol) was added at room temperature and slowly heated to 70° C. for 16 hours. The progress of the reaction was monitored by TLC. After completion of the reaction, the reaction mixture was diluted with water and extracted with ethyl acetate. The organic layer was dried over anhydrous sodium sulfate. The filtrate was concentrated under reduced pressure and the crude residue was purified by flash column chromatography with a gradient of 50-70% ethyl acetate in hexanes to afford methyl 3-(2-(4,4-difluoroazepan-1-yl)-1,6-naphthyridine-3-carboxamido)benzoate as a white solid.

### Synthesis 3-(2-(4,4-difluoroazepan-1-yl)-1,6-naphthyridine-3-carboxamido)benzoic acid

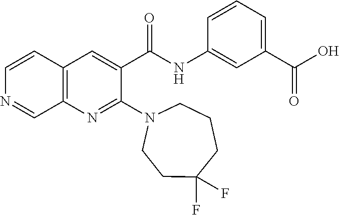 [SMILES: O=C(O)c1cccc(NC(=O)c2cc3ccncc3nc2N2CCCC(F)(F)CC2)c1]

To a solution of methyl 3-(2-(4,4-difluoroazepan-1-yl)-1,6-naphthyridine-3-carboxamido)benzoate (0.15 g, 0.34 mmol) in a mixture of methanol (5 mL), tetrahydrofuran (5 mL) was added a solution of lithium hydroxide (0.072 g, 1.7 mmol) in water (5 mL) at room temperature and stirred for 12 hours. The progress of reaction was monitored by TLC. After completion of reaction, the crude material was diluted with water and acidified with 1N hydrochloric acid solution and extracted with 10% isopropanol in chloroform. The organic layer was washed with brine solution, dried over anhydrous sodium sulfate and filtered. The filtrate was concentrated and the resultant crude material was triturated with diethyl ether and n-pentane to afford 3-(2-(4,4-difluoroazepan-1-yl)-1,6-naphthyridine-3-carboxamido)benzoic acid as a light yellow solid.

### Synthesis of N-(3-carbamoylphenyl)-2-(4,4-difluoroazepan-1-yl)-1,6-naphthyridine-3-carboxamide

To a stirred solution of 3-(2-(4,4-difluoroazepan-1-yl)-1,6-naphthyridine-3-carboxamido)benzoic acid (0.13 g, 0.31 mmol) in N,N-dimethylformamide (5 mL) was added ethylbis(propan-2-yl)amine (0.2 g, 1.5 mmol), Bis(dimethylamino)methylene]-1H-1,2,3-triazolo[4,5-b]pyridinium 3-oxide hexafluorophosphate (HATU) (0.17 g, 0.46 mmol) and ammonium chloride (0.16 g, 3.1 mmol) was added at 0° C. and the reaction mixture was stirred at room temperature for 16 hours. The progress of reaction mixture was monitored by TLC and LCMS. After completion of reaction, the reaction mixture was diluted with water and extracted with ethyl acetate. The organic layer was dried over anhydrous sodium sulfate, filtered and concentrated under reduced pressure to obtain the crude material which was purified by flash column chromatography with a gradient of 0-5% methanol in dichloromethane to afford N-(3-carbamoylphenyl)-2-(4,4-difluoroazepan-1-yl)-1,6-naphthyridine-3-carboxamide as as an off-white solid. MS (ESI): Calcd [M+H]⁺: 426.17, found: 426.35.

¹H NMR (400 MHz, DMSO-d₆): δ 11.94 (s, 1H), 10.88 (s, 1H), 9.00 (s, 1H), 8.37 (s, 1H), 8.35 (d, J=4 Hz, 1H), 8.18 (s, 1H), 7.97 (s, 1H), 7.86 (d, J=8 Hz, 1H), 7.75 (d, J=4 Hz, 1H), 7.47 (t, J=8 Hz, 1H), 7.38 (s, 1H), 3.80-3.78 (m, 2H), 3.67 (t, J=8 Hz, 2H), 2.42-2.39 (m, 2H), 2.03-2.02 (m, 2H), 1.97-1.90 (m, 2H).

### Compound 45

### Synthesis of 6-(4,4-difluoroazepan-1-yl)-N-(3-sulfamoylphenyl)-[1,3]dioxolo[4,5-g]quinoline-7-carboxamide

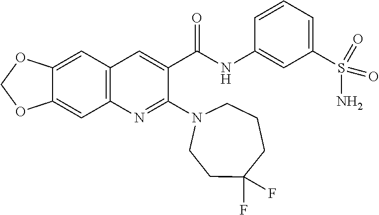 [SMILES: NS(=O)(=O)c1cccc(NC(=O)c2cc3cc4c(cc3nc2N2CCCC(F)(F)CC2)OCO4)c1]

### Synthesis of 6-chloro-[1,3]dioxolo[4,5-g]quinoline-7-carboxylic acid

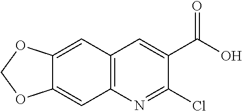 [SMILES: O=C(O)c1cc2cc3c(cc2nc1Cl)OCO3]

To a stirred solution of 6-chloro-[1,3]dioxolo[4,5-g]quinoline-7-carbaldehyde (2 g, 8.5 mmol) in ethanol (35 mL) was added silver nitrate (2.2 g, 13 mmol). To this mixture, a solution of sodium hydroxide (1.7 g, 42.4 mmol) in water (50 mL) was added dropwise. The reaction mixture was stirred at 80° C. for 2 hours. After completion of reaction, the reaction mixture was filtered through celite pad and solvent was removed in vacuo to obtain the crude mass which was diluted with water and acidified with 1N hydrochloric acid solution and extracted with ethyl acetate. The organic layer was separated, washed with brine solution, dried over anhydrous sodium sulfate and concentrated in vacuo to afford the crude material. The crude residue was purified through trituration with diethyl ether and n-pentane to afford 6-chloro-[1,3]dioxolo[4,5-g]quinoline-7-carboxylic acid as a brown solid.

### Synthesis of 6-(4,4-difluoroazepan-1-yl)-[1,3]dioxolo[4,5-g]quinoline-7-carboxylic acid

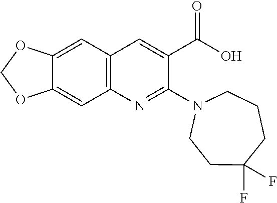 [SMILES: O=C(O)c1cc2cc3c(cc2nc1N1CCCC(F)(F)CC1)OCO3]

To a solution of 6-chloro-[1.3]dioxolo[4,5-g]quinoline-7-carboxylic acid (0.7 g, 2.8 mmol) in N,N-dimethyl formamide (20 mL) was added 4,4-difluoroazepane hydrogen chloride (0.56 g, 4.2 mmol) and potassium carbonate (1.54 g, 11.1 mmol). The mixture was heated at 70° C. for 18 hours under nitrogen atmosphere. The progress of reaction was monitored by TLC. After completion of the reaction, the crude material was diluted with water followed by treatment with 1N hydrochloric acid solution and extracted with ethyl acetate. The organic layer was washed with brine solution and dried over anhydrous sodium sulfate. The crude material was purified through trituration with diethyl ether and n-pentane to afford 6-(4,4-difluoroazepan-1-yl)-[1,3]dioxolo[4,5-g]quinoline-7-carboxylic acid as a light brown solid.

### Synthesis 6-(4,4-difluoroazepan-1-yl)-N-(3-sulfamoylphenyl)-[1,3]dioxolo[4,5-g]quinoline-7-carboxamide

To a stirred solution of 6-(4,4-difluoroazepan-1-yl)-[1,3]dioxolo[4,5-g]quinoline-7-carboxylic acid (0.3 g, 0.86 mmol) in dry dichloromethane (6.0 mL) was added Bis(dimethylamino)methylene]-1H-1,2,3-triazolo[4,5-b]pyridinium 3-oxide hexafluorophosphate (0.488 g, 1.28 mmol) and N,N-dimethylpyridin-4-amine (0.052 g, 0.428 mmol) was added at 0° C. The reaction mixture was stirred at room temperature for 16 hours. After completion of reaction, solvent was removed under the rotavapour and diluted with acetonitrile (6.0 mL) and 3-aminobenzene-1-sulfonamide (0.18 g, 1 mmol) was added to the above solution. The reaction mixture was stirred at 70° C. for 16 hours. The progress of the reaction was monitored by TLC. After completion of the reaction, the mixture was diluted with water and extracted with ethyl acetate. The organic layer was dried over anhydrous sodium sulfate and filtered. The filtrate was concentrated under reduced pressure and the resultant crude residue was purified by flash column chromatography with a gradient of 50-70% ethyl acetate in hexanes to afford 6-(4,4-difluoroazepan-1-yl)-N-(3-sulfamoylphenyl)-[1,3]dioxolo[4,5-g]quinoline-7-carboxamide as a white solid. MS (ESI): Calcd [M+H]⁺: 505.14, found: 505.25.

¹H NMR (400 MHz, DMSO-d₆): δ 10.84 (s, 1H), 8.32 (s, 1H), 8.14 (s, 1H), 7.85-7.82 (m, 1H), 7.56-7.55 (m, 2H), 7.40 (s, 2H), 7.26 (s, 1H), 7.03 (s, 1H), 6.13 (s, 2H), 3.69-3.67 (m, 2H), 3.55 (t, J=4 Hz, 2H), 2.49-2.37 (m, 2H), 2.00-1.92 (m, 2H), 1.86-1.85 (m, 2H).

### Compound 46

### Synthesis of 2-(4,4-difluoroazepan-1-yl)-6-methyl-N-(3-sulfamoylphenyl)-5,6,7,8-tetrahydroquinoline-3-carboxamide

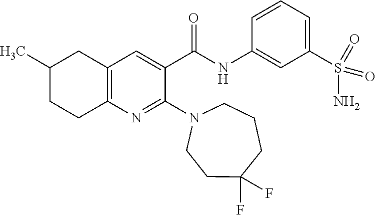 [SMILES: CC1CCc2nc(N3CCCC(F)(F)CC3)c(C(=O)Nc3cccc(S(N)(=O)=O)c3)cc2C1]

### Synthesis of (Z)-2-((dimethylamino)methylene)-5-methylcyclohexan-1-one

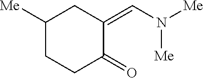 [SMILES: CC1CCC(=O)/C(=C\N(C)C)C1]

A mixture of 4-methylcyclohexan-1-one (10 g, 89 mmol) and 1-tert-butoxy-N,N,N′,N′-tetramethylmethanediamine (18.6 g, 107 mmol) was stirred at 70° C. for 18 hours. The progress of reaction was monitored by TLC and LCMS. After completion of reaction, the solvent was removed under rotatory to obtain the crude (Z)-2-((dimethylamino)methylene)-5-methylcyclohexan-1-one as a brown liquid which was used as such into next step without any purification.

### Synthesis of methyl 2-hydroxy-6-methyl-5,6,7,8-tetrahydroquinoline-3-carboxylate

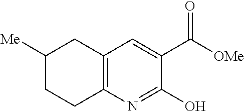 [SMILES: COC(=O)c1cc2c(nc1O)CCC(C)C2]

To the crude (Z)-2-((dimethylamino)methylene)-5-methylcyclohexan-1-one (12 g, 72 mmol) in methanol (60 mL) was added methyl 2-cyanoacetate (8.5 g, 86 mmol) and heated the reaction mixture at 80° C. for 12 hours. After completion of reaction, the solvent was removed under rotatory. The crude mass was purified by flash column chromatography with a gradient of 80% ethyl acetate in hexanes to afford methyl 2-hydroxy-6-methyl-5,6,7,8-tetrahydroquinoline-3-carboxylate as an off-white solid.

### Synthesis of methyl 2-chloro-6-methyl-5,6,7,8-tetrahydroquinoline-3-carboxylate

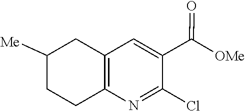 [SMILES: COC(=O)c1cc2c(nc1Cl)CCC(C)C2]

Methyl 2-hydroxy-6-methyl-5,6,7,8-tetrahydroquinoline-3-carboxylate (4 g, 18 mmol) was dissolved into phosphorus oxychloride (40 mL) at room temperature and slowly heated to 130° C. for 12 hours. After completion of reaction, the mixture was then cooled to room temperature and excess of phosphorus oxychloride was distilled out in vacuo. The residue thus obtained was dissolved into ethyl acetate and washed with sodium bicarbonate solution. The organic layer was separated, washed with brine solution, dried over anhydrous sodium sulfate and concentrated in vacuum to afford the crude material. The crude material was purified through trituration with diethyl ether and n-pentane to afford methyl 2-chloro-6-methyl-5,6,7,8-tetrahydroquinoline-3-carboxylate as a white solid.

### Synthesis of methyl 2-(4,4-difluoroazepan-1-yl)-6-methyl-5,6,7,8-tetrahydroquinoline-3-carboxylate

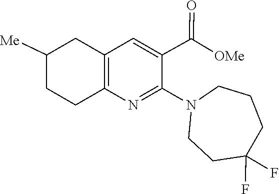 [SMILES: COC(=O)c1cc2c(nc1N1CCCC(F)(F)CC1)CCC(C)C2]

To a solution of 2-chloro-6-methyl-5,6,7,8-tetrahydroquinoline-3-carboxylate (2 g, 8.3 mmol) in N,N-dimethylformamide (20 mL) was added 4,4-difluoroazepane hydrogen chloride (2.15 g, 12.5 mmol) and cesium carbonate (10.9 g, 33.4 mmol). The reaction mixture was heated at 100° C. for 24 hours under nitrogen atmosphere. The progress of reaction was monitored by TLC. After completion of reaction, the crude material was diluted with ice-cold water and extracted with ethyl acetate. The organic layer was washed with brine solution and dried over anhydrous sodium sulfate. The crude material was purified by flash column chromatography with a gradient of 20% ethyl acetate in hexanes to afford methyl 2-(4,4-difluoroazepan-1-yl)-6-methyl-5,6,7,8-tetrahydroquinoline-3-carboxylate as a pale yellow liquid.

### Synthesis of 2-(4,4-difluoroazepan-1-yl)-6-methyl-5,6,7,8-tetrahydroquinoline-3-carboxylic acid

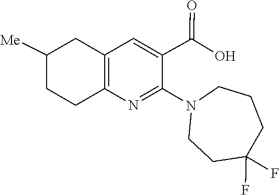 [SMILES: CC1CCc2nc(N3CCCC(F)(F)CC3)c(C(=O)O)cc2C1]

To a solution of methyl 2-(4,4-difluoroazepan-1-yl)-6-methyl-5,6,7,8-tetrahydroquinoline-3-carboxylate (0.94 g, 2.78 mmol) in tetrahydrofuran (10 mL) and methanol (10 mL) was added lithium hydroxide (0.47 g, 11 mmol) in water (10 mL) and stirred at room temperature for 12 hours. After completion of reaction, solvent was removed under rotatory and diluted with water, acidified with 1N hydrochloric solution and extracted with ethyl acetate. The organic layer was dried over anhydrous sodium sulfate and filtered. The filtrate was concentrated under reduced pressure and the resultant crude residue was purified by trituration with diethyl ether and n-pentane to afford 2-(4,4-difluoroazepan-1-yl)-5,6,7,8-tetrahydroquinoline-3-carboxylic acid as a white solid.

### Synthesis of 2-(4,4-difluoroazepan-1-yl)-6-methyl-N-(3-sulfamoylphenyl)-5,6,7,8-tetrahydroquinoline-3-carboxamide

To a solution of 2-(4,4-difluoroazepan-1-yl)-5,6,7,8-tetrahydroquinoline-3-carboxylic acid (0.5 g, 1.54 mmol) in dichloromethane (20 mL) was added (1-[Bis(dimethylamino)methylene]-1H-1,2,3-triazolo[4,5-b]pyridinium 3-oxide hexafluorophosphate (HATU) (0.88 g, 2.3 mmol) and 4-(dimethylamino)pyridine (0.094 g, 0.08 mmol) at 0° C. and stirred the mixture at room temperature for 12 hours. After completion of reaction, solvent was evaporated under nitrogen atmosphere and diluted with acetonitrile (20 mL) and to this, 3-aminobenzenesulfonamide (0.32 g, 1.85 mmol) was added and heated to 80° C. for 12 hours. The progress of reaction was monitored by TLC. After completion of reaction, acetonitrile was removed under rotatory. The crude mass was diluted with water and extracted with ethyl acetate. The organic layer was washed with brine solution, dried over anhydrous sodium sulfate and concentrated. The crude material was purified by flash column chromatography with a gradient of 40% ethyl acetate in hexanes to afford 2-(4,4-difluoroazepan-1-yl)-6-methyl-N-(3-sulfamoylphenyl)-5,6,7,8-tetrahydroquinoline-3-carboxamide as a white solid. MS (ESI): Calcd [M+H]⁺: 479.19, found: 479.30.

¹H NMR (400 MHz, DMSO-d₆): δ 10.59 (s, 1H), 8.30 (s, 1H), 7.78-7.81 (m, 1H), 7.50-7.53 (m, 2H), 7.41 (s, 1H), 7.37 (s, 2H), 3.53-3.62 (m, 2H), 3.36-3.45 (m, 2H), 2.69-2.74 (m, 3H), 2.24-2.30 (m, 3H), 1.81-2.0 (m, 6H), 1.39-1.49 (m, 1H), 1.56 (d, J=6.4 Hz, 3H).

### Compound 47

### Synthesis of 2-(4,4-difluoroazepan-1-yl)-6,7-dimethoxy-N-(3-sulfamoylphenyl)quinoline-3-carboxamide

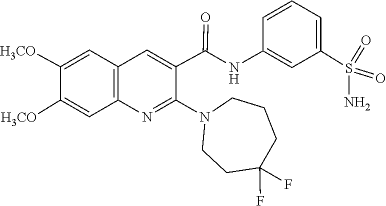 [SMILES: COc1cc2cc(C(=O)Nc3cccc(S(N)(=O)=O)c3)c(N3CCCC(F)(F)CC3)nc2cc1OC]

### Synthesis of 2-chloro-6,7-dimethoxyquinoline-3-carboxylic acid

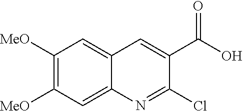 [SMILES: COc1cc2cc(C(=O)O)c(Cl)nc2cc1OC]

To a stirred solution of 2-chloro-6,7-dimethoxyquinoline-3-carbaldehyde (2 g, 8 mmol) in ethanol (35 mL) was added silver nitrate (2 g, 12 mmol). After 15 min, a solution of sodium hydroxide (2 g, 37.7 mmol) in water (45 mL) was added dropwise and stirred at 80° C. for 2 hours. After completion of reaction, the reaction mixture was filtered through a pad of Celite and filtrate was concentrated under vacuum to obtain a crude mass which was diluted with water and acidified with 1 N hydrochloric acid solution and extracted with ethyl acetate. The organic layer was separated, washed with brine, dried over anhydrous sodium sulfate and concentrated in vacuo to afford the crude material. The crude material was triturated with diethyl ether and n-pentane to afford 2-chloro-6,7-dimethoxyquinoline-3-carboxylic acid as a brown solid.

### Synthesis of 2-(4,4-difluoroazepan-1-yl)-6,7-dimethoxyquinoline-3-carboxylic acid

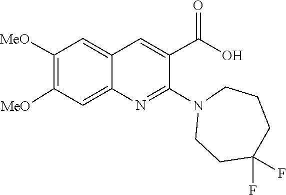 [SMILES: COc1cc2cc(C(=O)O)c(N3CCCC(F)(F)CC3)nc2cc1OC]

To a solution of 2-chloro-6,7-dimethoxyquinoline-3-carboxylic acid (1 g, 3.7 mmol) in N-methyl-2-pyrrolidone (10 mL) was added 4,4-difluoroazepane hydrogen chloride (0.76 g, 5.6 mmol) and potassium carbonate (2.1 g, 14.9 mmol). The reaction mixture was heated at 70° C. for 18 hours under nitrogen atmosphere. The progress of reaction was monitored by TLC. After completion of reaction, the reaction mixture was diluted with cold water followed by treatment with 1 N hydrochloric acid solution and extracted with ethyl acetate. The organic layer was washed with brine solution and dried over anhydrous sodium sulphate and concentrated. The crude material was triturated with diethyl ether and n-pentane to afford 2-(4,4-difluoroazepan-1-yl)-6,7-dimethoxyquinoline-3-carboxylic acid as a light brown solid.

### Synthesis of 2-(4,4-difluoroazepan-1-yl)-6,7-dimethoxy-N-(3-sulfamoylphenyl)quinoline-3-carboxamide

To a stirred solution of 2-(4,4-difluoroazepan-1-yl)-6,7-dimethoxyquinoline-3-carboxylic acid (0.3 g, 0.8 mmol) in dichloromethane (5 mL) was added Bis(dimethylamino)methylene]-1H-1,2,3-triazolo[4,5-b]pyridinium 3-oxide hexafluorophosphate (0.47 g, 1.23 mmol) (HATU) and N,N-dimethylpyridin-4-amine (DMAP) (0.050 g, 0.41 mmol) was added at 0° C. and stirred at room temperature for 16 hours. After completion of reaction, solvent was evaporated under rotatory and diluted with acetonitrile (5 mL) and to this, 3-aminobenzene-1-sulfonamide (0.17 g, 1 mmol). The reaction mixture was stirred at 70° C. for 16 h. The progress of the reaction was monitored by TLC. After completion the reaction mixture was diluted with water and extracted with ethyl acetate. The organic layer was dried over anhydrous sodium sulphate and filtered. The filtrate was concentrated under reduced pressure and the crude residue was purified by flash column chromatography with a gradient of 50-70% ethyl acetate in hexanes to afford 2-(4,4-difluoroazepan-1-yl)-6,7-dimethoxy-N-(3-sulfamoylphenyl)quinoline-3-carboxamide as an off-white solid. MS (ESI): Calcd [M+H]⁺: 521.17, found: 521.35.

¹H NMR (400 MHz, DMSO-d₆): δ 10.83 (s, 1H), 8.34 (s, 1H), 8.15 (s, 1H), 7.84-7.82 (m, 1H), 7.56-7.53 (m, 2H), 7.40 (s, 2H), 7.27 (s, 1H), 7.05 (s, 1H), 3.90 (s, 3H), 3.83 (s, 3H), 3.70-3.68 (m, 2H), 3.56 (t, J=4 Hz, 2H), 2.49-2.40 (m, 2H), 2.02-1.96 (m, 2H), 1.87-1.86 (m, 2H)

### Compound 48

### Synthesis of 2-(4,4-difluoroazepan-1-yl)-N-(3-(S-methylsulfonimidoyl)phenyl)-1,7-naphthyridine-3-carboxamide

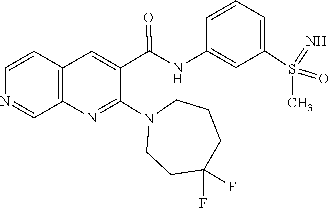 [SMILES: CS(=N)(=O)c1cccc(NC(=O)c2cc3ccncc3nc2N2CCCC(F)(F)CC2)c1]

### Synthesis 2-(4,4-difluoroazepan-1-yl)-N-(3-(methylsulfinyl)phenyl)-1,7-naphthyridine-3-carboxamide

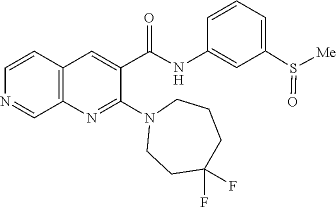 [SMILES: CS(=O)c1cccc(NC(=O)c2cc3ccncc3nc2N2CCCC(F)(F)CC2)c1]

To a stirred solution of 2-(4,4-difluoroazepan-1-yl)-1,7-naphthyridine-3-carboxylic acid (0.5 g, 1.6 mmol) in dry dichloromethane (50 mL) was added Bis(dimethylamino)methylene]-1H-1,2,3-triazolo[4,5-b]pyridinium 3-oxide hexafluorophosphate (HATU) (0.93 g, 2.4 mmol) and N,N-dimethylpyridin-4-amine (0.1 g, 0.8 mmol) was added at 0° C. The reaction mixture was stirred at room temperature for 16 hours. The progress of reaction mixture was monitored by TLC and LCMS. After completion of reaction, solvent was removed in vacuo and diluted with acetonitrile (25 mL) and 3-methanesulfinylaniline (0.3 g, 2 mmol) was added to the above solution at room temperature. The reaction mixture was stirred at 70° C. for 16 hours. The progress of the reaction was monitored by TLC. After completion of the reaction, the mixture was diluted with water and extracted with ethyl acetate. The organic layer was dried over anhydrous sodium sulfate and filtered. The filtrate was concentrated under reduced pressure. The crude residue was purified by flash column chromatography with a gradient of 50-70% ethyl acetate in hexanes to afford 2-(4,4-difluoroazepan-1-yl)-N-(3-(methylsulfinyl)phenyl)-1,7-naphthyridine-3-carboxamide as a white solid.

### Synthesis 2-(4,4-difluoroazepan-1-yl)-N-(3-(S-methylsulfonimidoyl)phenyl)-1,7-naphthyridine-3-carboxamide

To a stirred solution of 2-(4,4-difluoroazepan-1-yl)-N-(3-(methylsulfinyl)phenyl)-1,7-naphthyridine-3-carboxamide (0.30 g, 0.675 mmol) in Eaton's Reagent (5 mL) was added sodium azide (0.088 g, 1.35 mmol) at room temperature. The reaction mixture was stirred at 50° C. for 2 hours. The progress of reaction was monitored by TLC and LCMS. After completion of the reaction, the mixture was diluted with water and basified by using sodium carbonate and extracted with ethyl acetate. The organic layer was dried over anhydrous sodium sulfate and filtered. The filtrate was concentrated under reduced pressure and the resulting crude compound was purified by flash column chromatography with a gradient of 0-5% methanol in dichloromethane to afford 2-(4,4-difluoroazepan-1-yl)-N-(3-(S-methylsulfonimidoyl)phenyl)-1,7-naphthyridine-3-carboxamide as a white solid. MS (ESI): Calcd [M+H]⁺: 460.16, found: 460.30.

¹H NMR (400 MHz, DMSO-d₆) δ 11.08 (s, 1H), 9.00 (s, 1H), 8.40 (s, 1H), 8.36 (s, 1H), 8.34 (d, J=4 Hz, 1H), 7.98 (d, J=8 Hz, 1H), 7.75 (d, J=4 Hz, 1H), 7.70 (d, J=8 Hz, 1H), 7.63 (t, J=8 Hz, 1H), 4.23 (s, 1H), 3.80-3.77 (m, 2H), 3.65 (t, J=8 Hz, 2H), 3.07 (s, 3H), 2.43-2.37 (m, 2H), 2.01-2.00 (m, 2H), 1.96-1.90 (m, 2H).

### Compound 49

### Synthesis of 2-(4,4-difluoroazepan-1-yl)-N-(3-sulfamoylphenyl)-6,7,8,9-tetrahydro-5H-cyclohepta[b]pyridine-3-carboxamide

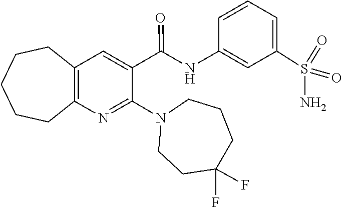 [SMILES: NS(=O)(=O)c1cccc(NC(=O)c2cc3c(nc2N2CCCC(F)(F)CC2)CCCCC3)c1]

### Synthesis of (Z)-2-((dimethylamino)methylene)cycloheptan-1-one

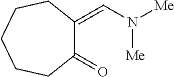 [SMILES: CN(C)/C=C1/CCCCCC1=O]

A solution of cycloheptanone (8.6 g, 77 mmol) in 1-tert-butoxy-N,N,N,N′-tetramethylmethanediamine (16 g, 77 mmol) was heated at 110° C. for 12 hours. After completion of reaction, the solvent was removed in vacuo to obtain (Z)-2-((dimethylamino)methylene)cycloheptan-1-one as a brown liquid which was used as such in the next step without purification.

### Synthesis of methyl 2-hydroxy-6,7,8,9-tetrahydro-5H-cyclohepta[b]pyridine-3-carboxylate

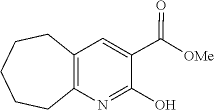 [SMILES: COC(=O)c1cc2c(nc1O)CCCCC2]

To a solution of (Z)-2-((dimethylamino)methylene)cycloheptan-1-one (12 g, 72 mmol) in methanol (60 mL) was added methyl 2-cyanoacetate (12.7 mL, 143.5 mmol) and slowly heated to 80° C. for 12 hours. After completion of reaction, the solvent was removed in vacuo to obtain the crude solid mass which was purified through trituration with acetonitrile and n-pentane to afford methyl 2-hydroxy-6,7,8,9-tetrahydro-5H-cyclohepta[b]pyridine-3-carboxylate as white solid.

### Synthesis of methyl 2-chloro-6,7,8,9-tetrahydro-5H-cyclohepta[b]pyridine-3-carboxylate

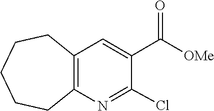 [SMILES: COC(=O)c1cc2c(nc1Cl)CCCCC2]

A solution of methyl 2-hydroxy-6,7,8,9-tetrahydro-5H-cyclohepta[b]pyridine-3-carboxylate (1 g, 4.5 mmol) in phosphorus oxychloride (10 mL) was heated at 130° C. for 12 hours. After completion of reaction, excess of phosphorus oxychloride was distilled out under rotatory. The crude residue was dissolved into ethyl acetate and washed with saturated sodium bicarbonate solution. The organic layer was separated, washed with brine, dried over anhydrous sodium sulfate, filtered and concentrated under reduced pressure. The crude material was purified by trituration with diethyl ether and n-pentane to afford methyl 2-chloro-6,7,8,9-tetrahydro-5H-cyclohepta[b]pyridine-3-carboxylate as a white solid.

### Synthesis of methyl 2-(4,4-difluoroazepan-1-yl)-6,7,8,9-tetrahydro-5H-cyclohepta[b]pyridine-3-carboxylate

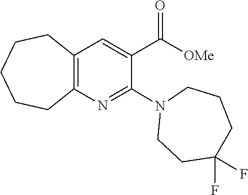 [SMILES: COC(=O)c1cc2c(nc1N1CCCC(F)(F)CC1)CCCCC2]

To a solution of methyl 2-chloro-6,7,8,9-tetrahydro-5H-cyclohepta[b]pyridine-3-carboxylate (1 g, 4.2 mmol) in N,N-dimethylformamide (15 mL) was added 4,4-difluoroazepane hydrogen chloride (0.86 g, 5 mmol) and potassium carbonate (2.3 g, 16.7 mmol). The mixture was heated at 100° C. for 48 hours under nitrogen atmosphere. The progress of reaction was monitored by TLC. After completion of reaction, the crude material was diluted with ice-cold water and extracted with ethyl acetate. The organic layer was washed with brine solution and dried over anhydrous sodium sulfate. The crude material was purified by flash column chromatography with a gradient of 20-30% ethyl acetate in hexanes to afford methyl 2-(4,4-difluoroazepan-1-yl)-6,7,8,9-tetrahydro-5H-cyclohepta[b]pyridine-3-carboxylate as a yellow liquid.

### Synthesis of 2-(4,4-difluoroazepan-1-yl)-6,7,8,9-tetrahydro-5H-cyclohepta[b]pyridine-3-carboxylic acid

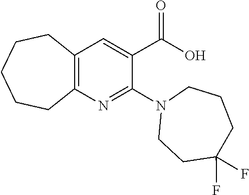 [SMILES: O=C(O)c1cc2c(nc1N1CCCC(F)(F)CC1)CCCCC2]

To a solution of methyl 2-(4,4-difluoroazepan-1-yl)-6,7,8,9-tetrahydro-5H-cyclohepta[b]pyridine-3-carboxylate (1 g, 3 mmol) in tetrahydrofuran (10 mL) and methanol (10 mL) was added lithium hydroxide (0.25 g, 6 mmol) and water (10 mL). The reaction mixture was stirred at room temperature for 12 hours. After completion of reaction, the solvent was evaporated under vacuum. The crude mass was diluted with water, acidified with 1N hydrochloric acid and extracted with ethyl acetate. The organic layer was washed with brine solution, dried over anhydrous sodium sulfate and filtered. The filtrate was concentrated under reduced pressure to obtain the crude residue which was purified by trituration with diethyl ether and n-pentane to afford 2-(4,4-difluoroazepan-1-yl)-6,7,8,9-tetrahydro-5H-cyclohepta[b]pyridine-3-carboxylic acid as a white solid.

### Synthesis of 2-(4,4-difluoroazepan-1-yl)-N-(3-sulfamoylphenyl)-6,7,8,9-tetrahydro-5H-cyclohepta[b]pyridine-3-carboxamide

To a solution of 2-(4,4-difluoroazepan-1-yl)-6,7,8,9-tetrahydro-5H-cyclohepta[b]pyridine-3-carboxylic acid (0.3 g, 0.93 mmol) in dichloromethane (10 mL) was added 1-[Bis(dimethylamino)methylene]-1H-1,2,3-triazolo[4,5-b]pyridinium 3-oxide hexafluorophosphate (HATU) (0.53 g, 1.4 mmol) and 4-Dimethylaminopyridine (0.006 g, 0.05 mmol) at 0° C. and stirred the reaction mixture at room temperature for 12 hours. The progress of the reaction was monitor by TLC. After completion of reaction, solvent was evaporated under reduced pressure. The resultant crude mass was dissolved into acetonitrile (10 mL) and to this solution, 3-aminobenzenesulfonamide (0.19 g, 1.1 mmol) was added and heated at 80° C. for 12 hours. After completion of reaction, acetonitrile was removed in vacuo and the crude mass was diluted with water and extracted with ethyl acetate. The organic layer was washed with brine solution and dried over anhydrous sodium sulfate, filtered and filtrate was concentrated. The crude material was purified by flash column chromatography with a gradient of 40-50% ethyl acetate in hexanes to afford 2-(4,4-difluoroazepan-1-yl)-N-(3-sulfamoylphenyl)-6,7,8,9-tetrahydro-5H-cyclohepta[b]pyridine-3-carboxamide as a white solid. MS (ESI): Calcd [M+H]⁺: 479.19, found: 479.25.

¹H NMR (400 MHz, DMSO-d₆): δ 10.58 (s, 1H), 8.30 (s, 1H), 7.80-7.78 (m, 1H), 7.52-7.46 (m, 2H), 7.46 (s, 1H), 7.37 (s, 2H), 3.59-3.58 (m, 2H), 3.39 (t, J=6.0 Hz, 2H), 2.87-2.84 (m, 2H), 2.70-2.69 (m, 2H), 2.30-2.26 (m, 2H), 1.97-1.94 (m, 2H), 1.80-1.79 (m, 4H), 1.59-1.57 (m, 4H).

### Compound 50

### Synthesis of 3-(4,4-difluoro-3-methylpiperidin-1-yl)-N-(2-sulfamoylpyridin-4-yl)-5,6,7,8-tetrahydroquinoxaline-2-carboxamide

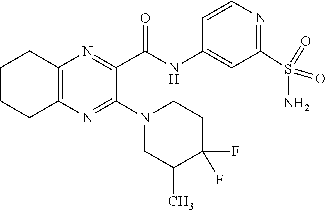 [SMILES: CC1CN(c2nc3c(nc2C(=O)Nc2ccnc(S(N)(=O)=O)c2)CCCC3)CCC1(F)F]

### Synthesis of ethyl 3-hydroxy-4a,5,6,7,8,8a-hexahydroquinoxaline-2-carboxylate

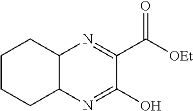 [SMILES: CCOC(=O)C1=NC2CCCCC2N=C1O]

A solution of cyclohexane-1,2-diamine (230 mg, 2 mmol) and diethyl 2-oxomalonate (305 μL, 2 mmol) in ethanol (10 mL) was stirred at 70° C. for 18 hours. The solvent was removed in vacuo and the product was used directly in the next step.

### Synthesis of ethyl 3-hydroxy-5,6,7,8-tetrahydroquinoxaline-2-carboxylate

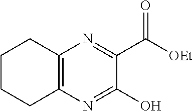 [SMILES: CCOC(=O)c1nc2c(nc1O)CCCC2]

Ethyl 3-hydroxy-4a,5,6,7,8,8a-hexahydroquinoxaline-2-carboxylate neat was heated to 110° C. for 72 hours under air then purified by flash column chromatography using a Combiflash with a gradient of 0-50% ethyl acetate in hexanes to afford ethyl 3-hydroxy-5,6,7,8-tetrahydroquinoxaline-2-carboxylate as a yellow solid.

### Synthesis of ethyl 3-chloro-5,6,7,8-tetrahydroquinoxaline-2-carboxylate

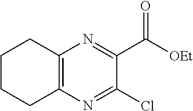 [SMILES: CCOC(=O)c1nc2c(nc1Cl)CCCC2]

A mixture of ethyl 3-hydroxy-5,6,7,8-tetrahydroquinoxaline-2-carboxylate (400 mg, 1.8 mmol) and phosphorous oxychloride (7 mL) was slowly heated to 110° C. for 72 hours. After completion of the reaction, the reaction mixture was quenched with ice-cold water and saturated sodium bicarbonate solution, then extracted with dichloromethane. The organic layers were combined then dried over anhydrous sodium sulphate and filtered. The filtrate was concentrated under reduced pressure and the crude residue was purified by flash column chromatography using a Combiflash with a gradient of 0-50% ethyl acetate in hexanes to afford ethyl 3-chloro-5,6,7,8-tetrahydroquinoxaline-2-carboxylate as a yellow oil.

### Synthesis of ethyl 3-(4,4-difluoro-3-methylpiperidin-1-yl)-5,6,7,8-tetrahydroquinoxaline-2-carboxylate

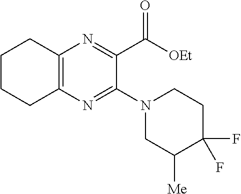 [SMILES: CCOC(=O)c1nc2c(nc1N1CCC(F)(F)C(C)C1)CCCC2]

To a solution of ethyl 3-chloro-5,6,7,8-tetrahydroquinoxaline-2-carboxylate (135 mg, 0.56 mmol) and 4,4-difluoro-3-methylpiperidine hydrogen chloride (97 mg, 0.56 mmol) in N,N-dimethylformamide (4 mL), potassium carbonate (390 mg, 2.8 mmol) was added at room temperature. The reaction mixture was stirred at 70° C. for 72 hours. The progress of the reaction was monitored by TLC and LCMS. After completion, the reaction mixture was diluted with water and hydrochloric acid 1 N then extracted with dichloromethane. The organic layers were dried over anhydrous sodium sulphate and filtered. The filtrate was concentrated under reduced pressure and the crude residue was purified by flash column chromatography with a gradient of ethyl acetate in hexanes to afford 3-chloro-5,6,7,8-tetrahydroquinoxaline-2-carboxylic acid as a yellow oil.

### Synthesis of 3-(4,4-difluoro-3-methylpiperidin-1-yl)-5,6,7,8-tetrahydroquinoxaline-2-carboxylic acid

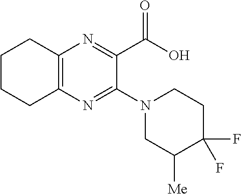 [SMILES: CC1CN(c2nc3c(nc2C(=O)O)CCCC3)CCC1(F)F]

To a stirred solution of ethyl 3-(4,4-difluoro-3-methylpiperidin-1-yl)-5,6,7,8-tetrahydroquinoxaline-2-carboxylate (0.94 g, 0.28 mmol) in methanol (2.8 mL) and tetrahydrofuran (12 mL) was added 1 N sodium hydroxide (2.8 mL). The reaction mixture was stirred at room temperature for 18 hours. The progress of reaction was monitored by TLC and LCMS. After completion of reaction, solvent was removed under rotatory and diluted with water, then acidified with 1 N hydrochloric acid solution and extracted with dichloromethane. The organic layers were combined, dried with sodium sulphate, filtered and concentrated to afford 3-(4,4-difluoro-3-methylpiperidin-1-yl)-5,6,7,8-tetrahydroquinoxaline-2-carboxylic acid as a light brown powder.

### Synthesis of 3-(4,4-difluoro-3-methylpiperidin-1-yl)-5,6,7,8-tetrahydroquinoxaline-2-carboxamide

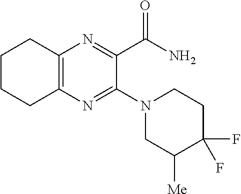 [SMILES: CC1CN(c2nc3c(nc2C(N)=O)CCCC3)CCC1(F)F]

To a stirred solution of 3-(4,4-Difluoro-3-methylpiperidin-1-yl)-5,6,7,8-tetrahydroquinoxaline-2-carboxylic acid (86 mg, 0.28. mmol), in dichloromethane (5 mL) was added (1-[Bis(dimethylamino)methylene]-1H-1,2,3-triazolo[4,5-b]pyridinium 3-oxide hexafluorophosphate (HATU) (0.16 g, 0.41 mmol) and ammonium carbonate (0.13 g, 1.4 mmol) at room temperature. The reaction mixture was stirred at room temperature for 18 hours. The progress of the reaction was monitored by TLC and LCMS. After completion of reaction, the reaction mixture was diluted with water and extracted dichloromethane. The organic layers were combined, dried over anhydrous sodium sulphate, filtered and concentrated to afford crude material. The crude residue was purified by flash column chromatography using a Combiflash with a gradient of 0-50% ethyl acetate in hexanes to afford 3-(4,4-difluoro-3-methylpiperidin-1-yl)-5,6,7,8-tetrahydroquinoxaline-2-carboxamide as a light yellow powder.

### Synthesis of N-(2-(N,N-bis(2,4-dimethoxybenzyl)sulfamoyl)pyridin-4-yl)-3-(4,4-difluoro-3-methylpiperidin-1-yl)-5,6,7,8-tetrahydroquinoxaline-2-carboxamide

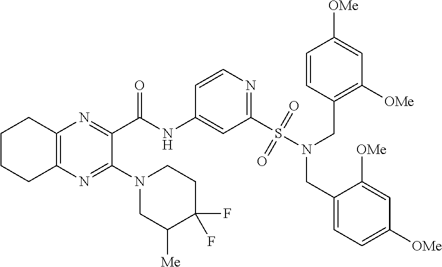 [SMILES: COc1ccc(CN(Cc2ccc(OC)cc2OC)S(=O)(=O)c2cc(NC(=O)c3nc4c(nc3N3CCC(F)(F)C(C)C3)CCCC4)ccn2)c(OC)c1]

3-(4,4-difluoro-3-methylpiperidin-1-yl)-5,6,7,8-tetrahydroquinoxaline-2-carboxamide (36 mg, 0.116 mmol), 4-bromo-N,N-bis(2,4-dimethoxybenzyl)pyridine-2-sulfonamide (62 mg, 0.116 mmol), cesium carbonate (76 mg, 0.23 mmol), and BrettPhos Pd G3 (21 mg, 0.023 mmol) were added to a flame dried round bottom flask and capped. The reaction vessel was evacuated and the atmosphere was replace with nitrogen. Then, degassed anhydrous dioxane (3 mL) was added to the reaction vessel and the mixture was stirred at 100° C. for 18 hours. Dioxane was removed in vacuo and the crude mixture was purified by flash column chromatography with a gradient of ethyl acetate in hexanes to afford N-(2-(N,N-bis(2,4-dimethoxybenzyl)sulfamoyl)pyridin-4-yl)-3-(4,4-difluoro-3-methylpiperidin-1-yl)-5,6,7,8-tetrahydroquinoxaline-2-carboxamide as a yellow oil.

### Synthesis of 3-(4,4-difluoro-3-methylpiperidin-1-yl)-N-(2-sulfamoylpyridin-4-yl)-5,6,7,8-tetrahydroquinoxaline-2-carboxamide

To a solution of N-(2-(N,N-bis(2,4-dimethoxybenzyl)sulfamoyl)pyridin-4-yl)-3-(4,4-difluoro-3-methylpiperidin-1-yl)-5,6,7,8-tetrahydroquinoxaline-2-carboxamide (27 mg, 0.1 mmol) in dichloromethane (2 mL) was added trifluoroacetic acid (155 μL). The reaction mixture was stirred for 18 hours then the volatiles were removed in vacuo. Volatiles were removed in vacuo and the resulting crude mixture was diluted with water and acetonitrile then purified using reversed-phase HPLC with a gradient of 5-95% acetonitrile in water to afford 3-(4,4-difluoro-3-methylpiperidin-1-yl)-N-(2-sulfamoylpyridin-4-yl)-5,6,7,8-tetrahydroquinoxaline-2-carboxamide as a white powder. LRMS (ESI): Calcd [M+H]⁺: 467.17, found: 466.9.

¹H NMR (400 MHz, CDCl₃) δ 8.30 (d, J=5.6 Hz, 1H), 8.03 (s, 1H), 7.82 (d, J=5.6 Hz, 1H), 3.69-3.54 (m, 2H), 3.04-2.95 (m, 1H), 2.79-2.70 (m, 1H), 2.69-2.57 (m, 4H), 2.08-1.82 (m, 3H), 1.76-1.61 (m, 4H), 0.79 (d, J=6.8 Hz, 3H).

### Compound 98

### Synthesis of 2-(4,4-difluoro-azepan-1-yl)-7-fluoro-N-(2-(S-methylsulfonimidoyl)pyridin-4-yl)quinoline-3-carboxamide

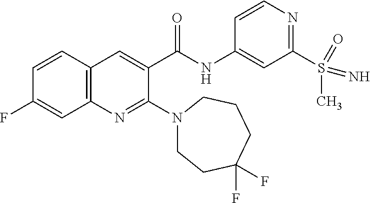 [SMILES: CS(=N)(=O)c1cc(NC(=O)c2cc3ccc(F)cc3nc2N2CCCC(F)(F)CC2)ccn1]

### Synthesis of 2-(4,4-difluoroazepan-1-yl)-7-fluoro-N-(2-(methylthio)pyridin-4-yl)quinoline-3-carboxamide

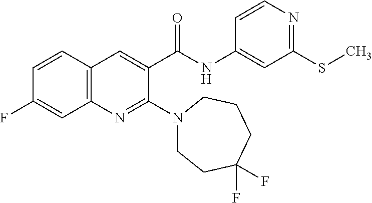 [SMILES: CSc1cc(NC(=O)c2cc3ccc(F)cc3nc2N2CCCC(F)(F)CC2)ccn1]

To a solution of 2-(4,4-difluoroazepan-1-yl)-7-fluoroquinoline-3-carboxamide (41 mg, 0.13 mmol), 4-chloro-2-(methylthio)pyridine (20.5 mg, 0.13 mmol), cesium carbonate (83.6 mg, 0.26 mmol), and BrettPhos Pd G3 (23 mg, 0.026 mmol) were added to a flame dried round bottom flask and capped. The reaction vessel was evacuated and the atmosphere was replace with nitrogen. Then, degassed anhydrous dioxane (2 mL) was added to the reaction vessel and the mixture was stirred at 100° C. for 18 hours. Dioxane was removed in vacuo and the crude mixture was purified by flash column chromatography with a gradient of ethyl acetate in hexanes to afford 2-(4,4-difluoroazepan-1-yl)-7-fluoro-N-(2-(methylthio)pyridin-4-yl)quinoline-3-carboxamide as a yellow oil.

### Synthesis of 2-(4,4-difluoroazepan-1-yl)-7-fluoro-N-(2-(5S-methylsulfonimidoyl)pyridin-4-yl)quinoline-3-carboxamide

To a stirred solution of 2-(4,4-difluoroazepan-1-yl)-7-fluoro-N-(2-(methylthio)pyridin-4-yl)quinoline-3-carboxamide (57 mg, 0.13 mmol) in MeOH (10 mL) was added ammonium carbonate (43 mg, 0.45 mmol) then (diacetoxyiodo)benzene (120 mg, 0.38 mmol). The reaction mixture was stirred at 50° C. for 24 hours. The progress of the reaction was monitored by LCMS. Methanol was removed in vacuo and the resulting crude mass was diluted with water and acetonitrile and purified using reversed-phase HPLC with a gradient of 5-95% acetonitrile in water to afford 2-(4,4-difluoroazepan-1-yl)-7-fluoro-N-(2-(S-methylsulfonimidoyl)pyridin-4-yl)quinoline-3-carboxamide as a white powder. LRMS (ESI): Calcd [M+H]⁺: 478.15, found 477.9.

### Example 2

### Na_{V} Inhibition Assay

Electrophysiology experiments were performed on Human Embryonic Kidney 293 cells (HEK) or Chinese hamster ovary cells (CHO) transfected with the full-length cDNA coding for the appropriate human Na_{V} sodium channel α-subunit Na_{V} 1.8. Similarly, experiments are performed on Human Embryonic Kidney 293 cells (HEK) or Chinese hamster ovary cells (CHO) transfected with the full-length cDNA coding for the appropriate human Na_{V} sodium channel α-subunit, including Na_{V} 1.7, Na_{V} 1.6, Na_{V} 1.5, and Na_{V} 1.4.

Sodium currents were measured using the patch-clamp technique in the whole-cell configuration with a HEKA EPC 9 amplifier (HEKA Elektronik Dr. Schulze GmbH, Germany) or can be measured using an IonFlux 16 automated patch clamp system (Fluxion Biosciences, South San Francisco, USA) as previously described by Moran. See, Moran O, Picollo A, Conti F (2003) Tonic and phasic guanidinium toxin-block of skeletal muscle Na channels expressed in Mammalian cells, Biophys J 84(5):2999-3006. For manual patch-clamp experiments, borosilicate glass micropipettes (Sutter Instruments, Novato, CA) were pulled to a tip diameter yielding a resistance of 1.0-2.0 MΩ in the working solutions. The composition of intracellular solution was (in mM): CsF 125, EGTA 10, HEPES 10, NaCl 10, and the pH was adjusted to 7.2 with CsOH. The composition of extracellular solution was (in mM): NaCl 135, KCl 4.5, CaCl₂ 2, MgCl₂ 1, HEPES 10, and the pH was adjusted to 7.4 with NaOH. For human Na_{V} 1.8, 0.3-1 μM tetrodotoxin (TTX) was added to the extracellular solution to inhibit native TTX-sensitive current. Peak currents were generally between 0.5-20 nA.

Lyophilized stock of each test article was stored at −20° C., solubilized in DMSO (maximum final concentration 1%) and diluted to the desired concentration with with the external solution prior to recording. Current measurements were recorded under continuous perfusion, controlled by syringe pump addition.

The output of the EPC 9 patch-clamp amplifier was filtered with a built-in low-pass, four-pole Bessel filter having a cutoff frequency of 10 kHz and sampled at 20-50 kHz. For both manual and automated recordings, the membrane was kept at a holding potential of between −120 and −80 mV. Pulse stimulation and data acquisition were controlled with the Pulse software (HEKA Elektronik Dr. Schulze GmbH, Germany) or the IonFlux software (Fluxion Biosciences, South San Francisco, USA). All measurements were performed at room temperature (about 20-22° C.). Recordings were made at least 5 min after establishing the whole-cell and voltage-clamp configuration to allow for stabilization of the voltage-dependent properties of the channels. Currents were elicited by 10-50 ms step depolarizations from a holding potential to a value between −40 and +10 mV. Peak currents after channel activation were recorded. These data were baseline-normalized, plotted against toxin concentration and analyzed in Microsoft Excel software. Data were fit to a four-parameter logistic equation with the Hill slope set to 1 to determine IC₅₀ values and expressed as mean.

### Table 1—Na_{V} Isoform Potency and Selectivity

For Table 1 and Table 2, all data were measured in HEK cells. Column 1 provides IC₅₀ data for Na_{V} 1.8 as measured using using the patch-clamp technique in the whole-cell configuration with a HEKA EPC 9 amplifier, where +++ indicates an IC₅₀ of less than or equal to 20 nM, ++ indicates an IC₅₀ of greater than 20 nM but less that or equal to 100 nM, and + indicates an IC₅₀ of greater than 100 nM but less that or equal to 1.5 M. ND means not detectable. NT means not tested.

TABLE 1
Compounds

| Compound No | Structure | NaV1.8 IC₅₀ (nM) |
|---|---|---|
| 1 | 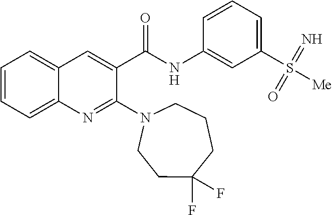 [SMILES: CS(=N)(=O)c1cccc(NC(=O)c2cc3ccccc3nc2N2CCCC(F)(F)CC2)c1] | +++ |
| 2 | 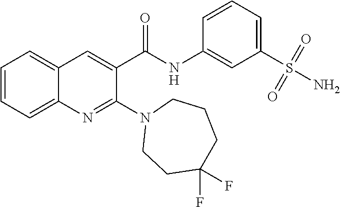 [SMILES: NS(=O)(=O)c1cccc(NC(=O)c2cc3ccccc3nc2N2CCCC(F)(F)CC2)c1] | +++ |
| 3 | 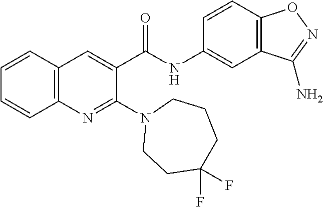 [SMILES: Nc1noc2ccc(NC(=O)c3cc4ccccc4nc3N3CCCC(F)(F)CC3)cc12] | +++ |
| 4 | 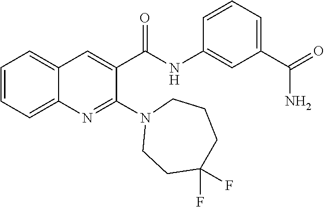 [SMILES: NC(=O)c1cccc(NC(=O)c2cc3ccccc3nc2N2CCCC(F)(F)CC2)c1] | +++ |
| 5 | 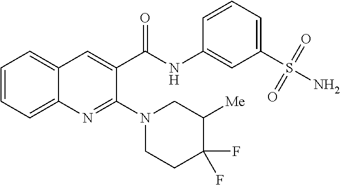 [SMILES: CC1CN(c2nc3ccccc3cc2C(=O)Nc2cccc(S(N)(=O)=O)c2)CCC1(F)F] | +++ |
| 6 | 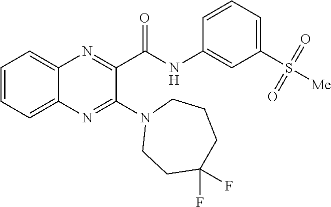 [SMILES: CS(=O)(=O)c1cccc(NC(=O)c2nc3ccccc3nc2N2CCCC(F)(F)CC2)c1] | +++ |
| 7 | 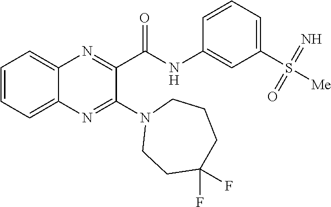 [SMILES: CS(=N)(=O)c1cccc(NC(=O)c2nc3ccccc3nc2N2CCCC(F)(F)CC2)c1] | ++ |
| 8 | 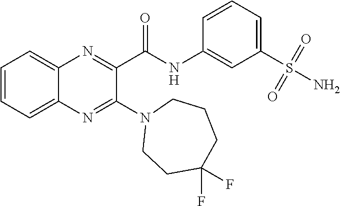 [SMILES: NS(=O)(=O)c1cccc(NC(=O)c2nc3ccccc3nc2N2CCCC(F)(F)CC2)c1] | ++ |
| 9 | 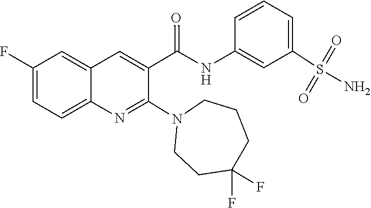 [SMILES: NS(=O)(=O)c1cccc(NC(=O)c2cc3cc(F)ccc3nc2N2CCCC(F)(F)CC2)c1] | ++ |
| 10 | 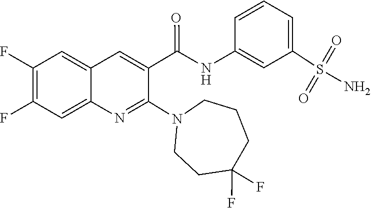 [SMILES: NS(=O)(=O)c1cccc(NC(=O)c2cc3cc(F)c(F)cc3nc2N2CCCC(F)(F)CC2)c1] | ++ |
| 11 | 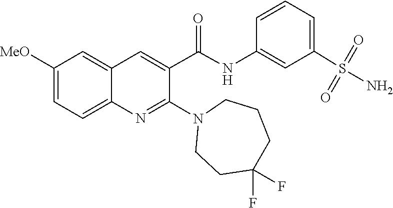 [SMILES: COc1ccc2nc(N3CCCC(F)(F)CC3)c(C(=O)Nc3cccc(S(N)(=O)=O)c3)cc2c1] | +++ |
| 12 | 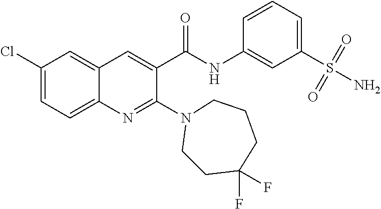 [SMILES: NS(=O)(=O)c1cccc(NC(=O)c2cc3cc(Cl)ccc3nc2N2CCCC(F)(F)CC2)c1] | +++ |
| 13 | 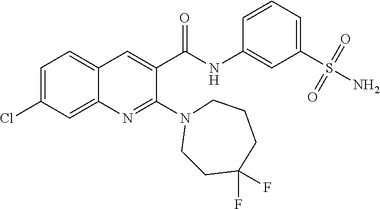 [SMILES: NS(=O)(=O)c1cccc(NC(=O)c2cc3ccc(Cl)cc3nc2N2CCCC(F)(F)CC2)c1] | ++ |
| 14 | 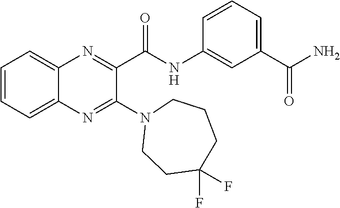 [SMILES: NC(=O)c1cccc(NC(=O)c2nc3ccccc3nc2N2CCCC(F)(F)CC2)c1] | ++ |
| 15 | 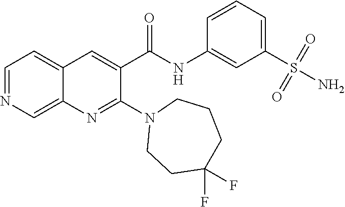 [SMILES: NS(=O)(=O)c1cccc(NC(=O)c2cc3ccncc3nc2N2CCCC(F)(F)CC2)c1] | +++ |
| 16 | 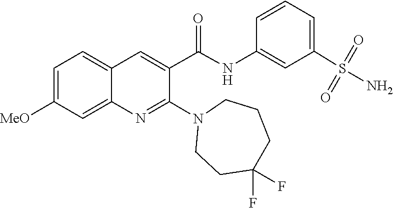 [SMILES: COc1ccc2cc(C(=O)Nc3cccc(S(N)(=O)=O)c3)c(N3CCCC(F)(F)CC3)nc2c1] | ++ |
| 17 | 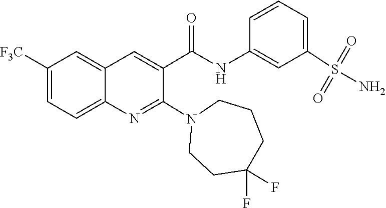 [SMILES: NS(=O)(=O)c1cccc(NC(=O)c2cc3cc(C(F)(F)F)ccc3nc2N2CCCC(F)(F)CC2)c1] | +++ |
| 18 | 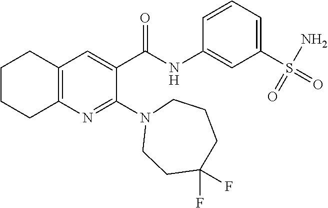 [SMILES: NS(=O)(=O)c1cccc(NC(=O)c2cc3c(nc2N2CCCC(F)(F)CC2)CCCC3)c1] | ++ |
| 19 | 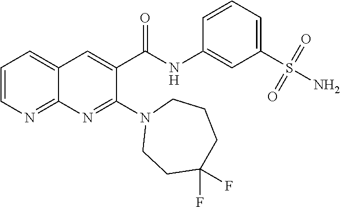 [SMILES: NS(=O)(=O)c1cccc(NC(=O)c2cc3cccnc3nc2N2CCCC(F)(F)CC2)c1] | + |
| 20 | 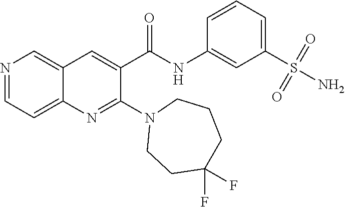 [SMILES: NS(=O)(=O)c1cccc(NC(=O)c2cc3cnccc3nc2N2CCCC(F)(F)CC2)c1] | ++ |
| 28 | 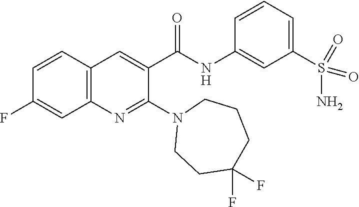 [SMILES: NS(=O)(=O)c1cccc(NC(=O)c2cc3ccc(F)cc3nc2N2CCCC(F)(F)CC2)c1] | +++ |

All publications, patents, and patent applications cited in this specification are herein incorporated by reference as if each individual publication, patent, or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the claimed subject matter is limited solely by the scope of the following claims, including equivalents thereof.

What is claimed is:

1. A compound of Formula (I): 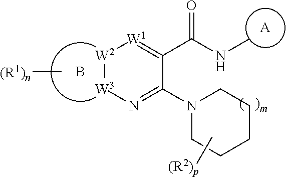
wherein:
R¹, independently in each instance, is selected from hydrogen, C₁-C₆ alkyl, C₁-C₆ alkoxy, halogen, C₁-C₆ haloalkyl, halo-C₁-C₆ alkoxy, C₃-C₆-cycloalkyl, and C₃-C₆-cycloalkylC₁-C₃-alkyl; or wherein two R¹ are attached on adjacent ring carbons in 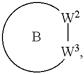
and together with the adjacent carbons to which they are attached form 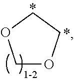
where * indicate the shared carbons in 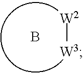
A is C₆-C₁₀ aryl substituted with R³ and optionally substituted with (R^{3a})_{q}; 5- to 10-membered heteroaryl substituted with R³ and optionally substituted with (R^{3a})_{q}; or 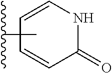
optionally substituted with (R^{3a})_{q1};
W¹ is —N═, —C(H)═, —C(halogen)=, —C(C₁-C₆ alkyl)=, or —C(cyclopropyl)=;
W² and W³ are each —C—, the dashed bond between W² and W³ is a double bond, and 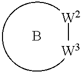
is a partially unsaturated 5 to 7-membered carbocyclic ring, a benzo ring, a partially unsaturated 5 to 7-membered heterocyclic ring, or a 5 or 6-membered heteroaromatic ring; or
one of W² and W³ is —C— and the other is —N—, the dashed bond between W² and W³ is a single bond, and 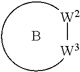
is a 5 or 6-membered heterocyclic ring optionally comprising an additional N and where the remaining ring atoms are C;
R², independently in each instance, is hydrogen, halogen, or C₁-C₆ alkyl; or
two R² are attached on adjacent carbons and together with the carbons to which they are attached form a 4- to 7-membered carbocyclic ring and the remaining R², independently in each instance, is hydrogen, halogen, or C₁-C₆ alkyl; or
two R² are attached on the same carbon and together with the carbon to which they are attached form a 4- to 7-membered carbocyclic ring and the remaining R², independently in each instance, is hydrogen, halogen, or C₁-C₆alkyl; R³ is hydrogen, —C(O)NH₂, —NH₂, —S(O)₂NHR, —S(O)₂C₁-C₆-alkyl, or 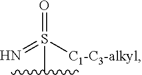
wherein R is hydrogen, C₁-C₃ alkyl or C₃-C₅ cycloalkyl;
R^{3a}, independently in each instance, is hydrogen, halogen, C₁-C₃ alkyl, or C₃-C₆ cycloalkyl;
m is 0, 1, 2, or 3;
n is 0, 1, 2, 3, or 4;
p is 0, 1, 2, 3, 4, 5, or 6;
q1 is 0, 1, or 2; and
q is 0, 1, 2, or 3;
or a pharmaceutically acceptable salt thereof and/or an isomer thereof.

2. The Compound of claim 1, or a pharmaceutically acceptable salt thereof and/or an isomer thereof, selected from the group consisting of the compounds provided in Table A.

3. A pharmaceutical composition comprising the compound of claim 1, or a pharmaceutically acceptable salt thereof and/or an isomer thereof, and a pharmaceutically acceptable carrier.

4. A method for the treatment of a condition associated with voltage-gated sodium channel function, including Naᵥ1.8, in a subject, comprising the administration of a therapeutically or prophylactically effective amount of the compound of claim 1 or the pharmaceutical composition of claim 3.

5. A method of inhibiting NAᵥ1.8 comprising contacting NAᵥ1.8 with a compound of claim 1.

6. A compound, or a salt thereof, according to any one of the following formulas: 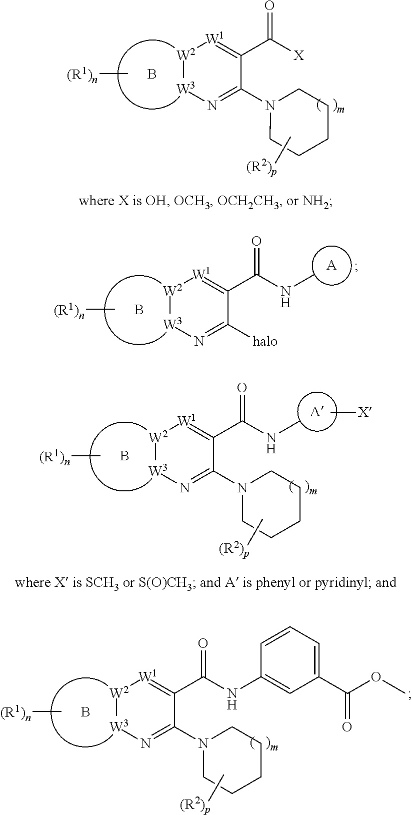
wherein
R¹, independently in each instance, is selected from hydrogen, C₁-C₆ alkyl, C₁-C₆ alkoxy, halogen, C₁-C₆ haloalkyl, halo-C₁-C₆ alkoxy, C₃-C₆-cycloalkyl, and C₃-C₆-cycloalkylC₁-C₃-alkyl; or
wherein two R¹ are attached on adjacent ring carbons in 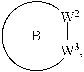
and together with the adjacent carbons to which they are attached form 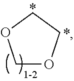
where * indicate the shared carbons in 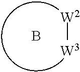
and any remaining R¹ are independently selected from hydrogen, C₁-C₆ alkyl, C₁-C₆ alkoxy, halogen, C₁-C₆ haloalkyl, halo-C₁-C₆ alkoxy, C₃-C₆-cycloalkyl, or C₃-C₆-cycloalkylC₁-C₃-alkyl;
A is C₆-C₁₀ aryl substituted with R³ and optionally substituted with (R^{3a})_{q}; 5- to 10-membered heteroaryl substituted with R³ and optionally substituted with (R^{3a})_{q}; or 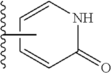
optionally substituted with (R^{3a})_{q1};
W¹ is —N═, —C(H)═, —C(halogen)=, —C(C₁-C₆ alkyl)=, or —C(cyclopropyl)=;
W² and W³ are each —C—, the dashed bond between W² and W³ is a double bond, and 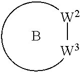
is a partially unsaturated 5 to 7-membered carbocyclic ring, a benzo ring, a partially unsaturated 5 to 7-membered heterocyclic ring, or a 5 or 6-membered heteroaromatic ring; or
one of W² and W³ is —C— and the other is —N—, the dashed bond between W² and W³ is a single bond, and 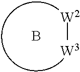
is a 5 or 6-membered heterocyclic ring optionally comprising an additional N and where the remaining ring atoms are C;
R², independently in each instance, is hydrogen, halogen, or C₁-C₆ alkyl; or
two R² are attached on adjacent carbons and together with the carbons to which they are attached form a 4- to 7-membered carbocyclic ring and the remaining R², independently in each instance, is hydrogen, halogen, or C₁-C₆alkyl; or
two R² are attached on the same carbon and together with the carbon to which they are attached form a 4- to 7-membered carbocyclic ring and the remaining R², independently in each instance, is hydrogen, halogen, or C₁-C₆alkyl;
m is 0, 1, 2, or 3;
n is 0, 1, 2, 3, or 4; and
p is 0, 1, 2, 3, 4, 5, or 6.

7. A method of preparing a Compound of Formula (I) of claim 1, according to any of the following schemes: 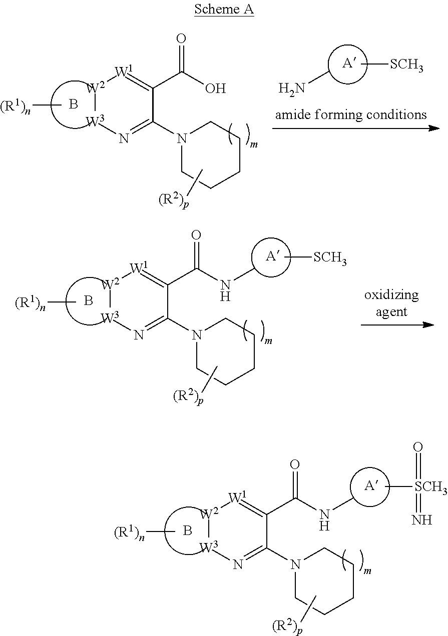 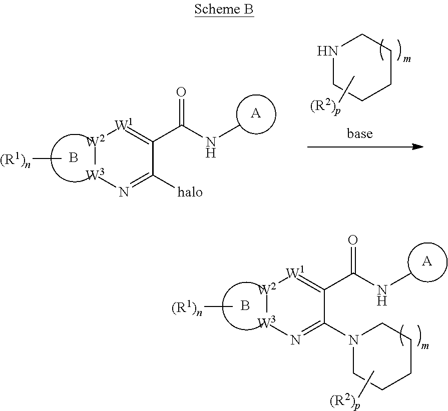 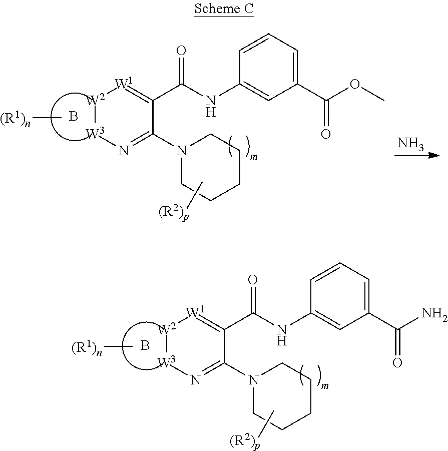 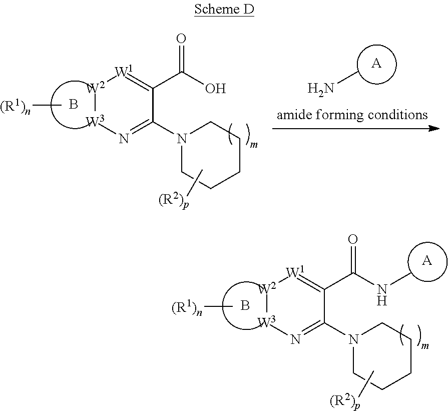 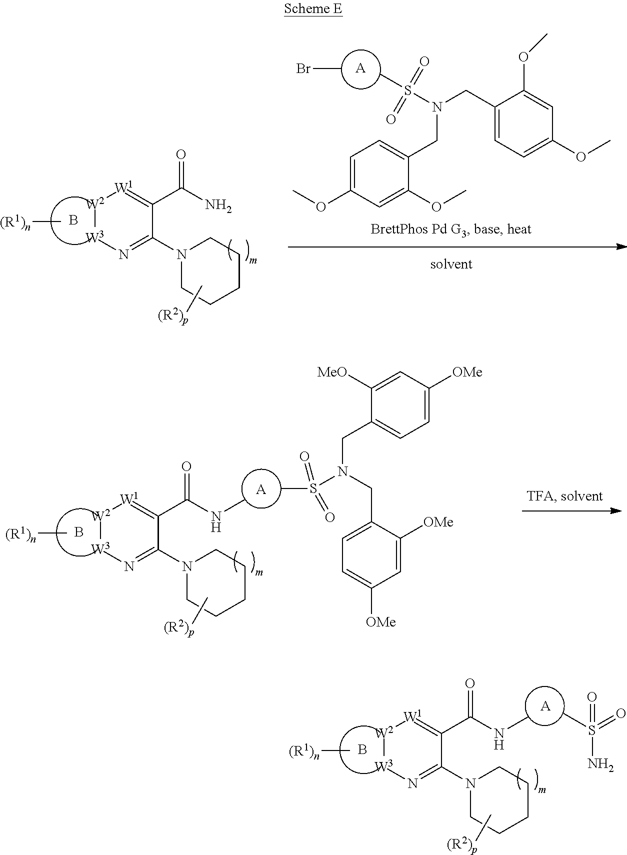
wherein
R¹, independently in each instances, is selected from hydrogen, C₁-C₆ alkyl, C₁-C₆ alkoxy, halogen, C₁-C₆ haloalkyl, halo-C₁-C₆ alkoxy, C₃-C₆-cycloalkyl, and C₃-C₆-cycloalkylC₁-C₃-alkyl; or
wherein two R¹ are attached on adjacent ring carbons in 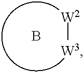
and together with the adjacent carbons to which they are attached form 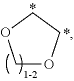
where * indicate the shared carbons in 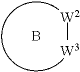
and any remaining R¹ are independently selected from hydrogen, C₁-C₆ alkyl, C₁-C₆ alkoxy, halogen, C₁-C₆ haloalkyl, halo-C₁-C₆ alkoxy, C₃-C₆-cycloalkyl, or C₃-C₆-cycloalkylC₁-C₃-alkyl;
A is C₆-C₁₀ aryl substituted with R³ and optionally substituted with (R^{3a})_{q}; 5- to 10-membered heteroaryl substituted with R³ and optionally substituted with (R^{3a})_{q}; or 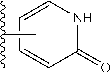
optionally substituted with (R^{3a})_{q1};
W¹ is —N═, —C(H)═, —C(halogen)=, —C(C₁-C₆alkyl)=, or —C(cyclopropyl)=;
W² and W³ are each —C—, the dashed bond between W² and W³ is a double bond, and 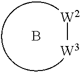
is a partially unsaturated 5 to 7-membered carbocyclic ring, a benzo ring, a partially unsaturated 5 to 7-membered heterocyclic ring, or a 5 or 6-membered heteroaromatic ring; or
one of W² and W³ is —C— and the other is —N—, the dashed bond between W² and W³ is a single bond, and 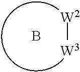
is a 5 or 6-membered heterocyclic ring optionally comprising an additional N and where the remaining ring atoms are C;
R², independently in each instance, is hydrogen, halogen, or C₁-C₆ alkyl; or
two R² are attached on adjacent carbons and together with the carbons to which they are attached form a 4- to 7-membered carbocyclic ring and the remaining R², independently in each instance, is hydrogen, halogen, or C₁-C₆alkyl; or
two R² are attached on the same carbon and together with the carbon to which they are attached form a 4- to 7-membered carbocyclic ring and the remaining R², independently in each instance, is hydrogen, halogen, or C₁-C₆alkyl;
m is 0, 1, 2, or 3;
n is 0, 1, 2, 3, or 4; and
p is 0, 1, 2, 3, 4, 5, or 6;
to yield a compound of Formula I; and optionally isolating the compound of Formula I.
